SEQUENCE LISTING (205 sequences)

SEQ ID NO: 1 - RNA, 12 nt, synthetic construct
  source 1..12: mol_type = other RNA
tttaaatttaaa

SEQ ID NO: 2 - RNA, 85 nt, Homo sapiens
  source 1..85: mol_type = genomic RNA
ccttagcagagctgtggagtgtgacaatggtgtttgtgtctaaactatcaaacgccattatcacactaaatagctactgctaggc

SEQ ID NO: 3 - RNA, 22 nt, Homo sapiens
  source 1..22: mol_type = genomic RNA
tggagtgtgacaatggtgtttg

SEQ ID NO: 4 - RNA, 22 nt, Homo sapiens
  source 1..22: mol_type = genomic RNA
aacgccattatcacactaaata

SEQ ID NO: 5 - RNA, 71 nt, Homo sapiens
  source 1..71: mol_type = genomic RNA
gccaacccagtgttcagactacctgttcaggaggctctcaatgtgtacagtagtctgcacattggttaggc

SEQ ID NO: 6 - RNA, 23 nt, Homo sapiens
  source 1..23: mol_type = genomic RNA
cccagtgttcagactacctgttc

SEQ ID NO: 7 - RNA, 22 nt, Homo sapiens
  source 1..22: mol_type = genomic RNA
acagtagtctgcacattggtta

SEQ ID NO: 8 - RNA, 86 nt, Homo sapiens
  source 1..86: mol_type = genomic RNA
tgccagtctctaggtccctgagaccctttaacctgtgaggacatccagggtcacaggtgaggttcttgggagcctggcgtctggcc

SEQ ID NO: 9 - RNA, 24 nt, Homo sapiens
  source 1..24: mol_type = genomic RNA
tccctgagaccctttaacctgtga

SEQ ID NO: 10 - RNA, 22 nt, Homo sapiens
  source 1..22: mol_type = genomic RNA
acaggtgaggttcttgggagcc

SEQ ID NO: 11 - RNA, 110 nt, Homo sapiens
  source 1..110: mol_type = genomic RNA
gccgagaccgagtgcacagggctctgacctatgaattgacagccagtgctctcgtctcccctctggctgccaattccataggtcacaggtatgttcgcctcaatgccagc

SEQ ID NO: 12 - RNA, 22 nt, Homo sapiens
  source 1..22: mol_type = genomic RNA
tctgacctatgaattgacagcc

SEQ ID NO: 13 - RNA, 22 nt, Homo sapiens
  source 1..22: mol_type = genomic RNA
ctgccaattccataggtcacag

SEQ ID NO: 14 - RNA, 83 nt, Homo sapiens
  source 1..83: mol_type = genomic RNA
cggggtgaggtagtaggttgtgtggtttcagggcagtgatgttgcccctcggaagataactatacaacctactgccttccctg

SEQ ID NO: 15 - RNA, 22 nt, Homo sapiens
  source 1..22: mol_type = genomic RNA
tgaggtagtaggttgtgtggtt

SEQ ID NO: 16 - RNA, 22 nt, Homo sapiens
  source 1..22: mol_type = genomic RNA
ctatacaacctactgccttccc

SEQ ID NO: 17 - RNA, 64 nt, Homo sapiens
  source 1..64: mol_type = genomic RNA
ccccgcgacgagcccctcgcacaaaccggacctgagcgttttgttcgttcggctcgcgtgaggc

SEQ ID NO: 18 - RNA, 23 nt, Homo sapiens
  source 1..23: mol_type = genomic RNA
gcgacgagcccctcgcacaaacc

SEQ ID NO: 19 - RNA, 22 nt, Homo sapiens
  source 1..22: mol_type = genomic RNA
tttgttcgttcggctcgcgtga

SEQ ID NO: 20 - RNA, 85 nt, Homo sapiens
  source 1..85: mol_type = genomic RNA
aggcctctctctccgtgttcacagcggaccttgatttaaatgtccatacaattaaggcacgcggtgaatgccaagaatggggctg

SEQ ID NO: 21 - RNA, 22 nt, Homo sapiens
  source 1..22: mol_type = genomic RNA
cgtgttcacagcggaccttgat

SEQ ID NO: 22 - RNA, 22 nt, Homo sapiens
  source 1..22: mol_type = genomic RNA
taaggcacgcggtgaatgccaa

SEQ ID NO: 23 - RNA, 106 nt, Homo sapiens
  source 1..106: mol_type = genomic RNA
gcgcagcgccctgtctcccagcctgaggtgcagtgctgcatctctggtcagttgggagtctgagatgaagcactgtagctcaggaagagagaagttgttctgcagc

SEQ ID NO: 24 - RNA, 22 nt, Homo sapiens
  source 1..22: mol_type = genomic RNA
ggtgcagtgctgcatctctggt

SEQ ID NO: 25 - RNA, 21 nt, Homo sapiens
  source 1..21: mol_type = genomic RNA
tgagatgaagcactgtagctc

SEQ ID NO: 26 - RNA, 87 nt, Homo sapiens
  source 1..87: mol_type = genomic RNA
gacagtgcagtcacccataaagtagaaagcactactaacagcactggagggtgtagtgtttcctactttatggatgagtgtactgtg

SEQ ID NO: 27 - RNA, 21 nt, Homo sapiens
  source 1..21: mol_type = genomic RNA
cataaagtagaaagcactact

SEQ ID NO: 28 - RNA, 23 nt, Homo sapiens
  source 1..23: mol_type = genomic RNA
tgtagtgtttcctactttatgga

SEQ ID NO: 29 - RNA, 110 nt, Homo sapiens
  source 1..110: mol_type = genomic RNA
gtgttggggactcgcgcgctgggtccagtggttcttaacagttcaacagttctgtagcgcaattgtgaaatgtttaggaccactagacccggcgggcgcggcgacagcga

SEQ ID NO: 30 - RNA, 25 nt, Homo sapiens
  source 1..25: mol_type = genomic RNA
agtggttcttaacagttcaacagtt

SEQ ID NO: 31 - RNA, 22 nt, Homo sapiens
  source 1..22: mol_type = genomic RNA
gtgaaatgtttaggaccactag

SEQ ID NO: 32 - RNA, 80 nt, Homo sapiens
  source 1..80: mol_type = genomic RNA
tgggatgaggtagtaggttgtatagttttagggtcacacccaccactgggagataactatacaatctactgtctttccta

SEQ ID NO: 33 - RNA, 22 nt, Homo sapiens
  source 1..22: mol_type = genomic RNA
tgaggtagtaggttgtatagtt

SEQ ID NO: 34 - RNA, 21 nt, Homo sapiens
  source 1..21: mol_type = genomic RNA
ctatacaatctactgtctttc

SEQ ID NO: 35 - RNA, 71 nt, Homo sapiens
  source 1..71: mol_type = genomic RNA
gcgactgtaaacatcctcgactggaagctgtgaagccacagatgggctttcagtcggatgtttgcagctgc

SEQ ID NO: 36 - RNA, 22 nt, Homo sapiens
  source 1..22: mol_type = genomic RNA
tgtaaacatcctcgactggaag

SEQ ID NO: 37 - RNA, 22 nt, Homo sapiens
  source 1..22: mol_type = genomic RNA
ctttcagtcggatgtttgcagc

SEQ ID NO: 38 - RNA, 71 nt, Homo sapiens
  source 1..71: mol_type = genomic RNA
tgggaaacatacttctttatatgcccatatggacctgctaagctatggaatgtaaagaagtatgtatctca

SEQ ID NO: 39 - RNA, 22 nt, Homo sapiens
  source 1..22: mol_type = genomic RNA
acatacttctttatatgcccat

SEQ ID NO: 40 - RNA, 22 nt, Homo sapiens
  source 1..22: mol_type = genomic RNA
tggaatgtaaagaagtatgtat

SEQ ID NO: 41 - RNA, 85 nt, Homo sapiens
  source 1..85: mol_type = genomic RNA
cgctggcgacgggacattattacttttggtacgcgctgtgacacttcaaactcgtaccgtgagtaataatgcgccgtccacggca

SEQ ID NO: 42 - RNA, 21 nt, Homo sapiens
  source 1..21: mol_type = genomic RNA
cattattacttttggtacgcg

SEQ ID NO: 43 - RNA, 22 nt, Homo sapiens
  source 1..22: mol_type = genomic RNA
tcgtaccgtgagtaataatgcg

SEQ ID NO: 44 - RNA, 22 nt, synthetic construct
  source 1..22: mol_type = other RNA
caaacaccattgtcacactcca

SEQ ID NO: 45 - RNA, 22 nt, synthetic construct
  source 1..22: mol_type = other RNA
taaccaatgtgcagactactgt

SEQ ID NO: 46 - RNA, 24 nt, synthetic construct
  source 1..24: mol_type = other RNA
tcacaggttaaagggtctcaggga

SEQ ID NO: 47 - RNA, 22 nt, synthetic construct
  source 1..22: mol_type = other RNA
ggctgtcaattcataggtcaga

SEQ ID NO: 48 - RNA, 22 nt, synthetic construct
  source 1..22: mol_type = other RNA
aaccacacaacctactacctca

SEQ ID NO: 49 - RNA, 22 nt, synthetic construct
  source 1..22: mol_type = other RNA
tcacgcgagccgaacgaacaaa

SEQ ID NO: 50 - RNA, 22 nt, synthetic construct
  source 1..22: mol_type = other RNA
ttggcattcaccgcgtgcctta

SEQ ID NO: 51 - RNA, 21 nt, synthetic construct
  source 1..21: mol_type = other RNA
gagctacagtgcttcatctca

SEQ ID NO: 52 - RNA, 23 nt, synthetic construct
  source 1..23: mol_type = other RNA
tccataaagtaggaaacactaca

SEQ ID NO: 53 - RNA, 22 nt, synthetic construct
  source 1..22: mol_type = other RNA
ctagtggtcctaaacatttcac

SEQ ID NO: 54 - RNA, 22 nt, synthetic construct
  source 1..22: mol_type = other RNA
aactatacaacctactacctca

SEQ ID NO: 55 - RNA, 22 nt, synthetic construct
  source 1..22: mol_type = other RNA
cttccagtcgaggatgtttaca

SEQ ID NO: 56 - RNA, 22 nt, synthetic construct
  source 1..22: mol_type = other RNA
atacatacttctttacattcca

SEQ ID NO: 57 - RNA, 22 nt, synthetic construct
  source 1..22: mol_type = other RNA
cgcattattactcacggtacga

SEQ ID NO: 58 - RNA, 1705 nt, synthetic construct
  source 1..1705: mol_type = other RNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaacaaacaccattgtcacactccatttaaaatacatacttctttacattccatttaaactagtggtcctaaacatttcactttaaacttccagtcgaggatgtttaca

SEQ ID NO: 59 - RNA, 1599 nt, Homo sapiens
  source 1..1599: mol_type = genomic RNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 60 - RNA, 541 nt, synthetic construct
  source 1..541: mol_type = other RNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtgacaaacaccattgtcacactccatttaaaatacatacttctttacattccatttaaactagtggtcctaaacatttcactttaaacttccagtcgaggatgtttaca

SEQ ID NO: 61 - RNA, 435 nt, Homo sapiens
  source 1..435: mol_type = genomic RNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 62 - RNA, 3822 nt, synthetic construct
  source 1..3822: mol_type = other RNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacccgccggaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 63 - RNA, 3928 nt, synthetic construct
  source 1..3928: mol_type = other RNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacccgccggaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataacaaacaccattgtcacactccatttaaaatacatacttctttacattccatttaaactagtggtcctaaacatttcactttaaacttccagtcgaggatgtttaca

SEQ ID NO: 64 - RNA, 3899 nt, synthetic construct
  source 1..3899: mol_type = other RNA
atgttcgttttcttggtcctgcttcccctggtgtcttcacagtgcgtgaatctgaccaccagaacacagctgcctccagcatacaccaacagcttcaccagaggcgtgtattatcctgacaaggtgtttcgctccagcgtgctgcacagcacccaggacctttttctgccttttttctccaacgtgacatggttccacgcaatccacgtgagcggaaccaacggaacgaagagattcgacaaccctgtgctgcccttcaacgacggagtgtacttcgccagcacagagaagagcaacatcatccggggctggatcttcggaaccaccctggacagcaaaacccaatctctgcttatcgtgaacaacgcaaccaacgtggtgatcaaggtgtgtgaattccaattttgtaacgacccattcctgggagtgtactaccataagaacaacaagagctggatggaaagcgagttccgggtgtacagcagcgccaacaactgcaccttcgagtacgtgagtcagccctttctgatggacctggaaggcaagcagggaaatttcaagaatctgagagagttcgtgttcaaaaacatcgatggctatttcaagatctatagcaagcacacccctatcaacctggtgagagatctgccccagggcttcagcgccctggagcctctggtagacctacctatcggcatcaacataacgagatttcagaccctgttggctcttcataggagctacctgacccccggcgattctagcagcggatggacagccggcgccgctgcctactacgttggctacctgcaacctcggacattcctgctgaaatacaatgagaacggcactatcaccgatgccgtggactgtgccctggatcctctgagcgaaaccaagtgcaccctgaagagctttaccgtggaaaagggcatctaccagaccagcaatttccgggtgcagcctacagagagcatcgtgagattccccaacatcaccaatctgtgtcctttcggcgaggtgttcaacgctacaagattcgcaagcgtgtacgcctggaatcggaagcggatcagtaactgtgtggccgattattcggtgctgtataattctgccagctttagcaccttcaagtgctacggtgtgagccctaccaaactcaacgacctgtgcttcaccaacgtgtatgccgactctttcgtgatccggggcgacgaggtgcggcagatcgcccccggacagacaggcaaaatcgccgactacaactacaagctgcctgacgacttcacagggtgcgttatcgcctggaacagcaacaatctggattcaaaggtgggcggaaactacaactacctgtacagactgttcagaaagtccaacctgaagccctttgagagagacatctctacagaaatctaccaggccggctccaccccatgcaacggcgtggaaggcttcaactgctacttccccctgcagagctacggctttcagcctaccaatggcgtcggttaccagccttaccgcgtggtcgttctatccttcgagctgctgcacgcccctgctacagtgtgcggacctaagaagagcacaaacctggtcaaaaacaagtgtgtcaacttcaacttcaacggcctgaccggcacaggcgtactgacagaaagcaacaagaagttcctgcctttccagcagtttggcagagatatcgctgataccacagacgccgtgcgggatcctcagacactggaaatcctggacatcacaccctgcagctttggcggcgtgtctgtcatcaccccaggcaccaacacgtccaaccaagtggccgtgctgtaccaggacgtcaactgcaccgaggtgcccgttgctatccacgccgatcagctaactcctacctggcgggtttatagcaccggatctaacgtgttccagaccagggccggatgtctgatcggcgctgaacacgtaaacaatagctacgagtgtgatatccctatcggagccggcatctgtgccagctaccagacccagacaaattctccacggagagctagaagcgtcgcctctcagagcatcatcgcctacaccatgagcctgggcgccgaaaactctgtggcctacagcaacaacagcatcgccattcccaccaactttacaatcagcgtgacaacagagatcctgcctgtgagcatgaccaaaaccagcgtggactgcaccatgtacatctgcggcgacagcaccgaatgctctaaccttctgctgcaatacggcagcttctgcactcagctgaacagagccctgaccggcatcgccgtggagcaggataagaacacccaggaggtgttcgcccaggtgaaacaaatctacaagacacctcccatcaaggacttcggcggatttaacttcagccagatcctgcctgacccatctaagcctagcaagcggtcctttatcgaggacttgctgttcaacaaggtgaccctggccgatgccggctttatcaagcagtacggcgactgccttggcgacatcgccgccagagacctgatctgcgcccagaagtttaacggcctgacagtgctgcctcctctgctgaccgacgaaatgatcgcccagtataccagcgctctgctggcgggtaccatcaccagcggctggaccttcggcgccggcgctgctctgcagatccctttcgccatgcagatggcctaccggttcaacggcattggcgtgacccagaacgtgctgtacgagaatcagaagctgatcgccaaccagttcaacagcgctatcggaaaaatccaggactctctgagctctactgcctctgctcttgggaaactgcaggacgtggttaatcagaacgcccaggccctcaataccctggtgaagcaactgtcgagcaattttggcgccatcagcagcgtgctgaatgacattctgtctagactggaccctccagaagctgaggtgcagattgacagactgatcacaggcagactgcagagcctgcagacctacgtgacccaacaactgatcagagccgccgagattagggcctctgccaatctggccgccacgaaaatgtccgagtgcgtcctgggccagtcaaagagagtggatttctgcggcaagggataccacctgatgagcttcccccagagcgctccgcacggcgtggtgtttctgcacgtgacctacgtgccagcccaggagaagaacttcaccacggcccctgccatctgccacgacggcaaggcccacttccccagagaaggagtgttcgtgagcaatggcacacactggttcgtgacacaaagaaacttctacgagcctcagatcatcacaaccgataacaccttcgtgagcggcaattgcgacgtggtgatcggcattgtgaacaacaccgtgtacgaccccctgcagcccgagctggatagcttcaaagaggaactggataagtacttcaagaaccacaccagccctgatgtggatctgggcgacatttctggcatcaacgcctctgtcgtgaacatccagaaagagatagatagactgaacgaggttgcaaagaacctgaacgaaagcctgatcgacttgcaggagctcggcaagtacgagcagtacatcaagtggccttggtacatttggctgggctttatcgccggactgatcgccatcgtgatggtcacaatcatgctgtgctgcatgacaagttgctgttcctgcctgaagggctgctgtagctgtggaagctgctgtaaattcgacgaagatgacagcgagcctgtgctgaagggcgtgaagctgcactacacatgacaaacaccattgtcacactccatttaaaggctgtcaattcataggtcagtttaaacttccagtcgaggatgtttaca

SEQ ID NO: 65 - RNA, 3890 nt, synthetic construct
  source 1..3890: mol_type = other RNA
atgttcgtgttcctggtgttactgcccctggtgtcttctcagtgcgtcaacttcaccacaagaacacagctgcctcctgcctatacaaacagctttacccggggagtgtactaccccgataaagtgttccggagctctgtgctgcacagcacacaggacttgttcctgcctttcttcagcaatgtgacatggttccacgccatccacgtctccggcacaaacggcaccaagaggttcgccaatcctgtgctgccattcaatgatggcgtgtatttcgccagcacagagaagtctaacatcatcagaggctggatcttcggcaccaccctcgactctaaaacccagagcctgctgatagtgaacaacgccacaaacgtggtgatcaaggtctgtgaattccagttctgcaacgacccattcctgggcgtgtactaccacaagaataacaagagctggatggaatctgagttcagagtgtattcatcagccaacaactgcacattcgagtacgtgtctcagccattcctgatggacctggaaggcaagcagggcaatttcaagaatctcagagagttcgtcttcaagaacatcgacggctactttaagatctatagcaagcacacccctatcaacctggttcggggcctgccccagggctttagcgccctggaacctctggtggatctgccaattggcatcaacatcacccggtttcagacactgcacatcagctacctgacacctggcgacagcagcagcggctggaccgccggcgccgccgcctactacgtcggctacctgcagccccggaccttcctgctgaagtacaatgaaaatggcaccatcacagacgccgtggattgcgccctggaccctctgtctgaaacaaagtgcaccctgaaaagcttcaccgtggaaaagggcatataccagacctccaacttccgggtgcagcctacagagtctatcgtgagattccccaacatcaccaatctgtgtccttttggcgaggtgttcaacgccaccagattcgcaagcgtgtacgcctggaaccggaagaggatcagcaactgcgtggcagattacagcgtgctctacaacagcgccagtttctctacctttaagtgctacggcgtcagccctacaaaactgaacgatctgtgcttcaccaacgtgtacgccgattcctttgtgatacggggcgacgaagttagacagatcgcccctggacagacaggaaatatcgccgactacaactataagctgcctgacgacttcaccggctgcgtcatcgcttggaactccaacaacctggattccaaggtgggcggaaactacaactacctgtacagactgttcagaaagagcaacctgaaacctttcgagagggacatcagcacagagatctaccaggccggcagcaccccctgtaatggagtcaaaggcttcaattgctacttccctctgcagtcttacggcttccagccaacatacggcgtgggctaccagccctaccgggtggttgtgctgtccttcgagctgctgcatgccccagccacagtatgcggtcctaagaaaagcaccaacctggtgaagaacaaatgtgtgaactttaactttaacggcctgaccggcaccggcgtgctgaccgaatccaataagaagttcctgccgttccagcagtttggcagagatatcgccgacaccacagacgccgtgagagacccccagaccctggaaatcctggacatcaccccttgctcctttggaggggtgagcgtgatcaccccgggcacaaacaccagcaaccaggtggccgtgctgtaccagggcgtgaattgtaccgaggtgcctgtggcgatccacgccgatcagctgacccctacctggcgggtgtacagcaccggatctaacgtgttccaaacaagagccggctgtctgatcggagctgaacacgtgaacaactcttacgagtgtgacattcctatcggcgccggcatctgcgcctcttatcagacccagaccaacagccccagacgtgccagatctgtggcctctcagagcatcatcgcctacaccatgtctctgggagtggaaaactccgtggcttacagcaacaattctatcgccatccccaccaactttacaatcagcgtgaccaccgagatactgcctgtgtccatgacaaagaccagcgtggactgcactatgtacatctgcggcgacagcacagaatgcagcaacctgctgctgcagtacggaagcttttgtactcagctgaacagagccctgactggcatcgctgttgagcaggataagaatactcaggaggtcttcgctcaagtgaagcagatctacaagacccctccaatcaaggacttcggcggcttcaacttcagccaaattctgcctgatcctagcaagcccagcaagcggagcttcatcgaggacctgctgtttaacaaagtgacacttgccgacgccggattcattaagcagtatggcgactgcctgggcgacatcgccgcgagagatttgatctgcgcccaaaagttcaacggcctcaccgtgctgcctcctcttctgaccgacgagatgatcgctcagtacaccagcgctcttctggccggcacaatcaccagcggctggacatttggcgctggtgccgccctccagatccctttcgccatgcagatggcctacagattcaacggcatcggcgtcacccaaaacgtgctctatgagaaccagaaacttatcgctaatcagttcaactctgccatcggcaagatccaagatagcctgtcctccaccgctagcgccctgggaaagctccaggacgtggtgaatcagaacgcccaagccctgaacaccctggtgaaacagctgagcagcaacttcggcgctatcagctccgttctgaacgacattctgtctagactggaccctcctgaggccgaggtccagatcgatagactgatcactggacgcctgcaatcactgcaaacatacgtgacccagcagctgattagagccgccgagatcagagcctcagcaaatctggccgccacgaagatgagcgagtgcgtgctgggccagagcaagagagtcgacttttgcggcaaaggctaccacctgatgagcttccctcagagcgccccacacggcgtggtgttcctgcatgtgacctacgtgcccgcccaggaaaagaactttaccaccgcccctgctatctgtcacgacggcaaggcccacttccctcgcgagggcgtgttcgtcagcaacggcacccactggttcgtgacacaacgtaacttctacgagcctcagatcataaccaccgataacacattcgtgagcggcaattgcgatgtggtgatcggaatcgtgaacaacaccgtgtacgacccgctgcagcccgagctggacagcttcaaagaggaactggataagtactttaagaaccacacttctccagacgtggacctgggcgatatcagcggaatcaacgcttccgtggtgaacatccagaaggaaatcgacagactgaacgaggtggctaaaaacctgaatgagagcctgatcgacctgcaggagctgggaaaatacgaacagtacatcaagtggccttggtacatctggctgggctttatcgctggcctgatcgccatcgtgatggtgaccatcatgctgtgctgtatgaccagctgttgtagctgcctgaagggttgctgttcctgcggaagctgctgcaagttcgacgaggatgacagcgagcccgtgctgaagggcgttaagctgcactacacctgacaaacaccattgtcacactccatttaaaggctgtcaattcataggtcagtttaaacttccagtcgaggatgtttaca

SEQ ID NO: 66 - RNA, 3922 nt, synthetic construct
  source 1..3922: mol_type = other RNA
atgttcgtcttcctggtgctgctgcccctggtgagctctcagtgcgtgaatctgagaacccggacacagctgcctcctgcctacacaaacagctttacaagaggcgtctactaccctgacaaggtgttccggtcgagcgtgctgcattctacccaggatttgtttcttccttttttcagtaacgtgacatggttccacgccatccacgtgtccggaaccaacggcaccaagagattcgacaaccctgtgctgcctttcaacgacggagtgtatttcgcctctaccgagaagagcaacatcattcggggatggatcttcggaaccaccctcgacagcaagacacagagcctgctaatagtcaacaacgctaccaacgtggtgattaaggtgtgcgagttccaattttgtaacgatcctttcctgggagtttattaccataagaacaataaaagctggatggaaagcgaggtgtacagcagcgcaaacaactgcacattcgagtatgtgagccaacctttcctgatggacctggaaggcaagcaggggaacttcaagaacctgagggaattcgtgtttaagaacatcgacggctacttcaagatctacagcaagcacacaccaattaacctcgttagagatctgccacagggcttcagtgccctggaacccctggtggatctgcccatcggaatcaacatcaccagattccagaccctcctggccctgcacagaagctatctgacccctggcgattctagctctggctggacagctggcgccgctgcttactacgtgggctacctgcagcctagaacattcctgctcaagtacaacgagaatggcacaatcaccgacgccgttgactgcgccctggatcctttgtctgagacaaagtgcactctgaagagcttcaccgtggaaaagggcatctaccagacatctaacttcagagtgcagcctacagagagcatcgtgcggttccccaacataacaaacctgtgtccattcggagaagtgtttaatgccaccagattcgctagcgtgtacgcctggaaccggaagagaatcagcaactgcgtcgccgactactccgtgctgtacaatagcgcctctttcagcacctttaagtgttacggcgtctctccaacaaagctgaacgacctgtgcttcacaaacgtgtacgccgacagcttcgtgatccggggcgacgaagtgcggcagattgcacctggtcagactgggaaaatcgcagattacaactacaagctgccagatgattttaccggctgtgtgatcgcctggaatagcaataacctggacagcaaagtgggcggcaattacaactaccggtacagactgttccggaagagcaatctgaagccttttgagagagacatctccacagagatctaccaggccggctctaagccttgcaacggcgtggaggggtttaattgctacttccctctgcagtcttacgggttccagcccaccaacggcgtgggctatcagccttacagagtggtggtgctgtctttcgaactgctgcacgcccctgctaccgtgtgcgggcctaagaagtccaccaaccttgtgaagaacaagtgtgtgaacttcaacttcaatggcctgaccggaaccggcgtgttgaccgaatctaacaagaaattcctgccgttccaacagttcggcagagatattgccgacaccaccgatgccgtgcgggacccccaaaccctggaaatcctggatatcaccccatgcagcttcggcggcgtgtctgttatcacccccggcacaaacacgagcaaccaggtcgccgtgctctaccagggcgtgaactgcacagaagtgcccgtggctatccacgccgatcagctgacacccacatggcgggtgtacagcacaggatctaacgttttccagacaagagctggctgccttattggcgctgaacacgtgaataacagctacgagtgtgacatcccaatcggcgccggcatctgcgcctcctaccagacccagaccaacagcagaaggagagcccggagcgtggccagccagtctatcatcgcctacacaatgagcctgggcgccgaaaactccgtggcctatagcaacaactccatcgctatccctaccaacttcaccatcagcgtgacaacggaaattctgcctgtgagcatgaccaagacctctgtggactgtacaatgtacatctgcggcgactctacagaatgcagcaacctgctgctgcagtacggcagcttttgcacccagcttaatagagccctgaccggaatcgccgtggaacaggacaagaacacccaggaggtcttcgcccaggtgaaacagatctacaagacccctcctattaaggacttcggcggatttaacttcagccagatcctgcctgaccctagcaagcccagcaaaagaagcttcatcgaggacctcctgttcaacaaagtgaccctggccgacgctggctttatcaagcagtatggcgactgcctgggcgacatcgctgctagggacctgatctgtgcccagaagttcaacggcctgacagtgctgcctcctctgctgaccgatgaaatgatcgcccagtacacaagcgccctgctggccggcaccatcaccagcggctggacctttggagccggcgccgccctgcagatcccctttgccatgcagatggcctatcggttcaacggaatcggcgtgacccaaaacgtactgtacgagaaccagaagctgatcgccaatcaatttaatagcgccatcggtaaaatccaggatagcctgagctccactgccagcgccctgggcaaactgcagaacgtggtgaaccagaacgcccaagctctgaacaccctggtgaagcagctgtcttccaactttggtgctatctctagcgtcctgaatgatatcctgagcagactggacccccccgaggccgaggtgcagatcgatagactgatcaccggcagactgcaatcgctgcaaacttacgtgacccagcagctgatcagagccgccgagatcagagctagcgccaacctggccgccactaagatgagcgagtgcgttctgggccagagtaagcgggtggacttctgtggcaagggataccacctgatgtcttttccacagagcgcccctcacggcgtggtgttcctgcacgttacctacgtgccagcccaggagaagaacttcaccacagcccctgccatctgccacgacggcaaggcccacttccctagagagggcgtgttcgtcagcaacggcacccactggttcgtgacgcaaagaaacttctacgagccccagatcattaccaccgacaataccttcgtatccggcaactgcgacgtggtgatcggcatcgtgaacaacacagtgtacgaccctctgcagcctgagctggactctttcaaggaagagctggacaagtatttcaagaaccacaccagccctgatgtggacctgggcgacatcagcggaatcaatgcctcagtggtgaacatccagaaagagatcgacagactgaacgaggtcgccaagaacctgaatgagagcctgatcgacctgcaggagctgggcaagtacgagcaatacatcaagtggccttggtacatctggctgggcttcatcgccggcctgattgccatcgtgatggtgaccatcatgctgtgttgcatgaccagttgctgtagttgcctgaaaggctgctgttcttgcggcagctgctgcaaattcgatgaggacgactccgagcccgtgctgaagggcgtgaagctgcactacacctgacaaacaccattgtcacactccatttaaaatacatacttctttacattccatttaaactagtggtcctaaacatttcactttaaacttccagtcgaggatgtttaca

SEQ ID NO: 67 - RNA, 3887 nt, synthetic construct
  source 1..3887: mol_type = other RNA
atgttcgtgtttctggtcctgctgcccctggtgtcctcccagtgcgtgaacctgacgaccagaacacaactgcctcctgcctacaccaacagctttacaagaggcgtctattaccccgacaaggtgttccggagctccgtcctgcactctacccaggaccttttcctgccttttttcagcaacgtgacatggttccacgccatcagcggtaccaacggcaccaagcgcttcgacaaccctgtgctgccatttaacgacggagtgtatttcgcctccacagaaaagtcgaacatcatcaggggctggatcttcggcaccacactggacagcaagacccagagcctgctgatcgtgaataacgccacaaacgtggtcatcaaagtctgcgagttccagttctgtaatgaccccttcctgggcgttcataaaaacaacaaaagctggatggaaagcgagttcagagtgtattctagcgccaataactgtacatttgagtacgtgtcccagcccttcctgatggacctggaaggcaagcaaggaaattttaagaacctgcgtgagtttgtgttcaagaacatcgatggttatttcaaaatctacagcaagcacacccccattaacctggtcagagacctgccccagggcttctctgccctggaacctctggtggacctgccgatcggaatcaacatcacacggttccagaccctgctagccctgcatagatcttacctgacccccggcgacagctcttccggctggacagccggcgccgctgcttactacgtgggctacctgcagcctagaaccttcctgctcaagtacaacgaaaatggcaccatcaccgacgccgtggactgcgccctggaccctcttagcgagacaaagtgcacactgaagagcttcaccgtggaaaagggcatctaccagacatcgaacttcagagtgcagcctaccgagtccatcgtgaggtttcctaacatcaccaacctgtgtcctttcggcgaggtgttcaacgccaccagattcgccagcgtgtacgcctggaatagaaagagaatctctaactgtgtggccgattacagcgtgctgtacaactctgccagctttagcacctttaaatgttacggcgtgagccctacaaagctgaacgatctgtgcttcaccaatgtgtacgccgattctttcgtgatccggggcgatgaggtgcggcagatcgccccaggccagacaggcaagatcgccgactacaattacaagctgcctgatgacttcaccggctgcgtgattgcctggaacagcaacaatctggacagcaaggtgggaggcaactacaactacctgtacagactcttccggaagagcaacctgaaacctttcgagagagatatctcaactgaaatctaccaggccggctcaacaccctgcaatggagttgagggcttcaattgctacttccccctgcagtcttacggctttcagcctacatacggcgtgggctaccagccttaccgggtggttgtgttgagcttcgaactgctgcacgcccctgctaccgtgtgcggtcctaagaaaagcaccaacctggtgaagaacaagtgcgtaaacttcaacttcaacggcctgactggaacaggcgtcctgaccgaaagcaacaagaagttcctgccttttcaacaatttggcagagatattgatgatacaacagatgctgtgcgggatcctcagacactggaaatcctggacatcaccccctgctccttcggcggagtcagcgtgataacccctggcactaacaccagcaatcaggtggccgtgctctaccagggcgtcaactgcaccgaagtccctgttgctatccacgctgaccagctgacacctacctggagagtgtatagcaccggttctaacgtcttccagacccgcgccggctgtctgatcggcgccgagcacgtgaacaacagctacgagtgcgacatccccatcggcgctggcatctgcgcctcttatcagacacagaccaacagccaccggagagctagaagcgtggcctctcagtcgatcattgcctacaccatgtccctgggcgccgagaactcggtggcctacagcaacaattctatcgccatccccatcaacttcaccatcagcgtgacaaccgaaattctgccagtgtccatgacgaagacatccgtggactgcacaatgtacatctgcggcgatagcacagaatgttctaatctgctgcttcaatatggatctttctgcacccagctgaaccgggccctgacaggcatcgccgtggaacaggacaaaaatacccaggaggtgtttgcccaggtgaagcagatctacaagaccccaccaatcaaggacttcggagggtttaatttcagccagatcctgcccgatcctagcaagccttccaagcggagtttcatcgaggacctgctgttcaacaaagtgaccctggctgatgccggcttcatcaagcagtacggcgactgcctgggcgacatcgccgccagagatctgatctgcgcccagaaatttaacgggctgaccgtgctgcctccactgctgaccgacgagatgatcgcacagtacaccagcgctttgctggcgggcaccatcacgagcggctggaccttcggggccggcgccgccctgcaaattcctttcgccatgcagatggcctaccggtttaacggcatcggcgtgacacagaacgtgctatacgagaaccagaagctgatagctaatcagtttaactctgccatcggcaagatccaggacagcctctccagcaccgccagcgccctgggtaagctgcaggacgtggtgaaccagaacgcccaagccctgaacaccctggttaagcagctgtccagcaatttcggcgctattagcagcgttctgaatgacatcctggccagactggacccacctgaggccgaggtgcagatcgatagactgatcacaggaagactgcagagcctgcagacctacgtcacccaacaactcatccgggccgccgaaatccgggccagcgccaaccttgcagccaccaagatgagcgagtgcgtgctcggccagagcaaaagagtggacttttgcggcaaaggctaccacctgatgtccttccctcagagcgccccacacggcgtggtgttcctgcacgtgacatatgtgcccgcacaggagaagaacttcacgactgctcccgccatctgccacgacggcaaggcccacttccccagagaaggcgtgttcgtgagtaacgggacccactggttcgtgacccagagaaacttctacgagcctcagatcatcacaacccacaacacattcgtgagcggaaactgcgatgtggtgatcggaatcgtgaacaataccgtgtacgaccctctgcagcctgagctggacagcttcaaagaggaactcgacaagtattttaagaaccacaccagccctgacgtggatctgggcgacatcagcggcatcaacgctagcgtggtgaacatccagaaggaaatcgacagactgaacgaggtggccaagaacctgaacgagagcctgatcgacctgcaggagctgggcaagtacgagcagtacatcaagtggccttggtacatttggctgggcttcatcgcagggctgatcgccatcgtgatggtgacaatcatgctgtgttgcatgacctcttgttgcagctgtcttaaaggctgctgcagctgtggaagctgctgcaagttcgacgaggatgatagcgaacccgtgctgaagggcgtcaagctgcactacacctgacaaacaccattgtcacactccatttaaaggctgtcaattcataggtcagtttaaacttccagtcgaggatgtttaca

SEQ ID NO: 68 - RNA, 1738 nt, synthetic construct
  source 1..1738: mol_type = other RNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctaacaaacaccattgtcacactccatttaaaatacatacttctttacattccatttaaactagtggtcctaaacatttcactttaaacttccagtcgaggatgtttaca

SEQ ID NO: 69 - RNA, 1497 nt, Haemophilus influenzae
  source 1..1497: mol_type = genomic RNA
atggcgtctcaaggcaccaaacgatcatatgaacaaatggagactggtggggagcgccaggataccacagaaatcagagcatctgttggaagaatgattggtggaatcgggagattctacatccaaatgtgcactgaactcaaactcagtgattatgatggacgactaatccagaatagcataacaatagagaggatggtgctttctgcttttgatgagagaagaaataaatacctagaagagcatccaagtgctgggaaggaccctaagaaaacaggaggacccatctatagaagaatagacggaaaatggacgagagaactcatcctttatgacaaagaagaaataaggagagtttggcgccaagcaaacaatggcgaagatgcaacagcaggtcttactcatatcatgatttggcattccaacctgaatgatgccacatatcagaggacaagagcacttgttcgcactggaatggatcccagaatgtgctctctaatgcaaggttcaacacttcccagaaggtctggtgccgcaggtgctgcagtgaaaggagttggaacaatagctatggagttaatcagaatgatcaaacgtggaatcaatgaccgaaatttctggaggggtgaaaatggacgaaggacaagagttgcttatgaaagaatgtgcaatatcctcaaaggaaaatttcaaacagctgcccagagggcaatgatggatcaagtaagagaaagtcgaaacccaggaaacgctgagattgaagacctcattttcctggcacggtcagcactcattctgagaggatcagttgcacataaatcctgcctgcctgcttgtgtgtatgggcttgcagtagcaagtggccatgactttgaaagggaagggtactcactggtcgggatagacccattcaaattactccaaaacagtcaagtggtcagcctgatgagaccaaatgaaaatccagctcacaagagtcaattggtatggatggcatgccactctgctgcatttgaagatttaagagtatcaagtttcataagaggaaagaaagtgatcccaagaggaaagctttccacaagaggggttcagattgcttcaaatgagaatgtggaaaccatggactccaataccctggaactaagaagcagatactgggccataagaaccaggagtggaggaaataccaatcaacagaaggcatccgcaggccagatcagtgtgcagcctacattctcagtgcagcgaaatctcccttttgaaagagcaaccgttatggcagcattcagcgggaacaatgaaggacggacatccgacatgagaacagaagttataagaatgatggaaagtgcaaagccagaggatttgtccttccaggggcggggagtcttcgagctctcggacgaaaaggcaacgaacccgatcgtgccttcctttgacatgagtaatgaagggtcttatttcttcggagacaatgcagaggagtatgacaattga

SEQ ID NO: 70 - RNA, 1410 nt, Haemophilus influenzae
  source 1..1410: mol_type = genomic RNA
atgaatccaaaccaaaagataataaccattggttcgatctgtatgacaattggaatggctaacttaatattacaaattggaaacataatctcaatatgggttagccactcaattcaaattggaaatcaaagccagattgaaacatgcaatcaaagcgtcattacttatgaaaacaacacttgggtaaatcagacatatgttaacatcagcaacaccaactttgctgctggacagtcagtggtttccgtgaaattagcgggcaattcctctctctgccctgttagtggatgggctatatacagtaaagacaacagtgtaagaatcggttccaagggggatgtgtttgtcataagggaaccattcatatcatgctctcccttggaatgcagaaccttcttcttgactcaaggggccttgctaaatgacaaacattccaatggaaccattaaagacaggagcccataccgaaccctaatgagctgtcctattggtgaagttccctctccatacaactcaagatttgagtcagtcgcttggtcagcaagtgcttgtcatgatggcatcaattggctaacaattggaatttctggcccagacagtggggcagtggctgtgttaaagtacaatggcataataacagacactatcaagagttggaggaacaatatattgagaacacaagagtctgaatgtgcatgtgtaaatggttcttgctttaccataatgaccgatggaccaagtgatggacaggcctcatacaaaatcttcagaatagaaaagggaaagataatcaaatcagtcgaaatgaaagcccctaattatcactatgaggaatgctcctgttaccctgattctagtgaaatcacatgtgtgtgcagggataactggcatggctcgaatcgaccgtgggtgtctttcaaccagaatctggaatatcagatgggatacatatgcagtggggttttcggagacaatccacgccctaatgataagacaggcagttgtggtccagtatcgtctaatggagcaaatggagtaaaaggattttcattcaaatacggcaatggtgtttggatagggagaactaaaagcattagttcaagaaaaggttttgagatgatttgggatccgaatggatggactgggactgacaataaattctcaataaagcaagatatcgtaggaataaatgagtggtcagggtatagcgggagttttgttcagcatccagaactaacagggctggattgtataagaccttgcttctgggttgaactaataagagggcgacccgaagagaacacaatctggactagcgggagcagcatatccttttgtggtgtaaacagtgacactgtgggttggtcttggccagacggtgctgagttgccatttaccattgacaagtaa

SEQ ID NO: 71 - RNA, 285 nt, Haemophilus influenzae
  source 1..285: mol_type = genomic RNA
atgagtcttctaaccgaggtcgaaacgcctaccagaagcgaatgggagtgcagatgcagcggttcaagtgatcctctcgtcattatcattgggatcttgcacctgatattgtggattactgatcgtctttttttcaaatgcatttatcgtcgctttaaatacggtttgaaaagagggccttctacggaaggagtgcctgagtccatgagggaagaatatcaacaggagcagcagagtgctgtggatgttgacgatggtcattttgtcaacatagagctagagtaa

SEQ ID NO: 72 - RNA, 798 nt, Haemophilus influenzae
  source 1..798: mol_type = genomic RNA
atgaaagtgaagctgctggtcctgctgtgcaccttcaccgccacatatgccgacaccatctgcatcggctaccacgccaacaacagcacagataccgtggacaccgtgctggagaagaacgtgaccgtgacacacagcgttaatctgctggaaaacggaggcggaggcaagtacgtgtgcagcgccaagctgagaatggtgaccggcctgagaaacaaacctagcaagcagagccaaggcctgttcggcgccatcgccggcttcaccgagggcggatggaccggcatggtcgacggctggtacggctatcaccaccagaacgagcagggcagcggctacgccgctgatcagaagtctacacaaaatgctattaacggcatcaccaacaaggtgaacagcgtgatcgagaagatgaatacccagtacaccgccatcggctgtgaatacaacaagtccgagcggtgtatgaaacagatcgaagataagatcgaggagatcgagagcaagatctggtgctacaacgccgagctgctcgtgctgctggaaaacgagagaacactggacttccacgattctaatgtgaagaacctgtacgagaaggtgaagagccagctgaagaacaacgctaaggaaatcggcaacggatgttttgagttctaccataagtgcaacgacgagtgcatggaatctgtgaagaatggaacatacgactaccccaagtacagcgaggaatccaagctgaaccgggaaaaaatcgacggcgtgaaactggaaagcatgggcgtgtaccagatctga

SEQ ID NO: 73 - RNA, 1410 nt, Haemophilus influenzae
  source 1..1410: mol_type = genomic RNA
atgaatccaaaccaaaagataataaccattggttctatctgtatgacaattggaacggctaacttaatattacaaattggaaacataatctcaatatgggttagccactcaattcaaattggaaatcaaagccagattgaaacatgcaataaaagcgtcattacttatgaaaacaacacttgggtaaatcagacatttgttaacatcagcaacactaactctgctgctagacagtcagtggcttccgtgaaattagcgggcaattcctctctctgccctgttagtggatgggctatatacagtaaagacaacagtgtaagaatcggttccaagggggatgtgtttgtcataagggaaccattcatatcatgctctcccttggaatgcagaaccttcttcttgactcaaggggctttgctaaatgacaaacattccaatggaaccattaaagacagaagcccatatcgaaccctaatgagctgtcctattggtgaagttccctctccatacaactcaagatttgagtcagtcgcttggtcagcaagtgcttgtcatgatggcaccaattggctaacaattggaatttctggcccagacagtggggcagtggctgtgttaaaatacaatggcataataacagacactatcaagagttggaggaacaagatattgagaacacaagagtctgaatgtgcatgtgtaaatggttcttgctttaccataatgaccgatggaccaagtgatggacaggcctcatacaaaatcttcagaatagaaaagggaaagataatcaaatcagtcgaaatgaaagcccctaattatcactatgaagaatgctcctgttaccctgattctagtgaaatcacatgtgtgtgcagggataactggcatggctcgaatcgaccgtgggtgtctttcaaccagaatctggaatatcagatgggatacatatgcagtggggttttcggagacaatccacgccctaatgataagacaggcagttgtggtccagtatcgtctaatggagcaaatggggtaaaaggattttcattcaaatacggcaatggtgtttggatagggagaactaagagcattagttcaagaaaaggttttgagatgatttgggatccgaatggatggactgggactgacaataaattctcaaaaaagcaagatatcgtaggaataaatgagtggtcagggtatagcgggagttttgttcagcatccagaactaacagggctgaattgtataagaccttgcttctgggttgaactaataagaggacgacccgaagagaacacaatctggactagcgggagcagcatatccttttgtggtgtagacagtgacattgtgggttggtcttggccagacggtgctgagttgccatttaccattgacaagtaa

SEQ ID NO: 74 - RNA, 1701 nt, Haemophilus influenzae
  source 1..1701: mol_type = genomic RNA
atgaaggcaatactagtagttatgctgtatacatttacaaccgcaaatgcagacacattatgtataggttatcatgcgaacaattcaacagacactgtggacacagtactagaaaagaatgtaacagtaacacactctgtcaatcttctggaagacaagcataacggaaaactatgcaaactaagaggggtagccccattgcatttgggtaaatgtaacattgctggctggatcctgggaaatccagagtgtgaatcactctccacagcaagatcatggtcctacattgtggaaacatctaattcagacaatggaacgtgttacccaggagatttcatcaattatgaggagctaagagagcaattgagctcagtgtcatcatttgaaaggtttgaaatattccccaagacaagttcatggcctaatcatgactcggacaatggtgtaacggcagcatgtcctcacgctggagcaaaaagcttctacaaaaacttgatatggctggttaaaaaaggaaaatcatacccaaagatcaaccaaacctacattaatgataaagggaaagaagtcctcgtgctgtggggcattcaccatccacctactattgctgaccaacaaagtctctatcagaatgcagatgcatatgtttttgtggggacatcaagatacagcaagaagttcaagccggaaatagcaacaagacccaaagtgagggatcaagaagggagaatgaactattactggacactagtagaaccgggagacaaaataacattcgaagcaactggtaatctagtggcaccgagatatgcattcacaatggaaagagatgctggatctggtattatcatttcagatacaccagtccacgattgcaatacaacttgtcagacacccgagggtgctataaacaccagcctcccatttcagaatgtacatccgatcacaattgggaaatgtccaaagtatgtaaaaagcacaaaattgagactggccacaggattgaggaatgtcccgtctattcaatctagaggcctattcggggccattgctggcttcatcgaaggggggtggacagggatggtagatggatggtacggttatcaccatcaaaatgagcaggggtcaggatatgcagccgatctgaagagcacacaaaatgccattgataagattactaacaaagtaaattctgttattgaaaagatgaatacacagttcacagcagttggtaaagagttcaaccaccttgaaaaaagaatagagaatctaaataaaaaggttgatgatggtttcctggacatttggacttacaatgccgaactgttggttctactggaaaacgaaagaactttggactatcacgattcaaatgtgaagaacttgtatgaaaaagtaagaaaccagttaaaaaacaatgccaaggaaattggaaacggctgctttgaattttaccacaaatgcgacaacacatgcatggaaagtgtcaagaatgggacttatgactacccaaaatactcagaggaagcaaaattaaacagagaaaaaatagatggagtaaagctggactcaacaaggatctaccagattttggcgatctattcaactgttgccagttcattggtactggtagtctccctgggggcaatcagcttctggatgtgctctaatgggtctctacagtgtagaatatgtatttaa

SEQ ID NO: 75 - RNA, 1401 nt, Haemophilus influenzae
  source 1..1401: mol_type = genomic RNA
atgctaccttcaactatacaaacgttaaccctatttctcacatcagggggagtattattatcactatatgtgtcagcttcattatcatacttactatattcggatatattgctaaaattctcaccaacagaaataactgcaccaacaatgccattggattgtgcaaacgcatcaaatgttcaggctgtgaaccgttctgcaacaaaaggggtgacacttcttctcccagaaccggagtggacatacccgcgtttatcttgcccgggctcaacctttcagaaagcacttctaattagccctcatagattcggagaaaccaaaggaaactcagctcccttgataataagggaaccttttgtagcttgtggaccaaatgaatgcaaacactttgctttaacccattatgctgcccaaccagggggatactataatggaacaagaggagacagaaacaagctgaggcatctaatttcagtcaaattgggcaaaatcccaacagtagagaactccattttccacatggcagcatggagcgggtccgcgtgccatgatggtaaggaatggacatatatcggagttgatggccctgacaataatgcattgctcaaagtaaaatatggagaagcatatactgacacataccattcctatgcaaacaacatcctaagaacacaagaaagtgcctgcaattgcatcgggggaaattgttatctaatgataactgatggctcagcttcaggtgttagtgaatgcagatttcttaagattcgagagggccgaataataaaagaaatatttccaacaggaagagtaaaacacactgaggagtgcacatgcggatttgccagcaataaaaccatagaatgtgcctgtagagacaacaggtacacagcaaaaagaccttttgtcaaattaaacgtggagactgatacagcagaaataaggttgatgtgcacagatacttatttggacacccccagaccaaatgatggaagcataacaggcccttgtgaatctgatggggacaaagggagtggaggcatcaagggaggatttgttcatcaaagaatgaaatccaagattggaaggtggtactctcgaacgatgtctaaaactgaaaggatggggatgggactgtatgtcaagtatggtggagacccatgggctgacagtgatgccctaacttttagtggagtaatggtttcaatgaaagaacctggttggtattcctttggcttcgaaataaaagataagaaatgcgatgtcccctgtattgggatagagatggtacatgatggtggaaaagagacttggcactcagcagcaacagccatttactgtttaatgggctcaggacagctgctgtgggacactgtcacaggtgttgacatggctctgtaa

SEQ ID NO: 76 - RNA, 1749 nt, Haemophilus influenzae
  source 1..1749: mol_type = genomic RNA
atgaaggcaataattgtactactcatggtagtaacatccaatgcagatcgaatctgcactgggataacatcgtcaaactcaccacatgtcgtcaaaactgctactcaaggggaggtcaacgtgaccggtgtaataccactgacaacaacacccaccaaatctcattttgcaaatctcaaaggaacagaaaccagggggaaactatgcccaaaatgcctcaactgcacagatctggatgtagccttgggcagaccaaaatgcacagggaaaataccctctgcaagggtttcaatactccatgaagtcagacctgttacatctgggtgctttcctataatgcacgatagaacaaaaattagacagctgcctaaccttctccgaggatacgaacatgtcaggttatcaactcacaacgttatcaatgcagaagatgcaccaggaagaccctacgaaattggaacctcagggtcttgccctaacattaccaatggaaacggattcttcgcaacaatggcttgggccgtcccaaaaaacaaaacagcaacaaatccattaacaatagaagtaccatacatttgtacagaaggagaagaccaaattaccgtttgggggttccactctgacagcgagacccaaatggcaaagctctatggggactcaaagccccagaagttcacctcatctgccaacggagtgaccacacattacgtttcacagattggtggcttcccaaatcaaacagaagacggaggactaccacaaagtggcagaattgttgttgattacatggtgcagaaatctggaaaaacaggaacaattacctatcaaagaggtattttattgcctcaaaaggtgtggtgcgcaagtggcaggagcaaggtaataaaaggatccttgcccttaattggagaagcagattgcctccatgaaaaatacggtggattaaacaaaagcaagccttactacacaggggaacatgcaaaggccataggaaattgcccaatatgggtgaaaacacccttgaagctggccaatggaaccaaatatagaccccctgcaaaactattaaaggaaagaggtttcttcggagccattgctggtttcttagagggaggatgggaaggaatgattgcaggttggcacggatacacatcccatggggcacatggagtagcggtggcagctgaccttaagagcactcaagaggccataaacaagataacaaaaaatctcaactctttgagtgagctggaagtaaagaatcttcaaagactaagcggtgccatggatgaactccacaacgaaatactagaactagatgagaaagtggatgatctcagagctgatacaataagctcacaaatagaactcgcagtcctgctttccaatgaaggaataataaacagtgaagatgaacatctcttggcgcttgaaagaaagctgaagaaaatgctgggcccctctgctgtagagatagggaatggatgctttgaaaccaaacacaagtgcaaccagacctgtctcgacagaatagctgctggtacctttgatgcaggagaattttctctccccacctttgattcactgaatattactgctgcatctttaaatgacgacggattggacaatcatactatactgctttactactcaactgctgcctccagtttggctgtaacactgatgatagctatctttgttgtttatatggtctccagagacaatgtttcttgctccatttgtctataa

SEQ ID NO: 77 - RNA, 687 nt, Haemophilus influenzae
  source 1..687: mol_type = genomic RNA
atgaagaccatcatcgccctgagctacatcctgtgcctggtgttcgcccagaagctgcccggcaacgacaacagcaccgccaccctgtgcctgggccaccacgccgtgcccaacggcaccatcgtgaagaccatcaccaacgaccagatcgaggtgaccaacgccaccgagctggtgttccccggctgcggcgtgctgaagctggccaccggcatgaggaacgtgcccgagaagcagaccaggggcatcttcggcgccatcgccggcttcatcgagaacggctgggagggcatggtggacggctggtacggcttcaggcaccagaacagcgagggcatcggccaggccgccgacctgaagagcacccaggccgccatcaaccagatcaacggcatggtgaacagggtgatcgagctgatggagcagggcggccccgactgctacctggccgagctgctggtggccctgctgaaccagcacaccatcgacctgaccgacagcgagatgaggaagctgttcgagaggaccaagaagcagctgagggagaacgccgaggacatgggcaacggctgcttcaagatctaccacaagtgcgacaacgcctgcatcggcagcatcaggaacggcacctacgaccacgacgtgtacagggacgaggccctgaacaacaggttccagatcaagtaa

SEQ ID NO: 78 - RNA, 666 nt, Haemophilus influenzae
  source 1..666: mol_type = genomic RNA
atgaacacccagatcctggtgttcgccctgatcgccatcatccccaccaacgccgacaagatctgcctgggccaccacgccgtgagcaacggcaccaaggtgaacaccctgaccgagaggggcgtggaggtggtgaacgccaccgagctggtgttccccggctgcggcgtgctgctgctggccaccggcatgaagaacgtgcccgagatccccaagggcaggggcctgttcggcgccatcgccggcttcatcgagaacggctgggagggcctgatcgacggctggtacggcttcaggcaccagaacgcccagggcgagggcaccgccgccgactacaagagcacccagagcgccatcgaccagatcaccggcatggtgaacagggtgatcgagctgatggagcagggcggccccgactgctacctggccgagctgctggtggccatgctgaaccagcacaccatcgacctggccgacagcgagatggacaagctgtacgagagggtgaagaggcagctgagggagaacgccgaggaggacggcaccggctgcttcgagatcttccacaagtgcgacgacgactgcatggccagcatcaggaacaacacctacgaccacagcaagtacagggaggaggccatgcagaacaggatccagatcgactaa

SEQ ID NO: 79 - RNA, 1725 nt, Human orthopneumovirus
  source 1..1725: mol_type = genomic RNA
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaaggaaaataagtgtaatggaacagatgctaaggtaaaattgataaaacaagaattagataaatataaaaatgctgtaacagaattgcagttgctcatgcaaagcacaccaccaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatgtaaggtcctgcacctagaaggggaagtgaacaagatcaaaagtgctctactatccacaaacaaggctgtagtcagcttatcaaatggagttagtgtcttaaccttcaaagtgttagacctcaaaaactatatagataaacaattgttacctattctgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtgcataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaaataaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaagggatggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 80 - RNA, 288 nt, Mycobacterium tuberculosis
  source 1..288: mol_type = genomic RNA
atgacagagcagcagtggaatttcgcgggtatcgaggccgcggcaagcgcaatccagggaaatgtcacgtccattcattccctccttgacgaggggaagcagtccctgaccaagctcgcagcggcctggggcggtagcggttcggaggcgtaccagggtgtccagcaaaaatgggacgccacggctaccgagctgaacaacgcgctgcagaacctggcgcggacgatcagcgaagccggtcaggcaatggcttcgaccgaaggcaacgtcactgggatgttcgcatag

SEQ ID NO: 81 - RNA, 978 nt, Mycobacterium tuberculosis
  source 1..978: mol_type = genomic RNA
atgacagacgtgagccgaaagattcgagcttggggacgccgattgatgatcggcacggcagcggctgtagtccttccgggcctggtggggcttgccggcggagcggcaaccgcgggcgcgttctcccggccggggctgccggtcgagtacctgcaggtgccgtcgccgtcgatgggccgcgacatcaaggttcagttccagagcggtgggaacaactcacctgcggtttatctgctcgacggcctgcgcgcccaagacgactacaacggctgggatatcaacaccccggcgttcgagtggtactaccagtcgggactgtcgatagtcatgccggtcggcgggcagtccagcttctacagcgactggtacagcccggcctgcggtaaggctggctgccagacttacaagtgggaaaccttcctgaccagcgagctgccgcaatggttgtccgccaacagggccgtgaagcccaccggcagcgctgcaatcggcttgtcgatggccggctcgtcggcaatgatcttggccgcctaccacccccagcagttcatctacgccggctcgctgtcggccctgctggacccctctcaggggatggggcctagcctgatcggcctcgcgatgggtgacgccggcggttacaaggccgcagacatgtggggtccctcgagtgacccggcatgggagcgcaacgaccctacgcagcagatccccaagctggtcgcaaacaacacccggctatgggtttattgcgggaacggcaccccgaacgagttgggcggtgccaacatacccgccgagttcttggagaacttcgttcgtagcagcaacctgaagttccaggatgcgtacaacgccgcgggcgggcacaacgccgtgttcaacttcccgcccaacggcacgcacagctgggagtactggggcgctcagctcaacgccatgaagggtgacctgcagagttcgttaggcgccggctga

SEQ ID NO: 82 - RNA, 291 nt, Mycobacterium tuberculosis
  source 1..291: mol_type = genomic RNA
atgtcgcaaatcatgtacaactaccccgcgatgttgggtcacgccggggatatggccggatatgccggcacgctgcagagcttgggtgccgagatcgccgtggagcaggccgcgttgcagagtgcgtggcagggcgataccgggatcacgtatcaggcgtggcaggcacagtggaaccaggccatggaagatttggtgcgggcctatcatgcgatgtccagcacccatgaagccaacaccatggcgatgatggcccgcgacacggccgaagccgccaaatggggcggctag

SEQ ID NO: 83 - RNA, 432 nt, Mycobacterium tuberculosis
  source 1..432: mol_type = genomic RNA
atgaccaccgcacgcgacatcatgaacgcaggtgtgacctgtgttggcgaacacgagacgctaaccgctgccgctcaatacatgcgtgagcacgacatcggcgcgttgccgatctgcggggacgacgaccggctgcacggcatgctcaccgaccgcgacattgtgatcaaaggcctggctgcgggcctagacccgaataccgccacggctggcgagttggcccgggacagcatctactacgtcgatgcgaacgcaagcatccaggagatgctcaacgtcatggaagaacatcaggtccgccgtgttccggtcatctcagagcaccgcttggtcggaatcgtcaccgaagccgacatcgcccgacacctgcccgagcacgccattgtgcagttcgtcaaggcaatctgctcgcccatggccctcgccagctag

SEQ ID NO: 84 - RNA, 1074 nt, Mycobacterium tuberculosis
  source 1..1074: mol_type = genomic RNA
atgaccgtcgacggaaccgcgatgcgggtgaccacgatgaaatcgcgggtgatcgacatcgtcgaagagaacgggttctcagtcgacgaccgcgacgacctgtatcccgcggccggcgtgcaggtccatgacgccgacaccatcgtgctgcggcgtagccgtccgctgcagatctcgctggatggtcacgacgctaagcaggtgtggacgaccgcgtcgacggtggacgaggcgctggcccaactcgcgatgaccgacacggcgccggccgcggcttctcgcgccagccgcgtcccgctgtccgggatggcgctaccggtcgtcagcgccaagacggtgcagctcaacgacggcgggttggtgcgcacggtgcacttgccggcccccaatgtcgcggggctgctgagtgcggccggcgtgccgctgttgcaaagcgaccacgtggtgcccgccgcgacggccccgatcgtcgaaggcatgcagatccaggtgacccgcaatcggatcaagaaggtcaccgagcggctgccgctgccgccgaacgcgcgtcgtgtcgaggacccggagatgaacatgagccgggaggtcgtcgaagacccgggggttccggggacccaggatgtgacgttcgcggtagctgaggtcaacggcgtcgagaccggccgtttgcccgtcgccaacgtcgtggtgaccccggcccacgaagccgtggtgcgggtgggcaccaagcccggtaccgaggtgcccccggtgatcgacggaagcatctgggacgccatcgcgcaatgcaaatccggcggcaattgggcggccaacaccggtaacgggttatacggtggtctgcagatcagccaggcggcgtgggattccaacggtggtgtcgggtcgccggcggccgcgagtccccagcaacagatcgaggtcgcagacaacattatgaaaaccgcaggcccgggtgcgtggccgaaatgtagttcttgtagtcagggagacgcaccgctgggctcgctcacccacatcctgacgttcctcgcggccgagactggaggttgttcggggagcagggacgattga

SEQ ID NO: 85-199 - skipped (115 entries)

SEQ ID NO: 200 - protein, 253 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 201 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 202 - protein, 239 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 203 - protein, 215 aa, Homo sapiens
MWPPGSASQPPPSPAAATGLHPAARPVSLQCRLSMCPARSLLLVATLVLLDHLSLARNLPVATPDPGMFPCLHHSQNLLRAVSNMLQKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 204 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 205 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

(19) United States
(12) Patent Application Publication
(10) Pub. No.: US 2025/0177515 A1
(43) Pub. Date: Jun. 5, 2025

(54) METHODS AND COMPOSITIONS RELATING TO IMMUNIZATION OF IMMUNE DISTINCT PATIENTS

(71) Applicant: THE CHILDREN'S MEDICAL CENTER CORPORATION, Boston, MA (US)

(72) Inventors: David J. DOWLING, Brighton, MA (US); Byron NF BROOK, Boston, MA (US)

(73) Assignee: THE CHILDREN'S MEDICAL CENTER CORPORATION, Boston, MA (US)

(21) Appl. No.: 18/727,421

(22) PCT Filed: Jan. 10, 2023
(86) PCT No.: PCT/US2023/010462

§ 371 Date: Jul. 9, 2024

Related U.S. Application Data
(60) Provisional application No. 63/297,881, filed on Jan. 10, 2022.

(51) Int. Cl. A61K 39/39; A61K 39/00; A61K 39/015; A61K 39/02; A61K 39/04; A61K 39/085; A61K 39/09; A61K 39/12; A61K 39/145; A61K 39/21; A61K 39/215; A61K 39/25; A61P 37/04

(52) U.S. Cl. CPC A61K 39/39; A61K 39/015; A61K 39/0291; A61K 39/04; A61K 39/085; A61K 39/092; A61K 39/12; A61K 39/145; A61K 39/21; A61K 39/215; A61K 39/25; A61P 37/04; A61K 2039/53; A61K 2039/55; A61K 2039/55522; A61K 2039/55527; A61K 2039/55533; A61K 2039/55538

(57) ABSTRACT

Described herein are methods and compositions relating to vaccinating, immunizing, or inducing an immune response in an immune distinct subject. In some embodiments, the methods and compositions relate to a first cytokine mRNA construct comprising a first open reading frame (ORF), wherein the first ORF encodes a proinflammatory cytokine; and optionally, one or more of: a first antigen mRNA construct comprising a second open reading frame (ORF), wherein the second ORF encodes an antigen; and an antigen polypeptide, antigen molecule, or killed or attenuated pathogenic agent. The methods and compositions described herein provide adjuvantation that overcomes the resistance of immune distinct patients to vaccination, permitting more effective vaccination, as well as the ability to reduce dosages, reduce the need for boosters, and permit antigen stacking to immunize more comprehensively.

### CROSS-REFERENCE TO RELATED APPLICATIONS

This application claims benefit under 35 U.S.C. § 119 (e) of U.S. Provisional Application No. 63/297,881 filed Jan. 10, 2022, the contents of which are incorporated herein by reference in their entirety.

### GOVERNMENT SUPPORT

This invention was made with government support under 75N93019C00044 awarded by the National Institutes of Health. The government has certain rights in the invention.

### TECHNICAL FIELD

The technology described herein relates to compositions comprising messenger ribonucleic acid (mRNA) adjuvants and optionally antigens, and methods of using these compositions in a variety of therapeutic and prophylactic indications.

### BACKGROUND

Vaccines typically rely upon adjuvants to stimulate the immune system and generate an effective response to the vaccine. Existing adjuvants, while effective in normal healthy adults, often give poor performance or are even counterproductive in “immune distinct” patients, i.e., those patients with immune systems that are distinct in functionality from a normal healthy adult. Immune distinct patients include the elderly and infants, who do not respond optimally to currently standard adjuvants. In order to successfully immunize immune distinct patients, and reduce the number of vaccine doses such patients receive, more effective adjuvants are necessary.

### SUMMARY

The inventors have demonstrated herein that the use of mRNA constructs encoding proinflammatory cytokines provides adjuvantation that overcomes the resistance of immune distinct patients to vaccination. This permits more effective vaccination, as well as the ability to reduce dosages, reduce the need for boosters, and permit antigen stacking to immunize more comprehensively.

In one aspect of any of the embodiments, described herein is a method for inducing an immune response in an immune distinct subject, the method comprising administering to the subject one or more compositions comprising:
a) a first cytokine mRNA construct comprising a first open reading frame (ORF), wherein the first ORF encodes a proinflammatory cytokine; and
b) optionally, one or more of:
   i) a first antigen mRNA construct comprising a second open reading frame (ORF), wherein the second ORF encodes an antigen; and
   ii) an antigen polypeptide, antigen molecule, or killed or attenuated pathogenic agent.
      Optionally, in some embodiments, the method of the above aspect is not a method for treatment of the human or animal body by surgery or therapy practiced on the human or animal body. In one aspect of any of the embodiments, described herein are one or more compositions comprising:
a) a first cytokine mRNA construct comprising a first open reading frame (ORF), wherein the first ORF encodes a proinflammatory cytokine; and
b) optionally, one or more of:
   a) a first antigen mRNA construct comprising a second open reading frame (ORF), wherein the second ORF encodes an antigen; and
   b) an antigen polypeptide, antigen molecule, or killed or attenuated pathogenic agent,
      for use in a method of inducing an immune response in an immune distinct subject, the method comprising administering the one or more compositions to the subject. In one aspect of any of the embodiments, described herein are one or more compositions comprising:
a) a first cytokine mRNA construct comprising a first open reading frame (ORF), wherein the first ORF encodes a proinflammatory cytokine; and
b) optionally, one or more of:
   c) a first antigen mRNA construct comprising a second open reading frame (ORF), wherein the second ORF encodes an antigen; and
   d) an antigen polypeptide, antigen molecule, or killed or attenuated pathogenic agent,
      for use in inducing an immune response in an immune distinct subject.

In some embodiments of any of the aspects, the immune response comprises an increase in IL-12 in the subject. In some embodiments of any of the aspects, the immune response comprises an increase in the active IL-12 heterodimer (referred to as ‘p70’) in the subject. In some embodiments of any of the aspects, the immune response comprises an increase in Ig levels in the subject. In some embodiments of any of the aspects, the Ig is IgG2, IgG3, or IgG2a. In some embodiments of any of the aspects, the IgG2a is IgG2a that specifically binds the antigen. In some embodiments of any of the aspects, the Ig is IgG1, IgG3, or IgG4. In some embodiments of any of the aspects, the IgG1, IgG3, or IgG4 is IgG1, IgG3, or IgG4 that specifically binds the antigen. In some embodiments of any of the aspects, the immune response comprises a CD4+ T cell response in the subject. In some embodiments of any of the aspects, the immune response comprises a CD8+ T cell response in the subject. In some embodiments of any of the aspects, the immune response comprises a NK cell response in the subject. In some embodiments of any of the aspects, the immune response comprises a Th1 response in the subject. In some embodiments of any of the aspects, the immune response stimulates the production of an interferon gamma (IFNγ) response from T cells in the subject. In some embodiments of any of the aspects, the immune response initiates phagocytosis via the Fc region of each IgG subclass via improved affinity for phagocyte membrane Fc-gamma-receptors (FcγR). In some embodiments of any of the aspects, the immune response comprises immunization of the subject against the antigen or an organism comprising the antigen. In some embodiments of any of the aspects, the immune response comprises activation of innate immune responses.

In one aspect of any of the embodiments, described herein is a method for treating or preventing a disease in an immune distinct subject, the method comprising administering to the subject one or more compositions comprising:
a) a first cytokine mRNA construct comprising a first open reading frame (ORF), wherein the first ORF encodes a proinflammatory cytokine; and
b) optionally, one or more of:
   i) a first antigen mRNA construct comprising a second open reading frame (ORF), wherein the second ORF encodes an antigen; and
   ii) an antigen polypeptide, antigen molecule, or killed or attenuated pathogenic agent.
      In one aspect of any of the embodiments, described herein are one or more compositions comprising:
a) a first cytokine mRNA construct comprising a first open reading frame (ORF), wherein the first ORF encodes a proinflammatory cytokine; and
b) optionally, one or more of:
   i) a first antigen mRNA construct comprising a second open reading frame (ORF), wherein the second ORF encodes an antigen; and
   ii) an antigen polypeptide, antigen molecule, or killed or attenuated pathogenic agent,
      for use in a method of treating or preventing a disease in an immune distinct subject, the method comprising administering the one or more compositions to the subject. In one aspect of any of the embodiments, described herein are one or more compositions comprising:
a) a first cytokine mRNA construct comprising a first open reading frame (ORF), wherein the first ORF encodes a proinflammatory cytokine; and
b) optionally, one or more of:
   iii) a first antigen mRNA construct comprising a second open reading frame (ORF), wherein the second ORF encodes an antigen; and
   iv) an antigen polypeptide, antigen molecule, or killed or attenuated pathogenic agent,
      for use in the treatment or prevention of a disease in an immune distinct subject.

In one aspect of any of the embodiments, described herein is a method for immunizing an immune distinct subject, the method comprising administering to the subject one or more compositions comprising:
a) a first cytokine mRNA construct comprising a first open reading frame (ORF), wherein the first ORF encodes a proinflammatory cytokine; and
b) optionally, one or more of:
   i) a first antigen mRNA construct comprising a second open reading frame (ORF), wherein the second ORF encodes an antigen; and
   ii) an antigen polypeptide, antigen molecule, or killed or attenuated pathogenic agent.
      Optionally, in some embodiments, the method of the above aspect is not a method for treatment of the human or animal body by surgery or therapy practiced on the human or animal body. In one aspect of any of the embodiments, described herein are one or more compositions comprising:
a) a first cytokine mRNA construct comprising a first open reading frame (ORF), wherein the first ORF encodes a proinflammatory cytokine; and
b) optionally, one or more of:
   i) a first antigen mRNA construct comprising a second open reading frame (ORF), wherein the second ORF encodes an antigen; and
   ii) an antigen polypeptide, antigen molecule, or killed or attenuated pathogenic agent,
      for use in a method of immunizing an immune distinct subject, the method comprising administering the one or more compositions to the subject. In one aspect of any of the embodiments, described herein are one or more compositions comprising:
a) a first cytokine mRNA construct comprising a first open reading frame (ORF), wherein the first ORF encodes a proinflammatory cytokine; and
b) optionally, one or more of:
   iii) a first antigen mRNA construct comprising a second open reading frame (ORF), wherein the second ORF encodes an antigen; and
   iv) an antigen polypeptide, antigen molecule, or killed or attenuated pathogenic agent,
      for use in immunizing an immune distinct subject.

In some embodiments of any of the aspects, the induction of an immune response, the treatment or prevention of a disease, or the immunizing effect is achieved by any one or more of the following: inducing an increase in IL-12 in the subject, inducing an increase in the active IL-12 heterodimer (referred to as ‘p70’) in the subject, inducing an increase in Ig levels in the subject (optionally wherein the Ig is IgG1, IgG3, or IgG4, optionally wherein the IgG1, IgG3, or IgG4 is IgG1, IgG3, or IgG4 that specifically binds the antigen), by inducing a CD4+ T cell response in the subject, by inducing a CD8+ T cell response in the subject, by inducing a NK cell response in the subject, by inducing a Th1 response in the subject, by stimulating the production of an interferon gamma (IFNγ) response from T cells in the subject, by initiating phagocytosis via the Fc region of each IgG subclass via improved affinity for phagocyte membrane Fc-gamma-receptors (FcγR) in the subject, e.g., by activating innate immune responses.

In some embodiments of any of the aspects, the immune distinct subject is a subject with immunosenescence. In some embodiments of any of the aspects, the immune distinct subject and/or subject with immunosenescence is a subject of 55 years of age or older. In some embodiments of any of the aspects, the immune distinct subject and/or subject with immunosenescence is a subject of 60 years of age or older. In some embodiments of any of the aspects, the immune distinct subject and/or subject with immunosenescence is a subject of 65 years of age or older. In some embodiments of any of the aspects, the immune distinct subject and/or subject with immunosenescence is a subject of 70 years of age or older. In some embodiments of any of the aspects, the immune distinct subject and/or subject with immunosenescence is a subject of 75 years of age or older. In some embodiments of any of the aspects, the immune distinct subject and/or subject with immunosenescence is a subject who has or is determined to have a reduced TNF response to immune stimuli. In some embodiments of any of the aspects, the immune distinct subject and/or subject with immunosenescence is a subject who has or is determined to have a reduced IL-12 response to immune stimuli. In some embodiments of any of the aspects, the immune stimuli is lipopolysaccharide (LPS).

In some embodiments of any of the aspects, the immune distinct subject is an infant. In some embodiments of any of the aspects, the immune distinct subject and/or infant is 2 years of age or younger. In some embodiments of any of the aspects, the immune distinct subject and/or infant is 1 year of age or younger. In some embodiments of any of the aspects, the immune distinct subject and/or infant is 28 days of age or younger. In some embodiments of any of the aspects, the immune distinct subject and/or infant is born preterm/is a preterm infant.

In some embodiments of any of the aspects, the immune distinct subject is elderly or an infant. In some embodiments of any of the aspects, the immune distinct subject is a subject of 55 years of age or older, 60 years of age or older, 65 years of age or older, 70 years of age or older, 75 years of age or older, or 2 years of age or younger, 1 year of age or younger, 28 days of age or younger, or is a preterm infant. In some embodiments of any of the aspects, the immune distinct subject is a subject of 55 years of age or older, or is 2 years of age or younger. In some embodiments of any of the aspects, the immune distinct subject is a subject of 55 years of age or older, or is 1 year of age or younger. In some embodiments of any of the aspects, the immune distinct subject is a subject of 55 years of age or older, or is 28 days of age or younger. In some embodiments of any of the aspects, the immune distinct subject is a subject of 55 years of age or older, or is a preterm infant. In some embodiments of any of the aspects, the immune distinct subject is a subject of 60 years of age or older, or is 2 years of age or younger. In some embodiments of any of the aspects, the immune distinct subject is a subject of 60 years of age or older, or is 1 year of age or younger. In some embodiments of any of the aspects, the immune distinct subject is a subject of 60 years of age or older, or is 28 days of age or younger. In some embodiments of any of the aspects, the immune distinct subject is a subject of 60 years of age or older, or is a preterm infant. In some embodiments of any of the aspects, the immune distinct subject is a subject of 65 years of age or older, or is 2 years of age or younger. In some embodiments of any of the aspects, the immune distinct subject is a subject of 65 years of age or older, or is 1 year of age or younger. In some embodiments of any of the aspects, the immune distinct subject is a subject of 65 years of age or older, or is 28 days of age or younger. In some embodiments of any of the aspects, the immune distinct subject is a subject of 65 years of age or older, or is a preterm infant. In some embodiments of any of the aspects, the immune distinct subject is a subject of 70 years of age or older, or is 2 years of age or younger. In some embodiments of any of the aspects, the immune distinct subject is a subject of 70 years of age or older, or is 1 year of age or younger. In some embodiments of any of the aspects, the immune distinct subject is a subject of 70 years of age or older, or is 28 days of age or younger. In some embodiments of any of the aspects, the immune distinct subject is a subject of 70 years of age or older, or is a preterm infant. In some embodiments of any of the aspects, the immune distinct subject is a subject of 75 years of age or older, or is 2 years of age or younger. In some embodiments of any of the aspects, the immune distinct subject is a subject of 75 years of age or older, or is 1 year of age or younger. In some embodiments of any of the aspects, the immune distinct subject is a subject of 75 years of age or older, or is 28 days of age or younger. In some embodiments of any of the aspects, the immune distinct subject is a subject of 75 years of age or older, or is a preterm infant.

In some embodiments of any of the aspects, the immune distinct subject is immunocompromised, has an HIV infection, has AIDS, has received a transplant, is undergoing immunosuppression, is immunosuppressed, has an infection, is diabetic, has an IgG subclass deficiency, has a substance abuse disorder, or is obese. In some embodiments of any of the aspects, the subject is a subject in a high density living environment. In some embodiments of any of the aspects, the high density living environment is an assisted living facility; a nursing home, a dormitory, or a hospital.

In some embodiments of any of the aspects, the subject is a subject who is:
a) at least 55 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) at least 60 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) at least 65 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) at least 70 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) at least 75 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) 2 years of age or younger; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) 1 year of age or younger; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) 28 days of age or younger; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) is a preterm infant; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) at least 55 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, and/or is obese.
   In some embodiments of any of the aspects, the subject is at least 60 years of age or older, at least 65 years of age or older, at least 70 years of age or older, or at least 75 years of age or older.

In some embodiments of any of the aspects, the composition comprises at least 5× less of the antigen than is required to induce an immune response in the absence of the first cytokine mRNA. In some embodiments of any of the aspects, the composition comprises at least 10× less of the antigen than is required to induce an immune response in the absence of the first cytokine mRNA. In some embodiments of any of the aspects, the composition comprises at least 20× less of the antigen than is required to induce an immune response in the absence of the first cytokine mRNA. In some embodiments of any of the aspects, the composition comprises at least 50× less of the antigen than is required to induce an immune response in the absence of the first cytokine mRNA. In some embodiments of any of the aspects, the composition comprises at least 100× less of the antigen than is required to induce an immune response in the absence of the first cytokine mRNA.

In some embodiments of any of the aspects, the method comprises administering each of the one or more compositions to the immune distinct subject no more frequently than once per year. In some embodiments of any of the aspects, the method comprises administering each of the one or more compositions to the immune distinct subject no more frequently than once every 2 years. In some embodiments of any of the aspects, the method comprises administering each of the one or more compositions to the immune distinct subject no more frequently than once every 3 years. In some embodiments of any of the aspects, the method comprises administering each of the one or more compositions to the immune distinct subject no more frequently than once every 4 years. In some embodiments of any of the aspects, the method comprises administering each of the one or more compositions to the immune distinct subject no more frequently than once every 5 years. In any of these embodiments, the administration may be intravenous.

In some embodiments of any of the aspects, the first antigen mRNA construct antigen is an antigen of a first infectious organism and the method comprises administering a composition comprising any antigen from the first infectious organism to the immune distinct subject no more frequently than once per year. In some embodiments of any of the aspects, the first antigen mRNA construct antigen is an antigen of a first infectious organism and the method comprises administering a composition comprising any antigen from the first infectious organism to the immune distinct subject no more frequently than once every 2 years. In some embodiments of any of the aspects, the first antigen mRNA construct antigen is an antigen of a first infectious organism and the method comprises administering a composition comprising any antigen from the first infectious organism to the immune distinct subject no more frequently than once every 3 years. In some embodiments of any of the aspects, the first antigen mRNA construct antigen is an antigen of a first infectious organism and the method comprises administering a composition comprising any antigen from the first infectious organism to the immune distinct subject no more frequently than once every 4 years. In some embodiments of any of the aspects, the first antigen mRNA construct antigen is an antigen of a first infectious organism and the method comprises administering a composition comprising any antigen from the first infectious organism to the immune distinct subject no more frequently than once every 5 years.

In some embodiments of any of the aspects, the proinflammatory cytokine is selected from the group consisting of: IL-12; IL-2; IL-4; IL-5; IL-6; IL-8; IL-10; IL-13; IL-27; IL-1 beta; TGFbeta; IFNγ; IFNα; IFNI3; TNFα; CCL2; CCL3; CCL4; CCL5; CCL8; CXCL12; GM-CSF; and a subunit, dimer, heterodimer, derivative, fragment, agonist or homologue thereof. In some embodiments of any of the aspects, the proinflammatory cytokine is selected from the group consisting of: IL-12; IL-2; IL-4; IL-5; IL-6; IL-7; IL-8; IL-10; IL-13; IL-15; IL-18; IL-21; IL-27; IL-1 beta; TGFbeta; IFNγ; IFNα; IFNI3; TNFα; CCL2; CCL3; CCL4; CCL5; CCL8; CCL27; CXCL12; CXCL13; G-CSF; GM-CSF; B-cell activating factor (BAFF); Keratinocyte growth factor (FGF7); and a subunit, dimer, heterodimer, derivative, fragment, agonist or homologue thereof. In some embodiments of any of the aspects, the proinflammatory cytokine is IL-12 or a subunit, dimer, heterodimer, derivative, fragment, agonist or homologue thereof. In some embodiments of any of the aspects, the first ORF comprises a sequence at least 90% identical to SEQ ID NO: 59. In some embodiments of any of the aspects, the proinflammatory cytokine is IL-12 or a subunit, of human, and other mammalian homology.

In some embodiments of any of the aspects, the one or more compositions further comprise one or more further cytokine mRNA constructs, each comprising a further open reading frame (ORF), wherein each further ORF encodes a proinflammatory cytokine distinct from the proinflammatory cytokine encoded by the first ORF. In some embodiments of any of the aspects, the composition comprises 1-9 further cytokine mRNA constructs. In some embodiments of any of the aspects, the first cytokine mRNA construct further comprises one or more further open reading frames (ORFs), wherein each further ORF encodes a proinflammatory cytokine distinct from the proinflammatory cytokine encoded by the first ORF. In some embodiments of any of the aspects, the first cytokine mRNA construct comprises 1-9 further ORFs encoding a proinflammatory cytokine distinct from the proinflammatory cytokine encoded by the first ORF. In some embodiments of any of the aspects, the first ORF encodes IL-12 or a subunit, derivative, fragment, agonist or homologue thereof and the one or more further ORFs encode IL-2; IL-4; IL-5; IL-6; IL-8; IL-10; IL-13; IL-27; IL-1β; TGFβ; IFNγ; IFNα; IFNβ; TNFα; CCL2; CCL3; CCL4; CCL5; CCL8; CXCL12; GM-CSF; or a subunit, derivative, fragment, agonist or homologue thereof. In some embodiments of any of the aspects, the first ORF encodes IL-12 or a subunit, derivative, fragment, agonist or homologue thereof and the one or more further ORFs encode IL-2; IL-4; IL-5; IL-6; IL-7; IL-8; IL-10; IL-13; IL-15; IL-18; IL-21; IL-27; IL-1β; TGFβ; IFNγ; IFNα; IFNβ; TNFα; CCL2; CCL3; CCL4; CCL5; CCL8; CCL27; CXCL12; CXCL13; G-CSF; GM-CSF; BAFF; FGF7; or a subunit, derivative, fragment, agonist or homologue thereof. Based on the underlying biology, pairwise combinations that may induce synergistic responses or associated function. include: IL-12 and/or IL-2, IL-6, IL-7, IL-15, IL-18, IL-21, IL-27, TNF, BAFF, G-CSF, CCL27, CXCL13, Keratinocyte growth factor (KGF), single-chain variable fragments (scFvs) of anti-CD3, anti-CD4 antibodies.

In some embodiments of any of the aspects, the composition further comprises one or more further antigen mRNA constructs, each comprising a further open reading frame (ORF), wherein each further ORF encodes an antigen distinct from the antigen encoded by the second ORF. In some embodiments of any of the aspects, the composition comprises 1-9 further antigen mRNA constructs. In some embodiments of any of the aspects, the antigen mRNA construct further comprises one or more further open reading frames (ORFs), wherein each further ORF encodes an antigen distinct from the antigen encoded by the second ORF. In some embodiments of any of the aspects, the composition comprises 1-9 further ORFs encoding an antigen distinct from the antigen encoded by the second ORF. In some embodiments of any of the aspects, the composition comprises a plurality of ORFs encoding a plurality of antigens, and the plurality of antigens comprises multiple antigens from a first organism. In some embodiments of any of the aspects, the composition comprises a plurality of ORFs encoding a plurality of antigens, and the plurality of antigens comprises one or more antigens from a first organism and one or more antigens from one or more further organisms. In some embodiments of any of the aspects, the composition comprises a plurality of ORFs encoding a plurality of antigens, and the plurality of antigens comprises one or more antigens from a coronavirus and one or more antigens from an influenza virus. In some embodiments of any of the aspects, the composition comprises a plurality of ORFs encoding a plurality of antigens, and the plurality of antigens comprises one or more spike protein antigens from a coronavirus and one or more antigens from an influenza virus.

In some embodiments of any of the aspects, the antigen is an antigen of an infectious organism and whereby transmission of the infectious organism to or by the subject is reduced as compared to administration of a composition not comprising the cytokine mRNA construct. In some embodiments of any of the aspects, the antigen is a pathogenic microbial protein or an epitope containing fragment thereof. In some embodiments of any of the aspects, the pathogenic microbial protein is selected from the group consisting of: a viral protein; a bacterial protein; a fungal protein; a parasite protein; and a prion. In some embodiments of any of the aspects, the antigen comprises a viral protein or an epitope containing fragment thereof. In some embodiments of any of the aspects, the antigen comprises a coronavirus spike protein. In some embodiments of any of the aspects, the antigen comprises a coronavirus receptor binding domain (RBD) protein. In some embodiments of any of the aspects, the antigen comprises a variant coronavirus spike protein. In some embodiments of any of the aspects, the antigen comprises a variant coronavirus receptor binding domain protein. In some embodiments of any of the aspects, the coronavirus spike protein is a MERS-COV spike or RBD protein. In some embodiments of any of the aspects, the coronavirus spike protein is a SARS-COV-1 spike or RBD protein. In some embodiments of any of the aspects, the coronavirus spike protein is a SARS-COV-2 spike or RBD protein. In some embodiments of any of the aspects wherein the antigen comprises a coronavirus protein or an epitope containing fragment thereof, the subject may have or be at risk of having a coronavirus infection, which may be Middle East Respiratory Syndrome, SARS-COV-1, SARS-COV-2, or SARS-COV-2 any variants of concern. In some such embodiments, the disease in the immune distinct subject may be a coronavirus infection. In some embodiments of any of the aspects wherein the antigen comprises a coronavirus protein or an epitope containing fragment thereof, the method or use may be for inducing an immune response against coronavirus, optionally to prevent coronavirus infection. In some embodiments of any of the aspects wherein the antigen comprises a coronavirus protein or an epitope containing fragment thereof, the method or use may be for prevention or treatment of a coronavirus infection. In some embodiments of any of the aspects wherein the antigen comprises a coronavirus protein or an epitope containing fragment thereof, the method or use may be for immunizing an immune distinct subject against coronavirus, optionally to prevent coronavirus infection.

In some embodiments of any of the aspects, the antigen comprises an influenza protein or a variant thereof, or an epitope containing fragment thereof. In some embodiments of any of the aspects, the influenza protein is selected from the group consisting of a hemagglutinin, a neuraminidase, a matrix-2 and/or a nucleoprotein. In some embodiments of any of the aspects, the influenza protein is selected from type A influenza, a type B influenza, or a subtype of type A influenza of H1, H2, H3, H4, H5, H6, H7, H8, H9, H10, H11, H12, H13, H14, H15 or H16.

In some embodiments of any of the aspects, the antigen comprises a respiratory syncytial virus (RSV) protein, or a variant thereof, or an epitope containing fragment thereof. In some embodiments of any of the aspects, the protein of the respiratory syncytial virus is the F glycoprotein or the G glycoprotein. In some embodiments of any of the aspects wherein the antigen comprises a respiratory syncytial virus (RSV) protein or a variant thereof, or an epitope containing fragment thereof, the subject may have or be at risk of having an RSV infection. In some such embodiments, the disease in the immune distinct subject may be an RSV infection In some embodiments of any of the aspects wherein the antigen comprises a respiratory syncytial virus (RSV) protein or a variant thereof, or an epitope containing fragment thereof, the method or use may be for inducing an immune response against RSV, optionally to prevent respiratory syncytial virus (RSV) infection. In some embodiments of any of the aspects wherein the antigen comprises a respiratory syncytial virus (RSV) protein or a variant thereof, or an epitope containing fragment thereof, the method or use may be for prevention or treatment of an RSV infection. In some embodiments of any of the aspects wherein the antigen comprises a respiratory syncytial virus (RSV) protein or a variant thereof, or an epitope containing fragment thereof, the method or use may be for immunizing an immune distinct subject against RSV, optionally to prevent respiratory syncytial virus (RSV) infection.

In some embodiments of any of the aspects, the antigen comprises a Human Immunodeficiency Virus (HIV) protein or an epitope containing fragment thereof. In some embodiments of any of the aspects, the HIV protein is the glycoprotein 120 neutralizing epitope or glycoprotein 145. In some embodiments of any of the aspects wherein the antigen comprises a Human Immunodeficiency Virus (HIV) protein or an epitope containing fragment thereof, the subject may have or be at risk of having an HIV infection or AIDS (acquired immune deficiency syndrome). In some such embodiments, the disease in the immune distinct subject may be an HIV infection or AIDs. In some embodiments of any of the aspects wherein the antigen comprises a Human Immunodeficiency Virus (HIV) protein or an epitope containing fragment thereof, the method or use may be for inducing an immune response against a Human Immunodeficiency Virus (HIV) optionally to prevent a Human Immunodeficiency Virus (HIV) infection or AIDS. In some embodiments of any of the aspects wherein the antigen comprises a Human Immunodeficiency Virus (HIV) protein or an epitope containing fragment thereof, the method or use may be for prevention or treatment of HIV infection, or AIDS. In some embodiments of any of the aspects wherein the antigen comprises a Human Immunodeficiency Virus (HIV) protein or an epitope containing fragment thereof, the method or use may be for immunizing an immune distinct subject against HIV, optionally to prevent HIV infection or AIDS.

In some embodiments of any of the aspects, the antigen comprises a protein from the Mycobacterium tuberculosis bacterium or an epitope containing fragment thereof. In some embodiments of any of the aspects, the protein from the Mycobacterium tuberculosis bacterium is selected from ESAT-6, Ag85B, TB10.4, Rv2626 and/or RpfD-B. In some embodiments of any of the aspects wherein the antigen comprises a protein from the Mycobacterium tuberculosis bacterium or an epitope containing fragment thereof, the subject may have tuberculosis infection. In some such embodiments, the disease in the immune distinct subject may be a tuberculosis infection In some embodiments of any of the aspects wherein the antigen comprises a protein from the Mycobacterium tuberculosis bacterium or an epitope containing fragment thereof, the method or use may be for inducing an immune response against tuberculosis, optionally to prevent tuberculosis infection. In some embodiments of any of the aspects wherein the antigen comprises a protein from the Mycobacterium tuberculosis bacterium or an epitope containing fragment thereof, the method or use may be for prevention or treatment of a tuberculosis infection. In some embodiments of any of the aspects wherein the antigen comprises a protein from the Mycobacterium tuberculosis bacterium or an epitope containing fragment thereof, the method or use may be for immunizing an immune distinct subject against tuberculosis, optionally to prevent tuberculosis infection.

In some embodiments of any of the aspects, one or more of the first, second, or further ORFs is operatively linked to at least one untranslated region (UTR), wherein each UTR comprises at least a first organ protection sequence (OPS), wherein each OPS comprises at least two micro-RNA (miRNA) target sequences, and wherein each of the at least two miRNA target sequences are optimised to hybridise with a corresponding miRNA sequence. In some embodiments of any of the aspects, each ORF of the composition is operatively linked to a UTR comprising at least one OPS. In some embodiments of any of the aspects, each OPS of the composition independently comprises at least three, at least four, or at least five miRNA target sequences. In some embodiments of any of the aspects, each OPS of the composition independently comprises at least three miRNA target sequences which are all different from each other. In some embodiments of any of the aspects, the first and second ORFs are operatively linked to the same OPS. In some embodiments of any of the aspects, the first and second ORFs are operatively linked to different OPSs. In some embodiments of any of the aspects, the OPS linked to the first ORF and the OPS linked to the second ORF comprise the same miRNA target sequences. In some embodiments of any of the aspects, the OPS linked to the first ORF and the OPS linked to the second ORF each comprise at least one miRNA target sequence not comprised by the other OPS. In some embodiments of any of the aspects, the OPS linked to the first ORF and the OPS linked to the second ORF each comprise at least three miRNA target sequences not comprised by the other OPS. In some embodiments of any of the aspects, the OPS operatively linked to the second ORF comprises miRNA sequences selected to protect one or more organs or tissues selected from the group consisting of muscle, liver, brain, breast, endothelium, pancreas, colon, kidney, lungs, spleen and skin, heart, gastrointestinal organs, reproductive organs, and esophagus. In some embodiments of any of the aspects, the OPS operatively linked to the first ORF comprises miRNA sequences selected to protect one or more organs or tissues selected from the group consisting muscle, liver, brain, breast, endothelium, pancreas, colon, kidney, lungs, spleen and skin. In some embodiments of any of the aspects, the OPS operatively linked to the first ORF comprises miRNA sequences selected to protect one or more organs selected from the group consisting of muscle, liver, kidney, lungs, spleen, skin, heart, gastrointestinal organs, reproductive organs, and esophagus.

In some embodiments of any of the aspects, one or more of the OPS independently comprises:
a) at least two miRNA target sequences selected from one or more sequences that bind to: miRNA-122; miRNA-125; miRNA-199; miRNA-124a; miRNA-126; miRNA-98; Let7 miRNA family; miRNA-375; miRNA-141; miRNA-142; miRNA-148a/b; miRNA-143; miRNA-145; miRNA-194; miRNA-200c; miRNA-203a; miRNA-205; miRNA-1; miRNA-133a; miRNA-206; miRNA-34a; miRNA-192; miRNA-194; miRNA-204; miRNA-215; miRNA-30 family; miRNA-877; miRNA-4300; miRNA-4720; and/or miRNA-6761;
b) sequences selected from one or more of SEQ ID NOs: 44-57;
c) at least two miRNA target sequences selected from sequences capable of binding with miRNA-1, miRNA133a, miRNA206, miRNA-122, miRNA203a, miRNA205, miRNA200c, miRNA30a, and/or let7a/b;
d) at least two miRNA target sequences selected from one or more sequences that bind to: miRNA-1, miRNA-122, miRNA-30a, miRNA-203a, let7b, miRNA-126, and/or miRNA-192;
e) at least two miRNA target sequences selected from sequences capable of binding with miRNA-1, miRNA-122, miR-30a and/or miR-203a;
f) miRNA target sequences capable of binding with miRNA-1, miRNA-122, miRNA-30a and miRNA-203a;
g) miRNA target sequences capable of binding with let7b, miRNA-126, and miRNA-30a;
h) miRNA target sequences capable of binding with miRNA-122, miRNA-192, and miRNA-30a; or
i) miRNA target sequences capable of binding with miRNA-192, miRNA-30a, and miRNA-124, and two miRNA target sequences capable of binding with miRNA 122.

In some embodiments of any of the aspects, the OPS operatively linked to the second ORF comprises miRNA target sequences capable of binding with miRNA-1, miRNA-122, miR-30a and/or miR-203a; and the OPS operatively linked to the first ORF comprises miRNA target sequences capable of binding with miRNA-122, miRNA-126, miRNA-192, and/or miRNA 30a.

In some embodiments of any of the aspects, the administration is intravenous, subcutaneous, intramuscular, intranasal, intra-arterial, or via inhalation. In some embodiments of any of the aspects, the administration is intravenous. In some embodiments of any of the aspects, the first, second, and/or further mRNA constructs are comprised within or adsorbed to an in vivo delivery composition. In some embodiments of any of the aspects, the delivery composition comprises delivery vectors selected from the group consisting of: a particle, such as a polymeric particle; a liposome; a lipidoid particle; and a viral vector. In some embodiments of any of the aspects, the disease is caused by a coronavirus, an intracellular pathogen, a latent infection, an active infection, an influenza virus, respiratory syncytial virus (RSV), human immunodeficiency virus (HIV), varicella zoster virus (VZV), or Mycobacterium tuberculosis; and/or one or more of the antigens are a coronavirus, an intracellular pathogen, a latent infection, an active infection, an influenza virus, respiratory syncytial virus (RSV), human immunodeficiency virus (HIV), varicella zoster virus (VZV), plasmodium (Malaria), Streptococcus pneumoniae, Streptococcus pyogenes, Yersinia pestis, Haemophilus influenzae, Staphylococcus aureus, Pseudomonas aeruginosa, Bordetella pertussis, Ebola virus, Lassa virus, Middle East Respiratory Syndrome coronavirus, SARS-COV-1, SARS-COV-2, SARS-COV-2 variants of concerns, Marburg virus, Nipah virus, Rift Valley Fever virus, Chikungunya virus or Mycobacterium tuberculosis antigen.

In some embodiments of any of the aspects, the disease is caused by a coronavirus and/or one or more of the antigens are a coronavirus antigen. In some embodiments of any of the aspects, the disease is caused by a coronavirus and one or more of the antigens are a coronavirus spike protein. In some embodiments of any of the aspects, the disease is caused by a coronavirus and one or more of the antigens are a coronavirus receptor binding domain protein. In some embodiments of any of the aspects, the disease is caused by MERS and one or more of the antigens are a MERS-COV spike protein. In some embodiments of any of the aspects, the disease is caused by MERS and one or more of the antigens are a MERS-COV receptor binding domain protein. In some embodiments of any of the aspects, the disease is caused by a SARS-COV-1 and one or more of the antigens are a SARS-CoV-1 spike protein. In some embodiments of any of the aspects, the disease is caused by a SARS-CoV-1 and one or more of the antigens are a SARS-COV-1 receptor binding domain protein. In some embodiments of any of the aspects, the disease is caused by a SARS-COV-2 and one or more of the antigens are a SARS-COV-2 spike protein. In some embodiments of any of the aspects, the disease is caused by a SARS-COV-2 and one or more of the antigens are a SARS-COV-2 receptor binding domain protein. In some embodiments of any of the aspects, the coronavirus is MERS-COV virus. In some embodiments of any of the aspects, the coronavirus is SARS-COV-1 virus. In some embodiments of any of the aspects, the coronavirus is SARS-COV-2 virus.

In some embodiments of any of the aspects, the disease is caused by an influenza virus and one or more of the antigens are a hemagglutinin protein. In some embodiments of any of the aspects, the disease is caused by an influenza virus and one or more of the antigens are a neuraminidase protein. In some embodiments of any of the aspects, the disease is caused by an influenza virus and one or more of the antigens are a matrix-2 protein. In some embodiments of any of the aspects, the disease is caused by an influenza virus and one or more of the antigens are a nucleoprotein.

In some embodiments of any of the aspects, the disease is caused by RSV and one or more of the antigens are F glycoprotein. In some embodiments of any of the aspects, the disease is caused by RSV and one or more of the antigens are G glycoprotein.

In some embodiments of any of the aspects, the disease is caused by HIV and one or more of the antigens are glycoprotein 120 neutralizing epitope. In some embodiments of any of the aspects, the disease is caused by HIV and one or more of the antigens are glycoprotein 145.

In some embodiments of any of the aspects, the disease is caused by Mycobacterium tuberculosis and one or more of the antigens are ESAT-6. In some embodiments of any of the aspects, the disease is caused by Mycobacterium tuberculosis and one or more of the antigens are Ag85B. In some embodiments of any of the aspects, the disease is caused by Mycobacterium tuberculosis and one or more of the antigens are TB10.4. In some embodiments of any of the aspects, the disease is caused by Mycobacterium tuberculosis and one or more of the antigens are Rv2626. In some embodiments of any of the aspects, the disease is caused by Mycobacterium tuberculosis and one or more of the antigens are RpfD-B.

### BRIEF DESCRIPTION OF THE DRAWINGS

FIGS. 1A-1G demonstrate that canonical non-adjuvanted LNP vaccines lack IL-12 induction and demonstrate reduced chemokine and Th-1 and Th-2 polarization in elder human whole blood stimulation. FIG. 1A) 24 hr-stimulated human whole blood (WB) with a dose titration of Comirnaty (BNT162b2) ranging from 0.3 μg to 3 μg induced IFNγ (panel 1), CXCL10 (panel 2), CCL3 (panel 3), CCL4 (panel 4), and IL-12p70 (right, n=12-17, detected by cytokine multiplex, adult <60 years old, elder >60). The graphs depict Log 10-transformation of the fold change of each analyte induced divided by the matched RPMI control, followed by a repeated measure ANOVA then one-sided T-test comparing doses of BNT162b2 to RPMI control (horizontal significance bars, color coded by age with adult in red, elder teal), and the two ages (vertical significance bars). GEEGLM analyses evaluating age-dependent functional differences demonstrated 7.4% less TH1 cytokine production in elder participant samples vs adult samples (p=0.027). Percent difference was calculated by exponentiating the point-estimate. Age-separated stacked barplot of the average of each subject's cytokine fold-change, grouped and stacked by (FIG. 1B) Th1-polarizing, (FIG. 1C) Th2-polarizing cytokines, and (FIG. 1D) chemokines. Not-significant (N.S.), and *p<0.05, **p<0.01, ***p<0.001 denoting significance. (FIG. 1A) Log 10-transformation of the fold change of each analyte induced divided by the matched RPMI control, followed by a repeated measure ANOVA then one-sided T-test comparing doses of BNT162b2 to RPMI control (horizontal significance bars, colour coded by age with adult in red, elder teal), and the two ages (vertical significance bars). (FIGS. 1B-1D) Graph denoted the average per group while significance denoted a comparison of generalized estimating equations linear model evaluating significant relationship between participant age and cytokine function on resulting cytokine production. (FIGS. 1E-1G) Each radar plot displayed the per-group average of Log 10-transformation of the fold change of analyte production divided by RPMI control (black) per spoke, in adult (orange-red lines) and elder (blue-teal lines) WBA with escalating BNT162b2 mRNA weights of 0.3 μg (left, FIG. 1E), 1 μg (FIG. 1F), and 3 μg (FIG. 1G). Significance presented above each analyte displays one-sided unpaired T-tests comparing each to negative control, with color-coded asterixis (orange adult, teal elder). Sample sizes for FIGS. 1E-1G were n=5-8, excluding samples with missingness. Significance is denoted by *p<0.05, **p<0.01, ***p<0.001, ****p<0.0001.

FIGS. 2A-2G demonstrate that elder impaired IgG, IgG2a, IgG1, and antibody neutralization in vivo in mice also associated with Th-1 polarized adult and Th-2 polarized elder observations. In vivo intramuscular (IM) vaccination of mice with 0.5 μg to 5 μg Comirnaty (Pfizer) vaccination was administered following a prime-boost schedule separated by 14 days, with serum samples obtained on days 14, 28, and 42 post-prime vaccination. The figures demonstrate age-associated impaired humoral immunity and reduced TH1 polarization is observable in vivo post-BNT162b2 vaccination. (FIG. 2A) On day 42 post-prime a significant induction of spike-specific total IgG (left panel), IgG2a (middle panel), and IgG1 (right panel) was observed in both adult (<6 months, red) and elder mice (>10 months, teal). Elder mice had consistently and significantly impaired antibody production with 3-13-fold lower averages than adult mice, across the 3 isotypes and doses. Adult (6-12 weeks old, red) and Elder (>10 months old, blue) mice were prime boost immunized by intramuscular injection with 0.5, 1, or 5 μg of BNT162b2 mRNA. Humoral immunity to wildtype receptor binding domain (RBD) of SARS-COV-2 spike antigen was quantified on Day 42 post-prime immunization with significant induction of (FIG. 2A) total IgG (left), IgG2a (middle), and IgG1 (right) in adults (red) and elders (teal). Elder mice demonstrated significantly lower Ab titers for each Ig isotype. Th-polarization was evaluated individually with IgG2a, a TH1 marker and IgG1, a TH2 marker. (FIG. 2B) Evaluation of Th-1 and Th-2 polarization supported an adult Th-1 polarized and elder Th-2 polarized phenotype following vaccination with 5 μg of mRNA in Comirnaty by observing IgG2a divided by median IgG1 responses (Pfizer, n=10/group). TH balance was also evaluated by fold-change IgG2a/IgG1 for periodic shifts in Th-polarization on Days 14, 28, and 42 post-prime immunization. (FIG. 2C) Antibody effectiveness was evaluated by pseudo neutralization of a recombinant RBD protein with mouse sera, followed by incubation with human ACE-2 coated ELISA plates, and detection of the amount of RBD capable of binding the ACE2. Ab efficacy was measured by a surrogate virus neutralization test (SVNT) measuring the ability of Ab to reduce RBD binding to human ACE2. A dose-dependent, elder-impairment of Ab efficacy was observed. (FIG. 2D) Correlation analyses of anti-spike IgG by SVNT was significantly correlated and similar in adult and elder mice. (FIG. 2E) Compared to adult mice, sera from aged mice were significantly impaired at neutralizing WA-1 SARS-COV-2 in an in vitro live virus neutralization assay quantifying the dilution required to lose 99% neutralization of cytopathic effects against Vero TMPRSS2 cells. Cellular immunity evaluated by spike-specific peptide restimulation of splenocytes identified SARS-COV-2 specific CD4+ and CD8+ T cell responses by flow cytometry, normalizing age groups by dividing by mean of control mice per age group. (FIG. 2F) CD4+ T cells were evaluated for IFNγ+, IL-2+, TNF+, and IL-4/5+ positivity (left to right), with significantly lower TH1 polarized IFNγ+ and TNF+ responses in elder mice. (FIG. 2G) CD8+ T cell TNF+ cell positivity was evaluated, and significant elder impairment was observed. Each dose in each age group significantly neutralized RBD binding ACE2 compared to control, but elder mice vaccinated with 1 μg or 5 μg were significantly impaired compared to adults vaccinated with the same doses. Sample sizes were n (FIGS. 2A-2D) 5-10, (FIG. 2E) 9, (FIGS. 2F-2G) and 7-8. Significance is denoted by *p<0.05, **p<0.01, ***p<0.001, ***p<0.0001. Statistical tests utilized included Shapiro-Wilk test for normality followed by a one-sided wilcoxon rank-sum test (FIG. 2A), and Grubbs test followed by a Wilcoxon Rank-sum of fold IgG2a over median IgG1 (FIGS. 2B-2C). Normality was determined in FIG. 2E with one-sided Wilcoxon rank-sum test, in FIG. 2D with locally estimated scatterplot smoothing (loess) modelling, in FIG. 2F with two-sided Wilcoxon rank-sum test, and in FIG. 2G with two-sided T-test.

FIGS. 3A-3B demonstrate that sequence-specific, bioactive IL-12 expressed from mRNA LNP in human DCs drives IFNγ induction. mRNA loaded LNP were incubated for FIG. 3A) 24 hr in MoDCs and FIG. 3B) 96 hr in PBMCs. An LNP titration-dependent expression of IL-12 was observed (FIG. 3A). Importantly, IL-12 was not detected using non-coding, scramble mRNA-loaded LNP, indicating sequence specific production (not via self-LNP-adjuvantation). IL-12 bioactivity was confirmed via titration-dependent IFNγ induction. Detection by ELISA, significance, *p<0.05, **p<0.01, ***p<0.001 by one-sided Wilcoxon rank-sum test, n=5.

FIG. 4 demonstrates that older human (60 y plus) PBMCs stimulated 96 hr in vitro displayed titratability in inducing IFNγ post IL-12 mRNA treatment. mRNA loaded LNP were incubated for 96 hr in elder PBMC displayed a dose-dependent induction of IFNγ compared to controls (RPMI and scramble mRNA loaded LNP), indicating functional induction of biologically active IL-12p70 in elder samples. Detection by ELISA, significance, *p<0.05 by one-sided Wilcoxon rank-sum test, n=3.

FIGS. 5A-5E demonstrate that IL-12 mRNA LNP adjuvantation enhanced antigen specific responses on day 28 post-prime immunization with a significant ˜100-fold increase in Th1-associated serology polarization. IL-12 adjuvanticity was evaluated on 2-week separated prime-boost mice with sample obtained day 28 post-prime immunization with mRNA encoding SARS-COV-2 spike antigen or single chain IL-12 heterodimer. Adult mice were IM-immunized with 5 μg mRNA encoding spike ±1 μg mRNA encoding IL-12. On day 28 post-prime immunization an ELISA of spike-specific (FIG. 5A) IgG, (FIG. 5B) IgG2a, and (FIG. 5C) IgG1 was measured with significant 3.7-fold greater IgG2a induced by IL-12 adjuvantation than non-adjuvanted Spike alone. (FIG. 5D) Fold of IgG2a (Th1) over median IgG1 (Th2) measuring functional polarization found significant ˜100-fold increased Th1 polarization with IL-12 adjuvantation comparing IL-12 adjuvantation to non-adjuvanted Spike-alone. IL-12 adjuvantation was also significantly induced over BNT162b2 control at the same timepoint. (FIG. 5E) On Day 42 post-prime a significant induction of spike-specific antibodies over control vaccinated mice, and adjuvantation effect of IL-12 inclusion to induce a significant 3.5-fold greater IgG2a levels was observed. Groups were evaluated by one-sided Wilcoxon rank-sum tests with nonsignificance denoted by N.S., and significance by *p<0.05, **p<0.01, ***p<0.001, ****p<0.0001 (n 4-5/group).

FIGS. 6A-6C demonstrate that IL-12 adjuvantation of CTx mRNA encoding spike antigen induced elder antigen specific responses in vivo, with a trend of adjuvanticity with significant adult 10-fold increase in Th1 linked serology polarization to vaccination with CTx mRNA encoding spike antigen. Elder and adult mice were vaccinated with 5 μg mRNA encoding spike ±5 μg mRNA encoding a single chain IL-12 heterodimer administered with Multi-organ protection for vaccines (MOPv). Following immunization and sample collection (as in FIG. 2), (FIG. 6A) day 42 anti-spike IgG (left), IgG2a (middle), IgG1 (right) antibody titers indicated significant antibody induction in both ages, with a non-significant trend towards adjuvantation in elders. Fold of IgG2a (Th1) over median IgG1 (Th2) functional polarization was evaluated from in vivo mice. The CTx Spk was administered alone, or with mRNA encoding IL-12 (‘adj CTx’) at 1 μg in adult mice, and 5 μg multi-organ protection for vaccines (MOPv) in elder mice. Serum drawn 42 days post-prime was evaluated for Th-polarization by dividing each mouse's observed IgG2a by median IgG1, with resulting medians greater than 1 considered to be Th1-polarized, and less than 1, Th2-polarized. (FIG. 6B) Non-adjuvanted adult mice administered either antigen source had Th-2 polarization which were rescued to a Th1 polarization by IL-12 adjuvantation. (FIG. 6C) Non-adjuvanted elder mice administered Pfizer antigen source induced Th-2 polarization, while elder mice administered non-adjuvanted CTx antigen source yielded a balanced response. IL-12 adjuvantation of elder mice significantly induced greater Th-1 polarization than control, trended towards greater induction that non-adjuvanted CTx control, and was significantly Th-1 polarized compared to Pfizer-sourced antigen. Significance denoted by Not Significant (N.S.), *p<0.05, **p<0.01, with n 4-5 per group, measured by one-sided (FIG. 6A), and two-sided (FIG. 6B) Wilcoxon rank-sum test.

FIG. 7 demonstrates restoration of elder immunogenicity with a tertiary vaccination of CTx mRNA encoding spike antigen. Elder and adult mice were vaccinated with 5 μg mRNA encoding spike. IM immunization was performed in a prime-boost schedule for adult and elder, as well as a prime-boost-boost schedule for elders with 14-day separation between injections to evaluate the ability of a tertiary dose to rescue elder immunosenescence. Serum samples were collected 42 days post-primary injection, with anti-spike IgG (left), IgG2a (middle), and IgG1 (right) quantitated by ELISA. A significant increase in elder immunogenicity was observed in 3-dose compared to 2-dose, and the elder 3-dose was non-inferior to adult 2-dose indicating an alternative mechanism to restoring elder immunogenicity.

FIGS. 8A-8B depict the effect of IL-12 adjuvantation on an alternative SARS-COV-2 mRNA spike antigen source delivered as a single immunization at a low- and medium-dose in vivo in young adult mice. (FIG. 8A) Mice administered a single (1×) immunization with a low-dose 0.05 μg mRNA of BNT162b2 (formulated by ‘Pfizer’)+1 μg IL-12 mRNA were compared to a 2-week separated prime-boost (2×) low-dose 0.05 μg non-adjuvanted Pfizer immunization. Serum was evaluated for anti-spike IgG (left), IgG2a (middle), and IgG1 (right) on day 42 post-prime immunization. IL-12 adjuvantation induced a significant 4.7-fold increase of IgG and 2.8-fold increase of IgG1 over non-adjuvanted control. Adjuvantation of a single shot low-dose immunization induced non-inferior IgG titres compared to prime-boost immunized mice. Mice administered a single (1×) immunization with a (FIG. 8B) medium-dose 0.5 μg mRNA of BNT162b2 (formulated by ‘Pfizer’)+1 μg IL-12 mRNA were compared to a 2-week separated prime-boost (2×) medium-dose 0.5 μg non-adjuvanted Pfizer immunization. Serum was evaluated for anti-spike IgG (left), IgG2a (middle), and IgG1 (right) on day 42 post-prime immunization. IL-12 adjuvantation induced a significant 4.2-fold increase of IgG2a over non-adjuvanted control. Adjuvantation of a single shot medium-dose immunization induced non-inferior IgG titres compared to prime-boost immunized mice. Significance was denoted with *p<0.05, **p<0.01, ***p<0.001, ****p<0.0001 or Not-Significant (N.S.). Statistical tests performed included Kruskal Test and a one-sided wilcoxon rank-sum test (FIGS. 8A, 8B).

FIGS. 9A-9C depict IL-12 adjuvantation of prime-boost low-dose canonical SARS-COV-2 mRNA spike vaccine in vivo in young adult mice on day 42 post-prime. Mice administered prime-boost immunization with a low-dose 0.05 μg mRNA of BNT162b2 (formulated by ‘Pfizer’)±1 μg MOPv-IL-12 mRNA were compared to mice immunized with a 10× and 100× higher dose (0.5 μg, 5 μg, respectively) of prime-boost non-adjuvanted Pfizer immunization. (FIG. 9A) Serum was evaluated for anti-spike IgG (left), IgG2a (middle), and IgG1 (right) on day 42 post-prime immunization. IL-12 adjuvantation induced a significant 8.2-fold increase of IgG, 13.4-fold increase of IgG2a, and 6-fold increase of IgG1 over non-adjuvanted Pfizer-alone control. Adjuvantation induced non-inferior IgG and IgG1 responses to 10× and 100× greater non-adjuvanted Pfizer-alone groups and non-inferior IgG2a responses to 10× greater non-adjuvanted Pfizer-alone group. Spike-specific peptide restimulation of T cells were observed by flow cytometry for (FIG. 9B) CD4+ Th-1 polarization (IFN+, far left panel; IL2+, middle left; TNF+, middle right) and Th-2 polarization (IL4+ IL5+, far right). IL-12 adjuvantation trended towards greater IFN, IL2, and TNF CD4+ cell positivity, and was significantly greater than negative control for all 3 cytokines while non-adjuvanted Pfizer-alone was only significantly greater than background for IFN+ cells. Th-2 polarization was not noted for any group. (FIG. 9C) Restimulated T cells were also evaluated for CD8+ T cell activity with IFN+ (left) and TNF+ (right) activity, with no significant induction observed at this limiting low dose. Significance was denoted with *p<0.05, **p<0.01, ***p<0.001, ****p<0.0001 or Not-Significant (N.S.). Statistical tests performed included Kruskal Test and a one-sided Wilcoxon rank-sum test (FIG. 9A), and Kruskal Test with two-sided Wilcoxon rank-sum tests (FIGS. 9B, 9C).

FIGS. 10A-10C depict IL-12 adjuvantation of prime-boost medium-dose canonical SARS-CoV-2 mRNA spike vaccine in vivo in young adult mice on day 42 post-prime. Mice administered prime-boost immunization with a medium-dose 0.5 μg mRNA of BNT162b2 (formulated by ‘Pfizer’)±1 μg IL-12 mRNA were compared to mice immunized with a 10× higher dose (5 μg) of prime-boost non-adjuvanted Pfizer immunization. (FIG. 10A) Serum was evaluated for anti-spike IgG (left), IgG2a (middle), and IgG1 (right) on day 42 post-prime immunization. IL-12 adjuvantation induced a significant 5.4-fold increase of IgG, and 11.8-fold increase of IgG2a over non-adjuvanted Pfizer-alone control. Adjuvantation induced non-inferior IgG, IgG2a, and IgG1 response to a 10× greater non-adjuvanted Pfizer-alone group. Spike-specific peptide restimulation of T cells were observed by flow cytometry for (FIG. 10B) CD4+ Th-1 polarization (IFN+, far left panel; IL2+, middle left; TNF+, middle right) and Th-2 polarization (IL4+ IL5+, far right). A significant induction of the Th-1 signature, IFN+ CD4+ T cells, was observed in adjuvanted mice compared to the non-adjuvanted Pfizer-alone group. A trend towards increased Th-1 associated IL-2+ and TNF+ CD4+ cells and Th-2 associated IL4+ IL5+ CD4+ cells were also observed. (FIG. 10C) Restimulated T cells were also evaluated for CD8+ T cell activity with IFN+ (left) and TNF+ (right) activity. Both non-adjuvanted and adjuvanted immunizations induced significant CD8 T cell responses compared to negative control, and IL-12 adjuvantation trended towards greater IFN+ and TNF+ CD8+ T cells than non-adjuvanted Pfizer-alone. Significance was denoted with *p<0.05, **p<0.01, ***p<0.001, ***p<0.0001 or Not-Significant (N.S.). Statistical tests performed included Kruskal Test and a one-sided Wilcoxon rank-sum test (FIG. 10A), and Kruskal Test with two-sided Wilcoxon rank-sum tests (FIGS. 10B, 10C).

FIGS. 11A-11C demonstrate that IL-12 adjuvantation promotes robust immunity against SARS-COV-2 Spike in elder mice. Elder mice (>10 months old) were intramuscularly immunized following a prime-boost, 14 day separated schedule and compared to adult (˜6 week). Immunizations were with control dPBS, or 0.05 to 5.0 μg of encapsulated mRNA encoding spike protein (Pfizer's BNT162b2, ‘Pfz’) with or without 5 or 1 μg of encapsulated mRNA encoding a single-chain IL-12 heterodimer. (FIG. 11A) On day 42 post-prime, 28 post-booster immunization, humoral immunity was evaluated in mouse sera for anti-spike antibody production, specifically total IgG (left), IgG2a (middle, Th1 marker), IgG1 (right, Th2 marker). Significant induction over negative control, and adjuvantation over antigen-alone was observed in each antibody isotype. Antibody induction was non-inferior to adults administered the same antigen dose, supporting restored function, and were non-inferior to a 10× higher dose in elders for total IgG and IgG2a, supporting dose sparing. (FIG. 11B) Splenocytes were collected on day 42 post-prime immunization, restimulated with spike-specific peptide, and a significant 2.1-fold greater frequency of parent (FoP) CD4+ cell IFN⁺ positivity was observed in adjuvanted versus non-adjuvanted groups. (FIG. 11C) CD4+ T cell production of IL-4 and/or IL-5 (IL4.5) as markers of Th2 polarization indicated that IL-12 adjuvantation significantly induced more cell positivity than non-adjuvanted elder control and more than non-adjuvanted adult control. Adjuvant-amplified IL4.5 positivity was non-inferior to mice with 10× and 100× the antigen dose. Significance was determined in n 5-10 mice in FIG. 11A by unpaired one-sided Wilcoxon rank-sum tests comparing to negative control and two-sided Wilcoxon rank-sum tests between adjuvanted and non-adjuvanted groups. In FIG. 11B-11C an n 5-10 mice were tested for normality by a Shapiro-Wilk test, followed by the non-parametric Kruskal-Wallis and unpaired two-sided Wilcoxon rank-sum tests. Significance was denoted by *p<0.05, **p<0.01, ***p<0.001, ****p<0.0001.

FIGS. 12A-12D: Waning immunity in non-adjuvanted BNT162b2-alone, and IL-12 adjuvant-sustained immune durability in adult mice through d259 post-prime vaccination. Adult mice (6-10 weeks at time of vaccination) administered a prime-boost immunization of 0.05 μg or 0.5 μg mRNA encapsulated within BNT162b2 (formulated by ‘Pfizer/BioNTech’)±1 μg mRNA encoding IL-12+/−MOP (formulated by CTx). Serum was evaluated for anti-spike IgG (left), IgG2a (middle), and IgG1 (right) on days 28, 42, 84, 168, and 259 post-prime immunization. (FIGS. 12A, 12B) Day 259 serum results of mice vaccinated with 0.05 μg BNT162b2 adjuvanted with 1 μg IL-12, +/−MOP to control adjuvant expression, had amplified total IgG, IgG2a, and IgG1 over non-adjuvanted BNT162b2-alone group. Additionally, IL-12 adjuvantation rescued 100% of mice, while non-adjuvanted, BNT162b2-alone mice, were 40% non-responsive (NR) for IgG, 60% NR for IgG2a, and 40% NR for IgG1. (FIGS. 12C, 12D) Day 259 serum results of mice vaccinated with a 10× higher mRNA encoding antigen dose, 0.5 μg of mRNA encapsulated in BNT162b2, with IL-12 adjuvantation had a significantly greater IgG2a compared to non-adjuvanted. N=9-10. Significance was denoted with *p<0.05, **p<0.01, ***p<0.001, ****p<0.0001 or Not-Significant (N.S.). Statistical tests performed included Kruskal Test and one-sided wilcoxon rank-sum tests (FIGS. 12A-12D).

FIGS. 13A-13D. Overcoming prolonged waning immunity in elder (immune-distinct) mice. Elder (>10 months of life) female mice were immunized with (FIGS. 13A-13B) 0.05 μg mRNA BNT162b2 or (FIGS. 13C-13D) 0.5 μg BNT162b2 with or without IL-12 adjuvantation alongside (FIGS. 13C-13D) 5.0 μg BNT162b2 as a benchmark for maximal immunity via a 14-day prime-boost schedule. Spike-specific IgG (left panels), IgG2a (middle panels), and IgG1 (right panels) antibody production was measured in sera. (FIG. 13A) Elder mice by day 168 post-prime 0.05 μg-immunization had significant waning immunity for isotypes IgG and IgG1. (FIG. 13B) 80-90% of nonadjuvanted 0.05 μg-immunized elder mice were nonresponsive (NR) by d168, while IL-12 adjuvantation kept this to 56% nonresponsive, significantly inducing greater antibody compared to negative control and non-adjuvanted. These adjuvanted elder mice were non-inferior to adult mice indicating restoration of elder immunity to a young-adult phenotype. (FIG. 13C) Elder waning immunity was also observed following 0.5 μg immunization for each isotype on days 42, 84, and 168 post-prime immunization, with a noted decrease between day 84 and 168 for IgG and IgG1. (FIG. 13D) By day 168 elder IL-12 adjuvantation led to more durable IgG, IgG2a, and IgG1 response over non-adjuvanted 0.5 μg immunized mice to a degree that was non-inferior to a 10× higher non-adjuvanted 5.0 μg immunization dose displaying sustained adjuvanticity and a durable dose-sparing effect. Significance was evaluated by (FIGS. 13A, 13C) one-sided Wilcoxon rank-sum tests comparing each day to day 28-post-prime immunization, and (FIGS. 13B, 13D) one-sided Wilcoxon rank-sum tests evaluating IL-12 adjuvanticity over non-adjuvanted or two-sided non-inferiority to either adult benchmarks (FIG. 13B) or 10× higher antigen dose (FIG. 13D). Significance was denoted with *p<0.05, **p<0.01, ***p<0.001, ****p<0.0001, with nonsignificance denoted by “N.S.” and nonresponsive mice (those at limit of detection) noted by “NR”.

FIGS. 14A-14F: Mechanistic investigation of IL-12 adjuvantation effects that supported amplified antibody production, isotype class switching, and cell polarization. (FIG. 14A) Draining lymph nodes were aseptically weighed by differential weight pre- and post-dissection, and IL-12 adjuvantation had significantly greater weight than non-adjuvanted and were noninferior to adult mice immunized with a non-adjuvanted formulation suggesting restored immunity. (FIG. 14B) DLN were collected 9 days post-booster immunization and evaluated by flow cytometry where dendritic cells were quantified as CD3-CD19-MHC.II+ CD11c+ CD14−. Back calculation of frequency to total cell content within the DLN identified adjuvant effects with IL-12 in elder mice, significantly greater than non-adjuvanted control. Follicular dendritic cells (FDC) were identified by CD21/CD35 detection, B cell zone by naïve B cell IgD expression, and Germinal centers by GL7. Significance was denoted with *p<0.05, **p<0.01, ***p<0.001, ****p<0.0001, with nonsignificance denoted by “N.S.”. Shapiro wilk test for normality was followed by either a one-sided T-test (FIG. 14C) or one-sided Wilcoxon test (FIGS. 14A, 14B, 14D-14F) hypothesizing adjuvanted greater than non-adjuvanted, or a two-sided test for evaluating inferiority.

FIGS. 15A-15B: extended potency effects of IL-12 adjuvantation on humoral and cell mediated immunity. Elder (>10 months of life) female mice were immunized with 0.1 μg mRNA in BNT162b2 with or without IL-12-MOP adjuvantation via a 14-day prime-boost schedule. (FIG. 15A) Serum from day 28-29 post-prime identified spike-specific IgG (left panel), IgG2a (middle panel), and IgG1 (right panel) antibody production. IL-12-MOP was conferred adjuvantation effects at 0.1 μg and 0.3 μg of mRNA encoding IL-12, up to 50× lower than some previous doses. (FIG. 15B) On day 28-29 post-prime immunization splenocytes were processed to a single cell suspension and had red blood cells lysed. Splenocytes were serially diluted and stimulated with spike-specific peptide in an ELISPOT experiment quantifying IFNγ secreting spot forming cells (SFC) to measure cellular immunity. IL-12 adjuvanted immune responses greater than antigen alone (0.1 μg Pfz), to a level non-inferior to a 10× higher antigen dose, (1.0 μg Pfz). Significance was evaluated by (FIGS. 15A, 15B) one-sided Wilcoxon rank-sum test evaluating IL-12 adjuvanticity over non-adjuvanted, and (FIG. 15B) evaluating inferiority to 10× higher antigen dose, 0.1 μg mRNA in BNT162b2 (Pfz). Significance was denoted with *p<0.05, **p<0.01, ***p<0.001, with percent of nonresponsive mice (those at limit of detection) noted by “NR”.

### DETAILED DESCRIPTION

The responsivity and efficacy of the human immune system varies with age and disease. Several populations/groups of humans share variations in their immune system function that render them more susceptible to infectious disease, as compared to healthy mature adults. These patients are collectively referred to as “immune distinct.” Immune distinct patients may be immunocompromised, but may also have immune systems optimized for non-infectious conditions and not be immunocompromised per se. For example, infants and elderly individuals are immune distinct and this variation in their immune systems is believed to provide other advantages, such as minimizing wasted inflammatory responses. This phenomenon of immune distinct patients is well known in the art and can be characterized by several different biomarkers and structural characteristics. For example, see Kollmann et al. Immunity 2012 37:771-783, which is incorporated by reference herein in its entirety. In some embodiments of any of the aspects, an immune distinct subject has increased IL-10 production. In some embodiments of any of the aspects, an immune distinct subject has decreased IL-12 production. In some embodiments of any of the aspects, an immune distinct subject has decreased IFN-alpha production. In some embodiments of any of the aspects, an immune distinct subject has decreased TNF production. In some embodiments of any of the aspects, an immune distinct subject has decreased IL-1 production. This weakened defense against infectious disease also manifests as a reduced response to vaccination. Accordingly, there is a need for improved methods of immunizing or vaccinating immune distinct patients.

Immune distinct patients demonstrate slow initiation, low immunogenicity and reduced persistence of functional antibodies (Abs) and cell-mediated responses in response to vaccination with standard adjuvants (Dowling D J, and Levy O. Trends Immunol. 2014; 35(7):299-310). Yet vaccine development has relied primarily upon traditional alum-based adjuvantation for most of the modern era (Rappuoli et al. Nat Rev Immunol. 2011; 11(12):865-72). While most adjuvants have become available in the 21^{st} century, these adjuvants are typically developed in and for normal health adults and show comparatively poor performance when utilized in immune distinct patients. The exceptions are primarily self-adjuvanted vaccines, which by their intrinsic nature cannot provide adjuvantation for a spectrum of antigens or emerging diseases. As described herein, the inventors have found that when proinflammatory cytokines are provided as an adjuvant, the immune response of an immune distinct subject to vaccination/immunization is surprisingly improved, providing much greater protective immune responses. The magnitude of the increase in the protective immune response is sufficient to permit much smaller doses or abbreviated administration regimes as compared to the absence of the proinflammatory cytokine adjuvants.

Accordingly, in one aspect of any of the embodiments, described herein is a method for a) inducing an immune response in an immune distinct subject, b) treating or preventing a disease in an immune distinct subject and/or c) immunizing an immune distinct subject, the method comprising administering to the subject one or more compositions comprising a first cytokine mRNA construct comprising a first open reading frame (ORF), wherein the first ORF encodes a proinflammatory cytokine. In some embodiments of any of the aspects, the method further comprises administering an antigen or antigens, in the same composition or a separate composition. As used herein to refer to an ORF, cytokine mRNA construct, or antigen mRNA, “first” refers to at least one element and any “further”, “second”, or “third” elements denote elements in addition to the “first” element, without being limited to a specific physical 5′ to 3′ order of the multiple elements.

In some embodiments of any of the aspects, a mRNA construct(s) encoding one or more proinflammatory cytokines can be administered in conjunction with one or more of a subunit, toxoid, mRNA, killed, or attenuated vaccine, e.g., a vaccine providing an antigen as a mRNA, peptide, protein, lipid, lipo-protein, carbohydrate/sugar, conjugate (protein-carbohydrate), hapten-protein, killed vaccines, attenuated vaccines, etc. In some embodiments of any of the aspects, a mRNA construct(s) encoding one or more proinflammatory cytokines can be administered in the same composition as a subunit, toxoid, mRNA, killed, or attenuated vaccine. In some embodiments of any of the aspects, a mRNA construct(s) encoding one or more proinflammatory cytokines can be administered in a separate composition as a subunit, toxoid, mRNA, killed, or attenuated vaccine. In some embodiments of any of the aspects, a mRNA construct(s) encoding one or more proinflammatory cytokines can be administered prior to a subunit, toxoid, mRNA, killed, or attenuated vaccine e.g., as a “priming” composition. In some embodiments of any of the aspects, a mRNA construct(s) encoding one or more proinflammatory cytokine scan be administered after a subunit, toxoid, mRNA, killed, or attenuated vaccine e.g., as a “booster” composition. In some embodiments of any of the aspects, the booster composition can be a heterologous booster, e.g., comprising a different antigen than the initial vaccine.

In some embodiments, the antigen(s) can be provided in a mRNA construct. Accordingly, in one aspect of any of the embodiments, described herein is a method for inducing an immune response in an immune distinct subject, the method comprising administering to the subject one or more compositions comprising:
a) a first cytokine mRNA construct comprising a first open reading frame (ORF), wherein the first ORF encodes a proinflammatory cytokine; and
b) a first antigen mRNA construct comprising a second open reading frame (ORF), wherein the second ORF encodes an antigen.
   In one aspect of any of the embodiments, described herein is a method for treating or preventing a disease in an immune distinct subject, the method comprising administering to the subject one or more compositions comprising:
a) a first cytokine mRNA construct comprising a first open reading frame (ORF), wherein the first ORF encodes a proinflammatory cytokine; and
b) a first antigen mRNA construct comprising a second open reading frame (ORF), wherein the second ORF encodes an antigen.
   In one aspect of any of the embodiments, described herein method for immunizing an immune distinct subject, the method comprising administering to the subject one or more compositions comprising:
a) a first cytokine mRNA construct comprising a first open reading frame (ORF), wherein the first ORF encodes a proinflammatory cytokine; and
b) a first antigen mRNA construct comprising a second open reading frame (ORF), wherein the second ORF encodes an antigen.
   A mRNA construct comprising a first open reading frame (ORF) encoding a proinflammatory cytokine is sometimes referred to herein as an adjuvant construct, as the proinflammatory cytokine functions as an adjuvant. A mRNA construct comprising a second open reading frame (ORF) encoding an antigen is sometimes referred to herein as an antigen construct.

In some embodiments of any of the aspects, the adjuvant construct and antigen construct are provided or administered in a single composition. In some embodiments of any of the aspects, the adjuvant construct and antigen construct are provided or administered in a single molecule, e.g., a mRNA molecule comprising multiple constructs. In some embodiments of any of the aspects, the adjuvant construct and antigen construct are provided or administered in separate compositions. In some embodiments of any of the aspects, the adjuvant construct and antigen construct are provided or administered concurrently. In some embodiments of any of the aspects, the adjuvant construct and antigen construct are provided or administered sequentially. In some embodiments of any of the aspects, the adjuvant construct and antigen construct are provided or administered sequentially, with the antigen construct being provided or administered first. In some embodiments of any of the aspects, the adjuvant construct and antigen construct are provided or administered sequentially, with the adjuvant construct being provided or administered first.

Where present on separate mRNA constructs, and formulated to be associated with delivery particles (as described elsewhere herein), these separate mRNA constructs may be co-formulated, such that different mRNA constructs may be associated with the same individual delivery particles, or separately formulated, such that different mRNA constructs may be associated with different delivery particles.

Delivery of mRNA directly to cells allows direct and controllable translation of the desired gene products such as polypeptides and/or proteins in the cells. Provision of mRNA specifically allows not only for the use of cell expression modulation mechanisms, such as miRNA mediated control (as detailed in specific embodiments below), but also represents a finite and exhaustible supply of the product, rather than the potentially permanent change to the transcriptome of a target cell, which an episomal or genomically inserted DNA vector might provide.

The term “vaccine” used herein is defined as a composition used to elicit an immune response against an antigen within the composition in order to protect or treat an organism against disease. In some embodiments of any of the aspects, the vaccine is a suspension of attenuated or killed microorganisms (e.g., viruses, bacteria, or rickettsia), or of antigenic proteins derived from them, administered for prevention, amelioration, or treatment of infectious diseases. Alternatively, the vaccine can comprise or be an mRNA composition/construct or vector comprising an mRNA composition/construct as described herein. The terms “vaccine composition” and “vaccine” are used interchangeably. The term “vaccinate” refers to the act of administering a vaccine to a subject.

The term “immunize” as used herein is defined as elicit an immune response, e.g., either a cellular (T-cell) or humoral (B-cell or antibody) response, or both, as measured by standard assays known to one skilled in the art.

As used herein in the context of immunization, immune response and vaccination, the term “adjuvant” refers to any substance than when used in combination with a specific antigen that produces a more robust immune response than the antigen alone. When incorporated into a vaccine formulation, an adjuvant acts generally to accelerate, prolong, or enhance the quality of specific immune responses to the vaccine antigen(s).

As used herein, an “immune response” refers to a response by a cell of the immune system, such as a B cell, T cell (CD4 or CD8), regulatory T cell, antigen-presenting cell, dendritic cell, monocyte, macrophage, NKT cell, NK cell, basophil, eosinophil, or neutrophil, to a stimulus (e.g., to an adjuvant). In some embodiments of the aspects described herein, the response is specific for a particular antigen (an “antigen-specific response”), and refers to a response by a CD4 T cell, CD8 T cell, or B cell via their antigen-specific receptor. In some embodiments of the aspects described herein, an immune response is a T cell response, such as a CD4+ response or a CD8+ response. Such responses by these cells can include, for example, cytotoxicity, proliferation, cytokine or chemokine production, trafficking, or phagocytosis, and can be dependent on the nature of the immune cell undergoing the response. Stimulation of an immune response refers to an induction or increase of the immune response.

An immune response to an antigen and/or adjuvant can be the development in a subject of a humoral and/or a cell-mediated immune response to molecules present in the antigen or vaccine composition of interest. For purposes of the present invention, a “humoral immune response” is an antibody-mediated immune response and involves the induction and generation of antibodies that recognize and bind with some affinity for the antigen in the immunogenic composition of the invention, while a “cell-mediated immune response” is one mediated by T-cells and/or other white blood cells. A “cell-mediated immune response” is elicited by the presentation of antigenic epitopes in association with Class I or Class II molecules of the major histocompatibility complex (MHC), CD1 or other non-classical MHC-like molecules. This activates antigen-specific CD4+ T helper cells or CD8+ cytotoxic lymphocyte cells (“CTLs”). CTLs have specificity for peptide antigens that are presented in association with proteins encoded by classical or non-classical MHCs and expressed on the surfaces of cells. CTLs help induce and promote the intracellular destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide or other antigens in association with classical or non-classical MHC molecules on their surface. A “cell-mediated immune response” also refers to the production of cytokines, chemokines and other such molecules produced by activated T-cells and/or other white blood cells, including those derived from CD4+ and CD8+ T-cells. The ability of a particular antigen or composition to stimulate a cell-mediated immunological response may be determined by a number of assays, such as by lymphoproliferation (lymphocyte activation) assays, CTL cytotoxic cell assays, by assaying for T-lymphocytes specific for the antigen in a sensitized subject, or by measurement of cytokine production by T cells in response to re-stimulation with antigen. Such assays are well known in the art. See, e.g., Erickson et al. (1993) J. Immunol. 151:4189-4199; and Doe et al. (1994) Eur. J. Immunol. 24:2369-2376.

In some embodiments of any of the aspects, the immune response comprises an increase in IL-12 in the subject. In some embodiments of any of the aspects, the immune response comprises an increase in active IL-12 heterodimer (referred to herein as “p70”) in the subject.

In some embodiments of any of the aspects, the immune response comprises an increase in Ig levels in the subject. Humans have four Ig subclasses; IgG1, IgG2, IgG3, and IgG4. IgG2 and IgG3 are most indicative of inflammatory and desired vaccine-induced responses, respectively. In some embodiments of any of the aspects, the Ig is IgG2, IgG3, or IgG2a. In some embodiments of any of the aspects, the Ig is IgG2. In some embodiments of any of the aspects, the Ig is IgG3. In some embodiments of any of the aspects, the Ig is IgG2a. In some embodiments of any of the aspects, the Ig is an Ig that specifically binds the antigen encoded by one or more of the antigen constructs administered to the subject. In some embodiments of any of the aspects, the Ig is an IgG2a that specifically binds the antigen encoded by one or more of the antigen constructs administered to the subject.

In some embodiments of any of the aspects, the immune response comprises a CD4+ T cell response in the subject. In some embodiments of any of the aspects, an immune response can be cytokine production by CD4+ T cells. In some embodiments of any of the aspects, cytokine production by a CD4+ T cell can comprise production of one or more of IL-2 (proliferation); IL-2, IFN-γ, TNF, TNF-β (Th1); IL-4, IL-5, IL-9 and IL-13 (Th2); IL-1-β, IL-17A, IL-17E, IL-17F, IL-21, IL-22, IL-23 (Th17); IL-6, IL21, (Tfh); TGF-β, IL-10, IL-35 (multiple and Tregs). In some embodiments of any of the aspects, an immune response can be an increase in the level of CD4+ T cells, e.g., antigen-specific CD4+ cells.

In some embodiments of any of the aspects, the immune response comprises a CD8+ T cell response in the subject. In some embodiments of any of the aspects, an immune response can be cytokine production by CD8+ T cells. In some embodiments of any of the aspects, cytokine production by a CD8+ T cell can comprise production of one or more of IL-2, IFN-γ, TNF, and IL-10. In some embodiments of any of the aspects, an immune response can be the release of perforin and/or granzymes by CD8+ T cells. In some embodiments of any of the aspects, an immune response can be an increase in the level of CD8+ T cells.

In some embodiments of any of the aspects, the immune response comprises a Th1 cell response in the subject. In some embodiments of any of the aspects, an immune response can be cytokine production by Th1 cells. In some embodiments of any of the aspects, an immune response can be an increase in the level of Th1 cells, e.g., antigen-specific Th1 cells.

In some embodiments of any of the aspects, the immune response comprises a NK cell response in the subject. In some embodiments of any of the aspects, an NK cell response comprises the production of one or more of IFN-γ and TNF. In some embodiments of any of the aspects, an immune response can be the release of perforin and/or granzymes by NK cells. In some embodiments of any of the aspects, an immune response can be an increase in the level of NK cells.

In some embodiments of any of the aspects, the immune response stimulates or is an increase of the production of an interferon gamma (IFNγ) response from T cells in the subject, e.g., an increase in IFNγ levels.

In some embodiments of any of the aspects, the immune response initiates or comprises an increase in phagocytosis via the Fc region of each IgG subclass via improved affinity for phagocyte membrane Fc-gamma-receptors (FcγR).

In some embodiments of any of the aspects, the immune response comprises immunization of the subject against the antigen or an organism comprising the antigen.

In some embodiments of any of the aspects, the immune distinct subject is a subject with immunosenescence. The terms “immunosenescence” or “immunosenescent” refer to a decrease in immune function resulting in impaired immune response, e.g., to cancer, vaccination, infectious pathogens, among others. It involves both the host's capacity to respond to infections and the development of long-term immune memory, especially by vaccination. It is considered a major contributory factor to the increased frequency of morbidity and mortality among the elderly.

Immunosenescence is a multifactorial condition leading to many pathologically significant health problems, e.g., in the aged population. Age-dependent biological changes such as depletion of hematopoietic stem cells, an increase in PD1+ lymphocytes, a decline in the total number of phagocytes and NK cells and a decline in humoral immunity contribute to the onset of immunosenescence. In one aspect, immunosenescence can be measured in an individual by measuring telomere length in immune cells (See, e.g., U.S. Pat. No. 5,741,677). Immunosenescence can also be determined by documenting in an individual a lower than normal number of naive CD4 and/or CD8 T cells, T cell repertoire, the number of PD1-expressing T cells, e.g., a lower than normal number of PD-1 negative T cells, or response to vaccination in a subject greater than or equal to 65 years of age.

In some embodiments of any of the aspects, the immune distinct subject and/or subject with immunosenescence is a subject of 55 years of age or older. In some embodiments of any of the aspects, the immune distinct subject and/or subject with immunosenescence is a subject of 60 years of age or older. In some embodiments of any of the aspects, the immune distinct subject and/or subject with immunosenescence is a subject of 65 years of age or older. In some embodiments of any of the aspects, the immune distinct subject and/or subject with immunosenescence is a subject of 70 years of age or older. In some embodiments of any of the aspects, the immune distinct subject and/or subject with immunosenescence is a subject of 75 years of age or older.

An immune distinct or immunosensent patient can be distinguished from a normal healthy adult in that they have a reduced TNF and/or IL-12 response to immune stimuli. In some embodiments of any of the aspects, the immune distinct subject and/or subject with immunosenescence is a subject who has or is determined to have a reduced TNF and/or IL-12 response to immune stimuli. In some embodiments, the immune stimuli is a microbe-associated molecular pattern (MAMP). In some embodiments, the immune stimuli is lipopolysaccharide (LPS).

Infants are also known to be immune distinct. In some embodiments of any of the aspects, the immune distinct subject is an infant. In some embodiments of any of the aspects, the immune distinct subject and/or infant is 2 years of age or younger. In some embodiments of any of the aspects, the immune distinct subject and/or infant is 1 year of age or younger. In some embodiments of any of the aspects, the immune distinct subject and/or infant is 28 days of age or younger. In some embodiments of any of the aspects, the immune distinct subject and/or infant is or was born preterm.

Patients suffering from certain conditions or undergoing certain procedures are also known to be immune distinct. In some embodiments of any of the aspects, the immune distinct subject is immunocompromised, has an HIV infection, has AIDS, has received a transplant, is undergoing immunosuppression, is immunosuppressed, has an infection, is diabetic, has an IgG subclass deficiency, has a substance abuse disorder, or is obese. An IgG subclass deficiency is a decrease in serum concentration of one or more subclasses of IgG in patient, compared to a normal healthy adult, while the patient's total IgG concentration remains the same as that found in a normal healthy adult.

An immune deficient subject is at significantly higher risk of contracting infectious disease and/or suffering severe symptoms of infectious disease when they are resident in a high density living environment. In some embodiments of any of the aspects, the subject, e.g., the immune deficient subject is a subject in/residing in a high density living environment. High density living environments are those environments in there are multiple dwelling units in single building and there are either communal living spaces (e.g., communal restrooms, recreational, and/or dining facilities), or non-residents have regular access to the dwelling units (e.g., nursing or maintenance/cleaning staff access to hospital rooms). Exemplary but non-limiting high density living environments include assisted living facilities; prisons or jails; nursing homes, dormitories; barracks; and hospitals.

In some embodiments of any of the aspects, the subject is a subject who is:
a) at least 55 years of age, at least 60 years of age, at least 65 years of age, at least 70 years of age, or at least 75 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
   In some embodiments of any of the aspects, the subject is a subject who is:
a) at least 55 years of age, at least 60 years of age, at least 65 years of age, at least 70 years of age, or at least 75 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder and/or is obese.
   In some embodiments of any of the aspects, the subject is a subject who is:
a) at least 55 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high-density living environment.
   In some embodiments of any of the aspects, the subject is a subject who is:
a) at least 55 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder and/or is obese.

As described elsewhere herein, the methods and compositions described herein permit lower dosing and/or reduced administration frequency in immune distinct patients, while still providing the same or improved immune responses.

In some embodiments of any of the aspects, the composition(s) comprises at least 5× less of the antigen than is required to induce an immune response in the absence of the first cytokine mRNA. In some embodiments of any of the aspects, the composition(s) comprises at least 10× less of the antigen than is required to induce an immune response in the absence of the first cytokine mRNA. In some embodiments of any of the aspects, the composition(s) comprises at least 15× less of the antigen than is required to induce an immune response in the absence of the first cytokine mRNA. In some embodiments of any of the aspects, the composition(s) comprises at least 20× less of the antigen than is required to induce an immune response in the absence of the first cytokine mRNA. In some embodiments of any of the aspects, the composition(s) comprises at least 50× less of the antigen than is required to induce an immune response in the absence of the first cytokine mRNA. In some embodiments of any of the aspects, the composition(s) comprises at least 100× less of the antigen than is required to induce an immune response in the absence of the first cytokine mRNA.

In some embodiments of any of the aspects, the method comprises administering each of the one or more compositions to the immune distinct subject no more frequently than twice per year. In some embodiments of any of the aspects, the method comprises administering each of the one or more compositions to the immune distinct subject no more frequently than once per year. In some embodiments of any of the aspects, the method comprises administering each of the one or more compositions to the immune distinct subject no more frequently than once every two years. In some embodiments of any of the aspects, the method comprises administering each of the one or more compositions to the immune distinct subject no more frequently than once every 3 years. In some embodiments of any of the aspects, the method comprises administering each of the one or more compositions to the immune distinct subject no more frequently than once every 4 years. In some embodiments of any of the aspects, the method comprises administering each of the one or more compositions to the immune distinct subject no more frequently than once every 5 years.

The advantages of the compositions described herein in stimulating effective immune responses in immune distinct patients, reduces the need for booster vaccines. In some embodiments of any of the aspects, the first antigen mRNA construct antigen is an antigen of a first infectious organism and the method comprises administering a composition comprising any antigen from the first infectious organism to the immune distinct subject no more frequently than once per year. In some embodiments of any of the aspects, the first antigen mRNA construct antigen is an antigen of a first infectious organism and the method comprises administering a composition comprising any antigen from the first infectious organism to the immune distinct subject no more frequently than once every 2 years. In some embodiments of any of the aspects, the first antigen mRNA construct antigen is an antigen of a first infectious organism and the method comprises administering a composition comprising any antigen from the first infectious organism to the immune distinct subject no more frequently than once every 3 years. In some embodiments of any of the aspects, the first antigen mRNA construct antigen is an antigen of a first infectious organism and the method comprises administering a composition comprising any antigen from the first infectious organism to the immune distinct subject no more frequently than once every 4 years. In some embodiments of any of the aspects, the first antigen mRNA construct antigen is an antigen of a first infectious organism and the method comprises administering a composition comprising any antigen from the first infectious organism to the immune distinct subject no more frequently than once every 5 years.

Cytokines are a broad category of small proteins important in cell signaling. Cytokines have been shown to be involved in autocrine, paracrine and endocrine signaling and are immunomodulating agents. Cytokines include chemokines, interferons, interleukins, lymphokines, and tumor necrosis factors. Cytokines are produced by a broad range of cells, including immune cells like macrophages, B lymphocytes, T lymphocytes and mast cells, as well as endothelial cells, fibroblasts, and various stromal cells; a given cytokine may be produced by more than one type of cell. They act through cell surface receptors and are especially important in the immune system; cytokines modulate the balance between humoral and cell-based immune responses, and they regulate the maturation, growth, and responsiveness of particular cell populations. Cytokines have been classed as interleukins, lymphokines, monokines, interferons, colony stimulating factors and chemokines.

In some embodiments of any of the aspects, the proinflammatory cytokine encoded by the first cytokine construct is selected from the group consisting of: IL-12; IL-2; IL-4; IL-5; IL-6; IL-8; IL-10; IL-13; IL-27; IL-1β; TGFβ; IFNγ; IFNα; IFNβ; TNFα; CCL2; CCL3; CCL4; CCL5; CCL8; CXCL12; GM-CSF; and a subunit, dimer, heterodimer, derivative, fragment, agonist or homologue thereof. The sequences for the foregoing genes, e.g., their genomic, coding, mRNA, and polypeptide sequences, are known in the art for a number of species. By way of non-limiting example, the human sequences can be found in the NCBI Database, e.g., under the following Gene ID numbers. In some embodiments of any of the aspects, the sequences are the sequences available for the indicated Gene ID numbers as of Jan. 10, 2022.

| Gene | NCBI Gene ID |
|---|---|
| IL-12 | 3593 and 3592 |
| IL-2 | 3558 |
| IL-4 | 3565 |
| IL-5 | 3567 |
| IL-6 | 3569 |
| IL-8 | 3576 |
| IL-10 | 3586 |
| IL-13 | 3596 |
| IL-27 | 346778 |
| IL-1β | 3553 |
| TGFβ | 7040 |
| IFNγ | 3458 |
| IFNα | 3439 |
| IFNβ | 3456 |
| TNFα | 7124 |
| CCL2 | 6347 |
| CCL3 | 6348 |
| CCL4 | 6351 |
| CCL5 | 6352 |
| CCL8 | 6355 |
| CXCL12 | 6387 |
| GM-CSF | 1437 |

Interleukins (ILs) are a group of cytokines (secreted proteins and signal molecules) that were first seen to be expressed by white blood cells (leukocytes). The function of the immune system depends in a large part on interleukins, and rare deficiencies of a number of them have been described, all featuring autoimmune diseases or immune deficiency. The majority of interleukins are synthesized by helper CD4 T lymphocytes, as well as through monocytes, macrophages, and endothelial cells. They promote the development and differentiation of T and B lymphocytes, and hematopoietic cells. Interleukins include interleukin 1 (IL-1), interleukin 2 (IL-2), interleukin 3 (IL-3), interleukin 4 (IL-4), interleukin 5 (IL-5), interleukin 6 (IL-6), interleukin 7 (IL-7), interleukin 8 (IL-8), interleukin 9 (IL-9), interleukin 10 (IL-1M, interleukin 11 (IL-11), interleukin 12 (IL-12), interleukin 13 (IL-13), interleukin 14 (IL-14), interleukin 15 (IL-15), interleukin 16 (IL-16), interleukin 17 (IL-17), interleukin 18 (IL-18), interleukin 19 (IL-19), interleukin 20 (IL-20), interleukin 21 (IL-21), interleukin 22 (IL-22), interleukin 23 (IL-23), interleukin 24 (IL-24), interleukin 25 (IL-25), interleukin 26 (IL-26), interleukin 27 (IL-27), interleukin 28 (IL-28), interleukin 29 (IL-29), interleukin 30 (IL-30), interleukin 31 (IL-31), interleukin 32 (IL-32), interleukin 33 (IL-33), interleukin 35 (IL-35) and interleukin 36 (IL-36).

IL-1 alpha and IL-1 beta are cytokines that participate in the regulation of immune responses, inflammatory reactions, and hematopoiesis. IL-2 is a lymphokine that induces the proliferation of responsive T cells. In addition, it acts on some B cells, via receptor-specific binding, as a growth factor and antibody production stimulant. IL-3 is a cytokine that regulates hematopoiesis by controlling the production, differentiation and function of granulocytes and macrophages. IL-4 induces proliferation and differentiation of B cells and T cell proliferation. IL-5 regulates eosinophil growth and activation. IL-6 plays an essential role in the final differentiation of B cells into immunoglobulin-secreting cells, as well as inducing myeloma/plasmacytoma growth, nerve cell differentiation, and, in hepatocytes, acute-phase reactants. IL-7 is a cytokine that serves as a growth factor for early lymphoid cells of both B- and T-cell lineages. IL-8 induces neutrophil chemotaxis. IL-9 is a cytokine that supports IL-2 independent and IL-4 independent growth of helper T cells. IL-10 is a protein that inhibits the synthesis of a number of cytokines, including IFNγ, IL-2, IL-3, TNF, and GM-CSF produced by activated macrophages and by helper T cells. IL-11 stimulates megakaryocytopoiesis, leading to an increased production of platelets, as well as activating osteoclasts, inhibiting epithelial cell proliferation and apoptosis, and inhibiting macrophage mediator production. IL-12 is involved in the stimulation and maintenance of Th1 cellular immune responses, including the normal host defense against various intracellular pathogens. IL-13 is a pleiotropic cytokine that may be important in the regulation of the inflammatory and immune responses. IL-14 controls the growth and proliferation of B cells and inhibits Ig secretion. IL-15 induces production of Natural killer cells. IL-16 is a CD4+ chemoattractant. IL-17 is a potent proinflammatory cytokine produced by activated memory T cells. IL-18 induces production of IFNγ and increased natural killer cell activity. IL-20 regulates proliferation and differentiation of keratinocytes. IL-21 co-stimulates activation and proliferation of CD8+ T cells, augments NK cytotoxicity, augments CD40-driven B cell proliferation, differentiation and isotype switching, promotes differentiation of Th17 cells. IL-22 stimulates production of defensins from epithelial cells and activates STAT1 and STAT3. IL-23 is involved in the maintenance of IL-17 producing cells and increases angiogenesis but reduces CD8 T-cell infiltration. IL-24 plays important roles in tumor suppression, wound healing and psoriasis by influencing cell survival, inflammatory cytokine expression. IL-25 induces the production IL-4, IL-5 and IL-13, which stimulate eosinophil expansion. IL-26 enhances secretion of IL-10 and IL-8 and cell surface expression of CD54 on epithelial cells. IL-27 regulates the activity of B lymphocyte and T lymphocytes. IL-28 plays a role in immune defense against viruses. IL-29 plays a role in host defenses against microbes. IL-30 forms one chain of IL-27. IL-31 may play a role in inflammation of the skin. IL-32 induces monocytes and macrophages to secrete TNF-α, IL-8 and CXCL2. IL-33 induces helper T cells to produce type 2 cytokines. IL-35 induces suppression of T helper cell activation. IL-36 regulates DC and T cell responses.

Lymphokines are a subset of cytokines that are produced by a type of immune cell known as a lymphocyte. They are protein mediators typically produced by T cells to direct the immune system response by signalling between its cells. Lymphokines have many roles, including the attraction of other immune cells, including macrophages and other lymphocytes, to an infected site and their subsequent activation to prepare them to mount an immune response. Lymphokines aid B cells to produce antibodies. Important lymphokines secreted by the T helper cell include IL2, IL3, IL4, IL5, IL6, granulocyte-macrophage colony-stimulating factor (GM-CSF) and interferon gamma (IFNγ).

GM-CSF stimulates stem cells to produce granulocytes (neutrophils, eosinophils, and basophils) and monocytes. Monocytes exit the circulation and migrate into tissue, whereupon they mature into macrophages and dendritic cells. Thus, it is part of the immune/inflammatory cascade, by which activation of a small number of macrophages can rapidly lead to an increase in their numbers, a process crucial for fighting infection. GM-CSF also enhances neutrophil migration and causes an alteration of the receptors expressed on the cells surface.

IFNγ is a cytokine that is critical for innate and adaptive immunity against infections. IFNγ is an activator of macrophages and inducer of major histocompatibility complex class II molecule expression. The importance of IFNγ in the immune system stems in part from its ability to inhibit viral replication directly, and most importantly from its immunostimulatory and immunomodulatory effects.

A monokine is a type of cytokine produced primarily by monocytes and macrophages. Some monokines include IL-1, tumor necrosis factor-alpha, alpha and beta interferon, and colony stimulating factors. Tumor necrosis factor (TNF) is a cytokine—a small protein used by the immune system for cell signaling. TNF is released to recruit other immune system cells as part of an inflammatory response to an infection. Interferons (IFNs) are a group of signalling proteins made and released by host cells in response to the presence of several viruses. IFN-α proteins are produced mainly by plasmacytoid dendritic cells (pDCs) and are mainly involved in innate immunity against viral infection. IFN-p proteins are produced in large quantities by fibroblasts and have antiviral activity that is involved mainly in innate immune response. Colony-stimulating factors (CSFs) are secreted glycoproteins that bind to receptor proteins on the surfaces of hemopoietic stem cells, thereby activating intracellular signalling pathways that can cause the cells to proliferate and differentiate into a blood cell.

Chemokines are a family of small cytokines that have the ability to induce directed chemotaxis in nearby responsive cells. Chemokines are functionally divided into those that are homeostatic and those that are inflammatory. Homeostatic chemokines are constitutively produced in certain tissues and are responsible for basal leukocyte migration and include: CCL14, CCL19, CCL20, CCL21, CCL25, CCL27, CXCL12 and CXCL13. Inflammatory chemokines are formed under pathological conditions and actively participate in the inflammatory response attracting immune cells to the site of inflammation and include CXCL-8, CCL2, CCL3, CCL4, CCL5, CCL11, CXCL10.

Interferons (IFNs) are a group of signaling proteins made and released by host cells in response to the presence of several viruses. IFN-α, IFN-p, IFN-E, IFN-K and IFN-w bind to the IFN-a/(3 receptor complex and bind to specific receptors on target cells, which leads to expression of proteins that will prevent the virus from producing and replicating its RNA and DNA. IFNγ is released by cytotoxic T cells and type-1 T helper cells, however, IFNγ blocks the proliferation of type-2 T helper cells.

In some embodiments of any of the aspects, the proinflammatory cytokine encoded by the first cytokine construct is IL-12 or a subunit, dimer, heterodimer, derivative, fragment, agonist or homologue thereof. In some embodiments of any of the aspects, the proinflammatory cytokine encoded by the first cytokine construct is IL-12 or a subunit of human or other mammalian homology. As mentioned above, interleukin 12 (IL-12) is an immune-stimulatory cytokine for immune cells including T cells and NK cells. IL-12 is a heterodimeric cytokine that is produced specifically by phagocytic cells as well as antigen-presenting cells and enhances anti-tumor immune responses. A consequence of the potent immune stimulatory properties of IL-12 is that systemic administration can lead to serious side effects that limit its clinical application in patients. Expression of IL-12 by engineered NK92 at tumor sites has been shown to increase the antitumor activities of chimeric antigen receptor (CAR)-modified T cells (Luo et al. Front Oncol. (2019) December 19; 9:1448). It is believed that IL-12 induced IFNγ accumulation in tumors also promotes the penetration of T-lymphocytes or other host immune cells (e.g. NK cells) into the tumors, thereby enhancing the therapeutic effects (Chinnasamy D. et al. Clin Cancer Res 2012:18/Chmielewski M. et al. Cancer Res 2011; 71/Kerkar S P. Et al. J Clin Invest 2011; 121/Jackson H J. Et al. Nat Rev Clin Oncol 2016; 13). The foregoing references are incorporated by reference herein in their entireties.

In embodiments of the present invention the compositions of the invention comprise an mRNA that include at least one ORF that encodes functional IL-12 or an analogue or derivative thereof. Since, wild type IL-12 is comprised of a heterodimer of 35 kDa IL-12A and 40 kDa IL-12B subunits, the ORF may comprise one of these subunits and be administered in combination with another mRNA encoding the other subunit thereby allowing the assembly of functional IL-12 in the cell. Alternatively, functional IL-12 may be in the form of a modified single chain version of IL-12 that comprises both subunits within a single ORF (for example, see SEQ ID NO: 59).

In some embodiments of any of the aspects, the proinflammatory cytokine encoded by the first cytokine construct comprises a sequence at least 80%, at least 85%, at least 90%, at least 95%, or at least 98% identical to SEQ ID NO: 59. In some embodiments of any of the aspects, the proinflammatory cytokine encoded by the first cytokine construct comprises the sequence of SEQ ID NO: 59. In some embodiments of any of the aspects, the first ORF comprises a sequence at least 80%, at least 85%, at least 90%, at least 95%, or at least 98% identical to SEQ ID NO: 59. In some embodiments of any of the aspects, the first ORF comprises the sequence of SEQ ID NO: 59.

In some embodiments of any of the aspects, the proinflammatory cytokine encoded by the first cytokine construct comprises a sequence at least 80%, at least 85%, at least 90%, at least 95%, or at least 98% identical to one or more of SEQ ID NOs: 200-203 (isoforms of the IL-12α subunit). In some embodiments of any of the aspects, the proinflammatory cytokine encoded by the first cytokine construct comprises the sequence of one or more of SEQ ID NOs: 200-203. In some embodiments of any of the aspects, the first ORF comprises a sequence encoding a polypeptide that is at least 80%, at least 85%, at least 90%, at least 95%, or at least 98% identical to one or more of SEQ ID NOs: 200-203. In some embodiments of any of the aspects, the first ORF comprises a sequence encoding the polypeptide of one or more of SEQ ID NOs: 200-203.

In some embodiments of any of the aspects, the proinflammatory cytokine encoded by the first cytokine construct comprises a sequence at least 80%, at least 85%, at least 90%, at least 95%, or at least 98% identical to one or more of SEQ ID NOs: 204-205 (isoforms of the IL-12β subunit). In some embodiments of any of the aspects, the proinflammatory cytokine encoded by the first cytokine construct comprises the sequence of one or more of SEQ ID NOs: 204-205. In some embodiments of any of the aspects, the first ORF comprises a sequence encoding a sequence with at least 80%, at least 85%, at least 90%, at least 95%, or at least 98% identical to one or more of SEQ ID NOs: 204-205. In some embodiments of any of the aspects, the first ORF comprises a sequence encoding of one or more of SEQ ID NOs: 204-205.

In some embodiments of any of the aspects, a proinflammatory cytokine comprises a sequence at least 80%, at least 85%, at least 90%, at least 95%, or at least 98% identical to one or more of SEQ ID NOs: 200-205 or the sequence encoded by SEQ ID NO: 59. In some embodiments of any of the aspects, a proinflammatory cytokine comprises the sequence of one or more of SEQ ID NOs: 200-205 or the sequence encoded by SEQ ID NO: 59. In some embodiments of any of the aspects, an ORF encoding a proinflammatory comprises a sequence at least 80%, at least 85%, at least 90%, at least 95%, or at least 98% identical to SEQ ID NOs: 59 or a sequence encoding one or more of SEQ ID NOs: 200-205. In some embodiments of any of the aspects, an ORF encoding a proinflammatory comprises the sequence of one or more of SEQ ID NOs: 59 or a sequence encoding one or more of SEQ ID NOs: 200-205.

In some embodiments of any of the aspects, the proinflammatory cytokine encoded by the first cytokine construct comprises a sequence at least 80%, at least 85%, at least 90%, at least 95%, or at least 98% identical to one or more of SEQ ID NOs: 200-203 (isoforms of the IL-12α subunit). In some embodiments of any of the aspects, the proinflammatory cytokine encoded by the first cytokine construct comprises the sequence of one or more of SEQ ID NOs: 200-203. In some embodiments of any of the aspects, the first ORF comprises a sequence at least 80%, at least 85%, at least 90%, at least 95%, or at least 98% identical to one or more of SEQ ID NOs: 200-203. In some embodiments of any of the aspects, the first ORF comprises the sequence of one or more of SEQ ID NOs: 200-203.

In some embodiments of any of the aspects, the proinflammatory cytokine encoded by the first cytokine construct comprises a sequence at least 80%, at least 85%, at least 90%, at least 95%, or at least 98% identical to one or more of SEQ ID NOs: 204-205 (isoforms of the IL-12β subunit). In some embodiments of any of the aspects, the proinflammatory cytokine encoded by the first cytokine construct comprises the sequence of one or more of SEQ ID NOs: 204-205. In some embodiments of any of the aspects, the first ORF comprises a sequence at least 80%, at least 85%, at least 90%, at least 95%, or at least 98% identical to one or more of SEQ ID NOs: 204-205. In some embodiments of any of the aspects, the first ORF comprises the sequence of one or more of SEQ ID NOs: 204-205.

Multiple proinflammatory cytokines can be administered to a single subject, e.g., to provide a stronger adjuvant effect. This can be referred to as “adjuvant stacking.” In some embodiments of any of the aspects, the method further comprises administering one or more further cytokine mRNA constructs, each further cytokine mRNA construct comprising a further open reading frame (ORF), wherein each further ORF encodes a proinflammatory cytokine distinct from the proinflammatory cytokine encoded by the first ORF.

In some embodiments of any of the aspects, the one or more further cytokine mRNA constructs is administered in the same composition as the first antigen construct. In some embodiments of any of the aspects, the one or more further cytokine mRNA constructs is administered in the same composition as the first cytokine construct. In some embodiments of any of the aspects, the one or more further cytokine mRNA constructs is administered a composition not comprising the first antigen construct and the first cytokine construct. In some embodiments of any of the aspects, the one or more further cytokine mRNA constructs is administered concurrently with the first antigen construct and/or the first cytokine construct. In some embodiments of any of the aspects, the one or more further cytokine mRNA constructs is administered sequentially with the first antigen construct and/or the first cytokine construct.

In some embodiments of any of the aspects, there are 1-9 further cytokine mRNA constructs, e.g., administered and/or present in a composition.

In some embodiments of any of the aspects, a single mRNA molecule comprises the first cytokine mRNA construct and further cytokine mRNA constructs.

In some embodiments of any of the aspects, the first cytokine mRNA construct further comprises one or more further open reading frames (ORFs), wherein each further ORF encodes a proinflammatory cytokine distinct from the proinflammatory cytokine encoded by the second ORF. In some embodiments of any of the aspects, the first ORF encodes IL-12 or a subunit, derivative, fragment, agonist or homologue thereof and the one or more further ORFs encode IL-2; IL-4; IL-5; IL-6; IL-8; IL-10; IL-13; IL-27; IL-1 beta; TGFbeta; IFNy; IFNa; IFNI3; TNFa; CCL2; CCL3; CCL4; CCL5; CCL8; CXCL12; GM-CSF; or a subunit, derivative, fragment, agonist, or homologue thereof. In some embodiments of any of the aspects, there are 1-9 further cytokine encoding ORFs, e.g., administered and/or present in a composition.

The antigen encoded by a mRNA construct described herein can be an antigen of, derived from, or specific to a pathogenic agent, pathogenic organism and/or a diseased cell. The mRNA constructs and compositions as described herein can be used in vaccine therapy, in the enhancement of the efficacy of a conventional vaccine, and/or as a novel vaccine form for use against infectious pathogens, such as viruses, bacteria, fungi, protozoa, prions, and helminths (worms). It is contemplated that mRNA constructs as described can be circularised by the (direct or indirect) linkage of the 5′ and 3′ ends and such circular or circularised RNA constructs are considered to be included by the term ‘mRNA construct’ as used herein; such constructs have been shown to be potentially effective as RNA-based vaccines, for example against SARS-COV-2 (Qu L. et al, bioRxiv 2021.03.16.435594; doi.org/10.1101/2021.03.16.435594; which is incorporated by reference herein in its entirety). As a result, mRNA constructs as described herein include circular or circularised RNA constructs which can be translated in cells.

Hence, the compositions of the present invention can be used in the prophylaxis or treatment of infectious pathogenic disease (e.g., caused by an agent or organism) and/or the methods described herein can relate to the prophylaxis or treatment of infectious pathogenic disease (e.g., caused by an agent or organism), either by way of inclusion within vaccine formulations or in the form of adjuvants (e.g. with an appropriate cytokine) that is administered in combination with a vaccine.

Examples of infectious bacterial organisms include Acetobacter aurantius, Acinetobacter baumannii, Actinomyces israelii, Agrobacterium radiobacter, Agrobacterium tumefaciens, Anaplasma phagocytophilum, Azorhizobium caulinodans, Azotobacter vinelandii, viridans streptococci, Bacillus anthracis, Bacillus brevis, Bacillus cereus, Bacillus fusiformis, Bacillus licheniformis, Bacillus megaterium, Bacillus mycoides, Bacillus stearothermophilus, Bacillus subtilis, Bacillus thuringiensis, Bacteroides fragilis, Bacteroides gingivalis, Bacteroides melaninogenicus, Prevotella melaninogenica, Bartonella henselae, Bartonella quintana, Bordetella bronchiseptica, Bordetella pertussis, Borrelia burgdorferi, Brucella abortus, Brucella melitensis, Brucella suis, Burkholderia mallei, Burkholderia pseudomallei, Burkholderia cepacian, Calymmatobacterium granulomatis, Campylobacter coli, Campylobacter fetus, Campylobacter jejuni, Campylobacter pylori, Chlamydia, Chlamydia trachomatis, Chlamydophila pneumoniae, Chlamydia pneumoniae, Chlamydophila psittaci, Chlamydia psittaci, Clostridium botulinum, Clostridium difficile, Clostridium perfringens, Clostridium welchii, Clostridium tetani, Corynebacterium diphtheriae, Corynebacterium fusiforme, Coxiella burnetiid, Ehrlichia chaffeensis, Ehrlichia ewingii, Eikenella corrodens, Enterobacter cloacae, Enterococcus avium, Enterococcus durans, Enterococcus faecalis, Enterococcus faecium, Enterococcus gallinarum, Enterococcus maloratus, Escherichia coli, Fusobacterium necrophorum, Fusobacterium nucleatum, Gardnerella vaginalis, Haemophilus ducreyi, Haemophilus influenzae, Haemophilus parainfluenzae, Haemophilus pertussis, Haemophilus vaginalis, Helicobacter pylori, Klebsiella pneumoniae, Lactobacillus acidophilus, Lactobacillus bulgaricus, Lactobacillus casei, Lactococcus lactis, Legionella pneumophila, Leishmania donovani, Leptospira interrogans, Leptospira noguchii, Listeria monocytogenes, Methanobacterium extroquens, Microbacterium multiforme, Micrococcus luteus, Moraxella catarrhalis, Mycobacterium avium, Mycobacterium bovis, Mycobacterium diphtheriae, Mycobacterium intracellulare, Mycobacterium leprae, Mycobacterium lepraemurium, Mycobacterium phlei, Mycobacterium smegmatis, Mycobacterium tuberculosis, Mycoplasma fermentans, Mycoplasma genitalium, Mycoplasma hominis, Mycoplasma penetrans, Mycoplasma pneumoniae, Mycoplasma mexican, Neisseria gonorrhoeae, Neisseria meningitidis, Pasteurella multocida, Pasteurella tularensis, Peptostreptococcus, Porphyromonas gingivalis, Prevotella melaninogenica, Bacteroides melaninogenicus, Pseudomonas aeruginosa, Rhizobium radiobacter, Rickettsia prowazekii, Rickettsia psittaci, Rickettsia quintana, Rickettsia, Rickettsia trachomae, Rochalimaea henselae, Rochalimaea quintana, Rothia dentocariosa, Salmonella enteritidis, Salmonella typhi, Salmonella typhimurium, Serratia marcescens, Shigella dysenteriae, Spirillum volutans, Staphylococcus aureus, Staphylococcus epidermidis, Stenotrophomonas maltophilia, Streptococcus, Streptococcus agalactiae, Streptococcus avium, Streptococcus bovis, Streptococcus cricetus, Streptococcus faceium, Streptococcus faecalis, Streptococcus ferus, Streptococcus gallinarum, Streptococcus lactis, Streptococcus mitior, Streptococcus mitis, Streptococcus mutans, Streptococcus oralis, Streptococcus pneumoniae, Streptococcus pyogenes, Streptococcus rattus, Streptococcus salivarius, Streptococcus sanguis, Streptococcus sobrinus, Treponema, Ureaplasma urealyticum, Vibrio cholerae, Vibrio comma, Vibrio parahaemolyticus, Vibrio vulnificus, Yersinia enterocolitica, Yersinia pestis and Yersinia pseudotuberculosis.

Examples of viral infectious agents include Adeno-associated virus; Aichi virus, Australian bat lyssavirus; BK polyomavirus; Banna virus; Barmah forest virus; Bunyamwera virus; Bunyavirus La Crosse; Bunyavirus snowshoe hare; Cercopithecine herpesvirus; Chandipura virus; Chikungunya virus; Cosavirus A; Cowpox virus; Coxsackievirus; Crimean-Congo hemorrhagic fever virus; Dengue virus; Dhori virus; Dugbe virus; Duvenhage virus; Eastern equine encephalitis virus; Ebolavirus; Echovirus; Encephalomyocarditis virus; Epstein-Barr virus; European bat lyssavirus; GB virus C/Hepatitis G virus; Hantaan virus; Hendra virus; Hepatitis A virus; Hepatitis B virus; Hepatitis C virus; Hepatitis E virus; Hepatitis delta virus; Horsepox virus; Human adenovirus; Human astrovirus; Human coronavirus; Human cytomegalovirus; Human enterovirus 68, 70; Human herpesvirus 1; Human herpesvirus 2; Human herpesvirus 6; Human herpesvirus 7; Human herpesvirus 8; Human immunodeficiency virus; Human papillomavirus 1; Human papillomavirus 2; Human papillomavirus 16,18; Human parainfluenza; Human parvovirus B19; Human respiratory syncytial virus; Human rhinovirus; Human SARS coronavirus; Human spumaretrovirus; Human T-lymphotropic virus; Human torovirus; Influenza A virus; Influenza B virus; Influenza C virus; Isfahan virus; JC polyomavirus; Japanese encephalitis virus; Junin arenavirus; KI Polyomavirus; Kunjin virus; Lagos bat virus; Lake Victoria Marburgvirus; Langat virus; Lassa virus; Lordsdale virus; Louping ill virus; Lymphocytic choriomeningitis virus; Machupo virus; Mayaro virus, MERS coronavirus; Measles virus; Mengo encephalomyocarditis virus; Merkel cell polyomavirus; Mokola virus; Molluscum contagiosum virus; Monkeypox virus; Mumps virus; Murray valley encephalitis virus; New York virus; Nipah virus; Norwalk virus; O′nyong-nyong virus; Orf virus; Oropouche virus; Pichinde virus; Poliovirus; Punta toro phlebovirus; Puumala virus; Rabies virus; Respiratory syncytial virus; Rift valley fever virus; Rosavirus A; Ross river virus; Rotavirus A; Rotavirus B; Rotavirus C; Rubella virus; Sagiyama virus; Salivirus A; Sandfly fever sicilian virus; Sapporo virus; SARS coronavirus 2 (COVID); Semliki forest virus; Seoul virus; Simian foamy virus; Simian virus 5; Sindbis virus; Southampton virus; St. louis encephalitis virus; Tick-borne powassan virus; Torque teno virus; Toscana virus; Uukuniemi virus; Vaccinia virus; Varicella-zoster virus; Variola virus; Venezuelan equine encephalitis virus; Vesicular stomatitis virus; Western equine encephalitis virus; WU polyomavirus; West Nile virus; Yaba monkey tumor virus; Yaba-like disease virus; Yellow fever virus; and Zika virus.

Examples of fungal infectious organisms include: Gymnopus spp., Rhodocollybia butyracea, Hypholo ma fasciculare, Saccharomyces cerevisiae, Tuber spp., Bothia castanella, Rhizosphere spp., Herpotrichiellaceae spp., Verrucariaceae spp., Marchandiomyces spp., Minimedusa spp., Marchandiobasidium aurantiacum, Marchandiomyces corallinus, Marchandiomyces lignicola, Burgoa spp., Athelia arachnoidea, Alternaria alternata, Alternaria spp., Boletus edulis, Leccinum aurantiacum, Trametes versicolor, Trametes spp., Sympodiomycopsis spp., Flavocetraria nivalis, Ampelomyces spp., Gymnopus biformis, Gymnopus spp., Gymnopus confluens, Gymnopus spongiosus, Collybia readii, Marasmiellus stenophyllus, Marasmiellus ramealis, Marasmius scorodonius, Collybia marasmioides, Micromphale brassicolens, Caripia montagnei, Rhodocollybia spp., Anthracophyllum lateritium, Anthracophyllum archeri, Anthracophyllum spp., Phan erochaete spp., Schizosaccharomyces pombe, Saccharomyces cerevisiae, Aspergillus fumigatus, Aspergillus flavus, Aspergillus niger, Aspergillus spp., Tricholoma imbricatum, Tricholoma flavovirens, Tomentella sublilacina, Rhizopogon spp., Laccaria spp., Inocybe spp., Hebeloma spp., Cortinarius spp., Clavulina spp., Xerocomus spp., Amanita spp., Eurotium herbariorum, Edyuillia athecia, Warcupiella spinulosa, Hemicarpenteles paradoxus, Hemicarpenteles acanthosporus, Hemicarpenteles spp., Chaetosartorya cremea, Petromyces spp., Graphium tectonae, Di plolaimelloides spp., Rhabdolaimus spp., Hohenbuehelia petalodes, Glomerella graminicola, Cryptococcus arboriformis, Cryptococcus neoformans, Cryptococcus spp., Gamsylella parvicollis, Monacrosporium haptotylum, Monacrosporium sichuanense, Monacrosporium Spp., Monacrosporium gephyropagum, Monacrosporium spp., Drechslerella coelobrocha, Drechslerella dactyloides, Drechslerella spp., Arthrobotrys musiformis, Arthrobotrys flagrans, Arthrobotrys hertziana, Arthrobotrys oligospora, Arthrobotrys vermicola, Arthrobotrys spp., Monacrosporium drechsleri, Vermispora spp., Pseudallescheria boydii (Scedosporium apiospermum), Scedosporium inflatum, Geosmithia spp., Glomerella cingulata, Lophodermium piceae, Fusarium asiaticum, Fusarium spp., Pleurotus eryngii, Cintractia sorghi-vulgaris, Cantharocybe gruberi, Bourdotia spp., Auricularia spp., Puccinia bartholomaei, Puccinia spp., Diaporthe phaseolorum, Melanconis stilbostoma, Xylaria spp., Trichophyton equinum, Trichophyton tonsurans, Trichophytum violaceum, Trichophytum rubrum, Trichophytum interdigitale, Trichophytum schoenleinii Trichophyton spp., Chlorophyllum agaricoides, Cenococcum geophilum, Helotiales spp., Rhizoscyphus ericae, Lactarius pubescens, Lactarius spp., Piloderma fallax, Suillus luteus, Amanita muscaria, Tricholoma spp., Laccaria cf bicolour, Cortinarius purpurascens, Seiridium spp., Apiospora montagnei, Chondrostereum purpureum, Botryobasidium subcoronatum, Boletellus shichianus, Boletellus spp., Hypocrea farinose, Hypocrea spp., Sarcostroma restionis, Sarcostroma spp., Truncatella betulae, Truncatella spp., Pestalotiopsis matildae, Paraconiothyrium spp., Phoma spp., Cunninghamella bainieri, Cunninghamella bertholletiae, Cantharellus cibarius, Apiospora bambusae, Apiospora spp., Discostroma botan, Cercophora caudate, Gnomonia ribicola, Faurelina elongate, Mycorrhiza fungi, Geomyces pannorum, Coprinus spp., Acremonium spp., Clonostachys spp., Phoma eupyrena, Tetracladium spp., Mortierella spp., Tulasnella calospora, Epulorhiza spp., Tulasnella calospora, Antarctomyces psych rotrophicus, Amphisphaeriaceae spp., Phomopsis spp., Trichoderma spp., Pestalotiopsis spp., Pestalotiopsis spp., Trichocomaceae spp., Coniochaetales spp., Tremellales spp., Dothideales spp., Phyllachoraceae spp., Saccharomycesles spp., Herpotrichiellaceae spp., Liliopsida spp., Trichosporonales spp., Trichosporon mycotoxinivorans, Trichosporon spp., Dothioraceae spp., Hypocreales spp., Mycosphaerellaceae spp., Sporidiobolales spp., Clavicipitaceae spp., Pleosporales spp., Ustilaginaceae spp., Phyllachoraceae spp., Mucoraceae spp., Sordariales spp., Filobasidiales spp., Calosphaeriaceae spp., Clavicipitaceae spp., Mucorales spp., Herpotrichiellaceae spp., Microdochium spp., Phyllachoraceae spp., Zopfiaceae spp., Botryosphaeriaceae spp., Helotiaceae spp., Bionectriaceae spp., Lachnocladiaceae spp., Di podascaceae spp. Caulerpaceae spp., Microstromatales spp., Aphyllophorales spp., Montagnulaceae spp., Gymnoascaceae spp., Cryphonectriaceae spp., Xylariales spp., Montagnulaceae spp., Chaetomiaceae spp., Xanthoria elegans, Rhizopus spp., Penicillium spp., Cetraria aculeate, Nephromopsis laureri, Tuckermannopsis chlorophylla, Cetraria ericetorum, Cetraria spp., Flavocetraria cucullata, Kaernefeltia merrillii, Amorosia littoralis, Quambalaria cyanescens, Cordyceps roseostromata, Cordyceps spp., Russula spp., Clavulina spp., Tuber quercicola, Gymnomyces spp., Tetrachaetum elegans, Anguillospora longissima, Hypocrea spp., Sirococcus conigenus, Rhizopogon roseolus, Rhizopogon olivaceotinctus, Rhizopogon spp., Pisolithus microcarpus, Rhizoscyphus ericae, Cortinarius glaucopus, Paxillus spp., Suillus variegates, Pyrobaculum aerophilum, Tulasnella spp., Hohenbuehelia spp., Cochliobolus lunatus, Plicaturopsis crispa, Bondarcevomyces taxi, Tapinella panuoides, Tapinella spp., Austropaxillus spp., Gomphidius roseus, Gyrodon lividus, Phylloporus pelletieri, Chamonixia caespitose, Porphyrellus porphyrosporus, Truncocolumella citrina, Tapinella atrotomentosa, Scleroderma leave, Suillus variegates, Suillus spp., Porphyrellus porphyrosporus, Pisolithus arrhizus, Phaeogyroporus portentosus, Melanogaster variegates, Leucogyrophana mollusca, Hydnomerulius pinastri, Gomphidius roseus, Gyrodon lividus, Gyroporus cyanescens, Chalciporus piperatus, Chamonixia caespitose, Bondarcevomyces taxi, Dendryphiella triticicola, Guignardia spp., Shiraia spp., Cladosporium spp., Phomopsis spp., Diaporthales spp., Pestalotiopsis spp., Lophiostoma spp., Verticillium chlamydosporium, Paecilomyces lilacinus, Paecilomyces varioti, Paecilomyces spp., Ceratorhiza oryzae-sativae, Geosmithia pallida, Geosmithia spp., Geosiphon pyriformis, Agonimia spp., Pyrgillus javanicus, Exophiala dermatitidis, Exophiala pisciphila, Exophiala spp., Ramichloridium anceps, Ramichloridium spp., Capronia pilosella, Isaria farinose, Pochonia suchlasporia, Lecanicillium psalliotae, Dothideomycete spp., Leotiomycete spp., Ustilaginoidea vixens, Hyphozyma lignicola, Coniochaeta malacotricha, Coniochaeta spp., Torrubiella confragosa, Isaria tenuipes, Microsporum canis, Microsporum audouinii, Microsporum spp., Epicoccum floccosum, Gigaspora rosea, Gigaspora spp., Ganoderma spp., Pseudoperonospora cubensis, Hyaloperonospora parasitica, Plectophomella spp., Aureobasidium pullulans, Gloeophyllum sepiarium, Gloeophyllum spp., Donkioporia expansa, Antrodia sinuosa, Phaeoacremonium rubrigenum, Phaeoacremonium spp., Albertiniella polyporicola, Cephalotheca sulfurea, Fragosphaeria reniformis, Fragosphaeria spp., Phialemonium dimorphosporum, Phialemonium spp., Pichia norvegensis, Pichia spp., Candida albicans, Candida tropicalis, Candida glabrata, Candida parapsilosis, Candida spp., Gondawanamyces spp., Graphium spp., Ambrosiella spp., Microglossum spp., Neobulgaria pura, Holwaya mucida, Chlorovibrissea spp., Chlorociboria spp., Thaxterogaster spp., Cortinarius spp., Setchelliogaster spp., Timgrovea spp., Descomyces spp., Hymenogaster arenarius,

Quadrispora tubercularis, Quadrispora spp., Protoglossum violaceum, Ceratostomella pyrenaica, Ceratosphaeria lampadophora, Fonsecaea pedrosoi, Phlebia acerina, Phlebia spp., Pestalotiopsis disseminata, Paracoccidioides brasiliensis, Racospermyces koae, Endoraecium acaciae, Uromycladium tepperianum, Uromycladium spp., Agaricus bisporus, Agaricus spp., Psilocybe quebecensis, Psilocybe merdaria, Psilocybe spp., Gymnopilus luteofolius, Gymnopilus liquiritiae, Gymnopilus spp., Hypholoma tuberosum, Melanotus hartii, Panaeolus uliginosus, Stropharia rugosoannulata, Dermocybe semisanguinea, Dermocybe spp., Helicoma monthpes, Helicoma spp., Tubeufia helicomyces, Tubeufia spp., Leohumicola verrucosa, Leptosphaerulina chartarum, Macrophoma spp., Marssonina rosae, Botryotinia fuckeliana, Pestalotiopsis spp., Chrysosporium carmichaelii, Chrysosporium spp., Dactylella oxyspora, Dactylellina lobatum, Cucurbitaceae spp., Chrysophyllum sparsiflorum, Chrysophyllum spp., Blumeria graminis, Sawadaea polyfida, Sawadaea spp., Parauncinula septata, Erysiphe mori, Erysiphe spp., Typhulochaeta japonica, Golovinomyces orontii, Golovinomyces spp., Podosphaera xanthii, Podosphaera spp., Arthrocladiella mougeotii, Neoerysiphe galeopsidis, Phyllactinia kakicola, Phyllactinia spp., Cyphellophora laciniata, Sphaerographium tenuirostrum, Microsphaera trifolii, Sphaerotheca spiraeae, Sphaerotheca spp., Uncinuliella australiana, Absidia corymbifera, Absidia spp., Geotrichum spp., Nectria curia, Anamika lactariolens, Hebeloma velutipes, Stropharia ambigua, Agrocybe praecox, Hydnum rufescens, Hydnum spp., Meliniomyces variabilis, Rhizoscyphus ericae, Cryptosporiopsis ericae, Hyalodendron spp., Leptographium lundbergii, Leptographium spp., Termitomyces spp., Coccidioides posadasii, Coccidioides immitis, Sclerotinia sclerotiorum, Phomopsis spp., Metarhizium anisopliae, Cordyceps spp., Tilletiopsis washingtonensis,

Cerrena unicolor, Stachybotrys chartarum, Phaeococcomyces nigricans, Ganoderma philippii, Ganoderma spp., Gloeophyllum sepiarium, Cystotheca lanestris, Leveillula taurica, Phyllactinia fraxini, Varicosporium elodeae, Rhinocladiella basitonum, Melanchlenus oligospermus, Clavispora lusitaniae, Rhizopus spp., Rhizomucor spp., Mucor spp., Conidiobolus coronatus, Conidiobolus spp., Basidiobolus ranarum, Basidiobolus spp., Ochronis spp., Histoplasma capsulatum, histoplasma spp., Wilcoxina mikolae, Lasiodiplodia spp., Physcia caesia, Physcia spp., Brachyconidiellopsis spp., Conocybe lacteal, Gastrocybe lateritia, Gastrocybe spp., Agrocybe semiorbicularis, Taphrina pruni, Taphrina spp., Asterophora parasitica, Asterophora spp., Eremothecium ashbyi, Tricladium splendens, Ramaria flava, Ramaria spp., Laccaria fraternal, Scutellospora spp., Illosporium carneum, Hobsonia christiansenii, Marchandiomyces corallinus, Fusicoccum luteum, Botryosphaeria ribis, Pseudozyma aphidis, Pseudozyma spp., Pesotum erubescens, Battarrea stevenii, Battarrea spp., Harposporium Janus, Harposporium spp., Hirsutella rhossiliensis, Arthroderma ciferrii, Arthroderma spp., Pucciniastrum goeppertianum, Cronartium occidentale, Cronartium arizonicum, Cronartium spp., Peridermium harknessii, Peridermium spp., Chrysomyxa arctostaphyli, Holleya sinecauda, Holleya spp., Zoophthora radicans, Smittium culisetae, Auxarthron zuffianum, Renispora flavissima, Ctenomyces serratus, and Sporothrix schenckii.

Examples of parasitic species as infectious agents can include helminths (worms) that may be selected from: cestodes: e.g. Anaplocephala spp.; Dipylidium spp.; Diphyllobothrium spp.; Echinococcus spp.; Moniezia spp.; Taenia spp.; trematodes e.g. Dicrocoelium spp.; Fasciola spp.; Paramphistomum spp.; Schistosoma spp.; or nematodes, e.g.; Ancylostoma spp.; Anecator spp.; Ascaridia spp.; Ascaris spp.; Brugia spp.; Bunostomum spp.; Capillaria spp.; Chabertia spp.; Cooperia spp.; Cyathostomum spp.; Cylicocyclus spp.; Cylicodontophorus spp.; Cylicostephanus spp.; Craterostomum spp.; Dictyocaulus spp.; Dipetalonema spp; Dirofilaria spp.; Dracunculus spp.; Enterobius spp.; Filaroides spp.; Habronema spp.; Haemonchus spp.; Heterakis spp.; Hyostrongylus spp.; Meta strongylus spp.; Meullerius spp. Necator spp.; Nematodirus spp.; Nippostrongylus spp.; Oesophagostomum spp.; Onchocerca spp.; Ostertagia spp.; Oxyuris spp.; Parascaris spp.; Stephanurus spp.; Strongylus spp.; Syngamus spp.; Toxocara spp.; Strongyloides spp.; Teladorsagia spp.; Toxascaris spp.; Trichinella spp.; Trichuris spp.; Trichostrongylus spp.; Triodontophorous spp.; Uncinaria spp., and/or Wuchereria spp.

Examples of parasitic species as infectious agents may include protozoa that are selected from: Leishmania species including Trypanosoma, Donovan Leishmania, Plasmodium spp. including, but not limited to, Plasmodium falciparum; Pneumocystis carini, Cryptosporidium parum, Rumble flagellate, Shigella amoeba, and Cyclosporanga canetenensis.

In some embodiments of any of the aspects, the disease is caused by a coronavirus, an intracellular pathogen, a latent infection, an active infection, an influenza virus, respiratory syncytial virus (RSV), human immunodeficiency virus (HIV), varicella zoster virus (VZV), or Mycobacterium tuberculosis; and/or one or more of the antigens are a coronavirus, an intracellular pathogen, a latent infection, an active infection, an influenza virus, respiratory syncytial virus (RSV), human immunodeficiency virus (HIV), varicella zoster virus (VZV), plasmodium (Malaria), Streptococcus pneumoniae, Streptococcus pyogenes, Yersinia pestis, Haemophilus influenzae, Staphylococcus aureus, Pseudomonas aeruginosa, Bordetella pertussis, Ebola virus, Lassa virus, Middle East Respiratory Syndrome coronavirus, SARS-COV-1, SARS-COV-2, SARS-COV-2 variants of concerns, Marburg virus, Nipah virus, Rift Valley Fever virus, Chikungunya virus or Mycobacterium tuberculosis antigen. In some embodiments of any of the aspects, the disease is caused by a coronavirus and/or one or more of the antigens are a coronavirus antigen, e.g., MERS-COV virus, SARS-COV-1 virus, or SARS-COV-2 virus.

The vaccine compositions and methods as discussed herein are non-exclusively contemplated for the treatment and prevention of diseases which may already be known to be susceptible to vaccination, particularly where an effective immunogenic protein is known.

Table 5 (below) provides an illustrative example of antigens that are selected to use in the compositions and methods of the present invention, for which an immune response is desired. One of skill in the art could readily obtain similar antigens/targets from public databases and publications and generate compositions of the invention. It should be understood that more than one antigen may be delivered to a subject depending on the state of disease, e.g., prophylactic prior to infection versus an active infection. By way of example, for a subject with an active tuberculosis disease, one might deliver the TB antigen that codes for a TB protein from the active phase (e.g., ESAT6 Ag85B), from latent phase (Rv2626), and/or from the resuscitation phase (RPfB-D). In this way, an active tuberculosis can be treated, particularly when it is desired to administer an adjuvant that elicits a Th1 response.

In one aspect of the invention, the compositions described herein are administered in combination with standard therapies, e.g., for an active bacterial or viral infection, antimicrobial agents or antiviral agents known in the field to treat such diseases can be administered. Such agents can be administered prior to, simultaneously with (either alone or as a fixed dose combination) or following treatment with a composition of the invention.

In some embodiments, the coding mRNA can code for an antigen against which an immune response is desired. Delivery of such antigens can be used to induce a local immune response as discussed above, or in order to provoke an adaptive immune response to the antigen itself—that is, to induce immunity against that antigen, similar to a vaccine. For example, the coding mRNA can encode a bacterial, viral or otherwise microbial protein against which an immune reaction is desired, in whole or part. Such encoded products are referred to for this discussion as ‘antigen products’ or ‘antigen’. In some cases, immunity can be generated against only part of a bacterial, viral or otherwise microbial protein (an ‘epitope’ or ‘antigenic determinant’), so the encoding of only those parts is also envisaged. In particular, parts of a microbial protein which are displayed externally can be selected as likely targets for immune recognition. As a result, an encoded antigen can be a bacterial, viral or otherwise microbial protein, but can be a partial sequence, part or fragment thereof, in particular, an ‘epitope containing fragment’ thereof. It is envisioned that more than one antigen for a particular microbe or pathogen can be provided, in the same or different mRNA constructs.

Vaccine compositions and methods as discussed herein are non-exclusively contemplated for the treatment and prevention of diseases which are already known to be susceptible to vaccination, particularly where an effective immunogenic protein is known, such as described Table 5. As a result, compositions and methods herein can use mRNA constructs which encode one or more of the below-described immunogenic proteins, or variants thereof as antigens

TABLE 5
Exemplary vaccine antigens for infectious diseases

| Pathogen | Antigens/targets | Reference |
|---|---|---|
| Chickenpox (varicella) | whole varicella-zoster virus (VZV) antigen, | Bergen, R. E., Diaz, P. S., |
| | VZV glycoprotein I (gpl), immediate early | & Arvin, A. M. |
| | protein (1E-62) | (1990). |
| | VZV gE: varicella-zoster virus glycoprotein | academic.oup.comijid/ |
| | E | article-abstract/ |
| | | 162/5/1049/829640 |
| | | doi.org/10.1517/ |
| | | 14712598.2016.1134481 |
| Chlamydia spp. | PmpG, Pmpl, PmpE, MOMP, PmpD, PmpH, | US10953080B2 |
| Cholera | rCTB | Price, G.A., McFann, |
| | LT | K., & Holmes, R.K. |
| | inactivated Vibro Cholerae O1 bacteria, | (2013). journals.plos.org/ |
| | enterotoxigenic Escherichia coli (ETEC) | plosone/article?id = |
| | CFA/I, CS3, CS5 and CS6, LCTBBA | 10.1371/ |
| | | journal.pone.0057269 |
| | | Dukoral, INN-cholera |
| | | vaccine (inactivated |
| | | oral) Annex I: |
| | | Summary of product |
| | | Characteristics |
| | | ema.europa.eu/en/ |
| | | documents/product- |
| | | information/dukoral- |
| | | epar-product- |
| | | information_en.pdf |
| | | Mottram, L., Lundgren, |
| | | A., Svennerholm, A. |
| | | M., & Leach, S. (2021) |
| | | frontiersin. |
| | | org/articles/10.3389/ |
| | | fimmu.2021.647873/ |
| | | full?utm_source = S- |
| | | TWT&utm_medium = |
| | | SNET&utm_campaign = |
| | | ECO_FIMMU_ |
| | | XXXXXXXX_auto- |
| | | dlvrit |
| Cryptococcus | Cda1, Cda2, Cda3, Fpd1, MP88, and Sod1 | Specht, C.A., et al., |
| neoformans | | (2017). journals.asm.org/ |
| | | doi/full/10.1128/ |
| | | mBio.01872-17 |
| Diphtheria | diphtheria toxoid | Stratton, K., Ford, A., |
| | Antigens for Corynebacterium diphtheriae | Rusch, E., Clayton, E. |
| | | W., & Committee to |
| | | Review Adverse |
| | | Effects of Vaccines. |
| | | (2011). ncbi.nlm.nih.gov/ |
| | | books/NBK190028/ |
| | | US20210023199A1 |
| Haemophilus | PRP | Kelly, D.F., Moxon, E. |
| Influenzae type b | | R., & Pollard, A.J. |
| | | (2004). ncbi.nlm.nih.gov/ |
| | | pmc/articles/ |
| | | PMC1782565/ |
| Hepatitis A | inactivated hepatitis A virus (strain HM175) | rxlist.com/twinrix- |
| | | drug.htm |
| Hepatitis B | noninfectious hepatitis B virus surface | See above |
| | antigen (HBsAg) | |
| Histoplasma | Hsp60 from Histoplasma capsulatum | Deepe, Jr., G.S., & |
| | | Gibbons, R.S. (2002). |
| | | journals.asm.org/ |
| | | doi/10.1128/ |
| | | IAI.70.7.3759-3767.2002 |
| Human | gag, pol, env, and nef | IP Nascimento and LCC |
| Immunodeficiency | | Leite, Braz J Med Biol |
| Virus (HIV) | | Res. 2012 doi: |
| | | 10.1590/SO100- |
| | | 879X2012007500142 |
| Human Papillomavirus | L1 major capsid protein of HPV | cdc.gov/vaccines/ |
| Infection | L2 major capsid protein of HPV | pubs/pinkbook/ |
| | | hpv.html |
| | | doi.org/10.1128/ |
| | | JVI.75.19.9201- |
| | | 9209.2001 |
| Influenza | Influenza A virus (HA, NA, NP, M2, M1 | US20210023199A1 |
| | antigens), influenza B virus (HA, NA | |
| | antigens), respiratory syncytial virus (F, G, | |
| | M, SH antigens), parainfluenza virus | |
| | (glycoprotein antigens) | |
| Leishmania spp. | Tagatose-6-phosphate kinase-like protein | John, L., John, G.J., & |
| | (XP_822202.1) | Kholia, T. (2012). |
| | Phosphatidylinositol 3-kinase-like protein | link.springer.com/ |
| | (XP_822211.1) | article/10.1007/ |
| | XP_001687567.1 is a surface antigen | s12010-012-9649-0 |
| | protein and is predicted to be an | doi.org/10.1371/ |
| | extracellular/secreted protein | journal.pntd.0005527 |
| | Phosphoglycan beta 1,3 | doi.org/10.1089/ |
| | galactosyltransferase 4 (XP_822217.1, | hum.1998.9.13-1899 |
| | XP_822221.1, and XP_001686570.1) and | doi.org/10.1084/ |
| | glycosomal membrane protein | jem.186.7.1137 |
| | (XP_843475.1) | |
| | Proteophosphoglycan ppg4 (XP_843162.1), | |
| | proteophosphoglycan ppg5 (XP_843163.1), | |
| | and proteophosphoglycan ppg1 | |
| | (XP_843164.1) | |
| | Tuzin protein (XP_001686384.1) | |
| | Receptor-type adenylate cyclase a-like | |
| | protein (XP_001686897.1) | |
| | KMP11: kinetoplastid membrane protein-11 | |
| | HASPB: hydrophilic acylated surface protein | |
| | B | |
| | GP63: glycoprotein 63 | |
| | LACK: Leishmania homologue for receptors | |
| | of activated C kinase | |
| | A2: Amastigote-specific A2 proteins | doi.org/10.1016/ |
| | NH: nucleoside hydrolase | j.micinf.2007.05.012 |
| | TSA: Thiol-specific antioxidant | doi.org/10.5812/ |
| | LeIF: Leishmania elongation initiation factor | jjm.8974 doi.org/10.1016/ |
| | LmSTI1: Leishmania major stress-inducible | j.vaccine.2011.02.096 |
| | protein 1 | |
| Listeria | listeriolysin O (LLO) | Calder0n-Gonzalez, R., |
| monocytogenes | glyceraldehyde-3-phosphate- | Frande-Cabanes, E., |
| | dehydrogenase (GAPDH) | Bronchalo-Vicente, L., |
| | | Lecea-Cuello, M.J., |
| | | Pareja, E., Bosch- |
| | | Martinez, A., . . . & |
| | | Alvarez-Dominguez, C. |
| | | (2014). frontiersin.org/ |
| | | articles/10.3389/ |
| | | fcimb.2014.00022/full |
| Measles | measles virus antigens (e.g., live attenuated | US9107831B2 |
| | virus antigens) | |
| Meningococcal | men ingococcal (Neisseria meningitidis) | US9107831 B2 |
| Disease | antigens, polysaccharide and conjugate | |
| | antigens (e.g., meningitis A, meningitis B, | |
| | meningitis C, meningitis W, meningitis Y) | |
| Mumps | mumps virus antigens (e.g., live, attenuated | US9107831B2 |
| | virus antigens) | |
| Mycobacteria | Early secretory antigenic target-6 (ESAT6) | Kwon, B.E., Ahn, J. |
| | | H., Park, E.K., Jeong, |
| | | H., Lee, H.J., Jung, Y. |
| | | J., . . . & Ko, H. J. |
| | | (2019). frontiersin.org/ |
| | | articles/10.3389/ |
| | | fimmu.2019.02542/full |
| Pertussis (whooping | pertussis toxin (PT), filamentous | US20200405839A1 |
| cough) | hemagglutinin (FHA), fimbriae (FIM), | |
| | pertactin (PRN), the siderophore receptor | |
| | protein FauA, the xenosiderophore receptor | |
| | protein BfeA, the hemophore receptor | |
| | protein BfuR | |
| Poliomyelitis | inactivated poliovirus serotypes, D-antigen | Bandyopadhyay, A.S., |
| | (D-Ag) | Garon, J., Seib, K., & |
| | polio virus (VP1-4) | Orenstein, W.A. |
| | | (2015). futuremedicine.com/ |
| | | doi/10.2217/fmb.15.19 |
| | | US20210023199A1 |
| Pneumococcal | pneumococcal (Streptococcus pneumoniae) | US91 07831B2 |
| Disease | antigens (e.g., polysaccharide and | |
| | conjugate antigens) | |
| | Streptococcus pneumoniae (Pht, PcsB, | US20210023199A1 |
| | StkP antigens) | doi.org/10.5863/1551- |
| | PspA: Pneumococcal surface protein A | 6776-21.1.27 |
| | PlyD1: genetically detoxified pneumolysin | doi.org/10.1016/ |
| | protein with 3 mutations | j.vaccine.2012.11.005 |
| | RrgB321: Fusion Protein of the Three | doi.org/10.1128/ |
| | Variants of the Pneumococcal Pilus | IAI.05780-11 |
| | Backbone RrgB | doi.org/10.5863/ |
| | PcsB | 1551-6776-21.1.27 |
| | StkP: serine-threonine protein kinase | doi.org/10.3390/ |
| | PsaA: pneumococcal surface adhesion | vaccines7010009 |
| | protein A | doi.org/10.3390/ |
| | PspC: Pneumococcal surface protein C | vaccines7010009 |
| | PhtD: pneumococcal histidine triad D | |
| | PcpA: pneumococcal-choline binding | |
| | protein A | |
| Legionella | recombinant peptidoglycan-associated | Mobarez, A.M. |
| pneumophila | lipoprotein (rPAL) | Rajabi, R.A., |
| | | Salmanian, A.H., |
| | | Khoramabadi, N., & |
| | | Doust, S.R.H. (2019). |
| | | pubmed.ncbi.nlm.nih.gov/ |
| | | 30550844/ |
| Protozoa | Fucose-Mannose-Ligand glycoprotein | Mcallister, M. M. |
| | antigen | (2014). ncbi.nlm.nih.gov/ |
| | Leishmania amastigote antigen (A2) | pmc/articles/PM |
| | Excreted Secreted Proteins (ESP) of L. | C3961066/ |
| | infantum | |
| Rickettsia spp. | rickettsia) outer membrane protein B | Chan, Y.G.Y., Riley, S. |
| | (rOmpB) | P., Chen, E., & |
| | | Martinez, J. J. (2011). |
| | | ncbi.nlm.nih.gov/pmc/ |
| | | articles/PMC3125829/ |
| Rotavirus | VP6 | Svensson, L., |
| | | Sheshberadaran, H., |
| | | Vesikari, T., Norrby, |
| | | E., & Wadell, G. |
| | | (1987). microbiology- |
| | | research.org/content/ |
| | | journal/jgv/10.1099/ |
| | | 0022-1317-68-7- |
| | | 1993?crawler = true |
| Rubella | rubella virus antigen (e.g., live, attenuated | US9107831B2 |
| | virus antigens) | |
| Salmonella | lipid-A free lipopolysaccharide (LFPS) | M.H., Liang, Y.H., |
| typhimurium | | Chiu, C.H., . . . & Lee, |
| | | Y.C. (2020). |
| | | biomedsci.biomed- |
| | | central.com/articles/ |
| | | 10.1186/s12929-020- |
| | | 00681-8 |
| Shingles (Herpes | lyophilized varicella zoster virus | Monslow, M.A., |
| Zoster) | glycoprotein E (gE) | Elbashir, S., Sullivan, |
| | | N. L., Thiriot, D.S., |
| | | Ahl, P., Smith, J., . . . & |
| | | Vora, K. A. (2020). |
| | | sciencedirect.com/science/ |
| | | article/pii/ |
| | | S0264410X20308483 |
| Smallpox | lyophilized preparation of infectious vaccinia | Walsh, S.R., & Dolin, R. |
| | virus | (2011). tandfonline.com/ |
| | | doi/abs/10.1586/erv.11.79 |
| Tetanus | tetanus toxoid | Stratton, K., Ford, A., |
| | Antigens for clostridium tetani | Rusch, E., Clayton, E. |
| | 8MTT (tetanus toxin genetically inactivated | W., & Committee to |
| | with 8 as mutations) | Review Adverse Effects |
| | TeNT-Hc (tetanus toxin fragment C) | of Vaccines. (2011). |
| | | ncbi.nlm.nih.gov/books/ |
| | | NBK1900 28/ |
| | | US20210023199A1 |
| | | Przedpelski et al. |
| | | mBio. 2020 Aug |
| | | 11; 11(4): e01668-20. |
| | | doi: 10.1128/ |
| | | mBio.01668-20. |
| | | Yu et al. |
| | | Toxins 2016, 8(7), |
| | | 194; doi: |
| | | 10.3390/toxins8070194 |
| Trypanosome spp. | VSG | Akhoon, B.A., Slathia, |
| | | P.S., Sharma, P., |
| | | Gupta, S.K., & Verma, |
| | | V. (2011). |
| | | sciencedirect.com/ |
| | | science/article/a bs/pii/ |
| | | S0882401011000222 |
| | IFX: invariant flagellum antigen from | doi.org/10.1038/ |
| | T. vivax | s41586-021-03597-x |
| | TcG2: Trypanosoma cruzi protein G2 | doi.org/10.3389/ |
| | TcG4: Trypanosoma cruzi protein G4 | fimmu.2019.01456 |
| | Enolase | doi.org/10.1155/ |
| | TSA1: trypomastigote surface antigen | 2018/8964085 |
| | Tc24: trypomastigote excretory-secretory | doi.org/10.1016/ |
| | protein 24 | j.actatropica.2019.105168 |
| | Tc52: trypomastigote excretory-secretory | doi.org/10.1111/j.1574- |
| | protein 52 | 695X.2007.00251.x |
| | ASP1: amastigote surface protein 1 | doi.org/10.1016/ |
| | ASP0: amastigote surface protein 2 | j.actatropica.2019.105168 |
| | ASP9: amastigote surface protein 9 | |
| Mycobacterium | ESAT6, Ag85B, peptide 190-198 of MPT64, | Liu, Xun, et al., (2016). |
| tuberculosis | Mtb8.4, latency antigen Rv2626c | pubmed.ncbi.nlm.nih.gov/ |
| | resuscitation phase (RPfB-D) | 26901244/Shin, S.J., et al., |
| | Rv2041c | (2009). |
| | ESAT-6; Ag85b; TB10.4; RpfB-D; Rv2626 | pubmed.ncbi.nl m.nih.gov/ |
| | | 19874550/ |
| | | WO2014009438 A2 |
| Typhoid Fever | Vi polysaccharide or the live attenuated | Ni, Y., Springer, M.J., |
| | strain Ty21a | Guo, J., Finger-Baker, |
| | steD: fimbrial subunit | I., Wilson, J.P., Cobb, |
| | T2544 gene: possible outer membrane | R. R., . . . & Tizard, I. |
| | adhesin | (2017). |
| | OmpC: Outer membrane porin C | ncbi.nlm.nih.gov/pmc/ |
| | C-ter of sopB (last 261aa): inositol | articles/PMC5754192/ |
| | phosphatase | doi.org/10.1016/ |
| | SseB: Secreted effector protein | j.ygeno.2020.06.022 |
| | Flagellin | doi.org/10.1016/ |
| | Mig-14 | j.vaccine.2017.07.035 |
| | | doi.org/10.5402/ |
| | | 2012/512848 |
| | | doi.org/10.1016/ |
| | | j.vaccine.2009.02.092 |
| | | doi.org/10.1111/ |
| | | imm.12327 |
| | | doi.org/10.4049/ |
| | | jimmunol.1601357 |
| | LptD: LPS-assembly protein D | doi.org/10.1073/ |
| | LptE: LPS-assembly protein E | pnas.0401283101 |
| | | doi.org/10.1101/ |
| | | 521518 |
| Vaccines for Sexually | Gag, gp120, Pol, Nef, Env, Tat, Rev, Vpr, | US10894078B2 |
| Transmitted Diseases | Vif, Vpu | |
| Yersinia pestis | Caf1 | Chalton, D.A., |
| | low calcium response protein V (LcrV) | Musson, J.A., Flick- |
| | rV10 | Smith, H., Walker, N., |
| | recombinant FI and V proteins | McGregor, A., Lamb, |
| | rF1-V: recombinant fusion protein of the FI | H. K., . . . & Lakey, J.H. |
| | capsular protein and the LcrV protein | (2006). |
| | YadC: Yersinia adhesin A | journals.asm.org/ |
| | | doi/10.1128/iai.00437- |
| | | 06?permanently =true |
| | | Sun, W., & Singh, A.K. |
| | | (2019). nature.com/ |
| | | articles/s41541-019- |
| | | 0105-9 |
| | | doi.org/10.1016/ |
| | | j.vaccine.2019.07.103 |
| | | doi.org/10.1007/ |
| | | 978-0-387-72124-8 37 |
| Yellow Fever | Live attenuated yellow fever antigens | Gibney, K.B., |
| | NS1: Non-structural protein 1 | Edupuganti, S., Panella, |
| | YFE: Yellow Fever Envelope | A.J., Kosoy, O.I., |
| | | Delorey, M.J., |
| | | Lanciotti, R.S., . . . & |
| | | Staples, J. E. (2012). |
| | | ncbi.nlm.nih.gov/pmc/ |
| | | articles/PMC3516084/ |
| | | doi.org/10.3390/ |
| | | vaccines9060622 |
| | | doi.org/10.4269/ |
| | | ajtmh. 16-0293 |
| Other | Antigens for human papillomavirus (L1, L2, | US20210023199A1 |
| | E6, E7), SARS CoV (spike protein), | |
| | Staphylococcus aureus (IsdA, IsdB, toxin | |
| | antigens), Bordetella pertussis (toxin), | |
| | Plasmodium (NANP, CSP protein, ssp2, | |
| | ama1, msp142 antigens), Rabies virus (G, N | |
| | antigens), Staphylococcus aureus (toxin | |
| | antigen), Clostridium difficile (toxin antigen), | |
| | Candida albicans | |

Vaccines as discussed herein are suitably (although not exclusively), envisioned for direction towards intracellular pathogens, whether cytoplasmic or vesicular. Examples in this regard of intracellular cytoplasmic pathogens are viruses, Chlamydia spp., Rickettsia spp., Listeria monocytogenes, and protozoal parasites such as Plasmodium spp. Examples of vesicular intracellular pathogens include mycobacteria, Salmonella typhimurium, Leish mania spp., Listeria spp., Trypanosoma spp., Legionella pneumophila, Cryptococcus neoformans, Histoplasma, and Yersinia pestis.

Multiple antigens can be administered to a single subject, e.g., to provide a broader, stronger, or longer-lasting immunizing effect. This can be referred to as “antigen stacking.” In some embodiments of any of the aspects, the method further comprises administering one or more further antigen mRNA constructs, each further antigen mRNA construct comprising a further open reading frame (ORF), wherein each further ORF encodes an antigen distinct from the antigen encoded by the second ORF.

In some embodiments of any of the aspects, the one or more further antigen mRNA constructs is administered in the same composition as the first antigen construct. In some embodiments of any of the aspects, the one or more further antigen mRNA constructs is administered in the same composition as the first cytokine construct. In some embodiments of any of the aspects, the one or more further antigen mRNA constructs is administered a composition not comprising the first antigen construct and the first cytokine construct. In some embodiments of any of the aspects, the one or more further antigen mRNA constructs is administered concurrently with the first antigen construct and/or the first cytokine construct. In some embodiments of any of the aspects, the one or more further antigen mRNA constructs is administered sequentially with the first antigen construct and/or the first cytokine construct.

In some embodiments of any of the aspects, there are 1-9 further antigen mRNA constructs, e.g., administered and/or present in a composition.

In some embodiments of any of the aspects, a single mRNA molecule comprises the first antigen mRNA construct and further antigen mRNA constructs.

In some embodiments of any of the aspects, the first antigen mRNA construct further comprises one or more further open reading frames (ORFs), wherein each further ORF encodes an antigen distinct from the antigen encoded by the second ORF. In some embodiments of any of the aspects, there are 1-9 further antigen encoding ORFs, e.g., administered and/or present in a composition.

Where a composition or method relates to ORFs encoding multiple antigens, the antigens can be antigens from or derived from the same or different pathogens. That is, the patient can be immunized against a single pathogen using multiple antigens (e.g., to provide a stronger response or a response less likely to be evaded by the pathogen), and/or the patient can be immunized against multiple pathogens. In some embodiments of any of the aspects, the composition(s) comprises a plurality of ORFs encoding a plurality of antigens, and the plurality of antigens comprises multiple antigens from a first organism. In some embodiments of any of the aspects, the composition(s) comprises a plurality of ORFs encoding a plurality of antigens, and the plurality of antigens comprises one or more antigens from a first organism and one or more antigens from one or more further organisms.

In some embodiments of any of the aspects, the composition(s) comprises a plurality of ORFs encoding a plurality of antigens, and the plurality of antigens comprises one or more antigens from a coronavirus and one or more antigens from an influenza virus. In some embodiments of any of the aspects, the composition(s) comprises a plurality of ORFs encoding a plurality of antigens, and the plurality of antigens comprises one or more spike protein antigens from a coronavirus and one or more antigens from an influenza virus.

In some embodiments of any of the aspects, the antigen(s) is an antigen of an infectious organism and whereby transmission of the infectious organism to or by the subject is reduced as compared to administration of a composition not comprising the cytokine mRNA construct.

As described herein, an “antigen” is a molecule that is specifically bound by a B cell receptor (BCR), T cell receptor (TCR), and/or antibody, thereby activating an immune response. An antigen can be pathogen-derived, or originate from a pathogen. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule. Exemplary but non-limiting antigens include a pathogenic microbial protein or an epitope containing fragment thereof.

Exemplary non-limiting pathogenic microbial proteins include a viral protein; a bacterial protein; a fungal protein; a parasite protein; and a prion. In some embodiments of any of the aspects, the antigen comprises a viral protein or an epitope containing fragment thereof.

In some embodiments, the mRNA encoding an antigen can encode one or more viral proteins of the Severe acute respiratory syndrome coronavirus, like the severe acute respiratory syndrome coronavirus 2 virus (SARS-COV-2), that is, the virus responsible for the Covid-19 pandemic. This virus has four structural proteins, the S (spike), E (envelope), M (membrane), and N (nucleocapsid) proteins. In some embodiments, the coding mRNA encodes all or part of the spike protein of SARS-COV-2. In some embodiments, the mRNA encodes the prefusion form of the S protein ectodomain (amino acids 1 to 1208 with proline substitutions at residues 986 and 987; GenBank MN908947). In some embodiments, the mRNA encodes the Spike protein's Receptor Binding Domain or RBD (residues 319 to 591; GenBank MN908947). As an external part of this protein, this is a likely location for epitopes which could be recognized by the immune system. In some embodiments, the mRNA encodes all or part of the spike protein of a variant of SARS-COV-2, for example, that of the Alpha, Beta, Gamma, Epsilon, Delta, Kappa, Eta, or Omicron variants. In some embodiments, the mRNA includes one or more of the sequences recited in Table 6A below (SEQ ID NOs: 62 to 67), or a sequence with at least 80%, at least 85%, at least 90%, at least 95%, at least 98%, or at least 99% similarity thereto.

In some embodiments, the mRNA encoding a spike protein or part thereof has been codon-optimised for expression in human or other mammalian cells. In some embodiments, one or more of the nucleosides used in the mRNA are been replaced by an isomer thereof. As example one, more or all of the uridine nucleosides in the mRNA construct are replaced by pseudouridine nucleosides. In one embodiment, the mRNA encodes the spike protein of the SARS-COV-2 Delta variant, and the organ protecting MOP sequence of the mRNA comprises target sites for each of miRNA 122, miRNA 192 and miRNA 30a, and in another embodiment further comprises a target site for miRNA let7b. In other embodiments of the invention described in more detail below, the mRNA encodes a prefusion spike protein of the SARS-COV-2 selected from non-codon optimized or human codon optimized Wuhan strain, beta variant or alpha variant, with or without a MOP sequence. The MOP sequence may be selected from one that comprises the following combinations of miRNA binding sequences: miRNA 122, miRNA 192 and miRNA 30a; and let7b, miRNA 126, and miRNA 30a; miRNA 122, miRNA 1, miRNA 203a, and miRNA 30a. It will be appreciated that other MOP sequences may be selected depending upon the particular context in which organ protection is required. As described herein, the selected MOP sequences may comprise miRNA binding sequences that are further optimised to ensure perfect match hybridisation with the respective target miRNA sequence in the body.

TABLE 6A
Exemplary mRNA constructs for a range of SARS-CoV-2 spike protein
variants suitable for use in vaccine compositions

Prefusion Spike mRNA sequence from Wuhan strain with K986P and V987P mutation [SEQ ID NO: 62]
The codons are not optimized for human cellular expression. This mRNA does not contain a MOP
sequence
AUGUUUGUUUUUCUUGUUUUAUUGCCACUAGUCUCUAGUCAGUGUGUUAAUCUUA
CAACCAGAACUCAAUUACCCCCUGCAUACACUAAUUCUUUCACACGUGGUGUU
UAUUACCCUGACAAAGUUUUCAGAUCCUCAGUUUUACAUUCAACUCAGGACUUGUUCUUAC
CUUUCUUUUCCAAUGUUACUUGGUUCCAUGCUAUACAUGUCUCUGGGACCAAUGGUACUAA
GAGGUUUGAUAACCCUGUCCUACCAUUUAAUGAUGGUGUUUAUUUUGCUUCCACUGAGAAG
UCUAACAUAAUAAGAGGCUGGAUUUUUGGUACUACUUUAGAUUCGAAGACCCAGUCCCUAC
UUAUUGUUAAUAACGCUACUAAUGUUGUUAUUAAAGUCUGUGAAUU
UCAAUUUUGUAAUGAUCCAUUUUUGGGUGUUUAUUACCACAAAA
ACAACAAAAGUUGGAUGGAAAGUGAGUUCAGAGUUUAUUCUAGUGCGAAUAAUUG
CACUUUUGAAUAUGUCUCUCAGCCUUUUCUUAUGGACCUUGAAGGAAAACAGGGU
AAUUUCAAAAAUCUUAGGGAAUUUGUGUUUAAGAAUAUUGAUGGUUAUUUUAAAAU
AUAUUCUAAGCACACGCCUAUUAAUUUAGUGCGUGAUCUCCCUCAGGGUUUUUCG
GCUUUAGAACCAUUGGUAGAUUUGCCAAUAGGUAUUAACAUCACUAGGUUUCAAA
CUUUACUUGCUUUACAUAGAAGUUAUUUGACUCCUGGUGAUUCUUCUUCAGGUUG
GACAGCUGGUGCUGCAGCUUAUUAUGUGGGUUAUCUUCAACCUAGGACUUUUCUA
UUAAAAUAUAAUGAAAAUGGAACCAUUACAGAUGCUGUAGACUGUGCACUUGACCC
UCUCUCAGAAACAAAGUGUACGUUGAAAUCCUUCACUGUAGAAAAAGGAAUCUAUC
AAACUUCUAACUUUAGAGUCCAACCAACAGAAUCUAUUGUUAGAUUUCCUAAUAUU
ACAAACUUGUGCCCUUUUGGUGAAGUUUUUAACGCCACCAGAUUUGCAUCUGUUU
AUGCUUGGAACAGGAAGAGAAUCAGCAACUGUGUUGCUGAUUAUUCUGUCCUAUA
UAAUUCCGCAUCAUUUUCCACUUUUAAGUGUUAUGGAGUGUCUCCUACUAAAUUA
AAUGAUCUCUGCUUUACUAAUGUCUAUGCAGAUUCAUUUGUAAUUAGAGGUGAUG
AAGUCAGACAAAUCGCUCCAGGGCAAACUGGAAAGAUUGCUGAUUAUAAUUAUAAA
UUACCAGAUGAUUUUACAGGCUGCGUUAUAGCUUGGAAUUCUAACAAUCUUGAUU
CUAAGGUUGGUGGUAAUUAUAAUUACCUGUAUAGAUUGUUUAGGAAGUCUAAUCU
CAAACCUUUUGAGAGAGAUAUUUCAACUGAAAUCUAUCAGGCCGGUAGCACACCU
UGUAAUGGUGUUGAAGGUUUUAAUUGUUACUUUCCUUUACAAUCAUAUGGUUUCC
AACCCACUAAUGGUGUUGGUUACCAACCAUACAGAGUAGUAGUACUUUCUUUUGA
ACUUCUACAUGCACCAGCAACUGUUUGUGGACCUAAAAAGUCUACUAAUUUGGUU
AAAAACAAAUGUGUCAAUUUCAACUUCAAUGGUUUAACAGGCACAGGUGUUCUUAC
UGAGUCUAACAAAAAGUUUCUGCCUUUCCAACAAUUUGGCAGAGACAUUGCUGAC
ACUACUGAUGCUGUCCGUGAUCCACAGACACUUGAGAUUCUUGACAUUACACCAU
GUUCUUUUGGUGGUGUCAGUGUUAUAACACCAGGAACAAAUACUUCUAACCAGGU
UGCUGUUCUUUAUCAGGAUGUUAACUGCACAGAAGUCCCUGUUGCUAUUCAUGCA
GAUCAACUUACUCCUACUUGGCGUGUUUAUUCUACAGGUUCUAAUGUUUUUCAAA
CACGUGCAGGCUGUUUAAUAGGGGCUGAACAUGUCAACAACUCAUAUGAGUGUGA
CAUACCCAUUGGUGCAGGUAUAUGCGCUAGUUAUCAGACUCAGACUAAUUCUCCU
CGGCGGGCACGUAGUGUAGCUAGUCAAUCCAUCAUUGCCUACACUAUGUCACUUG
GUGCAGAAAAUUCAGUUGCUUACUCUAAUAACUCUAUUGCCAUACCCACAAAUUUU
ACUAUUAGUGUUACCACAGAAAUUCUACCAGUGUCUAUGACCAAGACAUCAGUAGA
UUGUACAAUGUACAUUUGUGGUGAUUCAACUGAAUGCAGCAAUCUUUUGUUGCAA
UAUGGCAGUUUUUGUACACAAUUAAACCGUGCUUUAACUGGAAUAGCUGUUGAAC
AAGACAAAAACACCCAAGAAGUUUUUGCACAAGUCAAACAAAUUUACAAAACACCAC
CAAUUAAAGAUUUUGGUGGUUUUAAUUUUUCACAAAUAUUACCAGAUCCAUCAAAA
CCAAGCAAGAGGUCAUUUAUUGAAGAUCUACUUUUCAACAAAGUGACACUUGCAGA
UGCUGGCUUCAUCAAACAAUAUGGUGAUUGCCUUGGUGAUAUUGCUGCUAGAGAC
CUCAUUUGUGCACAAAAGUUUAACGGCCUUACUGUUUUGCCACCUUUGCUCACAG
AUGAAAUGAUUGCUCAAUACACUUCUGCACUGUUAGCGGGUACAAUCACUUCUGG
UUGGACCUUUGGUGCAGGUGCUGCAUUACAAAUACCAUUUGCUAUGCAAAUGGCU
UAUAGGUUUAAUGGUAUUGGAGUUACACAGAAUGUUCUCUAUGAGAACCAAAAAUU
GAUUGCCAACCAAUUUAAUAGUGCUAUUGGCAAAAUUCAAGACUCACUUUCUUCCA
CAGCAAGUGCACUUGGAAAACUUCAAGAUGUGGUCAACCAAAAUGCACAAGCUUUA
AACACGCUUGUUAAACAACUUAGCUCCAAUUUUGGUGCAAUUUCAAGUGUUUUAAA
UGAUAUCCUUUCACGUCUUGACCCGCCGGAGGCUGAAGUGCAAAUUGAUAGGUUG
AUCACAGGCAGACUUCAAAGUUUGCAGACAUAUGUGACUCAACAAUUAAUUAGAGC
UGCAGAAAUCAGAGCUUCUGCUAAUCUUGCUGCUACUAAAAUGUCAGAGUGUGUA
CUUGGACAAUCAAAAAGAGUUGAUUUUUGUGGAAAGGGCUAUCAUCUUAUGUCCU
UCCCUCAGUCAGCACCUCAUGGUGUAGUCUUCUUGCAUGUGACUUAUGUCCCUGC
ACAAGAAAAGAACUUCACAACUGCUCCUGCCAUUUGUCAUGAUGGAAAAGCACACU
UUCCUCGUGAAGGUGUCUUUGUUUCAAAUGGCACACACUGGUUUGUAACACAAAG
GAAUUUUUAUGAACCACAAAUCAUUACUACAGACAACACAUUUGUGUCUGGUAACU
GUGAUGUUGUAAUAGGAAUUGUCAACAACACAGUUUAUGAUCCUUUGCAACCUGA
AUUAGACUCAUUCAAGGAGGAGUUAGAUAAAUAUUUUAAGAAUCAUACAUCACCAG
AUGUUGAUUUAGGUGACAUCUCUGGCAUUAAUGCUUCAGUUGUAAACAUUCAAAA
AGAAAUUGACCGCCUCAAUGAGGUUGCCAAGAAUUUAAAUGAAUCUCUCAUCGAUC
UCCAAGAACUUGGAAAGUAUGAGCAGUAUAUAAAAUGGCCAUGGUACAUUUGGCU
AGGUUUUAUAGCUGGCUUGAUUGCCAUAGUAAUGGUGACAAUUAUGCUUUGCUGU
AUGACCAGUUGCUGUAGUUGUCUCAAGGGCUGUUGUUCUUGUGGAUCCUGCUGC
AAAUUUGAUGAAGACGACUCUGAGCCAGUGCUCAAAGGAGUCAAAUUACAUUACAC AUAA
Prefusion Spike mRNA sequence from Wuhan strain with MOPV in 3′ UTR [SEQ ID NO: 63]
Codons are not optimized for human cellular expression
MOPV sequence is shown at 3′ end with underlining comprises binding sequences
for miRNA-122-5P, miRNA-1-3P, miRNA-203a-3P, miRNA-30a-5P
AUGUUUGUUUUUCUUGUUUUAUUGCCACUAGUCUCUAGUCAGUGUGUUAAUCUUA
CAACCAGAACUCAAUUACCCCCUGCAUACACUAAUUCUUUCACACGUGGUGUUUAU
UACCCUGACAAAGUU UUCAGAUCCUCAGUUUUACAUUCAACUCAGGACUUGUUCU
UACCUUUCUUUUCCAAUGUUACUUGGUUCCAUGCUAUACAUGUCUCUGGGACCAA
UGGUACUAAGAGGUUUGAUAACCCUGUCCUACCAUUUAAUGAUGGUGUUUAUUUU
GCUUCCACUGAGAAGUCUAACAUAAUAAGAGGCUGGAUUUUUGGUACUACUUUAG
AUUCGAAGACCCAGUCCCUACUUAUUGUUAAUAACGCUACUAAUGUUGUUAUUAAA
GUCUGUGAAUUUCAAUUUUGUAAUGAUCCAUUUUUGGGUGUUUAUUACCACAAAA
ACAACAAAAGUUGGAUGGAAAGUGAGUUCAGAGUUUAUUCUAGUGCGAAUAAUUG
CACUUUUGAAUAUGUCUCUCAGCCUUUUCUUAUGGACCUUGAAGGAAAACAGGGU
AAUUUCAAAAAUCUUAGGGAAUUUGUGUUUAAGAAUAUUGAUGGUUAUUUUAAAAU
AUAUUCUAAGCACACGCCUAUUAAUUUAGUGCGUGAUCUCCCUCAGGGUUUUUCG
GCUUUAGAACCAUUGGUAGAUUUGCCAAUAGGUAUUAACAUCACUAGGUUUCAAA
CUUUACUUGCUUUACAUAGAAGUUAUUUGACUCCUGGUGAUUCUUCUUCAGGUUG
GACAGCUGGUGCUGCAGCUUAUUAUGUGGGUUAUCUUCAACCUAGGACUUUUCUA
UUAAAAUAUAAUGAAAAUGGAACCAUUACAGAUGCUGUAGACUGUGCACUUGACCC
UCUCUCAGAAACAAAGUGUACGUUGAAAUCCUUCACUGUAGAAAAAGGAAUCUAUC
AAACUUCUAACUUUAGAGUCCAACCAACAGAAUCUAUUGUUAGAUUUCCUAAUAUU
ACAAACUUGUGCCCUUUUGGUGAAGUUUUUAACGCCACCAGAUUUGCAUCUGUUU
AUGCUUGGAACAGGAAGAGAAUCAGCAACUGUGUUGCUGAUUAUUCUGUCCUAUA
UAAUUCCGCAUCAUUUUCCACUUUUAAGUGUUAUGGAGUGUCUCCUACUAAAUUA
AAUGAUCUCUGCUUUACUAAUGUCUAUGCAGAUUCAUUUGUAAUUAGAGGUGAUG
AAGUCAGACAAAUCGCUCCAGGGCAAACUGGAAAGAUUGCUGAUUAUAAUUAUAAA
UUACCAGAUGAUUUUACAGGCUGCGUUAUAGCUUGGAAUUCUAACAAUCUUGAUU
CUAAGGUUGGUGGUAAUUAUAAUUACCUGUAUAGAUUGUUUAGGAAGUCUAAUCU
CAAACCUUUUGAGAGAGAUAUUUCAACUGAAAUCUAUCAGGCCGGUAGCACACCU
UGUAAUGGUGUUGAAGGUUUUAAUUGUUACUUUCCUUUACAAUCAUAUGGUUUCC
AACCCACUAAUGGUGUUGGUUACCAACCAUACAGAGUAGUAGUACUUUCUUUUGA
ACUUCUACAUGCACCAGCAACUGUUUGUGGACCUAAAAAGUCUACUAAUUUGGUU
AAAAACAAAUGUGUCAAUUUCAACUUCAAUGGUUUAACAGGCACAGGUGUUCUUAC
UGAGUCUAACAAAAAGUUUCUGCCUUUCCAACAAUUUGGCAGAGACAUUGCUGAC
ACUACUGAUGCUGUCCGUGAUCCACAGACACUUGAGAUUCUUGACAUUACACCAU
GUUCUUUUGGUGGUGUCAGUGUUAUAACACCAGGAACAAAUACUUCUAACCAGGU
UGCUGUUCUUUAUCAGGAUGUUAACUGCACAGAAGUCCCUGUUGCUAUUCAUGCA
GAUCAACUUACUCCUACUUGGCGUGUUUAUUCUACAGGUUCUAAUGUUUUUCAAA
CACGUGCAGGCUGUUUAAUAGGGGCUGAACAUGUCAACAACUCAUAUGAGUGUGA
CAUACCCAUUGGUGCAGGUAUAUGCGCUAGUUAUCAGACUCAGACUAAUUCUCCU
CGGCGGGCACGUAGUGUAGCUAGUCAAUCCAUCAUUGCCUACACUAUGUCACUUG
GUGCAGAAAAUUCAGUUGCUUACUCUAAUAACUCUAUUGCCAUACCCACAAAUUUU
ACUAUUAGUGUUACCACAGAAAUUCUACCAGUGUCUAUGACCAAGACAUCAGUAGA
UUGUACAAUGUACAUUUGUGGUGAUUCAACUGAAUGCAGCAAUCUUUUGUUGCAA
UAUGGCAGUUUUUGUACACAAUUAAACCGUGCUUUAACUGGAAUAGCUGUUGAAC
AAGACAAAAACACCCAAGAAGUUUUUGCACAAGUCAAACAAAUUUACAAAACACCAC
CAAUUAAAGAUUUUGGUGGUUUUAAUUUUUCACAAAUAUUACCAGAUCCAUCAAAA
CCAAGCAAGAGGUCAUUUAUUGAAGAUCUACUUUUCAACAAAGUGACACUUGCAGA
UGCUGGCUUCAUCAAACAAUAUGGUGAUUGCCUUGGUGAUAUUGCUGCUAGAGAC
CUCAUUUGUGCACAAAAGUUUAACGGCCUUACUGUUUUGCCACCUUUGCUCACAG
AUGAAAUGAUUGCUCAAUACACUUCUGCACUGUUAGCGGGUACAAUCACUUCUGG
UUGGACCUUUGGUGCAGGUGCUGCAUUACAAAUACCAUUUGCUAUGCAAAUGGCU
UAUAGGUUUAAUGGUAUUGGAGUUACACAGAAUGUUCUCUAUGAGAACCAAAAAUU
GAUUGCCAACCAAUUUAAUAGUGCUAUUGGCAAAAUUCAAGACUCACUUUCUUCCA
CAGCAAGUGCACUUGGAAAACUUCAAGAUGUGGUCAACCAAAAUGCACAAGCUUUA
AACACGCUUGUUAAACAACUUAGCUCCAAUUUUGGUGCAAUUUCAAGUGUUUUAAA
UGAUAUCCUUUCACGUCUUGACCCGCCGGAGGCUGAAGUGCAAAUUGAUAGGUUG
AUCACAGGCAGACUUCAAAGUUUGCAGACAUAUGUGACUCAACAAUUAAUUAGAGC
UGCAGAAAUCAGAGCUUCUGCUAAUCUUGCUGCUACUAAAAUGUCAGAGUGUGUA
CUUGGACAAUCAAAAAGAGUUGAUUUUUGUGGAAAGGGCUAUCAUCUUAUGUCCU
UCCCUCAGUCAGCACCUCAUGGUGUAGUCUUCUUGCAUGUGACUUAUGUCCCUGC
ACAAGAAAAGAACUUCACAACUGCUCCUGCCAUUUGUCAUGAUGGAAAAGCACACU
UUCCUCGUGAAGGUGUCUUUGUUUCAAAUGGCACACACUGGUUUGUAACACAAAG
GAAUUUUUAUGAACCACAAAUCAUUACUACAGACAACACAUUUGUGUCUGGUAACU
GUGAUGUUGUAAUAGGAAUUGUCAACAACACAGUUUAUGAUCCUUUGCAACCUGA
AUUAGACUCAUUCAAGGAGGAGUUAGAUAAAUAUUUUAAGAAUCAUACAUCACCAG
AUGUUGAUUUAGGUGACAUCUCUGGCAUUAAUGCUUCAGUUGUAAACAUUCAAAA
AGAAAUUGACCGCCUCAAUGAGGUUGCCAAGAAUUUAAAUGAAUCUCUCAUCGAUC
UCCAAGAACUUGGAAAGUAUGAGCAGUAUAUAAAAUGGCCAUGGUACAUUUGGCU
AGGUUUUAUAGCUGGCUUGAUUGCCAUAGUAAUGGUGACAAUUAUGCUUUGCUGU
AUGACCAGUUGCUGUAGUUGUCUCAAGGGCUGUUGUUCUUGUGGAUCCUGCUGC
AAAUUUGAUGAAGACGACUCUGAGCCAGUGCUCAAAGGAGUCAAAUUACAUUACAC
AUAACAAACACCAUUGUCACACUCCAUUUAAAAUACAUACUUCUUUACAUUCCAUU
UAAACUAGUGGUCCUAAACAUUUCACUUUAAACUUCCAGUCGAGGAUGUUUACA
Prefusion Spike mRNA sequence from Wuhan strain with MOPC in 3′ UTR [SEQ ID NO: 64]
3′ MOP for miRNA-122-5P, miRNA-192-5P, miRNA-30a-5P (MOPC, underlined portion) Codons
optimized for human cellular expression
AUGUUCGUUUUCUUGGUCCUGCUUCCCCUGGUGUCUUCACAGUGCGUGAAUCUG
ACCACCAGAACACAGCUGCCUCCAGCAUACACCAACAGCUUCACCAGAGGCGUGU
AUUAUCCUGACAAGGUGUUUCGCUCCAGCGUGCUGCACAGCACCCAGGACCUUUU
UCUGCCUUUUUUCUCCAACGUGACAUGGUUCCACGCAAUCCACGUGAGCGGAACC
AACGGAACGAAGAGAUUCGACAACCCUGUGCUGCCCUUCAACGACGGAGUGUACU
UCGCCAGCACAGAGAAGAGCAACAUCAUCCGGGGCUGGAUCUUCGGAACCACCCU
GGACAGCAAAACCCAAUCUCUGCUUAUCGUGAACAACGCAACCAACGUGGUGAUC
AAGGUGUGUGAAUUCCAAUUUUGUAACGACCCAUUCCUGGGAGUGUACUACCAUA
AGAACAACAAGAGCUGGAUGGAAAGCGAGUUCCGGGUGUACAGCAGCGCCAACAA
CUGCACCUUCGAGUACGUGAGUCAGCCCUUUCUGAUGGACCUGGAAGGCAAGCAG
GGAAAUUUCAAGAAUCUGAGAGAGUUCGUGUUCAAAAACAUCGAUGGCUAUUUCA
AGAUCUAUAGCAAGCACACCCCUAUCAACCUGGUGAGAGAUCUGCCCCAGGGCUU
CAGCGCCCUGGAGCCUCUGGUAGACCUACCUAUCGGCAUCAACAUAACGAGAUUU
CAGACCCUGUUGGCUCUUCAUAGGAGCUACCUGACCCCCGGCGAUUCUAGCAGCG
GAUGGACAGCCGGCGCCGCUGCCUACUACGUUGGCUACCUGCAACCUCGGACAU
UCCUGCUGAAAUACAAUGAGAACGGCACUAUCACCGAUGCCGUGGACUGUGCCCU
GGAUCCUCUGAGCGAAACCAAGUGCACCCUGAAGAGCUUUACCGUGGAAAAGGGC
AUCUACCAGACCAGCAAUUUCCGGGUGCAGCCUACAGAGAGCAUCGUGAGAUUCC
CCAACAUCACCAAUCUGUGUCCUUUCGGCGAGGUGUUCAACGCUACAAGAUUCGC
AAGCGUGUACGCCUGGAAUCGGAAGCGGAUCAGUAACUGUGUGGCCGAUUAUUC
GGUGCUGUAUAAUUCUGCCAGCUUUAGCACCUUCAAGUGCUACGGUGUGAGCCCU
ACCAAACUCAACGACCUGUGCUUCACCAACGUGUAUGCCGACUCUUUCGUGAUCC
GGGGCGACGAGGUGCGGCAGAUCGCCCCCGGACAGACAGGCAAAAUCGCCGACU
ACAACUACAAGCUGCCUGACGACUUCACAGGGUGCGUUAUCGCCUGGAACAGCAA
CAAUCUGGAUUCAAAGGUGGGCGGAAACUACAACUACCUGUACAGACUGUUCAGA
AAGUCCAACCUGAAGCCCUUUGAGAGAGACAUCUCUACAGAAAUCUACCAGGCCG
GCUCCACCCCAUGCAACGGCGUGGAAGGCUUCAACUGCUACUUCCCCCUGCAGAG
CUACGGCUUUCAGCCUACCAAUGGCGUCGGUUACCAGCCUUACCGCGUGGUCGU
UCUAUCCUUCGAGCUGCUGCACGCCCCUGCUACAGUGUGCGGACCUAAGAAGAGC
ACAAACCUGGUCAAAAACAAGUGUGUCAACUUCAACUUCAACGGCCUGACCGGCAC
AGGCGUACUGACAGAAAGCAACAAGAAGUUCCUGCCUUUCCAGCAGUUUGGCAGA
GAUAUCGCUGAUACCACAGACGCCGUGCGGGAUCCUCAGACACUGGAAAUCCUGG
ACAUCACACCCUGCAGCUUUGGCGGCGUGUCUGUCAUCACCCCAGGCACCAACAC
GUCCAACCAAGUGGCCGUGCUGUACCAGGACGUCAACUGCACCGAGGUGCCCGU
UGCUAUCCACGCCGAUCAGCUAACUCCUACCUGGCGGGUUUAUAGCACCGGAUCU
AACGUGUUCCAGACCAGGGCCGGAUGUCUGAUCGGCGCUGAACACGUAAACAAUA
GCUACGAGUGUGAUAUCCCUAUCGGAGCCGGCAUCUGUGCCAGCUACCAGACCCA
GACAAAUUCUCCACGGAGAGCUAGAAGCGUCGCCUCUCAGAGCAUCAUCGCCUAC
ACCAUGAGCCUGGGCGCCGAAAACUCUGUGGCCUACAGCAACAACAGCAUCGCCA
UUCCCACCAACUUUACAAUCAGCGUGACAACAGAGAUCCUGCCUGUGAGCAUGAC
CAAAACCAGCGUGGACUGCACCAUGUACAUCUGCGGCGACAGCACCGAAUGCUCU
AACCUUCUGCUGCAAUACGGCAGCUUCUGCACUCAGCUGAACAGAGCCCUGACCG
GCAUCGCCGUGGAGCAGGAUAAGAACACCCAGGAGGUGUUCGCCCAGGUGAAACA
AAUCUACAAGACACCUCCCAUCAAGGACUUCGGCGGAUUUAACUUCAGCCAGAUC
CUGCCUGACCCAUCUAAGCCUAGCAAGCGGUCCUUUAUCGAGGACUUGCUGUUCA
ACAAGGUGACCCUGGCCGAUGCCGGCUUUAUCAAGCAGUACGGCGACUGCCUUG
GCGACAUCGCCGCCAGAGACCUGAUCUGCGCCCAGAAGUUUAACGGCCUGACAGU
GCUGCCUCCUCUGCUGACCGACGAAAUGAUCGCCCAGUAUACCAGCGCUCUGCUGGCGGGUA
CCAUCACCAGCGGCUGGACCUUCGGCGCCGGCGCUGCUCUGCAGAUC
CCUUUCGCCAUGCAGAUGGCCUACCGGUUCAACGGCAUUGGCGUGACCCAGAACG
UGCUGUACGAGAAUCAGAAGCUGAUCGCCAACCAGUUCAACAGCGCUAUCGGAAA
AAUCCAGGACUCUCUGAGCUCUACUGCCUCUGCUCUUGGGAAACUGCAGGACGUGGU
UAAUCAGAACGCCCAGGCCCUCAAUACCCUGGUGAAGCAACUGUCGAGCAAUU
UUGGCGCCAUCAGCAGCGUGCUGAAUGACAUUCUGUCUAGACUGGACCCUCCAGA
AGCUGAGGUGCAGAUUGACAGACUGAUCACAGGCAGACUGCAGAGCCUGCAGACC
UACGUGACCCAACAACUGAUCAGAGCCGCCGAGAUUAGGGCCUCUGCCAAUCUGG
CCGCCACGAAAAUGUCCGAGUGCGUCCUGGGCCAGUCAAAGAGAGUGGAUUUCUG
CGGCAAGGGAUACCACCUGAUGAGCUUCCCCCAGAGCGCUCCGCACGGCGUGGU
GUUUCUGCACGUGACCUACGUGCCAGCCCAGGAGAAGAACUUCACCACGGCCCCU
GCCAUCUGCCACGACGGCAAGGCCCACUUCCCCAGAGAAGGAGUGUUCGUGAGCA
AUGGCACACACUGGUUCGUGACACAAAGAAACUUCUACGAGCCUCAGAUCAUCACA
ACCGAUAACACCUUCGUGAGCGGCAAUUGCGACGUGGUGAUCGGCAUUGUGAACA
ACACCGUGUACGACCCCCUGCAGCCCGAGCUGGAUAGCUUCAAAGAGGAACUGGA
UAAGUACUUCAAGAACCACACCAGCCCUGAUGUGGAUCUGGGCGACAUUUCUGGC
AUCAACGCCUCUGUCGUGAACAUCCAGAAAGAGAUAGAUAGACUGAACGAGGUUG
CAAAGAACCUGAACGAAAGCCUGAUCGACUUGCAGGAGCUCGGCAAGUACGAGCA
GUACAUCAAGUGGCCUUGGUACAUUUGGCUGGGCUUUAUCGCCGGACUGAUCGC
CAUCGUGAUGGUCACAAUCAUGCUGUGCUGCAUGACAAGUUGCUGUUCCUGCCUG
AAGGGCUGCUGUAGCUGUGGAAGCUGCUGUAAAUUCGACGAAGAUGACAGCGAGC
CUGUGCUGAAGGGCGUGAAGCUGCACUACACAUGACAAACACCAUUGUCACACUC
CAUUUAAAGGCUGUCAAUUCAUAGGUCAGUUUAAACUUCCAGUCGAGGAUGUUUACA
Prefusion Spike mRNA sequence of the South Africa variant (B.1.351 or Beta variant) with MOPC
in 3′ UTR [SEQ ID NO: 65]
3′ MOP for miRNA-122-5P, miRNA-192-5P, miRNA-30a-5P (MOPC, underlined portion)
Codons optimized for human cellular expression
AUGUUCGUGUUCCUGGUGUUACUGCCCCUGGUGUCUUCUCAGUGCGUCAACUUC
ACCACAAGAACACAGCUGCCUCCUGCCUAUACAAACAGCUUUACCCGGGGAGUGU
ACUACCCCGAUAAAGUGUUCCGGAGCUCUGUGCUGCACAGCACACAGGACUUGUU
CCUGCCUUUCUUCAGCAAUGUGACAUGGUUCCACGCCAUCCACGUCUCCGGCACA
AACGGCACCAAGAGGUUCGCCAAUCCUGUGCUGCCAUUCAAUGAUGGCGUGUAUU
UCGCCAGCACAGAGAAGUCUAACAUCAUCAGAGGCUGGAUCUUCGGCACCACCCU
CGACUCUAAAACCCAGAGCCUGCUGAUAGUGAACAACGCCACAAACGUGGUGAUC
AAGGUCUGUGAAUUCCAGUUCUGCAACGACCCAUUCCUGGGCGUGUACUACCACA
AGAAUAACAAGAGCUGGAUGGAAUCUGAGUUCAGAGUGUAUUCAUCAGCCAACAA
CUGCACAUUCGAGUACGUGUCUCAGCCAUUCCUGAUGGACCUGGAAGGCAAGCAG
GGCAAUUUCAAGAAUCUCAGAGAGUUCGUCUUCAAGAACAUCGACGGCUACUUUA
AGAUCUAUAGCAAGCACACCCCUAUCAACCUGGUUCGGGGCCUGCCCCAGGGCUU
UAGCGCCCUGGAACCUCUGGUGGAUCUGCCAAUUGGCAUCAACAUCACCCGGUUU
CAGACACUGCACAUCAGCUACCUGACACCUGGCGACAGCAGCAGCGGCUGGACCG
CCGGCGCCGCCGCCUACUACGUCGGCUACCUGCAGCCCCGGACCUUCCUGCUGA
AGUACAAUGAAAAUGGCACCAUCACAGACGCCGUGGAUUGCGCCCUGGACCCUCU
GUCUGAAACAAAGUGCACCCUGAAAAGCUUCACCGUGGAAAAGGGCAUAUACCAG
ACCUCCAACUUCCGGGUGCAGCCUACAGAGUCUAUCGUGAGAUUCCCCAACAUCA
CCAAUCUGUGUCCUUUUGGCGAGGUGUUCAACGCCACCAGAUUCGCAAGCGUGUA
CGCCUGGAACCGGAAGAGGAUCAGCAACUGCGUGGCAGAUUACAGCGUGCUCUAC
AACAGCGCCAGUUUCUCUACCUUUAAGUGCUACGGCGUCAGCCCUACAAAACUGA
ACGAUCUGUGCUUCACCAACGUGUACGCCGAUUCCUUUGUGAUACGGGGCGACGA
AGUUAGACAGAUCGCCCCUGGACAGACAGGAAAUAUCGCCGACUACAACUAUAAG
CUGCCUGACGACUUCACCGGCUGCGUCAUCGCUUGGAACUCCAACAACCUGGAUU
CCAAGGUGGGCGGAAACUACAACUACCUGUACAGACUGUUCAGAAAGAGCAACCU
GAAACCUUUCGAGAGGGACAUCAGCACAGAGAUCUACCAGGCCGGCAGCACCCCC
UGUAAUGGAGUCAAAGGCUUCAAUUGCUACUUCCCUCUGCAGUCUUACGGCUUCC
AGCCAACAUACGGCGUGGGCUACCAGCCCUACCGGGUGGUUGUGCUGUCCUUCG
AGCUGCUGCAUGCCCCAGCCACAGUAUGCGGUCCUAAGAAAAGCACCAACCUGGU
GAAGAACAAAUGUGUGAACUUUAACUUUAACGGCCUGACCGGCACCGGCGUGCUG
ACCGAAUCCAAUAAGAAGUUCCUGCCGUUCCAGCAGUUUGGCAGAGAUAUCGCCG
ACACCACAGACGCCGUGAGAGACCCCCAGACCCUGGAAAUCCUGGACAUCACCCC
UUGCUCCUUUGGAGGGGUGAGCGUGAUCACCCCGGGCACAAACACCAGCAACCAG
GUGGCCGUGCUGUACCAGGGCGUGAAUUGUACCGAGGUGCCUGUGGCGAUCCAC
GCCGAUCAGCUGACCCCUACCUGGCGGGUGUACAGCACCGGAUCUAACGUGUUC
CAAACAAGAGCCGGCUGUCUGAUCGGAGCUGAACACGUGAACAACUCUUACGAGU
GUGACAUUCCUAUCGGCGCCGGCAUCUGCGCCUCUUAUCAGACCCAGACCAACAG
CCCCAGACGUGCCAGAUCUGUGGCCUCUCAGAGCAUCAUCGCCUACACCAUGUCU
CUGGGAGUGGAAAACUCCGUGGCUUACAGCAACAAUUCUAUCGCCAUCCCCACCA
ACUUUACAAUCAGCGUGACCACCGAGAUACUGCCUGUGUCCAUGACAAAGACCAG
CGUGGACUGCACUAUGUACAUCUGCGGCGACAGCACAGAAUGCAGCAACCUGCUG
CUGCAGUACGGAAGCUUUUGUACUCAGCUGAACAGAGCCCUGACUGGCAUCGCUG
UUGAGCAGGAUAAGAAUACUCAGGAGGUCUUCGCUCAAGUGAAGCAGAUCUACAA
GACCCCUCCAAUCAAGGACUUCGGCGGCUUCAACUUCAGCCAAAUUCUGCCUGAU
CCUAGCAAGCCCAGCAAGCGGAGCUUCAUCGAGGACCUGCUGUUUAACAAAGUGA
CACUUGCCGACGCCGGAUUCAUUAAGCAGUAUGGCGACUGCCUGGGCGACAUCG
CCGCGAGAGAUUUGAUCUGCGCCCAAAAGUUCAACGGCCUCACCGUGCUGCCUCC
UCUUCUGACCGACGAGAUGAUCGCUCAGUACACCAGCGCUCUUCUGGCCGGCACA
AUCACCAGCGGCUGGACAUUUGGCGCUGGUGCCGCCCUCCAGAUCCCUUUCGCC
AUGCAGAUGGCCUACAGAUUCAACGGCAUCGGCGUCACCCAAAACGUGCUCUAUG
AGAACCAGAAACUUAUCGCUAAUCAGUUCAACUCUGCCAUCGGCAAGAUCCAAGAU
AGCCUGUCCUCCACCGCUAGCGCCCUGGGAAAGCUCCAGGACGUGGUGAAUCAGA
ACGCCCAAGCCCUGAACACCCUGGUGAAACAGCUGAGCAGCAACUUCGGCGCUAU
CAGCUCCGUUCUGAACGACAUUCUGUCUAGACUGGACCCUCCUGAGGCCGAGGUC
CAGAUCGAUAGACUGAUCACUGGACGCCUGCAAUCACUGCAAACAUACGUGACCC
AGCAGCUGAUUAGAGCCGCCGAGAUCAGAGCCUCAGCAAAUCUGGCCGCCACGAA
GAUGAGCGAGUGCGUGCUGGGCCAGAGCAAGAGAGUCGACUUUUGCGGCAAAGG
CUACCACCUGAUGAGCUUCCCUCAGAGCGCCCCACACGGCGUGGUGUUCCUGCAU
GUGACCUACGUGCCCGCCCAGGAAAAGAACUUUACCACCGCCCCUGCUAUCUGUC
ACGACGGCAAGGCCCACUUCCCUCGCGAGGGCGUGUUCGUCAGCAACGGCACCC
ACUGGUUCGUGACACAACGUAACUUCUACGAGCCUCAGAUCAUAACCACCGAUAAC
ACAUUCGUGAGCGGCAAUUGCGAUGUGGUGAUCGGAAUCGUGAACAACACCGUGU
ACGACCCGCUGCAGCCCGAGCUGGACAGCUUCAAAGAGGAACUGGAUAAGUACUU
UAAGAACCACACUUCUCCAGACGUGGACCUGGGCGAUAUCAGCGGAAUCAACGCU
UCCGUGGUGAACAUCCAGAAGGAAAUCGACAGACUGAACGAGGUGGCUAAAAACC
UGAAUGAGAGCCUGAUCGACCUGCAGGAGCUGGGAAAAUACGAACAGUACAUCAA
GUGGCCUUGGUACAUCUGGCUGGGCUUUAUCGCUGGCCUGAUCGCCAUCGUGAU
GGUGACCAUCAUGCUGUGCUGUAUGACCAGCUGUUGUAGCUGCCUGAAGGGUUG
CUGUUCCUGCGGAAGCUGCUGCAAGUUCGACGAGGAUGACAGCGAGCCCGUGCU
GAAGGGCGUUAAGCUGCACUACACCUGACAAACACCAUUGUCACACUCCAUUUAAA
GGCUGUCAAUUCAUAGGUCAGUUUAAACUUCCAGUCGAGGAUGUUUACA
Prefusion Spike mRNA sequence of the Delta variant (B.1.617.2 first identified in India)
with MOPV in 3′ UTR [SEQ ID NO: 66]
3′ MOP for miRNA-122-5P, miRNA-1-3P, miRNA-203a-3P, miRNA-30a-5P (MOPV, underlined portion)
Codons optimized for human cellular expression
AUGUUCGUCUUCCUGGUGCUGCUGCCCCUGGUGAGCUCUCAGUGCGUGAAUCUG
AGAACCCGGACACAGCUGCCUCCUGCCUACACAAACAGCUUUACAAGAGGCGUCU
ACUACCCUGACAAGGUGUUCCGGUCGAGCGUGCUGCAUUCUACCCAGGAUUUGUU
UCUUCCUUUUUUCAGUAACGUGACAUGGUUCCACGCCAUCCACGUGUCCGGAACC
AACGGCACCAAGAGAUUCGACAACCCUGUGCUGCCUUUCAACGACGGAGUGUAUU
UCGCCUCUACCGAGAAGAGCAACAUCAUUCGGGGAUGGAUCUUCGGAACCACCCU
CGACAGCAAGACACAGAGCCUGCUAAUAGUCAACAACGCUACCAACGUGGUGAUU
AAGGUGUGCGAGUUCCAAUUUUGUAACGAUCCUUUCCUGGGAGUUUAUUACCAUA
AGAACAAUAAAAGCUGGAUGGAAAGCGAGGUGUACAGCAGCGCAAACAACUGCACA
UUCGAGUAUGUGAGCCAACCUUUCCUGAUGGACCUGGAAGGCAAGCAGGGGAACU
UCAAGAACCUGAGGGAAUUCGUGUUUAAGAACAUCGACGGCUACUUCAAGAUCUA
CAGCAAGCACACACCAAUUAACCUCGUUAGAGAUCUGCCACAGGGCUUCAGUGCC
CUGGAACCCCUGGUGGAUCUGCCCAUCGGAAUCAACAUCACCAGAUUCCAGACCC
UCCUGGCCCUGCACAGAAGCUAUCUGACCCCUGGCGAUUCUAGCUCUGGCUGGA
CAGCUGGCGCCGCUGCUUACUACGUGGGCUACCUGCAGCCUAGAACAUUCCUGC
UCAAGUACAACGAGAAUGGCACAAUCACCGACGCCGUUGACUGCGCCCUGGAUCC
UUUGUCUGAGACAAAGUGCACUCUGAAGAGCUUCACCGUGGAAAAGGGCAUCUAC
CAGACAUCUAACUUCAGAGUGCAGCCUACAGAGAGCAUCGUGCGGUUCCCCAACA
UAACAAACCUGUGUCCAUUCGGAGAAGUGUUUAAUGCCACCAGAUUCGCUAGCGU
GUACGCCUGGAACCGGAAGAGAAUCAGCAACUGCGUCGCCGACUACUCCGUGCUG
UACAAUAGCGCCUCUUUCAGCACCUUUAAGUGUUACGGCGUCUCUCCAACAAAGC
UGAACGACCUGUGCUUCACAAACGUGUACGCCGACAGCUUCGUGAUCCGGGGCGA
CGAAGUGCGGCAGAUUGCACCUGGUCAGACUGGGAAAAUCGCAGAUUACAACUAC
AAGCUGCCAGAUGAUUUUACCGGCUGUGUGAUCGCCUGGAAUAGCAAUAACCUGG
ACAGCAAAGUGGGCGGCAAUUACAACUACCGGUACAGACUGUUCCGGAAGAGCAA
UCUGAAGCCUUUUGAGAGAGACAUCUCCACAGAGAUCUACCAGGCCGGCUCUAAG
CCUUGCAACGGCGUGGAGGGGUUUAAUUGCUACUUCCCUCUGCAGUCUUACGGG
UUCCAGCCCACCAACGGCGUGGGCUAUCAGCCUUACAGAGUGGUGGUGCUGUCU
UUCGAACUGCUGCACGCCCCUGCUACCGUGUGCGGGCCUAAGAAGUCCACCAACC
UUGUGAAGAACAAGUGUGUGAACUUCAACUUCAAUGGCCUGACCGGAACCGGCGU
GUUGACCGAAUCUAACAAGAAAUUCCUGCCGUUCCAACAGUUCGGCAGAGAUAUU
GCCGACACCACCGAUGCCGUGCGGGACCCCCAAACCCUGGAAAUCCUGGAUAUCA
CCCCAUGCAGCUUCGGCGGCGUGUCUGUUAUCACCCCCGGCACAAACACGAGCAA
CCAGGUCGCCGUGCUCUACCAGGGCGUGAACUGCACAGAAGUGCCCGUGGCUAU
CCACGCCGAUCAGCUGACACCCACAUGGCGGGUGUACAGCACAGGAUCUAACGUU
UUCCAGACAAGAGCUGGCUGCCUUAUUGGCGCUGAACACGUGAAUAACAGCUACG
AGUGUGACAUCCCAAUCGGCGCCGGCAUCUGCGCCUCCUACCAGACCCAGACCAA
CAGCAGAAGGAGAGCCCGGAGCGUGGCCAGCCAGUCUAUCAUCGCCUACACAAUG
AGCCUGGGCGCCGAAAACUCCGUGGCCUAUAGCAACAACUCCAUCGCUAUCCCUA
CCAACUUCACCAUCAGCGUGACAACGGAAAUUCUGCCUGUGAGCAUGACCAAGAC
CUCUGUGGACUGUACAAUGUACAUCUGCGGCGACUCUACAGAAUGCAGCAACCUG
CUGCUGCAGUACGGCAGCUUUUGCACCCAGCUUAAUAGAGCCCUGACCGGAAUCG
CCGUGGAACAGGACAAGAACACCCAGGAGGUCUUCGCCCAGGUGAAACAGAUCUA
CAAGACCCCUCCUAUUAAGGACUUCGGCGGAUUUAACUUCAGCCAGAUCCUGCCU
GACCCUAGCAAGCCCAGCAAAAGAAGCUUCAUCGAGGACCUCCUGUUCAACAAAG
UGACCCUGGCCGACGCUGGCUUUAUCAAGCAGUAUGGCGACUGCCUGGGCGACA
UCGCUGCUAGGGACCUGAUCUGUGCCCAGAAGUUCAACGGCCUGACAGUGCUGC
CUCCUCUGCUGACCGAUGAAAUGAUCGCCCAGUACACAAGCGCCCUGCUGGCCGG
CACCAUCACCAGCGGCUGGACCUUUGGAGCCGGCGCCGCCCUGCAGAUCCCCUU
UGCCAUGCAGAUGGCCUAUCGGUUCAACGGAAUCGGCGUGACCCAAAACGUACUG
UACGAGAACCAGAAGCUGAUCGCCAAUCAAUUUAAUAGCGCCAUCGGUAAAAUCCA
GGAUAGCCUGAGCUCCACUGCCAGCGCCCUGGGCAAACUGCAGAACGUGGUGAAC
CAGAACGCCCAAGCUCUGAACACCCUGGUGAAGCAGCUGUCUUCCAACUUUGGUG
CUAUCUCUAGCGUCCUGAAUGAUAUCCUGAGCAGACUGGACCCCCCCGAGGCCGA
GGUGCAGAUCGAUAGACUGAUCACCGGCAGACUGCAAUCGCUGCAAACUUACGUG
ACCCAGCAGCUGAUCAGAGCCGCCGAGAUCAGAGCUAGCGCCAACCUGGCCGCCACUAAGAU
GAGCGAGUGCGUUCUGGGCCAGAGUAAGCGGGUGGACUUCUGUGGCA
AGGGAUACCACCUGAUGUCUUUUCCACAGAGCGCCCCUCACGGCGUGGUGUUCC
UGCACGUUACCUACGUGCCAGCCCAGGAGAAGAACUUCACCACAGCCCCUGCCAU
CUGCCACGACGGCAAGGCCCACUUCCCUAGAGAGGGCGUGUUCGUCAGCAACGG CACCCACUGG
UUCGUGACGCAAAGAAACUUCUACGAGCCCCAGAUCAUUACCACC
GACAAUACCUUCGUAUCCGGCAACUGCGACGUGGUGAUCGGCAUCGUGAACAACA
CAGUGUACGACCCUCUGCAGCCUGAGCUGGACUCUUUCAAGGAAGAGCUGGACAA
GUAUUUCAAGAACCACACCAGCCCUGAUGUGGACCUGGGCGACAUCAGCGGAAUC
AAUGCCUCAGUGGUGAACAUCCAGAAAGAGAUCGACAGACUGAACGAGGUCGCCA
AGAACCUGAAUGAGAGCCUGAUCGACCUGCAGGAGCUGGGCAAGUACGAGCAAUA
CAUCAAGUGGCCUUGGUACAUCUGGCUGGGCUUCAUCGCCGGCCUGAUUGCCAU
CGUGAUGGUGACCAUCAUGCUGUGUUGCAUGACCAGUUGCUGUAGUUGCCUGAA
AGGCUGCUGUUCUUGCGGCAGCUGCUGCAAAUUCGAUGAGGACGACUCCGAGCC
CGUGCUGAAGGGCGUGAAGCUGCACUACACCUGACAAACACCAUUGUCACACUCC
AUUUAAAAUACAUACUUCUUUACAUUCCAUUUAAACUAGUGGUCCUAAACAUUUCA
CUUUAAACUUCCAGUCGAGGAUGUUUACA
Prefusion Spike mRNA sequence of the UK/Kent variant (B.1.1.7 or Alpha variant) with MOPC
in 3′ UTR [SEQ ID NO: 67]
3′ MOP for miRNA-122-5P, miRNA-192-5P, miRNA-30a-5P (MOPC, underlined portion)
Codons optimized for human cellular expression
AUGUUCGUGUUUCUGGUCCUGCUGCCCCUGGUGUCCUCCCAGUGCGUGAACCUG
ACGACCAGAACACAACUGCCUCCUGCCUACACCAACAGCUUUACAAGAGGCGUCUA
UUACCCCGACAAGGUGUUCCGGAGCUCCGUCCUGCACUCUACCCAGGACCUUUUC
CUGCCUUUUUUCAGCAACGUGACAUGGUUCCACGCCAUCAGCGGUACCAACGGCA
CCAAGCGCUUCGACAACCCUGUGCUGCCAUUUAACGACGGAGUGUAUUUCGCCUC
CACAGAAAAGUCGAACAUCAUCAGGGGCUGGAUCUUCGGCACCACACUGGACAGC
AAGACCCAGAGCCUGCUGAUCGUGAAUAACGCCACAAACGUGGUCAUCAAAGUCU
GCGAGUUCCAGUUCUGUAAUGACCCCUUCCUGGGCGUUCAUAAAAACAACAAAAG
CUGGAUGGAAAGCGAGUUCAGAGUGUAUUCUAGCGCCAAUAACUGUACAUUUGAG
UACGUGUCCCAGCCCUUCCUGAUGGACCUGGAAGGCAAGCAAGGAAAUUUUAAGA
ACCUGCGUGAGUUUGUGUUCAAGAACAUCGAUGGUUAUUUCAAAAUCUACAGCAA
GCACACCCCCAUUAACCUGGUCAGAGACCUGCCCCAGGGCUUCUCUGCCCUGGAA
CCUCUGGUGGACCUGCCGAUCGGAAUCAACAUCACACGGUUCCAGACCCUGCUAG
CCCUGCAUAGAUCUUACCUGACCCCCGGCGACAGCUCUUCCGGCUGGACAGCCG
GCGCCGCUGCUUACUACGUGGGCUACCUGCAGCCUAGAACCUUCCUGCUCAAGUA
CAACGAAAAUGGCACCAUCACCGACGCCGUGGACUGCGCCCUGGACCCUCUUAGC
GAGACAAAGUGCACACUGAAGAGCUUCACCGUGGAAAAGGGCAUCUACCAGACAU
CGAACUUCAGAGUGCAGCCUACCGAGUCCAUCGUGAGGUUUCCUAACAUCACCAA
CCUGUGUCCUUUCGGCGAGGUGUUCAACGCCACCAGAUUCGCCAGCGUGUACGC
CUGGAAUAGAAAGAGAAUCUCUAACUGUGUGGCCGAUUACAGCGUGCUGUACAAC
UCUGCCAGCUUUAGCACCUUUAAAUGUUACGGCGUGAGCCCUACAAAGCUGAACG
AUCUGUGCUUCACCAAUGUGUACGCCGAUUCUUUCGUGAUCCGGGGCGAUGAGG
UGCGGCAGAUCGCCCCAGGCCAGACAGGCAAGAUCGCCGACUACAAUUACAAGCU
GCCUGAUGACUUCACCGGCUGCGUGAUUGCCUGGAACAGCAACAAUCUGGACAGC
AAGGUGGGAGGCAACUACAACUACCUGUACAGACUCUUCCGGAAGAGCAACCUGA
AACCUUUCGAGAGAGAUAUCUCAACUGAAAUCUACCAGGCCGGCUCAACACCCUG
CAAUGGAGUUGAGGGCUUCAAUUGCUACUUCCCCCUGCAGUCUUACGGCUUUCAG
CCUACAUACGGCGUGGGCUACCAGCCUUACCGGGUGGUUGUGUUGAGCUUCGAA
CUGCUGCACGCCCCUGCUACCGUGUGCGGUCCUAAGAAAAGCACCAACCUGGUGA
AGAACAAGUGCGUAAACUUCAACUUCAACGGCCUGACUGGAACAGGCGUCCUGAC
CGAAAGCAACAAGAAGUUCCUGCCUUUUCAACAAUUUGGCAGAGAUAUUGAUGAUA
CAACAGAUGCUGUGCGGGAUCCUCAGACACUGGAAAUCCUGGACAUCACCCCCUG
CUCCUUCGGCGGAGUCAGCGUGAUAACCCCUGGCACUAACACCAGCAAUCAGGUG
GCCGUGCUCUACCAGGGCGUCAACUGCACCGAAGUCCCUGUUGCUAUCCACGCU
GACCAGCUGACACCUACCUGGAGAGUGUAUAGCACCGGUUCUAACGUCUUCCAGA
CCCGCGCCGGCUGUCUGAUCGGCGCCGAGCACGUGAACAACAGCUACGAGUGCG
ACAUCCCCAUCGGCGCU GGCAUCUGCGCCUCUUAUCAGACACAGACCAACAGCCA
CCGGAGAGCUAGAAGCGUGGCCUCUCAGUCGAUCAUUGCCUACACCAUGUCCCUG
GGCGCCGAGAACUCGGUGGCCUACAGCAACAAUUCUAUCGCCAUCCCCAUCAACU
UCACCAUCAGCGUGACAACCGAAAUUCUGCCAGUGUCCAUGACGAAGACAUCCGU
GGACUGCACAAUGUACAUCUGCGGCGAUAGCACAGAAUGUUCUAAUCUGCUGCUU
CAAUAUGGAUCUUUCUGCACCCAGCUGAACCGGGCCCUGACAGGCAUCGCCGUGG
AACAGGACAAAAAUACCCAGGAGGUGUUUGCCCAGGUGAAGCAGAUCUACAAGAC
CCCACCAAUCAAGGACUUCGGAGGGUUUAAUUUCAGCCAGAUCCUGCCCGAUCCU AGCAAGCCU
UCCAAGCGGAGUUUCAUCGAGGACCUGCUGUUCAACAAAGUGACCC
UGGCUGAUGCCGGCUUCAUCAAGCAGUACGGCGACUGCCUGGGCGACAUCGCCG
CCAGAGAUCUGAUCUGCGCCCAGAAAUUUAACGGGCUGACCGUGCUGCCUCCACU
GCUGACCGACGAGAUGAUCGCACAGUACACCAGCGCUUUGCUGGCGGGCACCAUC
ACGAGCGGCUGGACCUUCGGGGCCGGCGCCGCCCUGCAAAUUCCUUUCGCCAUG
CAGAUGGCCUACCGGUUUAACGGCAUCGGCGUGACACAGAACGUGCUAUACGAGA
ACCAGAAGCUGAUAGCUAAUCAGUUUAACUCUGCCAUCGGCAAGAUCCAGGACAG
CCUCUCCAGCACCGCCAGCGCCCUGGGUAAGCUGCAGGACGUGGUGAACCAGAAC
GCCCAAGCCCUGAACACCCUGGUUAAGCAGCUGUCCAGCAAUUUCGGCGCUAUUA
GCAGCGUUCUGAAUGACAUCCUGGCCAGACUGGACCCACCUGAGGCCGAGGUGC
AGAUCGAUAGACUGAUCACAGGAAGACUGCAGAGCCUGCAGACCUACGUCACCCA
ACAACUCAUCCGGGCCGCCGAAAUCCGGGCCAGCGCCAACCUUGCAGCCACCAAG
AUGAGCGAGUGCGUGCUCGGCCAGAGCAAAAGAGUGGACUUUUGCGGCAAAGGC
UACCACCUGAUGUCCUUCCCUCAGAGCGCCCCACACGGCGUGGUGUUCCUGCAC
GUGACAUAUGUGCCCGCACAGGAGAAGAACUUCACGACUGCUCCCGCCAUCUGCC
ACGACGGCAAGGCCCACUUCCCCAGAGAAGGCGUGUUCGUGAGUAACGGGACCCA
CUGGUUCGUGACCCAGAGAAACUUCUACGAGCCUCAGAUCAUCACAACCCACAACA
CAUUCGUGAGCGGAAACUGCGAUGUGGUGAUCGGAAUCGUGAACAAUACCGUGUA
CGACCCUCUGCAGCCUGAGCUGGACAGCUUCAAAGAGGAACUCGACAAGUAUUUU
AAGAACCACACCAGCCCUGACGUGGAUCUGGGCGACAUCAGCGGCAUCAACGCUA
GCGUGGUGAACAUCCAGAAGGAAAUCGACAGACUGAACGAGGUGGCCAAGAACCU
GAACGAGAGCCUGAUCGACCUGCAGGAGCUGGGCAAGUACGAGCAGUACAUCAAG
UGGCCUUGGUACAUUUGGCUGGGCUUCAUCGCAGGGCUGAUCGCCAUCGUGAUG
GUGACAAUCAUGCUGUGUUGCAUGACCUCUUGUUGCAGCUGUCUUAAAGGCUGCU
GCAGCUGUGGAAGCUGCUGCAAGUUCGACGAGGAUGAUAGCGAACCCGUGCUGA
AGGGCGUCAAGCUGCACUACACCUGACAAACACCAUUGUCACACUCCAUUUAAAG
GCUGUCAAUUCAUAGGUCAGUUUAAACUUCCAGUCGAGGAUGUUUACA

In some embodiments of any of the aspects, the antigen comprises a coronavirus spike protein. In some embodiments of any of the aspects, the antigen comprises a coronavirus receptor binding domain (RBD) protein. In some embodiments of any of the aspects, the antigen comprises a variant coronavirus spike protein. In some embodiments of any of the aspects, the antigen comprises a variant coronavirus receptor binding domain protein. Coronavirus spike proteins include MERS-COV, SARS-COV-1, and SARS-COV-2 spike and RBD proteins.

In some embodiments, the coding mRNA can encode one or more viral proteins of the Human alpha-herpesvirus 3 (HHV-3), also known as the varicella-zoster virus (VZV). In particular embodiments, the coding mRNA can encode one or more glycoproteins of VZV, for example, glycoprotein E (VZVgE).

In some embodiments, the coding mRNA can encode one or more immunogenic viral proteins of the influenza virus (type A and B that cause epidemic seasonal flu) such as the hemagglutinin, the neuraminidase, the matrix-2 and/or the nucleoprotein. Hemagglutinin is highly variable between groups, types, and even subtypes of influenza, which is a factor in the difficulty of developing a universal flu vaccine. The Head domain of the Hemagglutinin is highly variable, but the membrane proximal stalk-domain of the Hemagglutinin is relatively well conserved within a group, but is immunosubdominant. Some vaccine strategies therefore use a reduced HA without the Head domain and it is accordingly contemplated that such a reduced HA may be provided in embodiments of the present invention.

It is considered to provide one or more immunogenic viral proteins from any group, type or subtype of influenza, for example, from influenza A Group 1: H1, H2, H5, H6, H8, H9, H11, H12, H13, H16, H17, H18 subtypes and N1, N4, N5, N8 subtypes; from Influenza A Group 2: H3, H4, H7, H10, H14, H15 subtypes+N2, N3, N6, N7, N9 subtypes; or from Influenza B. Influenza B viruses are not divided into subtypes, but instead are further classified into two lineages: B/Yamagata and B/Victoria.

Neuraminidase drifts more slowly than Hemagglutinin, and antibodies against Neuraminidase have been shown to be cross-protective within a subtype. Neuraminidase is immunosubdominant compared to Hemagglutinin. The matrix-2 and/or the nucleoprotein are more conserved than Hemagglutinin but are immunosubdominant.

Each year, the WHO recommends quadrivalent or trivalent influenza vaccines based on predictions. As a result, it is particularly envisioned to provide compositions and constructs which encode more than one influenza antigen, in order to provide broad protection.

In some embodiments, a mRNA encoding an antigen can encode one or more immunogenic viral proteins of the respiratory syncytial virus such as the F glycoprotein and/or the G glycoprotein. The F glycoprotein from A2 strain can be stabilized in prefusion conformation using the modification described by Mclellan et al., 2013, which induces cross-protection against RSV A (Long) and RSV B (18537) strains.

In some embodiments, a mRNA encoding an antigen can encode one or more immunogenic viral proteins of the human immunodeficiency virus such as the full length or part of the glycoprotein 120 neutralizing epitope (such as CD4BS 421-433 epitope) or the glycoprotein 145. Antigens from HIV such as gag, pol, env, and nef have been expressed in various vectors as possible vaccine candidates (I P Nascimento and L C C Leite, Braz J Med Biol Res. 2012 doi: 10.1590/S0100-879X2012007500142).

In some embodiments, a mRNA encoding an antigen can encode one or more immunogenic bacterial proteins, or parts thereof, of bacteria from the Mycobacterium genus. In particular, the coding mRNA may encode one or more bacterial proteins from the Mycobacterium tuberculosis and/or Mycobacterium leprae bacteria. In some embodiments, the mRNA encoding an antigen may encode one or more proteins from the active and/or latent and/or resuscitation phase of M tuberculosis. For example, the mRNA may encode one or more of the M. tuberculosis proteins selected from ESAT-6, Ag85B, TB10.4, Rv2626 and/or RpfD-B, or a part thereof.

Table 6B below shows examples of ORFs encoding antigens for a number of different potential pathogens, which can be used in the present invention. These ORFs can be present with further RNA sequences, most particularly OPS, as described herein, and/or used in combination with further mRNA constructs. Similar to the discussion above, in some embodiments, a mRNA encoding an antigen includes one or more of the sequences recited in Table 6B below (SEQ ID NOs: 69 to 84), or an epitope-containing fragment thereof, or a sequence with at least 80%, at least 85%, at least 90%, at least 95%, at least 98%, or at least 99% similarity thereto. In some embodiments, the mRNA encoding an antigen for the antigen or part thereof has been codon-optimised for expression in human or other mammalian cells. In some embodiments, one or more of the nucleosides used in the mRNA are been replaced by an isomer thereof. As example one, more or all of the uridine nucleosides in the mRNA construct are replaced by pseudouridine nucleosides.

TABLE 6B
Exemplary ORFs for antigen for several pathogens
suitable for use in vaccine compositions, not
being optimised for human cellular expression
or containing MOP sequences.

INFLUENZA MRNA ORF for Nucleoprotein (NP) from
A/Michigan/45/2015(H1N1) [SEQ ID NO: 69]
AUGGCGUCUCAAGGCACCAAACGAUCAUAUGAACAAAUGGAGACU
GGUGGGGAGCGCCAGGAUACCACAGAAAUCAGAGCAUCUGUUGGA
AGAAUGAUUGGUGGAAUCGGGAGAUUCUACAUCCAAAUGUGCACU
GAACUCAAACUCAGUGAUUAUGAUGGACGACUAAUCCAGAAUAGC
AUAACAAUAGAGAGGAUGGUGCUUUCUGCUUUUGAUGAGAGAAGA
AAUAAAUACCUAGAAGAGCAUCCAAGUGCUGGGAAGGACCCUAAG
AAAACAGGAGGACCCAUCUAUAGAAGAAUAGACGGAAAAUGGACG
AGAGAACUCAUCCUUUAUGACAAAGAAGAAAUAAGGAGAGUUUGG
CGCCAAGCAAACAAUGGCGAAGAUGCAACAGCAGGUCUUACUCAU
AUCAUGAUUUGGCAUUCCAACCUGAAUGAUGCCACAUAUCAGAGG
ACAAGAGCACUUGUUCGCACUGGAAUGGAUCCCAGAAUGUGCUCU
CUAAUGCAAGGUUCAACACUUCCCAGAAGGUCUGGUGCCGCAGGU
GCUGCAGUGAAAGGAGUUGGAACAAUAGCUAUGGAGUUAAUCAGA
AUGAUCAAACGUGGAAUCAAUGACCGAAAUUUCUGGAGGGGUGAA
AAUGGACGAAGGACAAGAGUUGCUUAUGAAAGAAUGUGCAAUAUC
CUCAAAGGAAAAUUUCAAACAGCUGCCCAGAGGGCAAUGAUGGAU
CAAGUAAGAGAAAGUCGAAACCCAGGAAACGCUGAGAUUGAAGAC
CUCAUUUUCCUGGCACGGUCAGCACUCAUUCUGAGAGGAUCAGUU
GCACAUAAAUCCUGCCUGCCUGCUUGUGUGUAUGGGCUUGCAGUA
GCAAGUGGCCAUGACUUUGAAAGGGAAGGGUACUCACUGGUCGGG
AUAGACCCAUUCAAAUUACUCCAAAACAGUCAAGUGGUCAGCCUG
AUGAGACCAAAUGAAAAUCCAGCUCACAAGAGUCAAUUGGUAUGG
AUGGCAUGCCACUCUGCUGCAUUUGAAGAUUUAAGAGUAUCAAGU
UUCAUAAGAGGAAAGAAAGUGAUCCCAAGAGGAAAGCUUUCCACA
AGAGGGGUUCAGAUUGCUUCAAAUGAGAAUGUGGAAACCAUGGAC
UCCAAUACCCUGGAACUAAGAAGCAGAUACUGGGCCAUAAGAACC
AGGAGUGGAGGAAAUACCAAUCAACAGAAGGCAUCCGCAGGCCAG
AUCAGUGUGCAGCCUACAUUCUCAGUGCAGCGAAAUCUCCCUUUU
GAAAGAGCAACCGUUAUGGCAGCAUUCAGCGGGAACAAUGAAGGA
CGGACAUCCGACAUGAGAACAGAAGUUAUAAGAAUGAUGGAAAGU
GCAAAGCCAGAGGAUUUGUCCUUCCAGGGGGGGGGAGUCUUCGAG
CUCUCGGACGAAAAGGCAACGAACCCGAUCGUGCCUUCCUUUGAC
AUGAGUAAUGAAGGGUCUUAUUUCUUCGGAGACAAUGCAGAGGAG
UAUGACAAUUGA
INFLUENZA MRNA Neuraminidase (NA) from
A/Michigan/45/2015(H1N1)
[SEQ ID NO: 70]
AUGAAUCCAAACCAAAAGAUAAUAACCAUUGGUUCGAUCUGUAUG
ACAAUUGGAAUGGCUAACUUAAUAUUACAAAUUGGAAACAUAAUC
UCAAUAUGGGUUAGCCACUCAAUUCAAAUUGGAAAUCAAAGCCAG
AUUGAAACAUGCAAUCAAAGCGUCAUUACUUAUGAAAACAACACU
UGGGUAAAUCAGACAUAUGUUAACAUCAGCAACACCAACUUUGCU
GCUGGACAGUCAGUGGUUUCCGUGAAAUUAGCGGGCAAUUCCUCU
CUCUGCCCUGUUAGUGGAUGGGCUAUAUACAGUAAAGACAACAGU
GUAAGAAUCGGUUCCAAGGGGGAUGUGUUUGUCAUAAGGGAACCA
UUCAUAUCAUGCUCUCCCUUGGAAUGCAGAACCUUCUUCUUGACU
CAAGGGGCCUUGCUAAAUGACAAACAUUCCAAUGGAACCAUUAAA
GACAGGAGCCCAUACCGAACCCUAAUGAGCUGUCCUAUUGGUGAA
GUUCCCUCUCCAUACAACUCAAGAUUUGAGUCAGUCGCUUGGUCA
GCAAGUGCUUGUCAUGAUGGCAUCAAUUGGCUAACAAUUGGAAUU
UCUGGCCCAGACAGUGGGGCAGUGGCUGUGUUAAAGUACAAUGGC
AUAAUAACAGACACUAUCAAGAGUUGGAGGAACAAUAUAUUGAGA
ACACAAGAGUCUGAAUGUGCAUGUGUAAAUGGUUCUUGCUUUACC
AUAAUGACCGAUGGACCAAGUGAUGGACAGGCCUCAUACAAAAUC
UUCAGAAUAGAAAAGGGAAAGAUAAUCAAAUCAGUCGAAAUGAAA
GCCCCUAAUUAUCACUAUGAGGAAUGCUCCUGUUACCCUGAUUCU
AGUGAAAUCACAUGUGUGUGCAGGGAUAACUGGCAUGGCUCGAAU
CGACCGUGGGUGUCUUUCAACCAGAAUCUGGAAUAUCAGAUGGGA
UACAUAUGCAGUGGGGUUUUCGGAGACAAUCCACGCCCUAAUGAU
AAGACAGGCAGUUGUGGUCCAGUAUCGUCUAAUGGAGCAAAUGGA
GUAAAAGGAUUUUCAUUCAAAUACGGCAAUGGUGUUUGGAUAGGG
AGAACUAAAAGCAUUAGUUCAAGAAAAGGUUUUGAGAUGAUUUGG
GAUCCGAAUGGAUGGACUGGGACUGACAAUAAAUUCUCAAUAAAG
CAAGAUAUCGUAGGAAUAAAUGAGUGGUCAGGGUAUAGCGGGAGU
UUUGUUCAGCAUCCAGAACUAACAGGGCUGGAUUGUAUAAGACCU
UGCUUCUGGGUUGAACUAAUAAGAGGGCGACCCGAAGAGAACACA
AUCUGGACUAGCGGGAGCAGCAUAUCCUUUUGUGGUGUAAACAGU
GACACUGUGGGUUGGUCUUGGCCAGACGGUGCUGAGUUGCCAUUU
ACCAUUGACAAGUAA
INFLUENZA MRNA Matrix-2 (M2) from A/Michigan/
45/2015(H1N1) with deleted amino acid residues
29-31 to inactivate ion-channel activity
and reduce cytotoxicity [SEQ ID NO: 71]
AUGAGUCUUCUAACCGAGGUCGAAACGCCUACCAGAAGCGAAUGG
GAGUGCAGAUGCAGCGGUUCAAGUGAUCCUCUCGUCAUUAUCAUU
GGGAUCUUGCACCUGAUAUUGUGGAUUACUGAUCGUCUUUUUUUC
AAAUGCAUUUAUCGUCGCUUUAAAUACGGUUUGAAAAGAGGGCCU
UCUACGGAAGGAGUGCCUGAGUCCAUGAGGGAAGAAUAUCAACAG
GAGCAGCAGAGUGCUGUGGAUGUUGACGAUGGUCAUUUUGUCAAC
AUAGAGCUAGAGUAA
INFLUENZA MRNA mini Hemagglutinin (HA)
for Group1 #4900 based on the HA
protein sequence from A/Brisbane/59/2007
(H1N1) (ACA28844)
[SEQ ID NO: 72]
AUGAAAGUGAAGCUGCUGGUCCUGCUGUGCACCUUCACCGCCACA
UAUGCCGACACCAUCUGCAUCGGCUACCACGCCAACAACAGCACA
GAUACCGUGGACACCGUGCUGGAGAAGAACGUGACCGUGACACAC
AGCGUUAAUCUGCUGGAAAACGGAGGCGGAGGCAAGUACGUGUGC
AGCGCCAAGCUGAGAAUGGUGACCGGCCUGAGAAACAAACCUAGC
AAGCAGAGCCAAGGCCUGUUCGGCGCCAUCGCCGGCUUCACCGAG
GGCGGAUGGACCGGCAUGGUCGACGGCUGGUACGGCUAUCACCAC
CAGAACGAGCAGGGCAGCGGCUACGCCGCUGAUCAGAAGUCUACA
CAAAAUGCUAUUAACGGCAUCACCAACAAGGUGAACAGCGUGAUC
GAGAAGAUGAAUACCCAGUACACCGCCAUCGGCUGUGAAUACAAC
AAGUCCGAGCGGUGUAUGAAACAGAUCGAAGAUAAGAUCGAGGAG
AUCGAGAGCAAGAUCUGGUGCUACAACGCCGAGCUGCUCGUGCUG
CUGGAAAACGAGAGAACACUGGACUUCCACGAUUCUAAUGUGAAG
AACCUGUACGAGAAGGUGAAGAGCCAGCUGAAGAACAACGCUAAG
GAAAUCGGCAACGGAUGUUUUGAGUUCUACCAUAAGUGCAACGAC
GAGUGCAUGGAAUCUGUGAAGAAUGGAACAUACGACUACCCCAAG
UACAGCGAGGAAUCCAAGCUGAACCGGGAAAAAAUCGACGGCGUG
AAACUGGAAAGCAUGGGCGUGUACCAGAUCUGA
INFLUENZA MRNA Neuramidinase (NA) from
WHO recommended Influenza A virus
(A/Wisconsin/588/2019(H1N1))
[SEQ ID NO: 73]
AUGAAUCCAAACCAAAAGAUAAUAACCAUUGGUUCUAUCUGUAUG
ACAAUUGGAACGGCUAACUUAAUAUUACAAAUUGGAAACAUAAUC
UCAAUAUGGGUUAGCCACUCAAUUCAAAUUGGAAAUCAAAGCCAG
AUUGAAACAUGCAAUAAAAGCGUCAUUACUUAUGAAAACAACACU
UGGGUAAAUCAGACAUUUGUUAACAUCAGCAACACUAACUCUGCU
GCUAGACAGUCAGUGGCUUCCGUGAAAUUAGCGGGCAAUUCCUCU
CUCUGCCCUGUUAGUGGAUGGGCUAUAUACAGUAAAGACAACAGU
GUAAGAAUCGGUUCCAAGGGGGAUGUGUUUGUCAUAAGGGAACCA
UUCAUAUCAUGCUCUCCCUUGGAAUGCAGAACCUUCUUCUUGACU
CAAGGGGCUUUGCUAAAUGACAAACAUUCCAAUGGAACCAUUAAA
GACAGAAGCCCAUAUCGAACCCUAAUGAGCUGUCCUAUUGGUGAA
GUUCCCUCUCCAUACAACUCAAGAUUUGAGUCAGUCGCUUGGUCA
GCAAGUGCUUGUCAUGAUGGCACCAAUUGGCUAACAAUUGGAAUU
UCUGGCCCAGACAGUGGGGCAGUGGCUGUGUUAAAAUACAAUGGC
AUAAUAACAGACACUAUCAAGAGUUGGAGGAACAAGAUAUUGAGA
ACACAAGAGUCUGAAUGUGCAUGUGUAAAUGGUUCUUGCUUUACC
AUAAUGACCGAUGGACCAAGUGAUGGACAGGCCUCAUACAAAAUC
UUCAGAAUAGAAAAGGGAAAGAUAAUCAAAUCAGUCGAAAUGAAA
GCCCCUAAUUAUCACUAUGAAGAAUGCUCCUGUUACCCUGAUUCU
AGUGAAAUCACAUGUGUGUGCAGGGAUAACUGGCAUGGCUCGAAU
CGACCGUGGGUGUCUUUCAACCAGAAUCUGGAAUAUCAGAUGGGA
UACAUAUGCAGUGGGGUUUUCGGAGACAAUCCACGCCCUAAUGAU
AAGACAGGCAGUUGUGGUCCAGUAUCGUCUAAUGGAGCAAAUGGG
GUAAAAGGAUUUUCAUUCAAAUACGGCAAUGGUGUUUGGAUAGGG
AGAACUAAGAGCAUUAGUUCAAGAAAAGGUUUUGAGAUGAUUUGG
GAUCCGAAUGGAUGGACUGGGACUGACAAUAAAUUCUCAAAAAAG
CAAGAUAUCGUAGGAAUAAAUGAGUGGUCAGGGUAUAGCGGGAGU
UUUGUUCAGCAUCCAGAACUAACAGGGCUGAAUUGUAUAAGACCU
UGCUUCUGGGUUGAACUAAUAAGAGGACGACCCGAAGAGAACACA
AUCUGGACUAGCGGGAGCAGCAUAUCCUUUUGUGGUGUAGACAGU
GACAUUGUGGGUUGGUCUUGGCCAGACGGUGCUGAGUUGCCAUUU
ACCAUUGACAAGUAA
INFLUENZA MRNA Hemagglutinin (HA) from
WHO recommended Influenza A virus
(A/Wisconsin/588/2019(H1 N1))
[SEQ ID NO: 74]
AUGAAGGCAAUACUAGUAGUUAUGCUGUAUACAUUUACAACCGCA
AAUGCAGACACAUUAUGUAUAGGUUAUCAUGCGAACAAUUCAACA
GACACUGUGGACACAGUACUAGAAAAGAAUGUAACAGUAACACAC
UCUGUCAAUCUUCUGGAAGACAAGCAUAACGGAAAACUAUGCAAA
CUAAGAGGGGUAGCCCCAUUGCAUUUGGGUAAAUGUAACAUUGCU
GGCUGGAUCCUGGGAAAUCCAGAGUGUGAAUCACUCUCCACAGCA
AGAUCAUGGUCCUACAUUGUGGAAACAUCUAAUUCAGACAAUGGA
ACGUGUUACCCAGGAGAUUUCAUCAAUUAUGAGGAGCUAAGAGAG
CAAUUGAGCUCAGUGUCAUCAUUUGAAAGGUUUGAAAUAUUCCCC
AAGACAAGUUCAUGGCCUAAUCAUGACUCGGACAAUGGUGUAACG
GCAGCAUGUCCUCACGCUGGAGCAAAAAGCUUCUACAAAAACUUG
AUAUGGCUGGUUAAAAAAGGAAAAUCAUACCCAAAGAUCAACCAA
ACCUACAUUAAUGAUAAAGGGAAAGAAGUCCUCGUGCUGUGGGGC
AUUCACCAUCCACCUACUAUUGCUGACCAACAAAGUCUCUAUCAG
AAUGCAGAUGCAUAUGUUUUUGUGGGGACAUCAAGAUACAGCAAG
AAGUUCAAGCCGGAAAUAGCAACAAGACCCAAAGUGAGGGAUCAA
GAAGGGAGAAUGAACUAUUACUGGACACUAGUAGAACCGGGAGAC
AAAAUAACAUUCGAAGCAACUGGUAAUCUAGUGGCACCGAGAUAU
GCAUUCACAAUGGAAAGAGAUGCUGGAUCUGGUAUUAUCAUUUCA
GAUACACCAGUCCACGAUUGCAAUACAACUUGUCAGACACCCGAG
GGUGCUAUAAACACCAGCCUCCCAUUUCAGAAUGUACAUCCGAUC
ACAAUUGGGAAAUGUCCAAAGUAUGUAAAAAGCACAAAAUUGAGA
CUGGCCACAGGAUUGAGGAAUGUCCCGUCUAUUCAAUCUAGAGGC
CUAUUCGGGGCCAUUGCUGGCUUCAUCGAAGGGGGGUGGACAGGG
AUGGUAGAUGGAUGGUACGGUUAUCACCAUCAAAAUGAGCAGGGG
UCAGGAUAUGCAGCCGAUCUGAAGAGCACACAAAAUGCCAUUGAU
AAGAUUACUAACAAAGUAAAUUCUGUUAUUGAAAAGAUGAAUACA
CAGUUCACAGCAGUUGGUAAAGAGUUCAACCACCUUGAAAAAAGA
AUAGAGAAUCUAAAUAAAAAGGUUGAUGAUGGUUUCCUGGACAUU
UGGACUUACAAUGCCGAACUGUUGGUUCUACUGGAAAACGAAAGA
ACUUUGGACUAUCACGAUUCAAAUGUGAAGAACUUGUAUGAAAAA
GUAAGAAACCAGUUAAAAAACAAUGCCAAGGAAAUUGGAAACGGC
UGCUUUGAAUUUUACCACAAAUGCGACAACACAUGCAUGGAAAGU
GUCAAGAAUGGGACUUAUGACUACCCAAAAUACUCAGAGGAAGCA
AAAUUAAACAGAGAAAAAAUAGAUGGAGUAAAGCUGGACUCAACA
AGGAUCUACCAGAUUUUGGCGAUCUAUUCAACUGUUGCCAGUUCA
UUGGUACUGGUAGUCUCCCUGGGGGCAAUCAGCUUCUGGAUGUGC
UCUAAUGGGUCUCUACAGUGUAGAAUAUGUAUUUAA
INFLUENZA MRNA Neuramidinase (NA) from
recommended Influenza B virus
(B/Washington/02/2019 (BNictoria lineage))
[SEQ ID NO: 75]
AUGCUACCUUCAACUAUACAAACGUUAACCCUAUUUCUCACAUCA
GGGGGAGUAUUAUUAUCACUAUAUGUGUCAGCUUCAUUAUCAUAC
UUACUAUAUUCGGAUAUAUUGCUAAAAUUCUCACCAACAGAAAUA
ACUGCACCAACAAUGCCAUUGGAUUGUGCAAACGCAUCAAAUGUU
CAGGCUGUGAACCGUUCUGCAACAAAAGGGGUGACACUUCUUCUC
CCAGAACCGGAGUGGACAUACCCGCGUUUAUCUUGCCCGGGCUCA
ACCUUUCAGAAAGCACUUCUAAUUAGCCCUCAUAGAUUCGGAGAA
ACCAAAGGAAACUCAGCUCCCUUGAUAAUAAGGGAACCUUUUGUA
GCUUGUGGACCAAAUGAAUGCAAACACUUUGCUUUAACCCAUUAU
GCUGCCCAACCAGGGGGAUACUAUAAUGGAACAAGAGGAGACAGA
AACAAGCUGAGGCAUCUAAUUUCAGUCAAAUUGGGCAAAAUCCCA
ACAGUAGAGAACUCCAUUUUCCACAUGGCAGCAUGGAGCGGGUCC
GCGUGCCAUGAUGGUAAGGAAUGGACAUAUAUCGGAGUUGAUGGC
CCUGACAAUAAUGCAUUGCUCAAAGUAAAAUAUGGAGAAGCAUAU
ACUGACACAUACCAUUCCUAUGCAAACAACAUCCUAAGAACACAA
GAAAGUGCCUGCAAUUGCAUCGGGGGAAAUUGUUAUCUAAUGAUA
ACUGAUGGCUCAGCUUCAGGUGUUAGUGAAUGCAGAUUUCUUAAG
AUUCGAGAGGGCCGAAUAAUAAAAGAAAUAUUUCCAACAGGAAGA
GUAAAACACACUGAGGAGUGCACAUGCGGAUUUGCCAGCAAUAAA
ACCAUAGAAUGUGCCUGUAGAGACAACAGGUACACAGCAAAAAGA
CCUUUUGUCAAAUUAAACGUGGAGACUGAUACAGCAGAAAUAAGG
UUGAUGUGCACAGAUACUUAUUUGGACACCCCCAGACCAAAUGAU
GGAAGCAUAACAGGCCCUUGUGAAUCUGAUGGGGACAAAGGGAGU
GGAGGCAUCAAGGGAGGAUUUGUUCAUCAAAGAAUGAAAUCCAAG
AUUGGAAGGUGGUACUCUCGAACGAUGUCUAAAACUGAAAGGAUG
GGGAUGGGACUGUAUGUCAAGUAUGGUGGAGACCCAUGGGCUGAC
AGUGAUGCCCUAACUUUUAGUGGAGUAAUGGUUUCAAUGAAAGAA
CCUGGUUGGUAUUCCUUUGGCUUCGAAAUAAAAGAUAAGAAAUGC
GAUGUCCCCUGUAUUGGGAUAGAGAUGGUACAUGAUGGUGGAAAA
GAGACUUGGCACUCAGCAGCAACAGCCAUUUACUGUUUAAUGGGC
UCAGGACAGCUGCUGUGGGACACUGUCACAGGUGUUGACAUGGCU
CUGUAA
INFLUENZA MRNA Hemagglutinin (HA) from WHO
recommended Influenza B virus
(B/Washington/02/2019 (BNictoria lineage))
[SEQ ID NO: 76]
AUGAAGGCAAUAAUUGUACUACUCAUGGUAGUAACAUCCAAUGCA
GAUCGAAUCUGCACUGGGAUAACAUCGUCAAACUCACCACAUGUC
GUCAAAACUGCUACUCAAGGGGAGGUCAACGUGACCGGUGUAAUA
CCACUGACAACAACACCCACCAAAUCUCAUUUUGCAAAUCUCAAA
GGAACAGAAACCAGGGGGAAACUAUGCCCAAAAUGCCUCAACUGC
ACAGAUCUGGAUGUAGCCUUGGGCAGACCAAAAUGCACAGGGAAA
AUACCCUCUGCAAGGGUUUCAAUACUCCAUGAAGUCAGACCUGUU
ACAUCUGGGUGCUUUCCUAUAAUGCACGAUAGAACAAAAAUUAGA
CAGCUGCCUAACCUUCUCCGAGGAUACGAACAUGUCAGGUUAUCA
ACUCACAACGUUAUCAAUGCAGAAGAUGCACCAGGAAGACCCUAC
GAAAUUGGAACCUCAGGGUCUUGCCCUAACAUUACCAAUGGAAAC
GGAUUCUUCGCAACAAUGGCUUGGGCCGUCCCAAAAAACAAAACA
GCAACAAAUCCAUUAACAAUAGAAGUACCAUACAUUUGUACAGAA
GGAGAAGACCAAAUUACCGUUUGGGGGUUCCACUCUGACAGCGAG
ACCCAAAUGGCAAAGCUCUAUGGGGACUCAAAGCCCCAGAAGUUC
ACCUCAUCUGCCAACGGAGUGACCACACAUUACGUUUCACAGAUU
GGUGGCUUCCCAAAUCAAACAGAAGACGGAGGACUACCACAAAGU
GGCAGAAUUGUUGUUGAUUACAUGGUGCAGAAAUCUGGAAAAACA
GGAACAAUUACCUAUCAAAGAGGUAUUUUAUUGCCUCAAAAGGUG
UGGUGCGCAAGUGGCAGGAGCAAGGUAAUAAAAGGAUCCUUGCCC
UUAAUUGGAGAAGCAGAUUGCCUCCAUGAAAAAUACGGUGGAUUA
AACAAAAGCAAGCCUUACUACACAGGGGAACAUGCAAAGGCCAUA
GGAAAUUGCCCAAUAUGGGUGAAAACACCCUUGAAGCUGGCCAAU
GGAACCAAAUAUAGACCCCCUGCAAAACUAUUAAAGGAAAGAGGU
UUCUUCGGAGCCAUUGCUGGUUUCUUAGAGGGAGGAUGGGAAGGA
AUGAUUGCAGGUUGGCACGGAUACACAUCCCAUGGGGCACAUGGA
GUAGCGGUGGCAGCUGACCUUAAGAGCACUCAAGAGGCCAUAAAC
AAGAUAACAAAAAAUCUCAACUCUUUGAGUGAGCUGGAAGUAAAG
AAUCUUCAAAGACUAAGCGGUGCCAUGGAUGAACUCCACAACGAA
AUACUAGAACUAGAUGAGAAAGUGGAUGAUCUCAGAGCUGAUACA
AUAAGCUCACAAAUAGAACUCGCAGUCCUGCUUUCCAAUGAAGGA
AUAAUAAACAGUGAAGAUGAACAUCUCUUGGCGCUUGAAAGAAAG
CUGAAGAAAAUGCUGGGCCCCUCUGCUGUAGAGAUAGGGAAUGGA
UGCUUUGAAACCAAACACAAGUGCAACCAGACCUGUCUCGACAGA
AUAGCUGCUGGUACCUUUGAUGCAGGAGAAUUUUCUCUCCCCACC
UUUGAUUCACUGAAUAUUACUGCUGCAUCUUUAAAUGACGACGGA
UUGGACAAUCAUACUAUACUGCUUUACUACUCAACUGCUGCCUCC
AGUUUGGCUGUAACACUGAUGAUAGCUAUCUUUGUUGUUUAUAUG
GUCUCCAGAGACAAUGUUUCUUGCUCCAUUUGUCUAUAA
INFLUENZA MRNA mini Hemagglutinin (HA) for
Group2 H3ssF_C based on the HA protein
sequence from A/Finland/486/2004 (H3N2)
[SEQ ID NO: 77]
AUGAAGACCAUCAUCGCCCUGAGCUACAUCCUGUGCCUGGUGUUC
GCCCAGAAGCUGCCCGGCAACGACAACAGCACCGCCACCCUGUGC
CUGGGCCACCACGCCGUGCCCAACGGCACCAUCGUGAAGACCAUC
ACCAACGACCAGAUCGAGGUGACCAACGCCACCGAGCUGGUGUUC
CCCGGCUGCGGCGUGCUGAAGCUGGCCACCGGCAUGAGGAACGUG
CCCGAGAAGCAGACCAGGGGCAUCUUCGGCGCCAUCGCCGGCUUC
AUCGAGAACGGCUGGGAGGGCAUGGUGGACGGCUGGUACGGCUUC
AGGCACCAGAACAGCGAGGGCAUCGGCCAGGCCGCCGACCUGAAG
AGCACCCAGGCCGCCAUCAACCAGAUCAACGGCAUGGUGAACAGG
GUGAUCGAGCUGAUGGAGCAGGGCGGCCCCGACUGCUACCUGGCC
GAGCUGCUGGUGGCCCUGCUGAACCAGCACACCAUCGACCUGACC
GACAGCGAGAUGAGGAAGCUGUUCGAGAGGACCAAGAAGCAGCUG
AGGGAGAACGCCGAGGACAUGGGCAACGGCUGCUUCAAGAUCUAC
CACAAGUGCGACAACGCCUGCAUCGGCAGCAUCAGGAACGGCACC
UACGACCACGACGUGUACAGGGACGAGGCCCUGAACAACAGGUUC
CAGAUCAAGUAA
INFLUENZA MRNA mini Hemagglutinin (HA) for
Group2 H7ssF_C based on the HA protein
sequence from A/Shanghai/2/2013 (H7N9)
[SEQ ID NO: 78]
AUGAACACCCAGAUCCUGGUGUUCGCCCUGAUCGCCAUCAUCCCC
ACCAACGCCGACAAGAUCUGCCUGGGCCACCACGCCGUGAGCAAC
GGCACCAAGGUGAACACCCUGACCGAGAGGGGCGUGGAGGUGGUG
AACGCCACCGAGCUGGUGUUCCCCGGCUGCGGCGUGCUGCUGCUG
GCCACCGGCAUGAAGAACGUGCCCGAGAUCCCCAAGGGCAGGGGC
CUGUUCGGCGCCAUCGCCGGCUUCAUCGAGAACGGCUGGGAGGGC
CUGAUCGACGGCUGGUACGGCUUCAGGCACCAGAACGCCCAGGGC
GAGGGCACCGCCGCCGACUACAAGAGCACCCAGAGCGCCAUCGAC
CAGAUCACCGGCAUGGUGAACAGGGUGAUCGAGCUGAUGGAGCAG
GGCGGCCCCGACUGCUACCUGGCCGAGCUGCUGGUGGCCAUGCUG
AACCAGCACACCAUCGACCUGGCCGACAGCGAGAUGGACAAGCUG
UACGAGAGGGUGAAGAGGCAGCUGAGGGAGAACGCCGAGGAGGAC
GGCACCGGCUGCUUCGAGAUCUUCCACAAGUGCGACGACGACUGC
AUGGCCAGCAUCAGGAACAACACCUACGACCACAGCAAGUACAGG
GAGGAGGCCAUGCAGAACAGGAUCCAGAUCGACUAA
RESPIRATORY SYNCYTIAL VIRUS MRNA RSV Prefusion
F Glycoprotein from A2 strain (S155C, S190F,
V207L, S290C mutations that stabilize the
prefusion conformation)
[SEQ ID NO: 79]
AUGGAGUUGCUAAUCCUCAAAGCAAAUGCAAUUACCACAAUCCUC
ACUGCAGUCACAUUUUGUUUUGCUUCUGGUCAAAACAUCACUGAA
GAAUUUUAUCAAUCAACAUGCAGUGCAGUUAGCAAAGGCUAUCUU
AGUGCUCUGAGAACUGGUUGGUAUACCAGUGUUAUAACUAUAGAA
UUAAGUAAUAUCAAGGAAAAUAAGUGUAAUGGAACAGAUGCUAAG
GUAAAAUUGAUAAAACAAGAAUUAGAUAAAUAUAAAAAUGCUGUA
ACAGAAUUGCAGUUGCUCAUGCAAAGCACACCACCAACAAACAAU
CGAGCCAGAAGAGAACUACCAAGGUUUAUGAAUUAUACACUCAAC
AAUGCCAAAAAAACCAAUGUAACAUUAAGCAAGAAAAGGAAAAGA
AGAUUUCUUGGUUUUUUGUUAGGUGUUGGAUCUGCAAUCGCCAGU
GGCGUUGCUGUAUGUAAGGUCCUGCACCUAGAAGGGGAAGUGAAC
AAGAUCAAAAGUGCUCUACUAUCCACAAACAAGGCUGUAGUCAGC
UUAUCAAAUGGAGUUAGUGUCUUAACCUUCAAAGUGUUAGACCUC
AAAAACUAUAUAGAUAAACAAUUGUUACCUAUUCUGAACAAGCAA
AGCUGCAGCAUAUCAAAUAUAGAAACUGUGAUAGAGUUCCAACAA
AAGAACAACAGACUACUAGAGAUUACCAGGGAAUUUAGUGUUAAU
GCAGGUGUAACUACACCUGUAAGCACUUACAUGUUAACUAAUAGU
GAAUUAUUGUCAUUAAUCAAUGAUAUGCCUAUAACAAAUGAUCAG
AAAAAGUUAAUGUCCAACAAUGUUCAAAUAGUUAGACAGCAAAGU
UACUCUAUCAUGUGCAUAAUAAAAGAGGAAGUCUUAGCAUAUGUA
GUACAAUUACCACUAUAUGGUGUUAUAGAUACACCCUGUUGGAAA
CUACACACAUCCCCUCUAUGUACAACCAACACAAAAGAAGGGUCC
AACAUCUGUUUAACAAGAACUGACAGAGGAUGGUACUGUGACAAU
GCAGGAUCAGUAUCUUUCUUCCCACAAGCUGAAACAUGUAAAGUU
CAAUCAAAUCGAGUAUUUUGUGACACAAUGAACAGUUUAACAUUA
CCAAGUGAAAUAAAUCUCUGCAAUGUUGACAUAUUCAACCCCAAA
UAUGAUUGUAAAAUUAUGACUUCAAAAACAGAUGUAAGCAGCUCC
GUUAUCACAUCUCUAGGAGCCAUUGUGUCAUGCUAUGGCAAAACU
AAAUGUACAGCAUCCAAUAAAAAUCGUGGAAUCAUAAAGACAUUU
UCUAACGGGUGCGAUUAUGUAUCAAAUAAAGGGAUGGACACUGUG
UCUGUAGGUAACACAUUAUAUUAUGUAAAUAAGCAAGAAGGUAAA
AGUCUCUAUGUAAAAGGUGAACCAAUAAUAAAUUUCUAUGACCCA
UUAGUAUUCCCCUCUGAUGAAUUUGAUGCAUCAAUAUCUCAAGUC
AACGAGAAGAUUAACCAGAGCCUAGCAUUUAUUCGUAAAUCCGAU
GAAUUAUUACAUAAUGUAAAUGCUGGUAAAUCCACCACAAAUAUC
AUGAUAACUACUAUAAUUAUAGUGAUUAUAGUAAUAUUGUUAUCA
UUAAUUGCUGUUGGACUGCUCUUAUACUGUAAGGCCAGAAGCACA
CCAGUCACACUAAGCAAAGAUCAACUGAGUGGUAUAAAUAAUAUU
GCAUUUAGUAACUAA
MYCOBACTERIUM TUBERCULOSIS MRNA Tuberculosis
ESAT-6 (Rv3875)-Active phase (sequence
from H37Rv strain) [SEQ ID NO: 80]
AUGACAGAGCAGCAGUGGAAUUUCGCGGGUAUCGAGGCCGCGGCA
AGCGCAAUCCAGGGAAAUGUCACGUCCAUUCAUUCCCUCCUUGAC
GAGGGGAAGCAGUCCCUGACCAAGCUCGCAGCGGCCUGGGGCGGU
AGCGGUUCGGAGGCGUACCAGGGUGUCCAGCAAAAAUGGGACGCC
ACGGCUACCGAGCUGAACAACGCGCUGCAGAACCUGGCGCGGACG
AUCAGCGAAGCCGGUCAGGCAAUGGCUUCGACCGAAGGCAACGUC
ACUGGGAUGUUCGCAUAG
MYCOBACTERIUM TUBERCULOSIS MRNA Tuberculosis
Ag85B (Rv1886)-Active phase (sequence
from H37Rv strain) [SEQ ID NO: 81]
AUGACAGACGUGAGCCGAAAGAUUCGAGCUUGGGGACGCCGAUUG
AUGAUCGGCACGGCAGCGGCUGUAGUCCUUCCGGGCCUGGUGGGG
CUUGCCGGCGGAGCGGCAACCGCGGGCGCGUUCUCCCGGCCGGGG
CUGCCGGUCGAGUACCUGCAGGUGCCGUCGCCGUCGAUGGGCCGC
GACAUCAAGGUUCAGUUCCAGAGCGGUGGGAACAACUCACCUGCG
GUUUAUCUGCUCGACGGCCUGCGCGCCCAAGACGACUACAACGGC
UGGGAUAUCAACACCCCGGCGUUCGAGUGGUACUACCAGUCGGGA
CUGUCGAUAGUCAUGCCGGUCGGCGGGCAGUCCAGCUUCUACAGC
GACUGGUACAGCCCGGCCUGCGGUAAGGCUGGCUGCCAGACUUAC
AAGUGGGAAACCUUCCUGACCAGCGAGCUGCCGCAAUGGUUGUCC
GCCAACAGGGCCGUGAAGCCCACCGGCAGCGCUGCAAUCGGCUUG
UCGAUGGCCGGCUCGUCGGCAAUGAUCUUGGCCGCCUACCACCCC
CAGCAGUUCAUCUACGCCGGCUCGCUGUCGGCCCUGCUGGACCCC
UCUCAGGGGAUGGGGCCUAGCCUGAUCGGCCUCGCGAUGGGUGAC
GCCGGCGGUUACAAGGCCGCAGACAUGUGGGGUCCCUCGAGUGAC
CCGGCAUGGGAGCGCAACGACCCUACGCAGCAGAUCCCCAAGCUG
GUCGCAAACAACACCCGGCUAUGGGUUUAUUGCGGGAACGGCACC
CCGAACGAGUUGGGCGGUGCCAACAUACCCGCCGAGUUCUUGGAG
AACUUCGUUCGUAGCAGCAACCUGAAGUUCCAGGAUGCGUACAAC
GCCGCGGGCGGGCACAACGCCGUGUUCAACUUCCCGCCCAACGGC
ACGCACAGCUGGGAGUACUGGGGCGCUCAGCUCAACGCCAUGAAG
GGUGACCUGCAGAGUUCGUUAGGCGCCGGCUGA
MYCOBACTERIUM TUBERCULOSIS MRNA Tuberculosis
TB10.4 (Rv0288)-Active phase (sequence
from H37Rv strain) [SEQ ID NO: 82]
AUGUCGCAAAUCAUGUACAACUACCCCGCGAUGUUGGGUCACGCC
GGGGAUAUGGCCGGAUAUGCCGGCACGCUGCAGAGCUUGGGUGCC
GAGAUCGCCGUGGAGCAGGCCGCGUUGCAGAGUGCGUGGCAGGGC
GAUACCGGGAUCACGUAUCAGGCGUGGCAGGCACAGUGGAACCAG
GCCAUGGAAGAUUUGGUGCGGGCCUAUCAUGCGAUGUCCAGCACC
CAUGAAGCCAACACCAUGGCGAUGAUGGCCCGCGACACGGCCGAA
GCCGCCAAAUGGGGCGGCUAG
MYCOBACTERIUM TUBERCULOSIS MRNA Tuberculosis
Hrp1 (Rv2626)-Latent phase (sequence from
H37Rv strain) [SEQ ID NO: 83]
AUGACCACCGCACGCGACAUCAUGAACGCAGGUGUGACCUGUGUU
GGCGAACACGAGACGCUAACCGCUGCCGCUCAAUACAUGCGUGAG
CACGACAUCGGCGCGUUGCCGAUCUGCGGGGACGACGACCGGCUG
CACGGCAUGCUCACCGACCGCGACAUUGUGAUCAAAGGCCUGGCU
GCGGGCCUAGACCCGAAUACCGCCACGGCUGGCGAGUUGGCCCGG
GACAGCAUCUACUACGUCGAUGCGAACGCAAGCAUCCAGGAGAUG
CUCAACGUCAUGGAAGAACAUCAGGUCCGCCGUGUUCCGGUCAUC
UCAGAGCACCGCUUGGUCGGAAUCGUCACCGAAGCCGACAUCGCC
CGACACCUGCCCGAGCACGCCAUUGUGCAGUUCGUCAAGGCAAUC
UGCUCGCCCAUGGCCCUCGCCAGCUAG
MYCOBACTERIUM TUBERCULOSIS MRNA Tuberculosis
RpfB-D hybrid-Resuscitation phase
(sequences from H37Rv strain).
[SEQ ID NO: 84]
AUGACCGUCGACGGAACCGCGAUGCGGGUGACCACGAUGAAAUCG
CGGGUGAUCGACAUCGUCGAAGAGAACGGGUUCUCAGUCGACGAC
CGCGACGACCUGUAUCCCGCGGCCGGCGUGCAGGUCCAUGACGCC
GACACCAUCGUGCUGCGGCGUAGCCGUCCGCUGCAGAUCUCGCUG
GAUGGUCACGACGCUAAGCAGGUGUGGACGACCGCGUCGACGGUG
GACGAGGCGCUGGCCCAACUCGCGAUGACCGACACGGCGCCGGCC
GCGGCUUCUCGCGCCAGCCGCGUCCCGCUGUCCGGGAUGGCGCUA
CCGGUCGUCAGCGCCAAGACGGUGCAGCUCAACGACGGCGGGUUG
GUGCGCACGGUGCACUUGCCGGCCCCCAAUGUCGCGGGGCUGCUG
AGUGCGGCCGGCGUGCCGCUGUUGCAAAGCGACCACGUGGUGCCC
GCCGCGACGGCCCCGAUCGUCGAAGGCAUGCAGAUCCAGGUGACC
CGCAAUCGGAUCAAGAAGGUCACCGAGCGGCUGCCGCUGCCGCCG
AACGCGCGUCGUGUCGAGGACCCGGAGAUGAACAUGAGCCGGGAG
GUCGUCGAAGACCCGGGGGUUCCGGGGACCCAGGAUGUGACGUUC
GCGGUAGCUGAGGUCAACGGCGUCGAGACCGGCCGUUUGCCCGUC
GCCAACGUCGUGGUGACCCCGGCCCACGAAGCCGUGGUGCGGGUG
GGCACCAAGCCCGGUACCGAGGUGCCCCCGGUGAUCGACGGAAGC
AUCUGGGACGCCAUCGCGCAAUGCAAAUCCGGCGGCAAUUGGGCG
GCCAACACCGGUAACGGGUUAUACGGUGGUCUGCAGAUCAGCCAG
GCGGCGUGGGAUUCCAACGGUGGUGUCGGGUCGCCGGCGGCCGCG
AGUCCCCAGCAACAGAUCGAGGUCGCAGACAACAUUAUGAAAACC
GCAGGCCCGGGUGCGUGGCCGAAAUGUAGUUCUUGUAGUCAGGGA
GACGCACCGCUGGGCUCGCUCACCCACAUCCUGACGUUCCUCGCG
GCCGAGACUGGAGGUUGUUCGGGGAGCAGGGACGAUUGA

It is contemplated herein to provide compositions, including pharmaceutical compositions, comprising mRNA which encode more than one antigen, for example, encoding the spike protein from more than one SARS-COV-2 spike protein. Multiple antigens can be provided by the same, or different mRNA constructs, as described elsewhere herein. In one embodiment, a composition is provided comprising mRNA constructs encoding the spike protein from at least two, at least three, or all four of the wild type SARS-COV-2, the Beta (South African) variant SARS-COV-2, the Delta variant SARS-COV-2, and the Omicron variant SARS-COV-2. These may be present on the same or different mRNA constructs. The mRNA construct(s) encoding these antigen may lack OPS, or one or more, or all of them have OPS as described elsewhere herein. In some embodiments, the OPS can comprise sequences capable of binding with miRNA-122, miRNA-1, miRNA-203a, and miRNA-30a; or sequences capable of binding with miRNA-122, miRNA-192, and miRNA-30a. In any of these embodiments, the composition may also comprise mRNA coding for an proinflammatory cytokine, as further discussed herein, e.g., IL-12. The proinflammatory cytokine mRNA may lack an OPS, or may comprise an OPS as described elsewhere herein. In some embodiments, the OPS can comprise sequences capable of binding with miRNA-122, miRNA-1, miRNA-203a, and miRNA-30a; or sequences capable of binding with miRNA-122, miRNA-192, and miRNA-30a. In specific embodiments, the mRNA construct(s) encoding the antigen (for example, two or more variant SARS-CoV-2 spike protein) may lack OPS, while the mRNA construct(s) encoding the proinflammatory cytokine (for example, IL-12) may include an OPS, as described.

In embodiments where mRNA coding for both antigen and proinflammatory cytokines are administered, these can be provided as separate mRNA constructs, which may be co-formulated, or separately formulated. In some embodiments one or other of the mRNA constructs may entirely lack miRNA binding site sequences. In other cases each mRNA construct may comprise one or more organ protection sequences as described herein. These organ protection sequences may be the same for each mRNA construct, or may be different. It is considered that given the different purposes and potential for off-target effects of antigen and proinflammatory cytokine products, use of different organ protection sequences for each of these products may be beneficial, in order to support a different pattern of differential expression for these products, and/or to extend protection to different tissues or cell types for each product. For example, it may be advantageous for antigen components to be expressed primarily by the myocytes as well as APC, so the organ protection sequences comprised in mRNA encoding these products may be selected to enable expression in these cell types, while protecting other healthy tissue. In some cases, it may be preferred for the antigen component to have organ protection sequences comprising target sequences for miRNA-122, miRNA-192, and/or miRNA 30a, or all three of these.

Proinflammatory cytokines such as IL-12, have the potential of producing off-target effects, so mRNA encoding these factors may be chosen to provide maximum protection to muscle, liver, kidney, lung, spleen and/or skin as discussed above (for example with target sequences for miRNA-1, miRNA-122, miRNA-30a and/or miRNA-203a, or all four of these), while the mRNA encoding the antigen component may comprise fewer miRNA binding site sequences, in order to increase the breadth of expression.

Constructs and compositions according to the above discussion, whether encoding antigen or a proinflammatory cytokines, can comprise any organ protection sequences as described herein. However, in particular embodiments, the organ protection sequences are selected to protect one or more of muscle, liver, kidney, lungs, spleen, and skin (for example, using target sequences for miRNA-1, miRNA-122, miRNA-192, miRNA-30a and/or miRNA-203a). In some embodiments, target sequences for all four of miRNA-1, miRNA-122, miRNA-30a and miRNA-203a are included in the organ protection sequences. Such a combination is thought to be effective in protecting muscle tissue (as compositions may be administered intramuscularly), as well as liver and kidney tissue. It is particularly considered in any embodiment where the protection of muscle tissue is desired, that target sequences for miRNA 133a and/or for miRNA 206 may be included instead of or in addition to miRNA 1, in accordance with Table 2. For example, such OPS could include target sequences for miRNA-133a, miRNA-122, miRNA-192, and miRNA-30a; or for miRNA-206, miRNA-122, miRNA-192, and miRNA-30a. Subcutaneous or intradermal administration is also common, and one or more of the miRNA target sequences associated with the skin (see Table 2) may also be used to protect cells of the skin.

It is thought that certain vaccines can have side-effects linked to interactions with endothelial tissue. In Goldman M, Hermans C (2021) PLOS Med 18(5): e1003648. doi.org/10.1371/joumal.pmed.1003648, the following mechanism was suggested: After intramuscular injection, vaccine adenoviruses infect endothelial cells, inducing their production of the SARS-COV-2 Spike protein. Heparan sulfate PG could bind the spike protein on the luminal side of endothelial cells or be released by damaged cells. Spike proteins would activate platelets via ACE2-dependent and ACE2-independent mechanisms. PF4 released by activated platelets would become immunogenic after binding heparan sulfate PG shed from endothelial cells.

In some embodiments, it may therefore be desired to include miRNA target sequences to protect endothelial tissue. As discussed in Table 2, miRNA-98 and/or miRNA-126 target sequences may therefore be included in OPS. This type of protection is thought to be of use with any mode of administration, and particularly where administration into blood vessels (intravenous, intraarterial, etc.) or intramuscular administration is used. In other embodiments target sequences may include any appropriate combinations of one or more sequences from Table 3 or 4 above. In specific embodiments, the OPS comprised within mRNA constructs encoding the immunomodulators can comprise sequences capable of binding with: miRNA-122, miRNA-1, miRNA-203a, and miRNA-30a; Let7b, miRNA-126, and miRNA-30a; miRNA-122, miRNA-192, and miRNA-30a; or sequences capable of binding with miRNA-192, miRNA-30a, and miRNA-124, with two sequences capable of binding with miRNA 122. It is also considered advantageous to avoid the use of miRNA-142 target sequences in such constructs and compositions, as this miRNA is abundant in cells of haematopoietic origin and immune cells, and therefore could lead to a reduction in expression in the cells anticipated to mediate the vaccine-mediated response.

In some embodiments, it is envisioned that a composition(s) may be provided which comprises mRNA encoding viral proteins from each of SARS-COV-2 (or a variant thereof) and influenza, for example, in order to provide a multivalent or joint vaccination against a seasonal, new, or emerging variant of one or both of these viruses. As described elsewhere, the different antigen may be provided on the same or different mRNA constructs, and these mRNA construct(s) may lack an OPS, or may comprise an OPS/MOP as described elsewhere. The compositions may further comprise mRNA coding for an proinflammatory cytokine, such as IL-12, as further discussed below. This mRNA may also comprise an OPS, as described.

In addition to the conventional preventive or prophylactic vaccinations, a newer field is that of therapeutic vaccines which aim to provoke an immune response against targets which are already present in the body, for example, against persistent infections. This has proven much more challenging, because in such cases the immune response has often been downregulated or otherwise restrained by tolerance mechanisms which act to protect the disease from the normal immune response (Melief et al Therapeutic cancer vaccines JCI 2015); which is incorporated by reference herein in its entirety.

In embodiments of the present invention, an mRNA sequence is provided that comprises a sequence that codes for at least one polypeptide in operative combination with one or more untranslated regions (UTRs) that may confer tissue specificity, and stability to the nucleic acid sequence as a whole. By ‘tissue specificity’, it is meant that translation of the protein product encoded by the mRNA is modulated according to the presence of the UTRs. Modulation may include permitting, reducing or even blocking detectable translation of the mRNA into a protein. The UTRs may be linked directly to the mRNA in cis—i.e., on the same polynucleotide strand. In an alternative embodiment, a first sequence that codes for a gene product is provided and a further second sequence, that hybridises to a portion of the first sequence, is provided that comprises one or more UTRs that confer tissue specificity to the nucleic acid sequence as a whole. In this latter embodiment, the UTR is operatively linked to the sequence that encodes the gene product in trans.

According to specific embodiments of the invention, an mRNA is provided that comprises such associated nucleic acid sequences operatively linked thereto as are necessary to prevent or reduce expression of a gene product in non-diseased tissue, e.g., in healthy hepatocytes, CNS, muscle, skin etc. The mRNA is hereafter referred to as a ‘coding mRNA’. As such, this coding mRNA construct, or transcript, is provided that comprises a 5′ cap and UTRs necessary for ribosomal recruitment and tissue and/or organ specific expression (typically, but not exclusively positioned 3′ to the ORF), as well as start and stop codons that respectively define one or more ORFs. When the construct is introduced systemically or via localised administration into non-diseased liver, lung, pancreas, breast, brain/CNS, kidney, spleen, muscle, skin and/or colon-GI tract, expression of the gene product is prevented or reduced. In contrast, neoplastic or otherwise diseased cells comprised within the aforementioned organs typically do not conform to normal non-diseased cell expression patterns, possessing a quite different miRNA transcriptome. The polypeptide(s) encoded by the mRNA is translated specifically in these aberrant cells but not- or to a lesser extent—in neighboring healthy or non-diseased cells. Delivery of the mRNA construct to the organs mentioned above may be achieved via a particulate delivery platform as described herein, or in any suitable way known in the art. Cell type specific expression can be mediated via microRNA modulation mechanisms such as those described in more detail elsewhere herein.

According to further embodiments of the invention, an mRNA is provided that comprises such associated nucleic acid sequences operatively linked thereto as are necessary to prevent or reduce expression of a gene product in tissues or organs not required to generate an immune response to an antigen, e.g., in hepatocytes, CNS, muscle, skin, kidney etc. The coding mRNA construct, or transcript, is provided that may or may not comprise a 5′ cap, as well as one or more UTRs necessary for ribosomal recruitment and tissue and/or organ specific expression (typically, but not exclusively positioned 3′ to the ORF), as well as start and stop codons that respectively define one or more ORFs. When the construct is introduced systemically or via localised administration into a subject, expression of the gene product is prevented or reduced in cells and tissues that are not typically required for an immune response. In contrast, immune cells, such as T cells, B Cells or antigen presenting cells (APCs), including different types of dendritic cells (DCs), comprised within the body or in the aforementioned organs possess a different miRNA transcriptome. The polypeptide(s) encoded by the mRNA is translated specifically in these immune cells but not- or to a lesser extent—in neighboring healthy cells and tissues. Delivery of the mRNA construct to the cells and tissues mentioned above may be achieved via a particulate delivery platform as described herein, or in any suitable way known in the art.

MicroRNAs (miRNAs) are a class of noncoding RNAs each containing around 20 to 25 nucleotides, some of which are believed to be involved in post-transcriptional regulation of gene expression by binding to complementary target sequences in the 3′ untranslated regions (3′ UTR) of target mRNAs, leading to their silencing. These miRNA complementary target sequences are also referred to herein as miRNA binding sites, or miRNA binding site sequences. Certain miRNAs are highly tissue-specific in their expression; for example, miRNA-122 and its variants are abundant in the liver and infrequently expressed in other tissues (Lagos-Quintana et al. Current Biology. 2002; 12:735-739).

The miRNA system therefore provides a robust platform by which nucleic acids introduced into cells can be silenced in selected cell types in a target tissue, and expressed in others. By including a target sequence for a particular given miRNA into an mRNA construct to be introduced into target cells, particularly within a UTR, expression of certain introduced genes can be reduced or substantially eliminated in some cell types, while remaining in others (Brown and Naldini, Nat Rev Genet. 2009; 10(8): 578-585).

In accordance with specific embodiments of the present invention it is contemplated that a plurality of such miRNA target sequences can be comprised within an organ protection sequence (OPS), which is then included in the mRNA construct. Where a plurality of miRNA target sequences are present, this plurality may include for example greater than two, greater than three, typically greater than four miRNA target sequences.

As used herein, the term ‘organ protection sequence’ (‘OPS’) refers to a sequence comprised of a plurality of microRNA (miRNA) target sequences of natural or synthetic origin and, optionally, one or more auxiliary sequences. Where an OPS confers protection to multiple organs it may be referred to as a multiple or ‘multi-organ’ protection (MOP) sequence.

These miRNA target sequences in an OPS or MOPS may be arranged sequentially, in tandem or at predetermined locations within, a specified UTR within the mRNA constructs. Multiple miRNA target sequences may be separated with auxiliary sequences that serve to support or facilitate the functioning of the organ protection sequence as a whole. By way of example, suitable auxiliary sequences may consist of a linker or spacer sequence, which may be randomized, or may comprise a particular sequence, for example, “uuuaaa”, although other spacer sequences can also be used. The length of the spacer can vary, and can comprise repetitions of a spacer sequence, for example the spacer “uuuaaa” can be included once (i.e., “uuuaaa”), twice (i.e., “uuuaaauuuaaa”—SEQ ID NO: 1), three times, four times, five times, or six times between each and any target sequence to be linked. In some embodiments, no spacer sequence may be present between binding site sequences.

In some embodiments of any of the aspects, one or more of the first, second, or further ORFs is operatively linked to at least one untranslated region (UTR), wherein each UTR comprises at least a first organ protection sequence (OPS), wherein each OPS comprises at least two micro-RNA (miRNA) target sequences, and wherein each of the at least two miRNA target sequences are optimised to hybridise with a corresponding miRNA sequence. In some embodiments of any of the aspects, each ORF of the composition is operatively linked to a UTR comprising at least one OPS.

miRNA-122, despite its abundance in healthy non-diseased liver tissue, is reduced in the majority of liver cancers as well as in diseased cells (Braconi et al. Semin Oncol. 2011; 38(6): 752-763, Brown and Naldini, Nat Rev Genet. 2009; 10(8): 578-585). By the above-mentioned method, it has been found that when the target tissue is the liver, translation of the introduced mRNA sequences can be facilitated in cancerous liver cells and reduced or substantially eliminated in transfected healthy cells, by including miRNA-122 target sequence (for example, SEQ ID NO: 1) in their 3′ UTRs.

In a similar way, differential translation of such mRNA is also possible between infected or diseased cells and healthy cells in other organs, by using other miRNA target sequences. Suitable candidates include (but are not limited to) target sites for: miRNA-1, miRNA-125, miRNA-199, miRNA-124a, miRNA-126, miRNA-Let7, miRNA-375, miRNA-141, miRNA-142, miRNA-143, miRNA-145, miRNA-148, miRNA-194, miRNA-200c, miRNA-34a, miRNA-192, miRNA-194, miRNA-204, miRNA-215 and miRNA-30 family (for example, miRNA-30 a, b, or c).

Table 2 demonstrates further (non-limiting) examples of miRNA sequences where expression has been demonstrated in particular organs and/or tissues, and in several cases where differential expression is demonstrated between healthy and diseased cells. miRNA-1, miRNA-133a and miRNA-206 have been described as examples of muscle and/or myocardium-specific miRNAs (Sempere et al. Genome Biology. 2004; 5: R13; Ludwig et al. Nucleic Acids Research. 2016; 44 (8): 3865-3877). miRNA-1 has also been demonstrated to be dysregulated in disease, for example downregulation of miRNA-1 has been detected in infarcted heart tissue (Bostjancic E, et al. Cardiology. 2010; 115 (3): 163-169), while a drastic reduction of miRNA-1 has also been detected in rhabdomyosarcoma cell lines (Rao, Prakash K et al. FASEB J. 2010; 24 (9): 3427-3437). Use of miRNA-1, miRNA-133a and miRNA-206 may be articularly considered where compositions according to the invention are to be administered intramuscularly, so to reduce expression in local normal myocytes, if desired.

miRNA-125 is expressed in a number of tissues as shown in Table 2, and is downregulated in several solid tumors, such as hepatocellular carcinoma (Coppola et al. Oncotarget 2017; 8); breast (Mattie et al. Mol Cancer 2006; 5), lung (Wang et al. FEBS J 2009), ovarian (Lee et al. Oncotarget 2016; 7), gastric (Xu et al. Mol Med Rep 2014; 10), colon (Tong et al. Biomed Pharmacother 2015; 75), and cervical cancers (Fan et al Oncotarget 2015; 6); neuroblastoma, medulloblastoma (Ferretti et al. Int J Cancer 2009; 124), glioblastoma (Cortez et al. Genes Chromosomes Cancer 2010; 49), and retinoblastoma (Zhang et al; Cell signal 2016; 28).

Several miRNA species are also differentially expressed in glioblastoma multiforme cells (Zhangh et al. J Miol Med 2009; 87/Shi et al. Brain Res 2008; 1236) compared to non-diseased brain cells (e.g. neurons), with miRNA-124a one of the most dysregulated (Karsy et al. Gene Cancer 2012; 3; Riddick et al. Nat Rev Neurol 2011; 7; Gaur et al. Cancer Res 2007; 67/Silber et al. BMC Med 2008; 6).

In lung cancer, a recent meta-analysis confirmed the downregulation of Let-7 (as well as miRNA-148a and miRNA-148b) in non-small-cell lung cancer (Lamichhane et al. Disease Markers 2018).

Similarly, miRNA-375 expression has been found to be downregulated in pancreatic cancer cells, compared to healthy pancreatic cells (Shiduo et al. Biomedical Reports 2013; 1). In the pancreas, miRNA-375 expression has been indicated to be high in normal pancreas cells but significantly lower in diseased and/or cancerous tissues (Song, Zhou et al. 2013). This expression has been shown to relate to the stage of cancer, with expression further reduced with more advanced cancer. It is thought that miRNA-375 is involved with the regulation of glucose-induced biological responses in pancreatic 13-cells, by targeting 3-phosphoinositide-dependent protein kinase-1 (PDK1) mRNA and so affecting the PI 3-kinase/PKB cascade (El Ouaamari et al. Diabetes 57:2708-2717, 2008). An anti-proliferative effect of miRNA-375 is implicated by this putative mode of action, which may explain its downregulation in cancer cells.

Table 2 discusses non-limiting examples of miRNAs associated with particular organs and/or tissues, which may be used in embodiments of the present invention. It will be appreciated, that the present invention is not limited only to instances where a given miRNA or class of miRNAs is downregulated in a first cell type versus a second cell type within a given organ or organ system. On the contrary, it is merely required that there exists a differential expression pattern of a regulatory miRNA between cell types, for example those comprised within an organ or organ system, or between different organs or organ systems. The differential expression of the miRNA system can be exploited using the compositions and methods described herein to enable corresponding differential translation of protein products between cells, thereby reducing undesired off-target side effects. This is of particular use in embodiments where differential expression of a mRNA between cell types or tissues is desired. For example, it may be advantageous to express an mRNA encoding a proinflammatory cytokine, if used as an adjuvant, primarily in immune cells but not in one or more healthy tissues where an increase in inflammation would not be desired-such as the skin, liver, kidney or colon.

TABLE 2
Exemplary miRNA associated with particular tissue/organ types

| Tissue | Implicated mRNA | Reference |
|---|---|---|
| Liver | miRNA-122 | Braconi et al. Seminars in Oncology. 2011; 38(6): |
| | | 752-763; |
| | | Brown and Naldini. Nature Reviews Genetics. |
| | | 2009; 10:578-585; |
| | | Fu and Colin. EBioMedicine. 2018; 17-18. |
| Liver | miRNA-125, | Coppola N et al. Oncotarget, 2017; 8(15): |
| | miRNA-199 | 25289-299 |
| | | Murakami Y. Oncogene 2006; 25 |
| | | Hou et al. Cancer Cell, 2011; 19(2): 232-243. |
| | | Shi et al. Medicine. 2017; 96(32): e7764 |
| Brain | miRNA-124a, Let7 | Mazzacurati L. Molecular Therapy 23, |
| | family | 2015; Gaur et al. Cancer Res. 2007; 67(5): |
| | | 2456-68. |
| Lung | Let-7 family | Edge RE et al. Mol Ther |
| | miRNA- 148a/b | 2008; 16(8):1437-43 Lamichhane et al. Disease |
| | miRNA-30 family | Markers. 2018; ID8309015 |
| | miRNA-126 | |
| Breast | Let7 family | Yu F. Cell 2007; 11431(6): 1109-23 |
| | | Takamizawa J. Cancer Res 2004; |
| | | 64(11): 37533756 |
| Pancreas | miRNA-375 | Song Set al. Biomed Rep. 2013 (3): 393- |
| | Let7 family | 398; Dai et al. Cancer Cell Int. 2020. 20:98. |
| | miRNA-142 | doi.org/10.1186/s12935-020-01185-z |
| | miRNA-145 | |
| | miRNA-217 | |
| | miRNA-122 | |
| | Let7 family | |
| Colon | miRNA-143, −145, | Michael MZ. Mol Cancer Res 2003; 334 |
| | −194, −34a, −126, | (1): 882-891; Ding et al. Int. J. Mol. Sci. |
| | −192, −215, Let7 | 2018; 19, 2719 |
| | family | |
| Kidney | miRNA-192, −194, | Sempere et al. Genome Biol 2004; |
| | −204, −215, −30 | 5(3): R13 |
| | family, −141, −200c, | Wu et al. Nat.Commun. Apr 4; 7: 11169.; |
| | Let7 family | Nakada et al. J Pathol. 2008; 216: 418-427; |
| | | Jiang et al. Oncology Letters (2018) 16, |
| | | 3038-3044; Khella et al. Carcinogenesis. 2013. |
| | | 34(10): 2231-2239; Chen. et al. Cell Death and |
| | | Disease (2017) 8, e2859; |
| | | doi: 10.1038/cddis.2017.252 |
| Skin | miRNA-877, −4300, | Aksenenko et al. BMC Dermatology. |
| | −4720, −6761 | 2019; 19: 1. |
| | miRNA-203a, −205, | Liu et al. Laboratory Investigation (2012) |
| | −200c, Let7 family | 92, 1084-1096 |
| Spleen | miRNA-142, | Chen et al. Science (2004); 303(5654): |
| | miRNA-126, | 83-86; Trissal et al. Cancer Res. 2018; |
| | Let7 family | 78(13): 3510-3521; Merkerova et al. Eur J |
| | | Haematol 2008; 81(4): 304-10 |
| Muscle and | miRNA-1, miRNA- | Bostjancic E, et al. Cardiology. |
| cardiac | 133a, miRNA-206, | 2010; 115(3): 163-169; Rao, Prakash K et al. |
| muscle | Let7 family | FASEB J. 2010; 24(9): 3427-3437; |
| | | Ma et al. Int. J. Biol. Sci. 2015; 11: 345-352. |
| Endothelium | miRNA-98, | Harris TA et al. PNAS. |
| | miRNA-126 | 2008.105(5): 1516-1521 Matarese A. et al. |
| | | Biomedicines. 2020, 8, 462; |
| | | doi: 10.3390/biomedicines8110462 |

Treating patients with immunotherapies may have safety issues due to the possibility of off-target effects. Even the expression of certain polypeptides by the provision of coding mRNA sequences can have negative effects on certain organs. Protecting healthy tissues, for example liver, brain, breast, lung, pancreas, colon/GI-tract, skin, muscle, and kidneys is thus paramount for successful clinical applications. miRNAs such as those described above can be used to reduce the expression of an administered mRNA in particular cell, tissue and/or organ types, to protect those cells, tissues and/or organs from any off-target effects.

For instance, target sequences for specific miRNA that are highly expressed in specific tissues can be used to protect healthy cells, such as miRNA-1, miRNA-133a and/or miRNA-206 to protect healthy muscle and/or myocardium tissues. As a result, it may be desired to use miRNA target sequences which are not necessarily associated with differential expression in diseased and healthy cells. For example, miRNA-142 and miRNA 145 have expression in pancreatic tissue, while miRNA-9 can be used for brain and lung protection because of its high expression in these tissues.

If more than one tissue is to be protected, a combination of multiple miRNA target sequence is used. For instance, the target sequence for miRNA-122, miRNA-203a, miRNA-1 and miRNA-30a is used together to protect cells of the liver, skin, muscle and kidney tissues.

Hence, the present compositions may represent an enabling technology platform for enhancing and facilitating the successful adoption of hitherto ‘experimental’ cellular or viral therapies.

As is evident from this disclosure, the present invention is envisioned to relate to a number of possible combinations of therapies, delivery platforms (such as different nanoparticle compositions), therapeutic agents (such as drugs, vaccines and/or viruses), encoded polypeptides and target cells, tissues or organs. Each and all of these possibilities have implications for the optimal expression for the encoded polypeptides supplied by the mRNA sequences.

It has been found that the optimisation of one or more characteristics of the miRNA target sequences can lead to particular efficacy at promoting differential expression and thereby healthy organ protection. By the same token, such characteristics can be controlled to increase or decrease the resultant differential expression in particular organ, tissue or cell types, according to the specific context. There may be situations where a variety of expression levels are desired in various different cell types, and it is intended that target sequences can be modified to allow for such an outcome, by varying one or more characteristics as described herein. Also, an miRNA target site sequence can be modified so it is subject to regulation by more than one miRNA, either within the same tissue or in different tissues.

Sequence matching: the degree to which the target sequences are an exact match with the complementary miRNA sequence (that is, the number of mismatches between the miRNA sequence and the binding site sequence) has been shown to impact the efficacy of resultant expression silencing. For example, an exact or perfect match has been shown to lead to more rapid degradation of the sequence possessing the miRNA binding site sequence (Brown and Naldini, Nat Rev Genet. 2009; 10(8): 578-585. Therefore, if complete, or close to complete silencing of a particular polypeptide product is required in a particular cell type, it may be desired to select an miRNA target sequence which is an exact match, or has at most no more than one base pair mismatch, with an miRNA sequence associated with that cell type.

Likewise, if reduced but not absent expression is desired in a particular cell type, an miRNA binding site sequence with an increased number of mismatches can be chosen to allow for this. Examples of several miRNA sequences mentioned herein, including the sequences of the stem-loop pre-miRNA with the eventual processed mature 5P or 3P miRNA and the sequences which form a duplex with the mature miRNA in the pre-miRNA underlined, as well as the mature miRNA sequences and duplex forming sequences themselves, are shown in Table 3 below. The mature miRNA expressed at significant levels in the cell (which can be either or both of the 5P and 3P strands) is marked (*). Table 4 shows the original, imperfectly matched, target sequence which forms the duplex in the pre-miRNA, followed by the mature miRNA sequence and the development of a modified complementary target sequence, which is designed to be a perfect match with the overexpressed mature miRNA sequence. The modified target sequence in the conventional 5′ to 3′ orientation is shown in bold.

TABLE 3
Optimisation of the miRNA target sequences
by testing 5P vs 3P mature binding sequences
(*overexpressed mature miRNA from
RNA-Seq database mirbase.org)

| | | Nucleotide Sequence |
|---|---|---|
| miRNA- | Pre-miRNA | 5-CCUUAGCAGAGCUGUGGAGUGUGACAAUGGUGUUUGUGU |
| 122 | [SEQ ID | CUAAACUAUCAAACGCCAUUAUCACACUAAAUAGCUACUGC |
| | NO: 2] | UAGGC-3 |
| | 5P mature* | 5-UGGAGUGUGACAAUGGUGUUUG-3 |
| | [SEQ ID | |
| | NO: 3] | |
| | 3P mature | 5-AACGCCAUUAUCACACUAAAUA-3 |
| | [SEQ ID | |
| | NO: 4] | |
| miRNA- | Pre-miRNA | 5-GCCAACCCAGUGUUCAGACUACCUGUUCAGGAGGCUCUCA |
| 199a | [SEQ ID | AUGUGUACAGUAGUCUGCACAUUGGUUAGGC-3 |
| | NO: 5] | |
| | 5P mature | 5-CCCAGUGUUCAGACUACCUGUUC-3 |
| | [SEQ ID | |
| | NO: 6] | |
| | 3P mature* | 5-ACAGUAGUCUGCACAUUGGUUA-3 |
| | [SEQ ID | |
| | NO: 7] | |
| miRNA- | Pre-miRNA | 5-UGCCAGUCUCUAGGUCCCUGAGACCCUUUAACCUGUGAGG |
| 125a | [SEQ ID | ACAUCCAGGGUCACAGGUGAGGUUCUUGGGAGCCUGGCGUC |
| | NO: 8] | UGGCC-3 |
| | 5P mature* | 5-UCCCUGAGACCCUUUAACCUGUGA-3 |
| | [SEQ ID | |
| | NO: 9] | |
| | 3P mature | 5-ACAGGUGAGGUUCUUGGGAGCC-3 |
| | [SEQ ID | |
| | NO: 10] | |
| | Pre-miRNA | 5-GCCGAGACCGAGUGCACAGGGCUCUGACCUAUGAAUUGACA |
| | [SEQ ID NO: | GCCAGUGCUCUCGUCUCCCCUCUGGCUGCCAAUUCCAUAGGU |
| | 11] | CACAGGUAUGUUCGCCUCAAUGCCAGC-3 |
| | 5P mature* | 5-UCUGACCUAUGAAUUGACAGCC-3 |
| | [SEQ ID NO: | |
| | 12] | |
| | 3P mature | 5-CUGCCAAUUCCAUAGGUCACAG-3 |
| | [SEQ ID NO: | |
| | 13] | |
| | Pre-miRNA | 5- |
| | [SEQ ID NO: | CGGGGUGAGGUAGUAGGUUGUGUGGUUUCAGGGCAGUGAUG |
| | 14] | UUGCCCCUCGGAAGAUAACUAUACAACCUACUGCCUUCCCUG- |
| | | 3 |
| | 5P mature* | 5-UGAGGUAGUAGGUUGUGUGGUU-3 |
| | [SEQ ID NO: | |
| | 15] | |
| | 3P mature | 5-CUAUACAACCUACUGCCUUCCC-3 |
| | [SEQ ID NO: | |
| | 16] | |
| | Pre-miRNA | 5- |
| | [SEQ ID NO: | CCCCGCGACGAGCCCCUCGCACAAACCGGACCUGAGCGUUUU |
| | 17] | GUUCGUUCGGCUCGCGUGAGGC-3 |
| | 5P mature | 5-GCGACGAGCCCCUCGCACAAACC-3 |
| | [SEQ ID NO: | |
| | 18] | |
| | 3P mature* | 5-UUUGUUCGUUCGGCUCGCGUGA-3 |
| | [SEQ ID NO: | |
| | 19] | |
| | Pre-miRNA | 5- |
| | [SEQ ID NO: | AGGCCUCUCUCUCCGUGUUCACAGCGGACCUUGAUUUAAAUG |
| | 20] | UCCAUACAAUUAAGGCACGCGGUGAAUGCCAAGAAUGGGGCU |
| | | G-3 |
| | 5P mature | 5-CGUGUUCACAGCGGACCUUGAU-3 |
| | [SEQ ID NO: | |
| | 21] | |
| | 3P mature* | 5-UAAGGCACGCGGUGAAUGCCAA-3 |
| | [SEQ ID NO: | |
| | 22] | |
| | Pre-miRNA | 5- |
| | [SEQ ID NO: | GCGCAGCGCCCUGUCUCCCAGCCUGAGGUGCAGUGCUGCAU |
| | 23] | CUCUGGUCAGUUGGGAGUCUGAGAUGAAGCACUGUAGCUCAG |
| | | GAAGAGAGAAGUUGUUCUGCAGC-3 |
| | 5P mature | 5-GGUGCAGUGCUGCAUCUCUGGU-3 |
| | [SEQ ID NO: | |
| | 24] | |
| | 3P mature* | 5-UGAGAUGAAGCACUGUAGCUC-3 |
| | [SEQ ID NO: | |
| | 25] | |
| | Pre-miRNA | 5- |
| | [SEQ ID NO: | GACAGUGCAGUCACCCAUAAAGUAGAAAGCACUACUAACAGCA |
| | 26] | CUGGAGGGUGUAGUGUUUCCUACUUUAUGGAUGAGUGUACUG |
| | | UG-3 |
| | 5P mature* | 5-CAUAAAGUAGAAAGCACUACU-3 |
| | [SEQ ID NO: | |
| | 27] | |
| | 3P mature* | 5-UGUAGUGUUUCCUACUUUAUGGA-3 |
| | [SEQ ID NO: | |
| | 28] | |
| | Pre-miRNA | 5- |
| | [SEQ ID NO: | GUGUUGGGGACUCGCGCGCUGGGUCCAGUGGUUCUUAACAG |
| | 29] | UUCAACAGUUCUGUAGCGCAAUUGUGAAAUGUUUAGGACCAC |
| | | UAGACCCGGCGGGCGCGGCGACAGCGA-3 |
| | 5P mature | 5-AGUGGUUCUUAACAGUUCAACAGUU-3 |
| | [SEQ ID | |
| | NO: 30] | |
| | 3P mature* | 5-GUGAAAUGUUUAGGACCACUAG-3 |
| | [SEQ ID NO: | |
| | 31] | |
| | Pre-miRNA | 5- |
| | [SEQ ID NO: | UGGGAUGAGGUAGUAGGUUGUAUAGUUUUAGGGUCACACCCA |
| | 32] | CCACUGGGAGAUAACUAUACAAUCUACUGUCUUUCCUA-3 |
| | 5P mature* | 5-UGAGGUAGUAGGUUGUAUAGUU-3 |
| | [SEQ ID | |
| | NO: 33] | |
| | 3P mature | 5-CUAUACAAUCUACUGUCUUUC-3 |
| | [SEQ ID NO: | |
| | 34] | |
| | Pre-miRNA | 5- |
| | [SEQ ID NO: | GCGACUGUAAACAUCCUCGACUGGAAGCUGUGAAGCCACAGA |
| | 35] | UGGGCUUUCAGUCGGAUGUUUGCAGCUGC-3 |
| | 5P mature* | 5-UGUAAACAUCCUCGACUGGAAG-3 |
| | [SEQ ID NO: | |
| | 36] | |
| | 3P mature | 5-CUUUCAGUCGGAUGUUUGCAGC-3 |
| | [SEQ ID NO: | |
| | 37] | |
| | Pre-miRNA | 5- |
| | [SEQ ID NO: | UGGGAAACAUACUUCUUUAUAUGCCCAUAUGGACCUGCUAAG |
| | 38] | CUAUGGAAUGUAAAGAAGUAUGUAUCUCA-3 |
| | 5P mature | 5-ACAUACUUCUUUAUAUGCCCAU-3 |
| | [SEQ ID NO: | |
| | 39] | |
| | 3P mature* | 5-UGGAAUGUAAAGAAGUAUGUAU-3 |
| | [SEQ ID NO: | |
| | 40] | |
| | Pre-miRNA | 5- |
| | [SEQ ID NO: | CGCUGGCGACGGGACAUUAUUACUUUUGGUACGCGCUGUGAC |
| | 41] | ACUUCAAACUCGUACCGUGAGUAAUAAUGCGCCGUCCACGGC |
| | | A-3 |
| | 5P mature | 5-CAUUAUUACUUUUGGUACGCG |
| | [SEQ ID NO: | |
| | 42] | |
| | 3P mature* | 5-UCGUACCGUGAGUAAUAAUGCG |
| | [SEQ ID NO: | |
| | 43] | |

TABLE 4
Optimization of the miRNA target sequences by modifying the nucleotides
sequence to obtain a perfect match with the miRNA
(*overexpressed mature miRNA from RNA-Seq database mirbase.org)

| | | Nucleotide Sequence |
|---|---|---|
| miRNA- | 3P = sequence within pre- | 5-AACGCCAUUAUCACACUAAAUA-3 |
| 122 | miRNA [SEQ ID NO: 4] | |
| | Perfect matching [SEQ ID | 5P* 5-UGGAGUGUGACAAUGGUGUUUG-3 |
| | NO: 3] | |
| | Target Sequence [SEQ ID | TS 3-ACCUCACACUGUUACCACAAAC-5 |
| | NO: 44] | |
| | Orientated 5′ -> 3′ | TS 5-CAAACACCAUUGUCACACUCCA-3 |
| | [SEQ ID NO: 44] | |
| miRNA- | 5P = Target sequence | 5-CCCAGUGUUCAGACUACCUGUUC-3 |
| 199a | within pre-miRNA [SEQ ID | |
| | NO: 6] | |
| | Perfect matching [SEQ ID | 3P* 5-ACAGUAGUCUGCACAUUGGUUA-3 |
| | NO: 7] | TS 3-UGUCAUCAGACGUGUAACCAAU-5 |
| | TS [SEQ ID NO: 45] | |
| | Orientated 5′ -> 3′ | TS 5-UAACCAAUGUGCAGACUACUGU-3 |
| | [SEQ ID NO: 45] | |
| miRNA- | 3P = sequence within pre- | 5-ACAGGUGAGGUUCUUGGGAGCC-3 |
| 125a | miRNA [SEQ ID NO: 10] | |
| | Perfect matching | 5P* 5-UCCCUGAGACCCUUUAACCUGUGA-3 |
| | [SEQ ID NO: 9] | |
| | TS [SEQ ID NO: 46] | TS 3-AGGGACUCUGGGAAAUUGGACACU-5 |
| | Orientated 5′ -> 3′ | TS 5-UCACAGGUUAAAGGGUCUCAGGGA-3 |
| | [SEQ ID NO: 46] | |
| miRNA- | 3P = sequence within pre- | 5-CUGCCAAUUCCAUAGGUCACAG-3 |
| 192 | miRNA [SEQ ID NO: 13] | |
| | Perfect matching [SEQ ID NO: | 5P* 5-UCUGACCUAUGAAUUGACAGCC-3 |
| | 12] | |
| | TS [SEQ ID NO: 47] | TS 3-AGACUGGAUACUUAACUGUCGG-5 |
| | | TS 5-GGCUGUCAAUUCAUAGGUCAGA-3 |
| | 3P = sequence within pre- | 5-CUAUACAACCUACUGCCUUCCC-3 |
| | miRNA [SEQ ID NO: 6] | |
| | Perfect matching [SEQ ID NO: | 5P* 5-UGAGGUAGUAGGUUGUGUGGUU-3 |
| | 15] | TS 3-ACUCCAUCAUCCAACACACCAA-5 |
| | 5P = sequence within pre- | 5-GCGACGAGCCCCUCGCACAAACC-3 |
| | miRNA [SEQ ID NO: 18] | |
| | Perfect matching [SEQ ID | 3P* 5-UUUGUUCGUUCGGCUCGCGUGA-3 |
| | | TS 3-AAACAAGCAAGCCGAGCGCACU-5 |
| | 5P = sequence within pre- | 5-CGUGUUCACAGCGGACCUUGAU-3 |
| | miRNA [SEQ ID NO: 21] | |
| | Perfect matching [SEQ ID NO: | 3P* 5-UAAGGCACGCGGUGAAUGCCAA-3 |
| | 22] | TS 3-AUUCCGUGCGCCACUUACGGUU-5 |
| | 5P = sequence within pre- | 5-GGUGCAGUGCUGCAUCUCUGGU-3 |
| | miRNA [SEQ ID NO: 24] | |
| | Perfect matching [SEQ ID NO: | 3P* 5-UGAGAUGAAGCACUGUAGCUC-3 |
| | 25] | |
| | TS SEQ ID NO: 51] | TS 3-ACUCUACUUCGUGACAUCGAG-5 |
| | | TS 5-GAGCUACAGUGCUUCAUCUCA-3 |
| | 5P = sequence within pre- | 5-CAUAAAGUAGAAAGCACUACU-3 |
| | miRNA [SEQ ID NO: 27] | |
| | Perfect matching | 3P* 5-UGUAGUGUUUCCUACUUUAUGGA-3 |
| | [SEQ ID NO: 28] | TS 3-ACAUCACAAAGGAUGAAAUACCU-5 |
| | TS [SEQ ID NO: 52] | TS 5-UCCAUAAAGUAGGAAACACUACA-3 |
| | 5P = sequence within pre- | 5-AGUGGUUCUUAACAGUUCAACAGUU-3 |
| | miRNA [SEQ ID NO: 30] | |
| | Perfect matching [SEQ ID NO: | 3P*5-GUGAAAUGUUUAGGACCACUAG-3 |
| | 31] | TS 3-CACUUUACAAAUCCUGGUGAUC-5 |
| | 3P = sequence within miRNA | 5-CUAUACAAUCUACUGUCUUUC-3 |
| | [SEQ ID NO: 34] | |
| | Perfect matching [SEQ ID NO: | 5P* 5-UGAGGUAGUAGGUUGUAUAGUU-3 |
| | 33] | TS 3-ACUCCAUCAUCCAACAUAUCAA-5 |
| | 3P = sequence within pre- | 5-CUUUCAGUCGGAUGUUUGCAGC-3 |
| | miRNA SEQ ID NO: 37] | |
| | Perfect matching [SEQ ID NO: | 5P* 5-UGUAAACAUCCUCGACUGGAAG-3 |
| | 36] | TS 3-ACAUUUGUAGGAGCUGACCUUC-5 |
| | Orientated 5′ -> 3′ | TS 5-CUUCCAGUCGAGGAUGUUUACA-3 |
| | [SEQ ID NO: 55] | |
| | 5P = sequence within pre- | 5-ACAUACUUCUUUAUAUGCCCAU-3 |
| | miRNA [SEQ ID NO: 39] | |
| | Perfect matching [SEQ ID NO: | 3P* 5-UGGAAUGUAAAGAAGUAUGUAU-3 |
| | 40] | TS 3-ACCUUACAUUUCUUCAUACAUA-5 |
| | 5P = sequence within pre- | 5-CAUUAUUACUUUUGGUACGCG-3 |
| | miRNA [SEQ ID NO: 39] | |
| | Perfect matching [SEQ ID NO: | 3P* 5-UCGUACCGUGAGUAAUAAUGCG-3 |
| | 40] | TS 3-AGCAUGGCACUCAUUAUUACGC-5 |

In various embodiments, the mRNA coding for an antigen product additionally comprises at least one OPS that protects multiple organs (i.e., a multi-organ protection sequence or “MOP”), wherein the OPS sequence comprises at least three (for example, at least a first, a second and a third) micro-RNA (miRNA) target sequences. One of the target sequences can be a sequence capable of binding with miRNA-1. The target sequences can comprise sequences capable of binding with one or more of miRNA-1, miRNA-133a, miRNA-206, miRNA-122, miRNA-192, miRNA-203a, miRNA-205, miRNA-200c, miRNA-30a/b/c, and/or Let7a/b, suitably with all of these. In various embodiments of any antigen-encoding mRNA, the OPS can comprise sequences capable of binding with miRNA-122, miRNA-1, miRNA-203a, and miRNA-30a; sequences capable of binding with Let7b, miRNA-126, and miRNA-30a; sequences capable of binding with miRNA-122, miRNA-192, and miRNA-30a; or sequences capable of binding with miRNA-192, miRNA-30a, and miRNA-124, with two sequences capable of binding with miRNA 122. Any OPS such as those described here may further include a sequence capable of binding with miRNA-124, for the protection of brain tissue, and/or a sequence capable of binding with Let7b. The order of the target sequences within an OPS (that is, their 5′ to 3′ arrangement) is not considered to be important, and any permutation may be considered.

It is known that variants and polymorphisms of miRNA sequences can be found, and that miRNA families exist with similar properties. In the present invention, it is envisioned that all suitable variants and family members of particular miRNA sequences and associated binding sites can be used where appropriate. On the other hand, apparently closely related miRNA sequences can have different expression profiles (Sun et al, World J Gastroenterol. 2017 Nov. 28), so in some situations it will be necessary to determine whether a specific substitution is appropriate, by reference to the literature. For example, Let-7 is part of a wider family with a number of related variants, which can be denoted as Let-7a to Let-7k, and so on.

As discussed above, such variants and polymorphisms may vary in their efficacy at allowing for miRNA-mediated silencing, and it is intended that particular selections can therefore be made to allow for the desired level of silencing in a particular cell type.

The presence of a plurality of miRNA target sequences in the mRNA construct enables improved efficacy of the differential expression of the supplied polypeptide or polypeptides. Without being bound by theory, it is thought that with an increased number of target sites, the likelihood of translation inhibition by the miRNA is increased. Multiple miRNA target sites can comprise multiple copies of substantially the same target sequence, thereby introducing redundancy. Alternatively or additionally, the multiple target sequences can comprise substantially different sequences, thereby allowing the mRNA construct to be targeted by more than one species of miRNA. In this way, differential expression of a supplied mRNA construct can be achieved for more than one cell type, and/or in more than one organ, as is evident from the discussion of organs and their associated specific miRNA expression above. Both approaches are considered to be possible within the same sequence or multiple sequences. An intermediate approach is also envisioned, wherein target sites are included which are intended to be targets for the same miRNA sequence, but have differences in order to bind different miRNA variants of the same family, e.g. Let7.

Some advantages associated with the use of multiple target sites include an increase in the efficiency of differential expression of polypeptides supplied by the mRNA sequences of the present invention, within a single organ. Use of different binding site sequences, or sequences which are applicable to more than one tissue or organ type can enable differential expression to be achieved in different cell types in more than one organ or tissue. This may be desirable when systemic administration of compositions according to the invention is used, and it is necessary to avoid off-target effects in more than one organ.

In some embodiments of any of the aspects, each OPS of the composition(s) independently comprises at least three, at least four, or at least five miRNA target sequences. In some embodiments of any of the aspects, each OPS of the composition(s) independently comprises at least three miRNA target sequences which are all different from each other. In some embodiments of any of the aspects, the first and second ORFs are operatively linked to the same OPS or to identical OPS. In some embodiments of any of the aspects, the first and second ORFs are operatively linked to different OPSs. In some embodiments of any of the aspects, the OPS linked to the first ORF and the OPS linked to the second ORF comprise the same miRNA target sequences.

In some embodiments of any of the aspects, the OPS linked to the first ORF and the OPS linked to the second ORF each comprise at least one miRNA target sequence not comprised by the other OPS. In some embodiments of any of the aspects, the OPS linked to the first ORF and the OPS linked to the second ORF each comprise at least three miRNA target sequences not comprised by the other OPS.

Even with localised or targeted administration, it is possible that supplied mRNA constructs may encounter or accumulate in organs, tissues, and/or cells for which they were not intended. In particular, liver and spleen tissue may accumulate administered compositions, due to the physiological function of these organs. In these cases, to avoid off-target effects, it may be advantageous for the supplied constructs to comprise miRNA target sequences which would permit reduced expression in these tissues. Conversely, it may be desirable for expression to be encouraged in some organs, tissues and/or cell types but not others, which can be achieved by the selection of miRNA target sequences accordingly.

Particular combinations of miRNA target sites can relate to particular combinations of target organs, which may be especially effective in different contexts. For example, administered compositions may accumulate in the liver and spleen, and therefore the use of miRNA target sequences associated with those organs can give directed protection to healthy cells which may be contacted with the compositions.

For example, the binding site sequences can provide one or more targets for each of miRNA-122 and miRNA-142, or any other combination of liver and spleen-associated miRNA sequences, for example any combination of those listed for these organs in Table 2. Such combinations could include, for example, at least one copy of at least one target site selected from miRNA-122, miRNA-125, and miRNA-199 (liver); at least one copy of at least one binding site sequence selected from miRNA-192, miRNA-194, miRNA-204, miRNA-215, and miRNA-30 a,b,c (kidney); and at least one copy of a binding site for miRNA-142 (spleen).

Such an approach may be especially advantageous for certain varieties of delivery nanoparticles. For instance, liposome-based nanoparticles may be prone to accumulate in the liver, kidneys and spleen. Other nanoparticle types or alternative administration approaches may accumulate in different organs or tissues, or the targeting of the compositions may cause particular organs or tissues to be in particular need of modulation of expression. For example, intramuscular administration may lead to accumulation in muscle tissue, and subcutaneous administration may lead to accumulation in skin tissue, with effects on which cell types would benefit from protection. It is therefore possible to select generic, likely longer, sequences comprising miRNA binding site sequences which give broad protection from unwanted expression in multiple organs, or to select particular miRNA binding site sequences to allow specific protection in one or more organs as required in a particular situation, which may allow for shorter sequences, and/or the inclusion of repeated binding site sequences (see below). In such a way, the delivered mRNA sequence can be optimised with respect to the mode of delivery (or vice versa).

In some cases, the miRNA target sequences used in the organ protection sequence may not be associated with the tissues or organs to be treated, and may not be designed to lead to differential expression between healthy and diseased cells within said tissues and organs. The miRNA binding sequences may rather be chosen to prevent off-target effects in organs which are not intended to be treated. In such cases, the miRNA target sequences may be chosen to accommodate for undesirable biodistribution and to prevent expression of the encoded mRNA within off-target organs. For instance, the use of miRNA target sequences associated with the liver, kidneys and spleen may be chosen, and so prevent expression within healthy cells comprised within these organs. Examples of potential combinations of miRNA target sequences which could allow for this are set out above.

It is also envisioned that since a perfect match between a binding site sequence and an miRNA sequence is not required for miRNA-mediated silencing to occur, and since some miRNA sequences (especially sequences which are present within similar cell types) have considerable similarity, it is possible that sequences could be devised that could provide a target for more than one miRNA sequence. For example, miRNA-122 and miRNA-199 have similar binding site sequences, and a sequence which is substantially complementary to both miRNA could be designed and included as a miRNA target sequence, for example by slightly modifying a miRNA-122 binding site sequence. In this way, both miRNA-122 and miRNA-199 could bind to such a sequence, increasing degradation of the mRNA. Similarly, a target sequence for the Let-7 miRNA could serve as a target sequence for other members of the Let-7 family. Binding site sequences for different miRNAs can be aligned with any suitable alignment technique and compared for shared nucleotides, whereupon a binding site sequence comprising those shared nucleotides can be designed.

In specific embodiments of the invention, the number of times a particular target site sequence is repeated within an mRNA may impact the efficacy of silencing mediated by the binding site sequences. For instance, an increased number of repeats of one miRNA target site can increase the likelihood of the relevant miRNA binding to it, and so the likelihood of translation inhibition or degradation before translation occurs. As a result, if more complete miRNA-mediated silencing is required in a particular cell type, more repeats of a suitable target sequence for an miRNA expressed in those cells can be used. Likewise, reduced but not absent expression can be achieved by including fewer binding site sequences, with or without any of the other approaches discussed herein. Therefore, the same binding site sequence can be provided in the mRNA once, twice, three times, four times, five times, or more, and can be provided alone or in combination with target site sequences for other miRNAs.

According to certain embodiments, the order of the miRNA target sites comprised within the mRNA sequence may affect the resultant organ protection efficacy. For example, the target sequences for miRNA-122, let 7b, miRNA-375, miRNA-192, miRNA-142, (present in liver, lung, breast, pancreas, kidney, and spleen cells) can be presented in this order, or in a number of other permutations, for example:
miRNA-122-miRNA-375-Let 7-miRNA-192-miRNA-142;
miRNA-122-miRNA-375-Let 7-miRNA-142-miRNA-192; or
miRNA-122-Let 7-miRNA-375-miRNA-142-miRNA-192.
   As another example, the target sequences for miRNA-122, Let 7a, miRNA-142, miRNA-30a, miRNA-143, (present in liver, lung/colon, spleen/haematopoietic cells, kidney, and colon cells) can be presented in this order, or in a number of other permutations, for example:
miRNA-122-Let7a-miRNA-142-miRNA-30a-miRNA-143;
miRNA-122-miRNA-142-Let7a-miRNA-143-miRNA-30a; or
miRNA-122-miRNA-30a-Let7a-miRNA-143-miRNA-142.
   In specific embodiments of the invention described in more detail below the target sequences for miRNA-122, miRNA-192 and miRNA-30a (present in liver, colon and kidney) can be presented in a variety of combinations such as:
miRNA-122-miRNA-192-miRNA-30a;
miRNA-122-miRNA-30a-miRNA-192; or
miRNA-192-miRNA-122-miRNA-30a
   In further embodiments of the invention described in more detail below the target sequences for Let7b, miRNA-126 and miRNA-30a (present in liver, colon, spleen, lung and kidney) can be presented in a variety of combinations such as:
Let7b-miRNA-126-miRNA-30a;
Let7b-miRNA-30a-miRNA-126; or
miRNA-126-Let7b-miRNA-30a
   Such combinations can be useful in protecting tissues likely to be affected by administration of compositions designed to be used in vaccine or adjuvant expression systems, as discussed herein.

As a further example, the target sequences for miRNA-122, miRNA-203a, miRNA-1, miRNA-30a (present in liver, skin, muscle/myocardium, and kidney) can be presented in this order, or in a number of other permutations, for example:
miRNA-122-miRNA-203a-miRNA-1-miRNA-30a;
miRNA-122-miRNA-1-miRNA-203a-miRNA-30a; or
miRNA-122-miRNA-30a-miRNA-1-miRNA-203a
   Such a combination can be useful in protecting tissues likely to be affected by administration of compositions designed to induce an immune response, as discussed below in relation to vaccines, adjuvants and similar approaches.

The present invention therefore allows different approaches to be selected which are tuneable to the coding sequence being delivered by the mRNA, and in which cell types. In other words, the differential expression allowed by the present invention is ‘configurable’ in order to allow for whatever level of expression or reduced expression is required.

In some embodiments, the delivered mRNA may code for a proinflammatory cytokine. In such cases, it may be desired to have maximal expression of the encoded product in the target diseased cells, but also to have reduced but still present expression in surrounding healthy tissue of the target organ. On the other hand, it may be desirable for expression of such immune-stimulating products in certain tissues (such as brain or other neural tissue) to be avoided completely, and/or for expression to be reduced in cells, tissues and organs where the composition is likely to accumulate, to prevent off-target immune responses and possible systemic reaction. Therefore, in one example the miRNA target sequences can be determined by one or more of the approaches discussed above to allow full expression in target diseased cells, partially reduce expression in healthy cells in the target organ, while more completely reducing expression in neural tissue and sites of accumulation.

In some embodiments, more than one different mRNA sequence may be provided in a single composition. These different sequences can encode different polypeptides, and/or different miRNA target sites. In this way, a single composition can allow for multiple different polypeptides to be expressed. By using different combinations of miRNA target sequences in the separate mRNA sequences, different cell types or target organs can express, or be protected from the expression of certain polypeptides, according to the desired objective. For instance, if healthy cells in liver and brain must be protected from the expression of a polypeptide ‘A’, but it is desired to express a polypeptide B′ in healthy brain, but not liver, a first mRNA sequence could comprise the sequence of ‘A’, with target sites for miRNA-122, miRNA-125a and miRNA-124a, while a second mRNA sequence could comprise the sequence of B′, with binding sites for miRNA-122 and miRNA-125a.

It can be appreciated that the person of skill in the art will be able to devise combinations of miRNA target sites, polypeptide sequences and multiple mRNA sequences in order to achieve any combination of expression in a given set of organ and cell types. The relevant organs and tissue types relating to these sequences are discussed above and in Table 2. An ORF can be preceded by a start codon and terminated with a stop codon, and a subsequent series of up to five or more binding site sequences are present in the 3′UTR. The miRNA target sites that define the OPS may be separated by spacers, or no spacer at all if preferred. The ORF can code for example for a polypeptide as described herein. Variability in the stop codon is envisioned in any embodiment, and there may in all embodiments be no stop codon between the ORF and the binding site sequences.

The UTR of the mRNA sequences supplied by the present invention can be selected to have similarity, for example greater than 90% similarity, to part or all of a UTR sequence expressed in one of the cell types within the target organ. Particular cell types can have genes which are up- or down-regulated in expression, and the UTR sequence can mediate this regulation, for instance through encouraging the stability or degradation of the relevant mRNA sequences. As an example, UTRs associated with genes which are known to be upregulated in diseased cells may have one or more features, such as miRNA binding site sequences, which encourage their stability and translation in these diseased cells. By incorporating similar sequences into supplied mRNA sequences, stability and translation can be improved in diseased cells but not healthy cells.

In certain situations, it is possible that more than one candidate for an miRNA sequence which exhibits differential expression in different cell types in a target tissue may exist. In such cases, it may be advantageous that a plurality of miRNA target sequences are included in the mRNA construct, and that these sequences may be substantially different sequences. However, it is also envisaged that each of the plurality of miRNA target sequences may be substantially the same sequence.

In some embodiments of any of the aspects, the OPS operatively linked to the second ORF comprises miRNA sequences selected to protect one or more organs or tissues selected from the group consisting of muscle, liver, brain, breast, endothelium, pancreas, colon, kidney, lungs, spleen and skin, heart, gastrointestinal organs, reproductive organs, and esophagus. In some embodiments of any of the aspects, the OPS operatively linked to the first ORF comprises miRNA sequences selected to protect one or more organs or tissues selected from the group consisting muscle, liver, brain, breast, endothelium, pancreas, colon, kidney, lungs, spleen and skin. In some embodiments of any of the aspects, the OPS operatively linked to the first ORF comprises miRNA sequences selected to protect one or more organs selected from the group consisting of muscle, liver, kidney, lungs, spleen, skin, heart, gastrointestinal organs, reproductive organs, and esophagus.

In some embodiments of any of the aspects, one or more of the OPS independently comprises:
a) at least two miRNA target sequences selected from one or more sequences that bind to: miRNA-122; miRNA-125; miRNA-199; miRNA-124a; miRNA-126; miRNA-98; Let7 miRNA family; miRNA-375; miRNA-141; miRNA-142; miRNA-148a/b; miRNA-143; miRNA-145; miRNA-194; miRNA-200c; miRNA-203a; miRNA-205; miRNA-1; miRNA-133a; miRNA-206; miRNA-34a; miRNA-192; miRNA-194; miRNA-204; miRNA-215; miRNA-30 family; miRNA-877; miRNA-4300; miRNA-4720; and/or miRNA-6761;
b) sequences selected from one or more of SEQ ID NOs: 44-57;
c) at least two miRNA target sequences selected from sequences capable of binding with miRNA-1, miRNA133a, miRNA206, miRNA-122, miRNA203a, miRNA205, miRNA200c, miRNA30a, and/or let7a/b;
d) at least two miRNA target sequences selected from one or more sequences that bind to: miRNA-1, miRNA-122, miRNA-30a, miRNA-203a, let7b, miRNA-126, and/or miRNA-192,
e) at least two miRNA target sequences selected from sequences capable of binding with miRNA-1, miRNA-122, miR-30a and/or miR-203a;
f) miRNA target sequences capable of binding with miRNA-1, miRNA-122, miRNA-30a and miRNA-203a;
g) miRNA target sequences capable of binding with let7b, miRNA-126, and miRNA-30a;
h) miRNA target sequences capable of binding with miRNA-122, miRNA-192, and miRNA-30a; or
i) miRNA target sequences capable of binding with miRNA-192, miRNA-30a, and miRNA-124, and two miRNA target sequences capable of binding with miRNA 122.

In some embodiments of any of the aspects, the OPS operatively linked to the second ORF comprises miRNA target sequences capable of binding with miRNA-1, miRNA-122, miR-30a and/or miR-203a; and the OPS operatively linked to the first ORF comprises miRNA target sequences capable of binding with miRNA-122, miRNA-126, miRNA-192, and/or miRNA 30a.

In some embodiments of any of the aspects, the MOP or OPS consists of, or consists essentially of the MOP or OPS of a sequence of Table 8. In some embodiments of any of the aspects, a mRNA construct encoding a proinflammatory sequence comprises, consists of, or consists essentially of a sequence of Table 8.

TABLE 8
ORF and 3′ UTR for IL-12 MOP and GM-CSF MOP

Human single chain IL-12 MOP (hscIL12-MOP)
with 3′ UTR perfect matching complementary
sequence to miRNA-122-5P, miRNA-1-3P,
miRNA-203a-3P, miRNA-30a-5P are underlined (MOPV)
[SEQ ID NO: 58]
AUGUGUCACCAGCAGUUGGUCAUCUCUUGGUUUUCCCUGGUUUUU
CUGGCAUCUCCCCUCGUGGCCAUAUGGGAACUGAAGAAAGAUGUU
UAUGUCGUAGAAUUGGAUUGGUAUCCGGAUGCCCCUGGAGAAAUG
GUGGUCCUCACCUGUGACACCCCUGAAGAAGAUGGUAUCACCUGG
ACCUUGGACCAGAGCAGUGAGGUCUUAGGCUCUGGCAAAACCCUG
ACCAUCCAAGUCAAAGAGUUUGGAGAUGCUGGCCAGUACACCUGU
CACAAAGGAGGCGAGGUUCUAAGCCAUUCGCUCCUGCUGCUUCAC
AAAAAGGAAGAUGGAAUUUGGUCCACUGAUAUUUUAAAGGACCAG
AAAGAACCCAAAAAUAAGACCUUUCUAAGAUGCGAGGCCAAGAAU
UAUUCUGGACGUUUCACCUGCUGGUGGCUGACGACAAUCAGUACU
GAUUUGACAUUCAGUGUCAAAAGCAGCAGAGGCUCUUCUGACCCC
CAAGGGGUGACGUGCGGAGCUGCUACACUCUCUGCAGAGAGAGUC
AGAGGGGACAACAAGGAGUAUGAGUACUCAGUGGAGUGCCAGGAG
GACAGUGCCUGCCCAGCUGCUGAGGAGAGUCUGCCCAUUGAGGUC
AUGGUGGAUGCCGUUCACAAGCUCAAGUAUGAAAACUACACCAGC
AGCUUCUUCAUCAGGGACAUCAUCAAACCUGACCCACCCAAGAAC
UUGCAGCUGAAGCCAUUAAAGAAUUCUCGGCAGGUGGAGGUCAGC
UGGGAGUACCCUGACACCUGGAGUACUCCACAUUCCUACUUCUCC
CUGACAUUCUGCGUUCAGGUCCAGGGCAAGAGCAAGAGAGAAAAG
AAAGAUAGAGUCUUCACGGACAAGACCUCAGCCACGGUCAUCUGC
CGCAAAAAUGCCAGCAUUAGCGUGCGGGCCCAGGACCGCUACUAU
AGCUCAUCUUGGAGCGAAUGGGCAUCUGUGCCCUGCAGUGGUGGC
GGUGGCGGCGGAUCUAGAAACCUCCCCGUGGCCACUCCAGACCCA
GGAAUGUUCCCAUGCCUUCACCACUCCCAAAACCUGCUGAGGGCC
GUCAGCAACAUGCUCCAGAAGGCCAGACAAACUCUAGAAUUUUAC
CCUUGCACUUCUGAAGAGAUUGAUCAUGAAGAUAUCACAAAAGAU
AAAACCAGCACAGUGGAGGCCUGUUUACCAUUGGAAUUAACCAAG
AAUGAGAGUUGCCUAAAUUCCAGAGAGACCUCUUUCAUAACUAAU
GGGAGUUGCCUGGCCUCCAGAAAGACCUCUUUUAUGAUGGCCCUG
UGCCUUAGUAGUAUUUAUGAAGACUUGAAGAUGUACCAGGUGGAG
UUCAAGACCAUGAAUGCAAAGCUUCUGAUGGAUCCUAAGAGGCAG
AUCUUUCUAGAUCAAAACAUGCUGGCAGUUAUUGAUGAGCUGAUG
CAGGCCCUGAAUUUCAACAGUGAGACUGUGCCACAAAAAUCCUCC
CUUGAAGAACCGGAUUUUUAUAAAACUAAAAUCAAGCUCUGCAUA
CUUCUUCAUGCUUUCAGAAUUCGGGCAGUGACUAUUGAUAGAGUG
AUGAGCUAUCUGAAUGCUUCCUAACAAACACCAUUGUCACACUCC
AUUUAAAAUACAUACUUCUUUACAUUCCAUUUAAACUAGUGGUCC
UAAACAUUUCACUUUAAACUUCCAGUCGAGGAUGUUUACA
Human single chain IL-12 without MOP
[SEQ ID NO: 59]
AUGUGUCACCAGCAGUUGGUCAUCUCUUGGUUUUCCCUGGUUUUU
CUGGCAUCUCCCCUCGUGGCCAUAUGGGAACUGAAGAAAGAUGUU
UAUGUCGUAGAAUUGGAUUGGUAUCCGGAUGCCCCUGGAGAAAUG
GUGGUCCUCACCUGUGACACCCCUGAAGAAGAUGGUAUCACCUGG
ACCUUGGACCAGAGCAGUGAGGUCUUAGGCUCUGGCAAAACCCUG
ACCAUCCAAGUCAAAGAGUUUGGAGAUGCUGGCCAGUACACCUGU
CACAAAGGAGGCGAGGUUCUAAGCCAUUCGCUCCUGCUGCUUCAC
AAAAAGGAAGAUGGAAUUUGGUCCACUGAUAUUUUAAAGGACCAG
AAAGAACCCAAAAAUAAGACCUUUCUAAGAUGCGAGGCCAAGAAU
UAUUCUGGACGUUUCACCUGCUGGUGGCUGACGACAAUCAGUACU
GAUUUGACAUUCAGUGUCAAAAGCAGCAGAGGCUCUUCUGACCCC
CAAGGGGUGACGUGCGGAGCUGCUACACUCUCUGCAGAGAGAGUC
AGAGGGGACAACAAGGAGUAUGAGUACUCAGUGGAGUGCCAGGAG
GACAGUGCCUGCCCAGCUGCUGAGGAGAGUCUGCCCAUUGAGGUC
AUGGUGGAUGCCGUUCACAAGCUCAAGUAUGAAAACUACACCAGC
AGCUUCUUCAUCAGGGACAUCAUCAAACCUGACCCACCCAAGAAC
UUGCAGCUGAAGCCAUUAAAGAAUUCUCGGCAGGUGGAGGUCAGC
UGGGAGUACCCUGACACCUGGAGUACUCCACAUUCCUACUUCUCC
CUGACAUUCUGCGUUCAGGUCCAGGGCAAGAGCAAGAGAGAAAAG
AAAGAUAGAGUCUUCACGGACAAGACCUCAGCCACGGUCAUCUGC
CGCAAAAAUGCCAGCAUUAGCGUGCGGGCCCAGGACCGCUACUAU
AGCUCAUCUUGGAGCGAAUGGGCAUCUGUGCCCUGCAGUGGUGGC
GGUGGCGGCGGAUCUAGAAACCUCCCCGUGGCCACUCCAGACCCA
GGAAUGUUCCCAUGCCUUCACCACUCCCAAAACCUGCUGAGGGCC
GUCAGCAACAUGCUCCAGAAGGCCAGACAAACUCUAGAAUUUUAC
CCUUGCACUUCUGAAGAGAUUGAUCAUGAAGAUAUCACAAAAGAU
AAAACCAGCACAGUGGAGGCCUGUUUACCAUUGGAAUUAACCAAG
AAUGAGAGUUGCCUAAAUUCCAGAGAGACCUCUUUCAUAACUAAU
GGGAGUUGCCUGGCCUCCAGAAAGACCUCUUUUAUGAUGGCCCUG
UGCCUUAGUAGUAUUUAUGAAGACUUGAAGAUGUACCAGGUGGAG
UUCAAGACCAUGAAUGCAAAGCUUCUGAUGGAUCCUAAGAGGCAG
AUCUUUCUAGAUCAAAACAUGCUGGCAGUUAUUGAUGAGCUGAUG
CAGGCCCUGAAUUUCAACAGUGAGACUGUGCCACAAAAAUCCUCC
CUUGAAGAACCGGAUUUUUAUAAAACUAAAAUCAAGCUCUGCAUA
CUUCUUCAUGCUUUCAGAAUUCGGGCAGUGACUAUUGAUAGAGUG
AUGAGCUAUCUGAAUGCUUCCUAA
Human GMCSF MOP mRNA (hGMCSF-MOP) perfect
matching complementary sequence to miRNA-122-5P,
miRNA-1-3P, miRNA-203a-3P, miRNA-30a-5P are
underlined (MOPV)
[SEQ ID NO: 60]
AUGUGGCUGCAGAGCCUGCUGCUCUUGGGCACUGUGGCCUGCAGC
AUCUCUGCACCCGCCCGCUCGCCCAGCCCCAGCACGCAGCCCUGG
GAGCAUGUGAAUGCCAUCCAGGAGGCCCGGCGUCUCCUGAACCUG
AGUAGAGACACUGCUGCUGAGAUGAAUGAAACAGUAGAAGUCAUC
UCAGAAAUGUUUGACCUCCAGGAGCCGACCUGCCUACAGACCCGC
CUGGAGCUGUACAAGCAGGGCCUGCGGGGCAGCCUCACCAAGCUC
AAGGGCCCCUUGACCAUGAUGGCCAGCCACUACAAGCAGCACUGC
CCUCCAACCCCGGAAACUUCCUGUGCAACCCAGAUUAUCACCUUU
GAAAGUUUCAAAGAGAACCUGAAGGACUUUCUGCUUGUCAUCCCC
UUUGACUGCUGGGAGCCAGUCCAGGAGUGACAAACACCAUUGUCA
CACUCCAUUUAAAAUACAUACUUCUUUACAUUCCAUUUAAACUAG
UGGUCCUAAACAUUUCACUUUAAACUUCCAGUCGAGGAUGUUUAC
A
Human GM-CSF without MOP
[SEQ ID NO: 61]
AUGUGGCUGCAGAGCCUGCUGCUCUUGGGCACUGUGGCCUGCAGC
AUCUCUGCACCCGCCCGCUCGCCCAGCCCCAGCACGCAGCCCUGG
GAGCAUGUGAAUGCCAUCCAGGAGGCCCGGCGUCUCCUGAACCUG
AGUAGAGACACUGCUGCUGAGAUGAAUGAAACAGUAGAAGUCAUC
UCAGAAAUGUUUGACCUCCAGGAGCCGACCUGCCUACAGACCCGC
CUGGAGCUGUACAAGCAGGGCCUGCGGGGCAGCCUCACCAAGCUC
AAGGGCCCCUUGACCAUGAUGGCCAGCCACUACAAGCAGCACUGC
CCUCCAACCCCGGAAACUUCCUGUGCAACCCAGAUUAUCACCUUU
GAAAGUUUCAAAGAGAACCUGAAGGACUUUCUGCUUGUCAUCCCC
UUUGACUGCUGGGAGCCAGUCCAGGAGUGA
Murine single chain IL-12 MOP (mscIL12-MOP),
without codon optimisation, with 3′ UTR perfect
matching complementary sequence to miRNA-122-5P,
miRNA-1-3P, miRNA-203a-3P, miRNA-30a-5P are
underlined (MOPV)
[SEQ ID NO: 68]
AUGUGUCCUCAGAAGCUAACCAUCUCCUGGUUUGCCAUCGUUUUG
CUGGUGUCUCCACUCAUGGCCAUGUGGGAGCUGGAGAAAGACGUU
UAUGUUGUAGAGGUGGACUGGACUCCCGAUGCCCCUGGAGAAACA
GUGAACCUCACCUGUGACACGCCUGAAGAAGAUGACAUCACCUGG
ACCUCAGACCAGAGACAUGGAGUCAUAGGCUCUGGAAAGACCCUG
ACCAUCACUGUCAAAGAGUUUCUAGAUGCUGGCCAGUACACCUGC
CACAAAGGAGGCGAGACUCUGAGCCACUCACAUCUGCUGCUCCAC
AAGAAGGAAAAUGGAAUUUGGUCCACUGAAAUUUUAAAAAAUUUC
AAAAACAAGACUUUCCUGAAGUGUGAAGCACCAAAUUACUCCGGA
CGGUUCACGUGCUCAUGGCUGGUGCAAAGAAACAUGGACUUGAAG
UUCAACAUCAAGAGCAGUAGCAGUUCCCCUGACUCUCGGGCAGUG
ACAUGUGGAAUGGCGUCUCUGUCUGCAGAGAAGGUCACACUGGAC
CAAAGGGACUAUGAGAAGUAUUCAGUGUCCUGCCAGGAGGAUGUC
ACCUGCCCAACUGCCGAGGAGACCCUGCCCAUUGAACUGGCGUUG
GAAGCACGGCAGCAGAAUAAAUAUGAGAACUACAGCACCAGCUUC
UUCAUCAGGGACAUCAUCAAACCAGACCCGCCCAAGAACUUGCAG
AUGAAGCCUUUGAAGAACUCACAGGUGGAGGUCAGCUGGGAGUAC
CCUGACUCCUGGAGCACUCCCCAUUCCUACUUCUCCCUCAAGUUC
UUUGUUCGAAUCCAGCGCAAGAAAGAAAAGAUGAAGGAGACAGAG
GAGGGGUGUAACCAGAAAGGUGCGUUCCUCGUAGAGAAGACAUCU
ACCGAAGUCCAAUGCAAAGGCGGGAAUGUCUGCGUGCAAGCUCAG
GAUCGCUAUUACAAUUCCUCAUGCAGCAAGUGGGCAUGUGUUCCC
UGCAGGGUCCGAUCCGGUGGCGGUGGCUCGGGCGGUGGUGGGUCG
GGUGGCGGCGGAUCUAGGGUCAUUCCAGUCUCUGGACCUGCCAGG
UGUCUUAGCCAGUCCCGAAACCUGCUGAAGACCACAGAUGACAUG
GUGAAGACGGCCAGAGAAAAACUGAAACAUUAUUCCUGCACUGCU
GAAGACAUCGAUCAUGAAGACAUCACACGGGACCAAACCAGCACA
UUGAAGACCUGUUUACCACUGGAACUACACAAGAACGAGAGUUGC
CUGGCUACUAGAGAGACUUCUUCCACAACAAGAGGGAGCUGCCUG
CCCCCACAGAAGACGUCUUUGAUGAUGACCCUGUGCCUUGGUAGC
AUCUAUGAGGACUUGAAGAUGUACCAGACAGAGUUCCAGGCCAUC
AACGCAGCACUUCAGAAUCACAACCAUCAGCAGAUCAUUCUAGAC
AAGGGCAUGCUGGUGGCCAUCGAUGAGCUGAUGCAGUCUCUGAAU
CAUAAUGGCGAGACUCUGCGCCAGAAACCUCCUGUGGGAGAAGCA
GACCCUUACAGAGUGAAAAUGAAGCUCUGCAUCCUGCUUCACGCC
UUCAGCACCCGCGUCGUGACCAUCAACAGGGUGAUGGGCUAUCUG
AGCUCCGCCUAACAAACACCAUUGUCACACUCCAUUUAAAAUACA
UACUUCUUUACAUUCCAUUUAAACUAGUGGUCCUAAACAUUUCAC
UUUAAACUUCCAGUCGAGGAUGUUUACA

If mRNA coding for both antigen and proinflammatory cytokine are administered, these can be formulated as separate mRNA constructs, or together on the same, polycistronic mRNA, as described above. Where separate mRNA constructs are used for these products, the separate constructs can each comprise the same set of miRNA binding site sequences (that is, they may each comprise the same OPS), or may comprise different sets of miRNA binding site sequences (different OPS), as further discussed elsewhere herein. In some cases, one or other of the mRNA constructs may entirely lack miRNA binding site sequences. It can be appreciated that mRNA a proinflammatory cytokine can be used in combination with any type of vaccine as known to the person of skill in the art, i.e., combination with protein-based (toxoid, recombinant, conjugated vaccines), RNA, mRNA and DNA-based vaccines (including circular or circularised RNA constructs as described above), live-attenuated vaccines, inactivated vaccines, or recombinant-vector based vaccines (e.g. MVA or adenovirus platform). In this way, the immune response to a co-administered mRNA-encoded antigen or other type of vaccine can be enhanced in a controllable, versatile way. Another advantage with this approach is the expectation that with the administration of the proinflammatory cytokine to enhance the immune response, there is the potential to provide multiple polypeptides in a single composition.

The introduction of coding nucleotide sequences into a target cell often requires the use of a delivery agent or ‘in vivo delivery composition’ to transfer the desired substance from the extracellular space to the intracellular environment. Frequently, such delivery agents/compositions may comprise delivery particles. Delivery particles may undergo phagocytosis and/or fuse with a target cell. Delivery particles may contain the desired substance by encapsulation or by comprising the substance within a matrix or structure. In some embodiments of any of the aspects, the first, second, and/or further mRNA constructs are comprised within or adsorbed to an in vivo delivery composition. In some embodiments of any of the aspects, the delivery composition comprises delivery vectors selected from the group consisting of: a particle, such as a polymeric particle; a liposome; a lipidoid particle; and a viral vector.

The term ‘delivery particle’ as used herein refers to drug or biological molecule delivery systems that comprise particles which can comprise therapeutic components by encapsulation, holding within a matrix, the formation of complex, surface adsorption or by other means. These systems can deliver a therapeutic component such as a coding nucleic acid sequence into a target cell. Compared to direct administration of a molecule or substance, the use of delivery particles may improve not only the efficacy of delivery, but also safety, by controlling the amount, time and/or release kinetics of the substance to be delivered at the site of action. Delivery particle systems are also adept at crossing biologic membranes to enable the substance or drug to get to the desired therapeutic target location.

Delivery particles may be on the micro-scale, but in specific embodiments may typically be on the nanoscale—i.e., nanoparticles. Nanoparticles are typically sized at least 50 nm (nanometres), suitably at least approximately 100 nm and typically at most 150 nm, 200 nm, although optionally up to 300 nm in diameter. In one embodiment of the invention the nanoparticles have a mean diameter of approximately at least 60 nm. An advantage of these sizes is that this means that the particles are below the threshold for reticuloendothelial system (mononuclear phagocyte system) clearance, i.e., the particle is small enough not to be destroyed by phagocytic cells as part of the body's defense mechanism. This facilitates the use of intravenous delivery routes for the compositions of the invention. The routes used to administer and deliver active substances comprised within delivery particles to their target tissue are a highly relevant factor when treating a disease, particularly an infectious disease. These routes may have different levels of efficacy depending on how they are applied.

In specific embodiments of the present invention the administration of the delivery particles is normally systemic, such as via sub-cutaneous, intravenous or intra-arterial administration. Occasionally, due to the type or severity of the disease delivery particles may be applied directly to an affected organ or tissue.

Alternative possibilities for the composition of the nanoparticles include polylactic acid (PLA), poly(lactic-co-glycolic acid) (PLGA), polycaprolactones, lipid- or phospholipid-based particles such as liposomes or exosomes; particles based on proteins and/or glycoproteins such as collagen, albumin, gelatin, elastin, gliadin, keratin, legumain, zein, soy proteins, milk proteins such as casein, and others (Lohcharoenkal et al. BioMed Research International; Volume 2014 (2014)); colloidal nanoparticles; and particles based on metals or metallic compounds such as gold, silver, aluminium, copper oxides, metal-organic cycles and cages (MOCs) and so on. In specific embodiments poly(lactic-co-glycolic acid) (PLGA) may be used in delivery particles of the invention due to its high biocompatibility and biodegradability. PLGA was approved for clinical use in 1989, by the US Food and Drug Administration (FDA). It has been favoured for sustained release formulations of a wide range of drugs and biomolecules since that time. PLGA may be co-formulated with polyvinyl alcohol (PVA) in order to create micelle based nanoparticles as well. Micelles may also be prepared using a diblock copolymer of PLGA and PEG, or a PEG-PLGA-PEG triblock copolymer.

In particular, polymers comprising polyethyleneimine (PEI) have been investigated for the delivery of nucleic acids. Nanoparticle vectors composed of poly(1-amino esters) (PBAEs) have also been shown to be suitable for nucleic acid delivery, especially in coformulation with polyethylene glycol (PEG) (Kaczmarek J C et al Angew Chem Int Ed Engl. 2016; 55 (44): 13808-13812). Dendrimers are also contemplated for use. Particles of such coformulations have been used to deliver mRNA to the lung.

Also considered are particles based on polysaccharides and their derivatives, such as cellulose, chitin, cyclodextrin, and chitosan. Chitosan is a cationic linear polysaccharide obtained by partial deacetylation of chitin, with nanoparticles comprising this substance possessing promising properties for drug delivery such as biocompatibility, low toxicity and small size (Felt et al., Drug Development and Industrial Pharmacy, Volume 24, 1998-Issue 11). It is envisioned that combinations between the above constituents may be used. In specific embodiments of the invention the nanoparticles comprise chitosan which exhibits excellent mucoadhesion and penetration properties that make it ideal for sustained release biomolecule delivery in mucosa.

Delivery particles may include lipid-based, such as niosomal or liposomal, nanoparticle delivery systems. Lipid nanoparticles are multicomponent lipid systems typically containing a phospholipid, an ionizable lipid, cholesterol, and a PEGylated lipid. The PEGylated lipids on the particle surface can help to reduce particle aggregation and prolong the circulation time in vivo. Suitable liposomal formulations may include L-a-phosphatidylcholine and PEG-DMG (1,2-dimyristoyl-rac-glycero-3-methoxypolyethylene glycol). Alternative liposomal formulations comprising an ionizable lipid that are particularly, suitable for delivery of a nucleic acid may comprise DSPC (1,2-distearoyl-sn-glycero-3-phosphocholine) and Dlin-MC3-DMA (6Z, 9Z, 28Z, 31Z)-heptatriacont-6,9,28,31-tetraene-19-yl 4-(dimethylamino) butanoate. Another determinant for the potency of lipid-based nanoparticle is the lipid pKa. An optimal lipid pKa for the delivery of mRNA cargo is in the range of 6.6-6.8.

The delivery particles may comprise aminoalcohol lipidoids. These compounds may be used in the formation of particles including nanoparticles, liposomes and micelles, which are particularly suitable for the delivery of nucleic acids. An illustrative example for the production of nanoformulations comprising aminoalcohol lipidoid particles according to some embodiments of the invention may be found in the Examples. In embodiments of the invention, lipid nanoparticles (LNPs) comprised of dipalmitoylphosphatidylcholine (DPPC), cholesterol, and dioleoylglycerophosphate-diethylenediamine conjugate (DOP-DEDA) are positively charged at pH of 6.0, neutral at pH of 7.4 and negatively charged at pH of 8.0. This delivery system is neutral in the bloodstream to minimize degradation by plasma proteins and protect the encapsulated mRNA cargo. When delivered in vivo these LNP vehicles bind to apolipoproteins (e.g., apoE3) at their hydrophobic lipid regions, which can promote cellular uptake.

The delivery particles may be targeted to the cells of the target tissue. This targeting may be mediated by a targeting agent on the surface of the delivery particles, which may be a protein, peptide, carbohydrate, glycoprotein, lipid, small molecule, nucleic acid, etc. The targeting agent may be used to target specific cells or tissues or may be used to promote endocytosis or phagocytosis of the particle. Examples of targeting agents include, but are not limited to, antibodies, fragments of antibodies, low-density lipoproteins (LDLs), transferrin, asialycoproteins, gp120 envelope protein of the human immunodeficiency virus (HIV), carbohydrates, receptor ligands, sialic acid, aptamers etc. Targeted liposomes, for example, modified by active targeting ligands can significantly improve liposome capacity by increasing accumulation at the target tissues/organs/cells without releasing the cargo, such as mRNA, to other sites.

Lipid-based nanoparticles may also act advantageously as an adjuvant in themselves. a broad range of lipids are reported to possess the strong inherent adjuvant activity. Cationic lipids such as dimethyldioctadecylammonium bromide (DDA) show the deposition of antigen at the injection site as well as the enhancement of a cellular antigen internalization. Solid lipid nanoparticles structured by DDA demonstrate high antigen adsorption efficiency, in vitro antigen trafficking, in vivo distribution, and high antibody response (Anderluzzi et al. J. Control Release 2020, 330, 933-944). As a result, efforts to improve adjuvanticity in mRNA delivery vaccines that utilise LNPs as a delivery system tend to focus on engineering the lipids used in the nanoparticles. As mentioned above, however, there is a trade-off between lipid properties and suitability for encapsulation of mRNA as a cargo as well as in terms of biodistribution, release kinetics and cellular uptake.

In embodiments where multiple different mRNA molecules are comprised in one or more delivery system, it is contemplated that each delivery system—e.g., particle, liposome, viral vector system—may comprise one or more than one type of mRNA molecule as the ‘payload’; that is, not every delivery payload in a particular embodiment will necessarily comprise all of the mRNA molecules provided in said embodiment. In this way, it is also considered possible to direct different delivery systems and their associated sequences to different target cells, with the targeting agents described herein.

Similarly, in any embodiments where separate mRNA constructs are provided, and in which they are formulated to be associated with delivery particles (as described elsewhere herein), these may be co-formulated (that is, the different mRNA may be packaged with the delivery particles together in the same process), such that different mRNA constructs may be associated with the same delivery particles, or separately formulated, such that different mRNA constructs may be associated with different delivery particles.

The mRNA constructs of certain embodiments of the invention may be synthesised from a polynucleotide expression construct, which may be for example a DNA plasmid. This expression construct may comprise any promoter sequence necessary for the initiation of transcription and a corresponding termination sequence, such that transcription of the mRNA construct can occur. Such polynucleotide expression constructs are contemplated to comprise embodiments of the invention in their own right.

In some embodiments of any of the aspects, administration is intravenous, subcutaneous, intramuscular, intranasal, intra-arterial, or via inhalation. In some embodiments of any of the aspects, the composition(s) is formulated for administration by intravenous, subcutaneous, intramuscular, intranasal, intra-arterial, or via inhalation means.

When administered to a subject, a therapeutic component is suitably administered as part of the in vivo delivery composition and may further comprise a pharmaceutically acceptable vehicle in order to create a pharmaceutical composition. Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are preferably sterile. Water is a suitable vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skimmed milk, glycerol, propylene, glycol, water, ethanol and the like. Pharmaceutical compositions, if desired, can also contain minor amounts of wetting or emulsifying agents, or buffering agents.

The medicaments and pharmaceutical compositions of the invention can take the form of liquids, solutions, suspensions, gels, modified-release formulations (such as slow or sustained-release), emulsions, capsules (for example, capsules containing liquids or gels), liposomes, microparticles, nanoparticles or any other suitable formulations known in the art. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

For any compound or composition described herein, the therapeutically effective amount can be initially determined from in vitro cell culture assays. Target concentrations will be those concentrations of active component(s) that are capable of achieving the methods described herein, as measured using the methods described herein or known in the art.

As is well known in the art, therapeutically effective amounts for use in human subjects can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

It is contemplated that embodiments of the invention may include compositions formulated for use in medicine. As such, the composition of the invention may be suspended in a biocompatible solution to form a composition that can be targeted to a location on a cell, within a tissue or within the body of a patient or animal (i.e., the composition can be used in vitro, ex vivo or in vivo). Suitably, the biocompatible solution may be phosphate buffered saline or any other pharmaceutically acceptable carrier solution. One or more additional pharmaceutically acceptable carriers (such as diluents, adjuvants, excipients or vehicles) may be combined with the composition of the invention in a pharmaceutical composition. Suitable pharmaceutical carriers are described in ‘Remington's Pharmaceutical Sciences’ by E. W. Martin. Pharmaceutical formulations and compositions of the invention are formulated to conform to regulatory standards and can be administered orally, intravenously, topically, or subcutaneously, or via other standard routes. Administration can be systemic or local or intranasal or intrathecal. In particular, compositions according to the invention can be administered intravenously, intralesionally, subcutaneously, intramuscularly, intranasally, intrathecally, intra-arterially and/or through inhalation.

It can be appreciated that the above-mentioned approach is particular suitable for preparing and/or using vaccine therapeutic compositions similar to typical ‘toxoid’ vaccines, where an immune response is induced against an inactivated toxin produced by a bacterium or other organism, or ‘subunit’ vaccines, where an immune response is induced against a fragment of a target micro-organism.

It is contemplated that the compositions and methods as described herein may act to induce an immune response against disease or infection from a pathogenic organism. A ‘therapeutic component’ or ‘therapeutic agent’ as defined herein refers to a molecule, substance, cell or organism that when administered to an individual human or other animal as part of a therapeutic intervention, contributes towards a therapeutic effect upon that individual human or other animal. The therapeutic effect may be caused by the therapeutic component itself, or by another component of the therapeutic intervention. The therapeutic component may be a coding nucleic acid component, in particular an mRNA. The coding nucleic acid component(s) may code for therapeutic enhancement factors, as defined herein. A therapeutic component may also comprise a drug, such as a small molecule or monoclonal antibody (or fragment thereof). In other embodiments of the invention, the therapeutic agent comprises a therapeutic virus, such as a viral vector.

The term ‘therapeutic effect’ refers to a local or systemic effect in an animal subject, typically a human, caused by a pharmacologically or therapeutically active agent that comprises a substance, molecule, composition, cell or organism that has been administered to the subject, and the term ‘therapeutic intervention’ refers to the administration of such a substance, molecule, composition, cell or organism. The term thus means any agent intended for use in the diagnosis, cure, mitigation, treatment or prevention of disease or in the enhancement of desirable physical or mental development and conditions in an animal or human subject. The phrase ‘therapeutically-effective amount’ means that amount of such an agent that produces a desired local or systemic effect at a reasonable benefit/risk ratio applicable to any treatment. In certain embodiments, a therapeutically effective amount of an agent will depend on its therapeutic index, solubility, and the like. For example, certain therapeutic agents of the present invention may be administered in a sufficient amount to produce a reasonable benefit/risk ratio applicable to such treatment. In the specific context of treatment of disease including infectious disease, a ‘therapeutic effect’ can be manifested by various means, including but not limited to, a decrease in infectious pathogenic organism titre, an increase in beneficial cellular biomarkers (e.g. an increase in white cell count), and/or amelioration of various physiological symptoms associated with the condition. In the specific context of the treatment of a viral, bacterial or parasitic infection, such as by prophylaxis through vaccination, a ‘therapeutic effect’ may be shown by full or partial resistance to pathogen challenge, presence of circulating antibodies to the pathogen in the human or animal subject, or other known measures of vaccine efficacy.

In one embodiment, the subject to whom therapy is administered is a mammal (e.g. rodent, primate, non-human mammal, domestic animal or livestock, such as a dog, cat, rabbit, guinea pig, cow, horse, sheep, goat and the like), and is suitably a human. In a further embodiment, the subject is an animal model of disease, such as an infectious disease. For example, the animal model may be infected with one or more viruses, bacteria, fungi, prions or eukaryotic parasites, or is to be infected with such pathogens.

In a specific embodiment of the methods of the present invention, the subject has not yet undergone a therapeutic treatment. In still another embodiment, the subject has undergone a therapeutic treatment. In yet a further embodiment, the subject is undergoing a therapeutic treatment.

In some embodiments, the provided coding mRNA construct may code for a ‘therapeutic enhancement factor’. According to the present invention therapeutic enhancement factors are gene products or polypeptides that may enhance or facilitate the ability of another, co-administered therapeutic agent, to exert a therapeutic effect upon a given cell, suitably the target cell. When introduced into or in the vicinity of the target cell, expression of the therapeutic enhancement factor may cooperate with a co-administered therapeutic agent thereby enabling or enhancing the therapeutic activity of the agent. In other embodiments the therapeutic enhancement factor may act as an adjuvant for a co- or sequentially administered vaccine. Adjuvants are pharmacological or immunological substances that may be used to activate the innate immune system of a subject. In this way they permit the innate immune system of the subject to respond to infection from a pathogen more rapidly. Adjuvants may also serve to stimulate adaptive immune responses that are specific to particular infectious agents, such as viral or bacterial infections. Some adjuvants may also be effective in directing effective antigen presentation and stimulating and enhancing T helper type-1 (Th1) immune responses. Alternatively, the therapeutic enhancement factor may act as an adjuvant for a co- or sequentially administered attenuated or modified virus, such as a modified adenovirus utilised in a vaccine formulation. Inactivated virus or live attenuated virus vaccines will typically need adjuvants in order to promote immune response. In addition, the inherent immunogenicity of recombinant protein-based subunit vaccines is also relatively low, and co-administered adjuvants are desirable. Hence, in specific embodiments of the invention the role of an adjuvant composition is to increase the level of neutralising antibodies produced by immune cells in response to a presented antigen.

Multiple therapeutic enhancement factors may be combined in compositions according to specific embodiments of the present invention. In such embodiments, the coding sequences for each therapeutic enhancement factor may be present in separate mRNA molecules. In some embodiments, sequences for more than one therapeutic enhancement factor may be present on the same mRNA molecule. In such cases the polycistronic mRNA molecule further comprises sequences as necessary for the expression of all coded sequences, such as internal ribosome entry sites (IRES).

In embodiments of the invention viruses may be selected from any one of the Groups I-VII of the Baltimore classification of viruses (Baltimore D (1971). “Expression of animal virus genomes”. Bacteriol Rev. 35 (3): 235-41). In specific embodiments of the invention suitable viruses may be selected from Baltimore Group I, which are characterised as having double stranded DNA viral genomes; Group II, which are characterized as having positive single stranded DNA genomes, Group III, which are characterized as having double stranded RNA viral genomes, Group IV, which have single stranded positive RNA genomes; and Group V, which have single stranded negative RNA genomes.

The term “treatment” (including variations thereof, e.g., “treat” or “treated”) as used herein means any one or more of the following: (i) the prevention of infection or re-infection, as in a traditional vaccine, (ii) the reduction in the severity of, or, in the elimination of symptoms, and (iii) the substantial or complete elimination of the pathogen or disorder in question. Hence, treatment may be effected prophylactically (prior to infection) or therapeutically (following infection). In the present invention, prophylactic treatment is the preferred mode. According to a particular embodiment of the present invention, compositions and methods are provided that treat, including prophylactically and/or therapeutically immunize, a host animal against an infection (e.g., a bacterium or virus). The methods of the present invention are useful for conferring prophylactic and/or therapeutic immunity to a subject. The methods of the present invention can also be practiced on subjects for biomedical research applications.

The compositions and methods described herein can be administered to a subject in need of vaccination, immunization, and/or stimulation of an immune response. In some embodiments of any of the aspects, the methods described herein comprise administering an effective amount of compositions described herein, e.g. to a subject in order to stimulate an immune response or provide protection against the relevant pathogen the antigen was derived from. Providing protection against the relevant pathogen is stimulating the immune system such that later exposure to the antigen (e.g., on or in a live pathogen) triggers a more effective immune response than if the subject was naïve to the antigen. Protection can include faster clearance of the pathogen, reduced severity and/or time of symptoms, and/or lack of development of disease or symptoms. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or topical, administration. Administration can be local or systemic. In some embodiments of any of the aspects, the administration can be intramuscular or subcutaneous.

The term “effective amount” as used herein refers to the amount of adjuvant needed to stimulate the immune system, or in combination with an antigen, to provide a protective effect against subsequent infections, and relates to a sufficient amount of pharmacological composition to provide the desired effect. The term “therapeutically effective amount” therefore refers to an amount of the adjuvant (and optionally, the antigen) that is sufficient to provide a particular immune stimulatory effect when administered to a typical subject. An effective amount as used herein, in various contexts, would also include an amount sufficient to delay the development of a symptom of the disease, alter the course of a symptom of the disease (for example but not limited to, slowing the progression of a symptom of the disease), or prevent a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of a composition which achieves a half-maximal inhibition of symptoms or induction of desired responses) as determined in cell culture, or in an appropriate animal model. Levels in plasma can be measured, for example, by high performance liquid chromatography. The effects of any particular dosage can be monitored by a suitable bioassay, e.g., assay for antibody titers, among others. The dosage can be determined by a physician and adjusted, as necessary, to suit observed effects of the treatment.

In some embodiments of any of the aspects, the technology described herein relates to a pharmaceutical composition comprising an adjuvant comprising a mRNA construct encoding a proinflammatory cytokine, and optionally an antigen or mRNA construct encoding an antigen as described herein, and optionally a pharmaceutically acceptable carrier.

In some embodiments of any of the aspects, the active ingredients of the pharmaceutical composition comprises a mRNA construct encoding a proinflammatory cytokine, and an antigen or mRNA construct encoding an antigen as described herein. In some embodiments of any of the aspects, the active ingredients of the pharmaceutical composition consist essentially of a mRNA construct encoding a proinflammatory cytokine, and an antigen or mRNA construct encoding an antigen as described herein. In some embodiments of any of the aspects, the active ingredients of the pharmaceutical composition consist of an adjuvant comprising a mRNA construct encoding a proinflammatory cytokine, and an antigen or mRNA construct encoding an antigen as described herein.

In some embodiments of any of the aspects, the active ingredients of the pharmaceutical composition comprises a mRNA construct encoding a proinflammatory cytokine as described herein. In some embodiments of any of the aspects, the active ingredients of the pharmaceutical composition consist essentially of a mRNA construct encoding a proinflammatory cytokine as described herein. In some embodiments of any of the aspects, the active ingredients of the pharmaceutical composition consist of an adjuvant comprising a mRNA construct encoding a proinflammatory cytokine as described herein.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments of any of the aspects, the carrier inhibits the degradation of the active agent as described herein.

In some embodiments of any of the aspects, the pharmaceutical composition comprising an adjuvant comprising a mRNA construct encoding a proinflammatory cytokine, and optionally an antigen or mRNA construct encoding an antigen as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of an adjuvant as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an composition or construct as disclosed herein can also be incorporated into the parenteral dosage forms of the disclosure, including conventional and controlled-release parenteral dosage forms.

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments of any of the aspects, the adjuvant can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the methods described herein can further comprise administering a second agent and/or treatment to the subject, e.g. as part of a combinatorial therapy.

In some embodiments of any of the aspects, an effective dose of a composition comprising an adjuvant comprising a mRNA construct encoding a proinflammatory cytokine, and optionally an antigen or mRNA construct encoding an antigen as described herein can be administered to a patient once. In some embodiments of any of the aspects, an effective dose of the composition can be administered to a patient repeatedly. For systemic administration, subjects can be administered a therapeutic amount of the composition, such as, e.g., 0.01 mg/kg, 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In one aspect of any of the embodiments, the subject is administered a) a dose of the first cytokine mRNA construct and b) a dose of the first antigen mRNA construct and/or the antigen polypeptide, antigen molecule, or killed or attenuated pathogenic agent. In some embodiments of any of the aspects, the dose of the first cytokine mRNA construct is no more than 20% of the dose of the first antigen mRNA construct and/or the antigen polypeptide, antigen molecule, or killed or attenuated pathogenic agent by weight. In some embodiments of any of the aspects, the dose of the first cytokine mRNA construct is no more than 10% of the dose of the first antigen mRNA construct and/or the antigen polypeptide, antigen molecule, or killed or attenuated pathogenic agent by weight. In some embodiments of any of the aspects, the dose of the first cytokine mRNA construct is from 0.5% to 20% of the dose of the first antigen mRNA construct and/or the antigen polypeptide, antigen molecule, or killed or attenuated pathogenic agent by weight. In some embodiments of any of the aspects, the dose of the first cytokine mRNA construct is from 1% to 10% of the dose of the first antigen mRNA construct and/or the antigen polypeptide, antigen molecule, or killed or attenuated pathogenic agent by weight. In some embodiments of any of the aspects, the dose of the first cytokine mRNA construct is no more than 20% of the dose of the first antigen mRNA construct by weight. In some embodiments of any of the aspects, the dose of the first cytokine mRNA construct is no more than 10% of the dose of the first antigen mRNA construct by weight. In some embodiments of any of the aspects, the dose of the first cytokine mRNA construct is from 0.5% to 20% of the dose of the first antigen mRNA construct by weight. In some embodiments of any of the aspects, the dose of the first cytokine mRNA construct is from 1% to 10% of the dose of the first antigen mRNA construct by weight. In some embodiments of any of the aspects, the subject is a human subject and is administered a dose of the first cytokine mRNA construct of from 0.10 μg to 10 μg. In some embodiments of any of the aspects, the subject is a human subject and is administered a dose of the first cytokine mRNA construct of from 0.15 μg to 6 μg. In some embodiments of any of the aspects, the subject is a human subject and is administered a dose of the first cytokine mRNA construct of from 0.3 μg to 3 μg.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the adjuvant and/or the antigen. The desired dose or amount of activation can be administered at one time or divided into subdoses, e.g., 2-4 subdoses and administered over a period of time, e.g., at appropriate intervals through the day or other appropriate schedule. In some embodiments of any of the aspects, administration can be chronic, e.g., one or more doses over a period of weeks or months.

Effective amounts, toxicity, and therapeutic efficacy can be determined by standard pharmaceutical procedures in cell cultures or experimental animals, e.g., for determining the minimal effective dose and/or maximal tolerated dose. The dosage can vary depending upon the dosage form employed and the route of administration utilized. A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for immune response among others. The dosage can be determined by a physician and adjusted, as necessary, to suit observed effects of the treatment.

The dosage ranges for the administration of an adjuvant comprising a mRNA construct encoding a proinflammatory cytokine, and optionally an antigen or mRNA construct encoding an antigen according to the methods described herein depend upon, for example, the form of the adjuvant, its potency, and the extent to which symptoms, markers, or indicators of a response described herein are desired to be induced, for example the percentage indication desired for an immune response. The dosage should not be so large as to cause adverse side effects, such as inflammatory responses. Generally, the dosage will vary with the age, condition, and sex of the patient and can be determined by one of skill in the art. The dosage can also be adjusted by the individual physician in the event of any complication.

The efficacy of the adjuvant comprising a mRNA construct encoding a proinflammatory cytokine, and optionally an antigen or mRNA construct encoding an antigen as described herein in, e.g. to induce a response as described herein (e.g. an immune response or immunization) can be determined by the skilled clinician. However, a treatment is considered “effective treatment,” as the term is used herein, if one or more of the signs or symptoms of a condition described herein are altered in a beneficial manner, other clinically accepted signs or symptoms are improved, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Immune responses can be detected by a variety of methods known to those skilled in the art, including but not limited to, antibody production, cytotoxicity assay, proliferation assay and cytokine release assays. For example, samples of blood can be drawn from the immunized mammal and analyzed for the presence of antibodies against the antigen administered in the respective vaccine and the titer of these antibodies can be determined by methods known in the art.

Efficacy of an agent can be determined by assessing physical indicators of a desired response, (e.g., immune response, cytokine production, antibody titers, etc.). It is well within the ability of one skilled in the art to monitor efficacy of administration and/or treatment by measuring any one of such parameters, or any combination of parameters. Efficacy can be assessed in animal models of a condition described herein, for example immunization of monkeys. When using an experimental animal model, efficacy of treatment is evidenced when a statistically significant change in a marker is observed.

In vitro and animal model assays are provided herein which allow the assessment of a given dose of an adjuvant and/or antigen. By way of non-limiting example, the effects of a dose of adjuvant can be assessed by measuring the antibody titers or cytokine production.

The efficacy of a given dosage combination can also be assessed in an animal model, e.g., immunization of animals as described in the Examples herein.

In one aspect of any of the embodiments, described herein is a combination or kit comprising a) a dose of the first cytokine mRNA construct and b) a dose of the first antigen mRNA construct and/or the antigen polypeptide, antigen molecule, or killed or attenuated pathogenic agent. In some embodiments, a) the dose of the first cytokine mRNA construct and b) the dose of the first antigen mRNA construct and/or the antigen polypeptide, antigen molecule, or killed or attenuated pathogenic agent are provided in separate formulations. In some embodiments, the a) the dose of the first cytokine mRNA construct and b) the dose of the first antigen mRNA construct and/or the antigen polypeptide, antigen molecule, or killed or attenuated pathogenic agent are provided in the same formulation. In one aspect of any of the embodiments, described herein is a combination or kit comprising a) at least one dose of the first cytokine mRNA construct and b) at least one dose of the first antigen mRNA construct and/or the antigen polypeptide, antigen molecule, or killed or attenuated pathogenic agent. In some embodiments, a) the at least one dose of the first cytokine mRNA construct and b) the at least one dose of the first antigen mRNA construct and/or the antigen polypeptide, antigen molecule, or killed or attenuated pathogenic agent are provided in separate formulations. In some embodiments, the a) the at least one dose of the first cytokine mRNA construct and b) the at least one dose of the first antigen mRNA construct and/or the antigen polypeptide, antigen molecule, or killed or attenuated pathogenic agent are provided in the same formulation.

In some embodiments of any of the aspects, the dose (or at least one dose) of the first cytokine mRNA construct is no more than 20% of the dose (or at least one dose) of the first antigen mRNA construct and/or the antigen polypeptide, antigen molecule, or killed or attenuated pathogenic agent by weight. In some embodiments of any of the aspects, the dose (or at least one dose) of the first cytokine mRNA construct is no more than 10% of the dose (or at least one dose) of the first antigen mRNA construct and/or the antigen polypeptide, antigen molecule, or killed or attenuated pathogenic agent by weight. In some embodiments of any of the aspects, the dose (or at least one dose) of the first cytokine mRNA construct is from 0.5% to 20% of the dose (or at least one dose) of the first antigen mRNA construct and/or the antigen polypeptide, antigen molecule, or killed or attenuated pathogenic agent by weight. In some embodiments of any of the aspects, the dose (or at least one dose) of the first cytokine mRNA construct is from 1% to 10% of the dose (or at least one dose) of the first antigen mRNA construct and/or the antigen polypeptide, antigen molecule, or killed or attenuated pathogenic agent by weight. In some embodiments of any of the aspects, the dose (or at least one dose) of the first cytokine mRNA construct is no more than 20% of the dose (or at least one dose) of the first antigen mRNA construct by weight. In some embodiments of any of the aspects, the dose (or at least one dose) of the first cytokine mRNA construct is no more than 10% of the dose (or at least one dose) of the first antigen mRNA construct by weight. In some embodiments of any of the aspects, the dose (or at least one dose) of the first cytokine mRNA construct is from 0.5% to 20% of the dose (or at least one dose) of the first antigen mRNA construct by weight. In some embodiments of any of the aspects, the dose (or at least one dose) of the first cytokine mRNA construct is from 1% to 10% of the dose (or at least one dose) of the first antigen mRNA construct by weight. In some embodiments of any of the aspects, the subject is a human subject and the dose (or each dose) of the first cytokine mRNA construct is from 0.10 μg to 10 μg. In some embodiments of any of the aspects, the subject is a human subject and the dose (or each dose) of the first cytokine mRNA construct is from 0.15 μg to 6 μg. In some embodiments of any of the aspects, the subject is a human subject and the dose (or each dose) of the first cytokine mRNA construct is from 0.3 μg to 3 μg.

As used herein “combination” refers to a group of two or more substances for use together, e.g., for use in inducing an immune response. The two or more substances can be present in the same formulation in any molecular or physical arrangement, e.g., in an admixture, in a lipid-nanoparticle, in a solution, in a mixture, in a suspension, in a colloid, in an emulsion. The formulation can be a homogeneous or heterogenous mixture. In some embodiments of any of the aspects, the two or more substances can be comprised by the same or different superstructures, e.g., nucleic acid molecules, vectors, nanoparticles, liposomes, cells, scaffolds, or the like, and said superstructure is in solution, mixture, admixture, suspension with a solvent, carrier, or some of the two or more substances. Alternatively, the two or more substances can be present in two or more separate formulations, e.g., in a kit or package comprising multiple formulations in separate containers, to be mixed or brought into contact with each other when a method or administration is to be performed.

In one aspect of any of the embodiments, described herein is a kit comprising an adjuvant comprising a mRNA construct encoding a proinflammatory cytokine, and optionally an antigen or mRNA construct encoding an antigen as described herein. The adjuvant and antigen can be present in the same formulation of the kit or in separate formulations of the kit, e.g., for separate administration or for mixing prior to administration.

A kit is any manufacture (e.g., a package or container) comprising at least one reagent, e.g., an adjuvant comprising a mRNA construct encoding a proinflammatory cytokine, and optionally an antigen or mRNA construct encoding an antigen as described herein, the manufacture being promoted, distributed, or sold as a unit for performing the methods described herein. The kits described herein can optionally comprise additional components useful for performing the methods described herein. By way of example, the kit can comprise fluids and compositions (e.g., buffers, needles, syringes etc.) suitable for performing one or more of the administrations according to the methods described herein, an instructional material which describes performance of a method as described herein, and the like. Additionally, the kit may comprise an instruction leaflet.

In some embodiments of any of the aspects, the compositions and methods described herein can be used in conjunction with the compositions described in PCT/US2021/19028 and PCT/US2021/43975, which are incorporated by reference herein in their entireties.

In one aspect of any of the embodiments, described herein is a composition(s) comprising a first cytokine mRNA construct comprising an ORF encoding a first proinflammatory cytokine; and a second cytokine mRNA construct comprising an ORF encoding a second proinflammatory cytokine. In one aspect of any of the embodiments, described herein is a composition(s) comprising a first cytokine mRNA construct comprising an ORF encoding a first proinflammatory cytokine; a second cytokine mRNA construct comprising an ORF encoding a second proinflammatory cytokine; and a one or more further cytokine mRNA constructs each comprising an ORF encoding a further proinflammatory cytokine. In some embodiments of any of the aspects, the first ORF encodes IL-12 or a subunit, derivative, fragment, agonist or homologue thereof and the second and optionally further ORFs each encode a cytokine selected from: IL-2; IL-4; IL-5; IL-6; IL-8; IL-10; IL-13; IL-27; IL-1β; TGFβ; IFNγ; IFNα; IFNβ; TNFα; CCL2; CCL3; CCL4; CCL5; CCL8; CXCL12; GM-CSF; or a subunit, derivative, fragment, agonist or homologue thereof.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

The term ‘diseased’ as used herein, as in ‘diseased cells’ and/or ‘diseased tissue’ indicates tissues and organs (or parts thereof) and cells which exhibit an aberrant, non-healthy or disease pathology. For instance, diseased cells may be infected with a virus, bacterium, prion, fungi or eukaryotic parasite; and/or may comprise deleterious mutations. Infection may comprise a pathogen that is internalised and resides within the cell for a significant portion of its life cycle. Diseased cells may comprise an altered intra-cellular miRNA environment when compared to otherwise normal or so-called healthy cells. In certain instances, diseased cells may be pathologically normal but comprise an altered intra-cellular miRNA environment that represents a precursor state to disease. Diseased tissues may comprise healthy tissues that have been infiltrated by diseased cells from another organ or organ system. By way of example, many inflammatory diseases comprise pathologies where otherwise healthy organs are subjected to infiltration with immune cells such as T cells and neutrophils. By way of a further example, organs and tissues subjected to stenotic or cirrhotic lesions may comprise both healthy and diseased cells in close proximity.

The term ‘healthy’ as used herein, as in ‘healthy cells’ and/or ‘healthy tissue’ indicates tissues and organs (or parts thereof) and cells which are not themselves diseased and/or approximate to a typically normal functioning phenotype. It can be appreciated that in the context of the invention the term ‘healthy’ is relative, as, for example, non-infected cells in a tissue affected by a pathogen may well not be entirely healthy in an absolute sense. Therefore ‘non-healthy cells’ means cells which are not themselves infected but which may be inflamed or otherwise diseased for example. Similarly, ‘healthy or non-healthy tissue’ means tissue, or parts thereof, without the present of pathogenic organisms or their products; or other diseases as mentioned above; regardless of overall health. Models used for approximation of normal functioning phenotypes for ‘healthy’ cells may include immortalised cell lines that are otherwise close to the originator cells in terms of cellular function and gene expression.

In an alternative embodiment, the health status of a cell, cell type, tissue and/or organ is determined by the quantification of miRNA expression. In certain disease types the expression of particular miRNA species is affected, and can be up- or down-regulated compared to unaffected cells. This difference in the miRNA transcriptome can be used to identify relative states of health, and/or to track the progression of healthy cells, cell types, tissues and/or organs towards a disease state. In embodiments of the present invention the differential variations in the miRNA transcriptome of cell types comprised within a given organ or organ system is leveraged in order to control protein expression in the different cell types.

As used herein, the term ‘organ’ is synonymous with an ‘organ system’ and refers to a combination of tissues and/or cell types that may be compartmentalised within the body of a subject to provide a biological function, such as a physiological, anatomical, homeostatic or endocrine function. Suitably, organs or organ systems may mean a vascularized internal organ, such as a liver or pancreas. Typically, organs comprise at least two tissue types, and/or a plurality of cell types that exhibit a phenotype characteristic of the organ. Tissues or tissue systems may cooperate but not formally be considered as an organ. For example, blood is generally considered a tissue, or even a liquid tissue, but depending upon the definition used may not be regarded as an organ in the strict sense. Nevertheless, the compositions and methods of the invention in certain embodiments may serve to exhibit a protective effect in respect of organs, tissues and tissue systems including the blood, haematopoietic and lymphoid tissue.

As used herein, a “subject” means a human or animal. Usually the animal is a vertebrate such as a primate, rodent, domestic animal or game animal. Primates include chimpanzees, cynomolgous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of immune distinct subjects, or subjects having an infectious disease. A subject can be male or female.

A subject can be one who has been previously diagnosed with or identified as suffering from or having a condition in need of treatment (e.g., being immune distinct, or having an infectious disease) or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. A protein or polypeptide can be produced naturally or in vitro by synthetic means. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. Polypeptides can also undergo maturation or post-translational modification processes that may include, but are not limited to: glycosylation, proteolytic cleavage, lipidation, signal peptide cleavage, pro peptide cleavage, phosphorylation, and such like.

The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing. The terms also refer to fragments or variants of the polypeptide that maintain at least 50% of the activity or effect, e.g. proinflammatory or immune-stimulating effects of the full length polypeptide. Conservative substitution variants that maintain the activity of a wildtype protein will include a conservative substitution as defined herein. The identification of amino acids most likely to be tolerant of conservative substitution while maintaining at least 50% of the activity of the wildtype is guided by, for example, sequence alignment with homologs or paralogs from other species. Amino acids that are identical between homologs are less likely to tolerate change, while those showing conservative differences are obviously much more likely to tolerate conservative change in the context of an artificial variant. Similarly, positions with non-conservative differences are less likely to be critical to function and more likely to tolerate conservative substitution in an artificial variant. Variants, fragments, and/or fusion proteins can be tested for activity, for example, by administering the variant to an appropriate animal model of infectious disease as described herein.

In some embodiments, a polypeptide, can be a variant of a sequence described herein. In some embodiments, the variant is a conservative substitution variant. Variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Polypeptide-encoding DNA sequences encompass sequences that comprise one or more additions, deletions, or substitutions of nucleotides when compared to a native or reference DNA sequence, but that encode a variant protein or fragment thereof that retains the relevant biological activity relative to the reference protein, e.g., at least 50% of the wildtype protein. As to amino acid sequences, one of skill will recognize that individual substitutions, deletions or additions to a nucleic acid, peptide, polypeptide, or protein sequence which alters a single amino acid or a small percentage, (i.e., 5% or fewer, e.g. 4% or fewer, or 3% or fewer, or 1% or fewer) of amino acids in the encoded sequence is a “conservatively modified variant” where the alteration results in the substitution of an amino acid with a chemically similar amino acid. It is contemplated that some changes can potentially improve the relevant activity, such that a variant, whether conservative or not, has more than 100% of the activity of the wildtype, e.g., 110%, 125%, 150%, 175%, 200%, 500%, 1000% or more.

One method of identifying amino acid residues which can be substituted is to align, for example, human to a homolog from one or more non-human species. Alignment can provide guidance regarding not only residues likely to be necessary for function but also, conversely, those residues likely to tolerate change. Where, for example, an alignment shows two identical or similar amino acids at corresponding positions, it is more likely that that site is important functionally. Where, conversely, alignment shows residues in corresponding positions to differ significantly in size, charge, hydrophobicity, etc., it is more likely that that site can tolerate variation in a functional polypeptide. The variant amino acid or DNA sequence can be at least 90%, at least 95%, at least 96%, at least 97%, at least 98%, at least 99%, or more, identical to a native or reference sequence, e.g., a nucleic acid encoding one of those amino acid sequences. The degree of homology (percent identity) between a native and a mutant sequence can be determined, for example, by comparing the two sequences using freely available computer programs commonly employed for this purpose on the world wide web. The variant amino acid or DNA sequence can be at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g., BLASTp or BLASTn (available on the world wide web at blast.ncbi.nlm.nih.gov), with default parameters set.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., the activity and/or specificity of a native or reference polypeptide is retained.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity of a native or reference polypeptide is retained. Conservative substitution tables providing functionally similar amino acids are well known in the art. Such conservatively modified variants are in addition to and do not exclude polymorphic variants, interspecies homologs, and alleles consistent with the disclosure.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser(S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu. Typically conservative substitutions for one another also include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

In some embodiments, a polypeptide can comprise one or more amino acid substitutions or modifications. In some embodiments, the substitutions and/or modifications can prevent or reduce proteolytic degradation and/or prolong half-life of the polypeptide in a subject. In some embodiments, a polypeptide can be modified by conjugating or fusing it to other polypeptide or polypeptide domains such as, by way of non-limiting example, transferrin (WO06096515A2), albumin (Yeh et al., 1992), growth hormone (US2003104578AA); cellulose (Levy and Shoseyov, 2002); and/or Fc fragments (Ashkenazi and Chamow, 1997). The references in the foregoing paragraph are incorporated by reference herein in their entireties.

In some embodiments, a polypeptide as described herein can comprise at least one peptide bond replacement. A polypeptide as described herein can comprise one type of peptide bond replacement or multiple types of peptide bond replacements, e.g., 2 types, 3 types, 4 types, 5 types, or more types of peptide bond replacements. Non-limiting examples of peptide bond replacements include urea, thiourea, carbamate, sulfonyl urea, trifluoroethylamine, ortho-(aminoalkyl)-phenylacetic acid, para-(aminoalkyl)-phenylacetic acid, meta-(aminoalkyl)-phenylacetic acid, thioamide, tetrazole, boronic ester, olefinic group, and derivatives thereof.

In some embodiments, a polypeptide as described herein can comprise naturally occurring amino acids commonly found in polypeptides and/or proteins produced by living organisms, e.g. Ala (A), Val (V), Leu (L), Ile (I), Pro (P), Phe (F), Trp (W), Met (M), Gly (G), Ser(S), Thr (T), Cys (C), Tyr (Y), Asn (N), Gln (Q), Asp (D), Glu (E), Lys (K), Arg (R), and His (H). In some embodiments, a polypeptide as described herein can comprise alternative amino acids. Non-limiting examples of alternative amino acids include, D-amino acids; beta-amino acids; homocysteine, phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine (3-mercapto-D-valine), ornithine, citruline, alpha-methyl-alanine, para-benzoylphenylalanine, para-amino phenylalanine, p-fluorophenylalanine, phenylglycine, propargylglycine, sarcosine, and tert-butylglycine), diaminobutyric acid, 7-hydroxy-tetrahydroisoquinoline carboxylic acid, naphthylalanine, biphenylalanine, cyclohexylalanine, amino-isobutyric acid, norvaline, norleucine, tert-leucine, tetrahydroisoquinoline carboxylic acid, pipecolic acid, phenylglycine, homophenylalanine, cyclohexylglycine, dehydroleucine, 2,2-diethylglycine, 1-amino-1-cyclopentanecarboxylic acid, 1-amino-1-cyclohexanecarboxylic acid, amino-benzoic acid, amino-naphthoic acid, gamma-aminobutyric acid, difluorophenylalanine, nipecotic acid, alpha-amino butyric acid, thienyl-alanine, t-butylglycine, trifluorovaline; hexafluoroleucine; fluorinated analogs; azide-modified amino acids; alkyne-modified amino acids; cyano-modified amino acids; and derivatives thereof.

In some embodiments, a polypeptide can be modified, e.g., by addition of a moiety to one or more of the amino acids that together comprise the peptide. In some embodiments, a polypeptide as described herein can comprise one or more moiety molecules, e.g., 1 or more moiety molecules per polypeptide, 2 or more moiety molecules per polypeptide, 5 or more moiety molecules per polypeptide, 10 or more moiety molecules per polypeptide or more moiety molecules per polypeptide. In some embodiments, a polypeptide as described herein can comprise one more types of modifications and/or moieties, e.g. 1 type of modification, 2 types of modifications, 3 types of modifications or more types of modifications. Non-limiting examples of modifications and/or moieties include PEGylation; glycosylation; HESylation; ELPylation; lipidation; acetylation; amidation; end-capping modifications; cyano groups; phosphorylation; albumin, and cyclization. In some embodiments, an end-capping modification can comprise acetylation at the N-terminus, N-terminal acylation, and N-terminal formylation. In some embodiments, an end-capping modification can comprise amidation at the C-terminus, introduction of C-terminal alcohol, aldehyde, ester, and thioester moieties. The half-life of a polypeptide can be increased by the addition of moieties, e.g., PEG, albumin, or other fusion partners (e.g. Fc fragment of an immunoglobin).

Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established. Alterations of the original amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Khudyakov et al. “Artificial DNA: Methods and Applications” CRC Press, 2002; Braman “In Vitro Mutagenesis Protocols” Springer, 2004; and Rapley “The Nucleic Acid Protocols Handbook” Springer 2000; which are herein incorporated by reference in their entireties. In some embodiments, a polypeptide as described herein can be chemically synthesized and mutations can be incorporated as part of the chemical synthesis process.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

Nucleic acids may further include modified DNA or RNA, for example DNA or RNA that has been methylated, or RNA that has been subject to post-translational modification, for example 5′-capping with 7-methylguanosine, 3′-processing such as cleavage and polyadenylation, and splicing. Nucleic acids may also include synthetic nucleic acids (XNA), such as hexitol nucleic acid (HNA), cyclohexene nucleic acid (CeNA), threose nucleic acid (TNA), glycerol nucleic acid (GNA), locked nucleic acid (LNA) and peptide nucleic acid (PNA).

According to the present invention, homology to the nucleic acid sequences described herein is not limited simply to 100% sequence identity. In this regard, the term “substantially similar”, relating to two sequences, means that the sequences have at least 70%, 80%, 90%, 95% or 100% similarity. Likewise, the term “substantially complementary”, relating to two sequences, means that the sequences are completely complementary, or that at least 70%, 80%, 90%, 95% or 99% of the bases are complementary. That is, mismatches can occur between the bases of the sequences which are intended to hybridise, which can occur between at least 1%, 5%, 10%, 20% or up to 30% of the bases. However, it may be desired in some cases to distinguish between two sequences which can hybridise to each other but contain some mismatches—an “inexact match”, “imperfect match”, or “inexact complementarity”—and two sequences which can hybridise to each other with no mismatches—an “exact match”, “perfect match”, or “exact complementarity”. Further, possible degrees of mismatch are considered.

The term ‘target sequence’ refers to a sequence comprised within a mRNA sequence, such as within an untranslated region (UTR), that is targeted for binding by a specified miRNA. Binding occurs by way of nucleic acid hybridisation between complementary base pairs comprised within the miRNA and the corresponding target sequence. The binding interaction may be optimised such that no mismatches between the specified miRNA and the target sequence occur, or mismatches are limited to no more than a single base pair mismatch across the length of the target sequence. In an embodiment of the invention a single base mismatch is limited to the 5′ or 3′ end of the target sequence. Optimised sequences can also be described as being perfectly matched to the target miRNA that is present in the cell and may differ from the wild type binding sequence by two or more base pairs. Wild type sequences that comprise more than two naturally occurring mismatches are deemed to be un-perfectly or im-perfectly matched to the corresponding complementary miRNA sequence.

The term ‘operatively linked’, when applied to nucleic acid sequences, for example in an expression construct, indicates that the sequences are arranged so that they function cooperatively in order to achieve their intended purposes. By way of example, in a DNA vector a promoter sequence allows for initiation of transcription that proceeds through a linked coding sequence as far as a termination sequence. In the case of RNA sequences, one or more untranslated regions (UTRs) may be arranged in relation to a linked polypeptide coding sequence referred to as an open reading frame (ORF). A given mRNA as disclosed herein may comprise more than one ORFs, a so-called polycistronic RNA. An mRNA may encode more than one polypeptide, and may as a result include cleavage sites or other sequences necessary to result in the production of multiple functional products, as known in the art. The control elements, e.g., a promoter, need not be contiguous with the coding sequence, so long as they function to direct the expression thereof. Thus, for example, intervening untranslated yet transcribed sequences can be present between a promoter sequence and the coding sequence, and the promoter sequence can still be considered “operably linked” to the coding sequence.

A UTR may be located 5′ or 3′ in relation to an operatively linked coding sequence ORF. UTRs may comprise sequences typically found in mRNA sequences found in nature, such as any one or more of: Kozak consensus sequences, initiation codons, cis-acting translational regulatory elements, cap-independent translation initiator sequences, poly-A tails, internal ribosome entry sites (IRES), structures regulating mRNA stability and/or longevity, sequences directing the localization of the mRNA, and so on. An mRNA may comprise multiple UTRs that are the same or different. The one or more UTRs may comprise or be located proximate or adjacent to an OPS. UTRs may comprise linear sequences that provide translational or stability control over the mRNA, such as Kozak sequences, or they may also comprise one or more sequences that promote the formation of localized secondary structure, particularly within a 5′ UTR. In one embodiment of the invention, a 5′ UTR that has a lower-than-average GC content may be utilized to promote efficient translation of the mRNA.

The term ‘expressing a polypeptide’ in the context of the present invention refers to production of a polypeptide for which the polynucleotide sequences described herein code. Typically, this involves translation of the supplied mRNA sequence—i.e., the ORF—by the ribosomal machinery of the cell to which the sequence is delivered. The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

In some embodiments, the expression of a biomarker(s), target(s), or gene/polypeptide described herein is/are tissue-specific. In some embodiments, the expression of a biomarker(s), target(s), or gene/polypeptide described herein is/are global. In some embodiments, the expression of a biomarker(s), target(s), or gene/polypeptide described herein is systemic.

The term ‘gene product’ as used herein refers to the peptide or polypeptide encoded by at least one coding sequence or Open Reading Frame (ORF) comprised within an mRNA construct of the invention as described herein. A polycistronic mRNA construct may be used, which results in the production of multiple gene products encoded by multiple ORFs located on the same polynucleic strand. It will be appreciated that multiple ORFs may lead to the production in situ of a variety of products—e.g. proteins, peptides or polypeptides—that may cooperate functionally, or may form complexes and/or multimeric proteins with diverse biological and potentially therapeutic effects. The gene product encoded by the mRNA is typically a peptide, polypeptide or protein. Where a particular protein consists of more than one subunit, the mRNA may code for one or more than one subunit within one or more ORFs. In alternative embodiments, a first mRNA may code for a first subunit, whilst a second co-administered mRNA may code for a second subunit that, when translated in situ, leads to assembly of a multi-subunit protein gene product. Translation of the gene product within the target cell allows for localized post-translational modification appropriate to the cell type to be applied. Such modifications may regulate folding, localization, interactions, degradation, and activity of the gene product. Typical post translational modifications may include cleavage, refolding and/or chemical modification such as methylation, acetylation or glycosylation.

“Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

In some embodiments, the methods described herein relate to measuring, detecting, or determining the level of at least one marker. As used herein, the term “detecting” or “measuring” refers to observing a signal from, e.g., a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell is typically still referred to as “engineered” even though the actual manipulation was performed on a prior entity.

In some embodiments of any of the aspects, the mRNA construct and/or vector described herein is exogenous. In some embodiments of any of the aspects, the mRNA construct and/or vector described herein is ectopic. In some embodiments of any of the aspects, the mRNA construct/and/or vector described herein is not endogenous.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes a substance, such as a polypeptide or nucleic acid that is not naturally found or expressed in a given cell in its natural environment.

In some embodiments, a nucleic acid as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g., an infectious disease. The term “treating” includes reducing or alleviating at least one adverse effect or symptom of a condition, disease or disorder associated with an infectious disease. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to an infection or infectious disease. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g., a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A method for inducing an immune response in an immune distinct subject, the method comprising administering to the subject one or more compositions comprising:
   a) a first cytokine mRNA construct comprising a first open reading frame (ORF), wherein the first ORF encodes a proinflammatory cytokine; and
   b) optionally, one or more of:
      i) a first antigen mRNA construct comprising a second open reading frame (ORF), wherein the second ORF encodes an antigen; and
      ii) an antigen polypeptide, antigen molecule, or killed or attenuated pathogenic agent.
2. The method of paragraph 1, wherein the immune response comprises an increase in IL-12 in the subject.
3. The method of paragraph 1, wherein the immune response comprises an increase in the active IL-12 heterodimer (referred to as ‘p70’) in the subject.
4. The method of paragraph 1, wherein the immune response comprises an increase in Ig levels in the subject.
5. The method of paragraph 4, wherein the Ig is IgG2, IgG3, or IgG2a.
6. The method of paragraph 5, wherein the IgG2a is IgG2a that specifically binds the antigen.
7. The method of paragraph 1, wherein the immune response comprises a CD4+ T cell response in the subject.
8. The method of paragraph 1, wherein the immune response comprises a CD8+ T cell response in the subject.
9. The method of paragraph 1, wherein the immune response comprises a NK cell response in the subject.
10. The method of paragraph 1, wherein the immune response comprises a Th1 response in the subject.
11. The method of paragraph 1, wherein the immune response stimulates the production of an interferon gamma (IFNγ) response from T cells in the subject.
12. The method of paragraph 1, wherein the immune response initiates phagocytosis via the Fc region of each IgG subclass via improved affinity for phagocyte membrane Fc-gamma-receptors (FcγR)
13. The method of paragraph 1, wherein the immune response comprises immunization of the subject against the antigen or an organism comprising the antigen.
14. A method for treating or preventing a disease in an immune distinct subject, the method comprising administering to the subject one or more compositions comprising:
   a) a first cytokine mRNA construct comprising a first open reading frame (ORF), wherein the first ORF encodes a proinflammatory cytokine; and
   b) optionally, one or more of:
      i) a first antigen mRNA construct comprising a second open reading frame (ORF), wherein the second ORF encodes an antigen; and
      ii) an antigen polypeptide, antigen molecule, or killed or attenuated pathogenic agent.
15. A method for immunizing an immune distinct subject, the method comprising administering to the subject one or more compositions comprising:
   a) a first cytokine mRNA construct comprising a first open reading frame (ORF), wherein the first ORF encodes a proinflammatory cytokine; and
   b) optionally, one or more of:
      i) a first antigen mRNA construct comprising a second open reading frame (ORF), wherein the second ORF encodes an antigen; and
      ii) an antigen polypeptide, antigen molecule, or killed or attenuated pathogenic agent.
16. The method of any of the preceding paragraphs, wherein the immune distinct subject is a subject with immunosenescence.
17. The method of any of the preceding paragraphs, wherein the immune distinct subject and/or subject with immunosenescence is a subject of 55 years of age or older.
18. The method of any of the preceding paragraphs, wherein the immune distinct subject and/or subject with immunosenescence is a subject of 60 years of age or older.
19. The method of any of the preceding paragraphs, wherein the immune distinct subject and/or subject with immunosenescence is a subject of 65 years of age or older.
20. The method of any of the preceding paragraphs, wherein the immune distinct subject and/or subject with immunosenescence is a subject of 70 years of age or older.
21. The method of any of the preceding paragraphs, wherein the immune distinct subject and/or subject with immunosenescence is a subject of 75 years of age or older.
22. The method of any of the preceding paragraphs, wherein the immune distinct subject and/or subject with immunosenescence is a subject who has or is determined to have a reduced TNF response to immune stimuli.
23. The method of any of the preceding paragraphs, wherein the immune distinct subject and/or subject with immunosenescence is a subject who has or is determined to have a reduced IL-12 response to immune stimuli.
24. The method of paragraph 23, wherein the immune stimuli is lipopolysaccharide (LPS).
25. The method of any one of paragraphs 1-15, wherein the immune distinct subject is an infant.
26. The method of paragraph 25, wherein the immune distinct subject and/or infant is 2 years of age or younger.
27. The method of paragraph 25, wherein the immune distinct subject and/or infant is 1 year of age or younger.
28. The method of paragraph 25, wherein the immune distinct subject and/or infant is 28 days of age or younger.
29. The method of paragraph 25, wherein the immune distinct subject and/or infant is born preterm.
30. The method of any of the preceding paragraphs, wherein the immune distinct subject is immunocompromised, has an HIV infection, has AIDS, has received a transplant, is undergoing immunosuppression, is immunosuppressed, has an infection, is diabetic, has an IgG subclass deficiency, has a substance abuse disorder, or is obese.
31. The method of any of the preceding paragraphs, wherein the subject is a subject in a high density living environment.
32. The method of paragraph 31, wherein the high density living environment is an assisted living facility; a nursing home, a dormitory, or a hospital.
33. The method of any of the preceding paragraphs, wherein the subject is a subject who is:
   a) at least 55 years of age; and
   b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
34. The method of any of the preceding paragraphs, wherein the subject is a subject who is:
   a) at least 55 years of age; and
   b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, and/or is obese.
35. The method of any of paragraphs 33-34, wherein the subject is at least 60 years of age or older.
36. The method of any of paragraphs 33-34, wherein the subject is at least 65 years of age or older.
37. The method of any of paragraphs 33-34, wherein the subject is at least 70 years of age or older.
38. The method of any of paragraphs 33-34, wherein the subject is at least 75 years of age or older.
39. The method of any of the preceding paragraphs, wherein the composition comprises at least 5× less of the antigen than is required to induce an immune response in the absence of the first cytokine mRNA.
40. The method of any of the preceding paragraphs, wherein the composition comprises at least 10× less of the antigen than is required to induce an immune response in the absence of the first cytokine mRNA.
41. The method of any of the preceding paragraphs, wherein the composition comprises at least 20× less of the antigen than is required to induce an immune response in the absence of the first cytokine mRNA.
42. The method of any of the preceding paragraphs, wherein the composition comprises at least 50× less of the antigen than is required to induce an immune response in the absence of the first cytokine mRNA.
43. The method of any of the preceding paragraphs, wherein the composition comprises at least 100× less of the antigen than is required to induce an immune response in the absence of the first cytokine mRNA.
44. The method of any of the preceding paragraphs, wherein the method comprises administering each of the one or more compositions to the subject no more frequently than once per year.
45. The method of any of the preceding paragraphs, wherein the method comprises administering each of the one or more compositions to the subject no more frequently than once every 2 years.
46. The method of any of the preceding paragraphs, wherein the method comprises administering each of the one or more compositions to the subject no more frequently than once every 3 years.
47. The method of any of the preceding paragraphs, wherein the method comprises administering each of the one or more compositions to the subject no more frequently than once every 4 years.
48. The method of any of the preceding paragraphs, wherein the method comprises administering each of the one or more compositions to the subject no more frequently than once every 5 years.
49. The method of any of the preceding paragraphs, wherein the first antigen mRNA construct antigen is an antigen of a first infectious organism and the method comprises administering a composition comprising any antigen from the first infectious organism to the subject no more frequently than once per year.
50. The method of any of the preceding paragraphs, wherein the first antigen mRNA construct antigen is an antigen of a first infectious organism and the method comprises administering a composition comprising any antigen from the first infectious organism to the subject no more frequently than once every 2 years.
51. The method of any of the preceding paragraphs, wherein the first antigen mRNA construct antigen is an antigen of a first infectious organism and the method comprises administering a composition comprising any antigen from the first infectious organism to the subject no more frequently than once every 3 years.
52. The method of any of the preceding paragraphs, wherein the first antigen mRNA construct antigen is an antigen of a first infectious organism and the method comprises administering a composition comprising any antigen from the first infectious organism to the subject no more frequently than once every 4 years.
53. The method of any of the preceding paragraphs, wherein the first antigen mRNA construct antigen is an antigen of a first infectious organism and the method comprises administering a composition comprising any antigen from the first infectious organism to the subject no more frequently than once every 5 years.
54. The method of any of the preceding paragraphs, wherein the proinflammatory cytokine is selected from the group consisting of: IL-12; IL-2; IL-4; IL-5; IL-6; IL-8; IL-10; IL-13; IL-27; IL-1 beta; TGFbeta; IFNγ; IFNα; IFNI3; TNFα; CCL2; CCL3; CCL4; CCL5; CCL8; CXCL12; GM-CSF; and a subunit, dimer, heterodimer, derivative, fragment, agonist or homologue thereof.
55. The method of any of the preceding paragraphs, wherein the proinflammatory cytokine is IL-12 or a subunit, dimer, heterodimer, derivative, fragment, agonist or homologue thereof.
56. The method of paragraph 55, wherein the first ORF comprises a sequence at least 90% identical to SEQ ID NO: 59.
57. The method of any of the preceding paragraphs, wherein the proinflammatory cytokine is IL-12 or a subunit, of human, and other mammalian homology.
58. The method of any of the preceding paragraphs, wherein the one or more compositions further comprise one or more further cytokine mRNA constructs, each comprising a further open reading frame (ORF), wherein each further ORF encodes a proinflammatory cytokine distinct from the proinflammatory cytokine encoded by the first ORF.
59. The method of paragraph 58, wherein the composition comprises 1-9 further cytokine mRNA constructs.
60. The method of any of the preceding paragraphs, wherein the first cytokine mRNA construct further comprises one or more further open reading frames (ORFs), wherein each further ORF encodes a proinflammatory cytokine distinct from the proinflammatory cytokine encoded by the first ORF.
61. The method of paragraph 60, wherein the first cytokine mRNA construct comprises 1-9 further ORFs encoding a proinflammatory cytokine distinct from the proinflammatory cytokine encoded by the first ORF.
62. The method of any of paragraphs 58-61, wherein the first ORF encodes IL-12 or a subunit, derivative, fragment, agonist or homologue thereof and the one or more further ORFs encode IL-2; IL-4; IL-5; IL-6; IL-8; IL-10; IL-13; IL-27; IL-1β; TGFβ; IFNγ; IFNα; IFNβ; TNFα; CCL2; CCL3; CCL4; CCL5; CCL8; CXCL12; GM-CSF; or a subunit, derivative, fragment, agonist or homologue thereof.
63. The method of any of the preceding paragraphs, wherein the composition further comprises one or more further antigen mRNA constructs, each comprising a further open reading frame (ORF), wherein each further ORF encodes an antigen distinct from the antigen encoded by the second ORF.
64. The method of paragraph 63, wherein the composition comprises 1-9 further antigen mRNA constructs.
65. The method of any of the preceding paragraphs, wherein the antigen mRNA construct further comprises one or more further open reading frames (ORFs), wherein each further ORF encodes an antigen distinct from the antigen encoded by the second ORF.
66. The method of paragraph 65, wherein the composition comprises 1-9 further ORFs encoding an antigen distinct from the antigen encoded by the second ORF.
67. The method of any one of paragraphs 63-66, wherein the composition comprises a plurality of ORFs encoding a plurality of antigens, and the plurality of antigens comprises multiple antigens from a first organism.
68. The method of any one of paragraphs 63-67, wherein the composition comprises a plurality of ORFs encoding a plurality of antigens, and the plurality of antigens comprises one or more antigens from a first organism and one or more antigens from one or more further organisms.
69. The method of paragraph 68, wherein the composition comprises a plurality of ORFs encoding a plurality of antigens, and the plurality of antigens comprises one or more antigens from a coronavirus and one or more antigens from an influenza virus.
70. The method of paragraph 68, wherein the composition comprises a plurality of ORFs encoding a plurality of antigens, and the plurality of antigens comprises one or more spike protein antigens from a coronavirus and one or more antigens from an influenza virus.
71. The method of any of the preceding paragraphs, wherein the antigen is an antigen of an infectious organism and whereby transmission of the infectious organism to or by the subject is reduced as compared to administration of a composition not comprising the cytokine mRNA construct.
72. The method of any of the preceding paragraphs, wherein the antigen is a pathogenic microbial protein or an epitope containing fragment thereof.
73. The method of paragraph 72, wherein the pathogenic microbial protein is selected from the group consisting of: a viral protein; a bacterial protein; a fungal protein; a parasite protein; and a prion.
74. The method of any of the preceding paragraphs, wherein the antigen comprises a viral protein or an epitope containing fragment thereof.
75. The method of any of paragraph 74, wherein the antigen comprises a coronavirus spike protein.
76. The method of any of paragraph 74, wherein the antigen comprises a coronavirus receptor binding domain (RBD) protein.
77. The method of any of paragraph 74, wherein the antigen comprises a variant coronavirus spike protein.
78. The method of any of paragraph 74, wherein the antigen comprises a variant coronavirus receptor binding domain protein.
79. The method of any one of paragraphs 75-78, wherein the coronavirus spike protein is a MERS-COV spike or RBD protein.
80. The method of any one of paragraphs 75-78, wherein the coronavirus spike protein is a SARS-COV-1 spike or RBD protein.
81. The method of any one of paragraphs 75-78, wherein the coronavirus spike protein is a SARS-COV-2 spike or RBD protein.
82. The method of paragraph 74, wherein the antigen comprises an influenza protein or a variant thereof, or an epitope containing fragment thereof.
83. The method of paragraph 82, wherein the influenza protein is selected from the group consisting of a hemagglutinin, a neuraminidase, a matrix-2 and/or a nucleoprotein.
84. The method of paragraph 82, wherein the influenza protein is selected from type A influenza, a type B influenza, or a subtype of type A influenza of H1, H2, H3, H4, H5, H6, H7, H8, H9, H10, H11, H12, H13, H14, H15 or H16.
85. The method of paragraph 74, wherein the antigen comprises a respiratory syncytial virus (RSV) protein, or a variant thereof, or an epitope containing fragment thereof.
86. The method of paragraph 85, wherein the protein of the respiratory syncytial virus is the F glycoprotein or the G glycoprotein.
87. The method of paragraph 74, wherein the antigen comprises a Human Immunodeficiency Virus (HIV) protein or an epitope containing fragment thereof.
88. The method of paragraph 87, wherein the HIV protein is the glycoprotein 120 neutralizing epitope or glycoprotein 145.
89. The method of paragraph 72, wherein the antigen comprises a protein from the Mycobacterium tuberculosis bacterium or an epitope containing fragment thereof.
90. The method of paragraph 89, wherein the protein from the Mycobacterium tuberculosis bacterium is selected from ESAT-6, Ag85B, TB10.4, Rv2626 and/or RpfD-B.
91. The method of any of the preceding paragraphs, wherein one or more of the first, second, or further ORFs is operatively linked to at least one untranslated region (UTR), wherein each UTR comprises at least a first organ protection sequence (OPS), wherein each OPS comprises at least two micro-RNA (miRNA) target sequences, and wherein each of the at least two miRNA target sequences are optimised to hybridise with a corresponding miRNA sequence.
92. The method of paragraph 91, wherein each ORF of the composition is operatively linked to a UTR comprising at least one OPS.
93. The method of any one of paragraphs 91-92, wherein each OPS of the composition independently comprises at least three, at least four, or at least five miRNA target sequences.
94. The method of any one of paragraphs 91-92, wherein each OPS of the composition independently comprises at least three miRNA target sequences which are all different from each other.
95. The method of any one of paragraphs 91-94, wherein the first and second ORFs are operatively linked to the same OPS.
96. The method of any one of paragraphs 91-94, wherein the first and second ORFs are operatively linked to different OPSs.
97. The method of any one of paragraphs 91-96, wherein the OPS linked to the first ORF and the OPS linked to the second ORF comprise the same miRNA target sequences.
98. The method of any one of paragraphs 91-96, wherein the OPS linked to the first ORF and the OPS linked to the second ORF each comprise at least one miRNA target sequence not comprised by the other OPS.
99. The method of any one of paragraphs 91-96, wherein the OPS linked to the first ORF and the OPS linked to the second ORF each comprise at least three miRNA target sequences not comprised by the other OPS.
100. The method of any one of paragraphs 91-99, wherein the OPS operatively linked to the second ORF comprises miRNA sequences selected to protect one or more organs or tissues selected from the group consisting of muscle, liver, brain, breast, endothelium, pancreas, colon, kidney, lungs, spleen and skin, heart, gastrointestinal organs, reproductive organs, and esophagus.
101. The method of any one of paragraphs 91-100, wherein the OPS operatively linked to the first ORF comprises miRNA sequences selected to protect one or more organs or tissues selected from the group consisting muscle, liver, brain, breast, endothelium, pancreas, colon, kidney, lungs, spleen and skin.
102. The method of any one of paragraphs 91-100, wherein the OPS operatively linked to the first ORF comprises miRNA sequences selected to protect one or more organs selected from the group consisting of muscle, liver, kidney, lungs, spleen, skin, heart, gastrointestinal organs, reproductive organs, and esophagus.
103. The method of any one of paragraphs 91-102, wherein one or more of the OPS independently comprises:
   a) at least two miRNA target sequences selected from one or more sequences that bind to: miRNA-122; miRNA-125; miRNA-199; miRNA-124a; miRNA-126; miRNA-98; Let7 miRNA family; miRNA-375; miRNA-141; miRNA-142; miRNA-148a/b; miRNA-143; miRNA-145; miRNA-194; miRNA-200c; miRNA-203a; miRNA-205; miRNA-1; miRNA-133a; miRNA-206; miRNA-34a; miRNA-192; miRNA-194; miRNA-204; miRNA-215; miRNA-30 family; miRNA-877; miRNA-4300; miRNA-4720; and/or miRNA-6761;
   b) sequences selected from one or more of SEQ ID NOs: 44-57;
   c) at least two miRNA target sequences selected from sequences capable of binding with miRNA-1, miRNA133a, miRNA206, miRNA-122, miRNA203a, miRNA205, miRNA200c, miRNA30a, and/or let7a/b;
   d) at least two miRNA target sequences selected from one or more sequences that bind to: miRNA-1, miRNA-122, miRNA-30a, miRNA-203a, let7b, miRNA-126, and/or miRNA-192;
   e) at least two miRNA target sequences selected from sequences capable of binding with miRNA-1, miRNA-122, miR-30a and/or miR-203a;
   f) miRNA target sequences capable of binding with miRNA-1, miRNA-122, miRNA-30a and miRNA-203a;
   g) miRNA target sequences capable of binding with let7b, miRNA-126, and miRNA-30a;
   h) miRNA target sequences capable of binding with miRNA-122, miRNA-192, and miRNA-30a; or
   i) miRNA target sequences capable of binding with miRNA-192, miRNA-30a, and miRNA-124, and two miRNA target sequences capable of binding with miRNA 122.
104. The method of any one of paragraphs 91-103, wherein the OPS operatively linked to
   the second ORF comprises miRNA target sequences capable of binding with miRNA-1, miRNA-122, miR-30a and/or miR-203a; and
   the OPS operatively linked to the first ORF comprises miRNA target sequences capable of binding with miRNA-122, miRNA-126, miRNA-192, and/or miRNA 30a.
105. The method of any of the preceding paragraphs, wherein the administration is intravenous, subcutaneous, intramuscular, intranasal, intra-arterial, or via inhalation.
106. The method of any of the preceding paragraphs, wherein the first, second, and/or further mRNA constructs are comprised within or adsorbed to an in vivo delivery composition.
107. The method of paragraph 106, wherein the delivery composition comprises delivery vectors selected from the group consisting of: a particle, such as a polymeric particle; a liposome; a lipidoid particle; and a viral vector.
108. The method of any of the preceding paragraphs, wherein the disease is caused by a coronavirus, an intracellular pathogen, a latent infection, an active infection, an influenza virus, respiratory syncytial virus (RSV), human immunodeficiency virus (HIV), varicella zoster virus (VZV), or Mycobacterium tuberculosis; and/or one or more of the antigens are a coronavirus, an intracellular pathogen, a latent infection, an active infection, an influenza virus, respiratory syncytial virus (RSV), human immunodeficiency virus (HIV), varicella zoster virus (VZV), plasmodium (Malaria), Streptococcus pneumoniae, Streptococcus pyogenes, Yersinia pestis, Haemophilus influenzae, Staphylococcus aureus, Pseudomonas aeruginosa, Bordetella pertussis, Ebola virus, Lassa virus, Middle East Respiratory Syndrome coronavirus, SARS-COV-1, SARS-COV-2, SARS-COV-2 variants of concerns, Marburg virus, Nipah virus, Rift Valley Fever virus, Chikungunya virus or Mycobacterium tuberculosis antigen.
109. The method of any of the preceding paragraphs, wherein the disease is caused by a coronavirus and/or one or more of the antigens are a coronavirus antigen.
110. The method of paragraph 109, wherein the coronavirus is MERS-COV virus.
111. The method of paragraph 109, wherein the coronavirus is SARS-COV-1 virus.
112. The method of paragraph 109, wherein the coronavirus is SARS-COV-2 virus.

In some embodiments, the present technology may be defined in any of the following numbered paragraphs:
1. A method for inducing an immune response in an immune distinct subject, the method comprising administering to the subject one or more compositions comprising:
   a) a first cytokine mRNA construct comprising a first open reading frame (ORF), wherein the first ORF encodes a proinflammatory cytokine; and
   b) optionally, one or more of:
      i) a first antigen mRNA construct comprising a second open reading frame (ORF), wherein the second ORF encodes an antigen; and
      ii) an antigen polypeptide, antigen molecule, or killed or attenuated pathogenic agent.
2. The method of paragraph 1, wherein the immune response comprises an increase in IL-12 in the subject.
3. The method of paragraph 1, wherein the immune response comprises an increase in the active IL-12 heterodimer (referred to as ‘p70’) in the subject.
4. The method of paragraph 1, wherein the immune response comprises an increase in Ig levels in the subject.
5. The method of paragraph 4, wherein the Ig is IgG2, IgG3, or IgG2a
6. The method of paragraph 5, wherein the IgG2a is IgG2a that specifically binds the antigen.
7. The method of paragraph 1, wherein the immune response comprises a CD4+ T cell response in the subject.
8. The method of paragraph 1, wherein the immune response comprises a CD8+ T cell response in the subject.
9. The method of paragraph 1, wherein the immune response comprises a NK cell response in the subject.
10. The method of paragraph 1, wherein the immune response comprises a Th1 response in the subject.
11. The method of paragraph 1, wherein the immune response stimulates the production of an interferon gamma (IFNγ) response from T cells in the subject.
12. The method of paragraph 1, wherein the immune response initiates phagocytosis via the Fc region of each IgG subclass via improved affinity for phagocyte membrane Fc-gamma-receptors (FcγR)
13. The method of paragraph 1, wherein the immune response comprises immunization of the subject against the antigen or an organism comprising the antigen.
14. A method for treating or preventing a disease in an immune distinct subject, the method comprising administering to the subject one or more compositions comprising:
   a) a first cytokine mRNA construct comprising a first open reading frame (ORF), wherein the first ORF encodes a proinflammatory cytokine; and
   b) optionally, one or more of:
      i) a first antigen mRNA construct comprising a second open reading frame (ORF), wherein the second ORF encodes an antigen; and
      ii) an antigen polypeptide, antigen molecule, or killed or attenuated pathogenic agent.
15. A method for immunizing an immune distinct subject, the method comprising administering to the subject one or more compositions comprising:
   a) a first cytokine mRNA construct comprising a first open reading frame (ORF), wherein the first ORF encodes a proinflammatory cytokine; and
   b) optionally, one or more of:
      i) a first antigen mRNA construct comprising a second open reading frame (ORF), wherein the second ORF encodes an antigen; and
      ii) an antigen polypeptide, antigen molecule, or killed or attenuated pathogenic agent.
16. The method of any of the preceding paragraphs, wherein the immune distinct subject is a subject with immunosenescence.
17. The method of any of the preceding paragraphs, wherein the immune distinct subject and/or subject with immunosenescence is a subject of 55 years of age or older.
18. The method of any of the preceding paragraphs, wherein the immune distinct subject and/or subject with immunosenescence is a subject of 60 years of age or older.
19. The method of any of the preceding paragraphs, wherein the immune distinct subject and/or subject with immunosenescence is a subject of 65 years of age or older.
20. The method of any of the preceding paragraphs, wherein the immune distinct subject and/or subject with immunosenescence is a subject of 70 years of age or older.
21. The method of any of the preceding paragraphs, wherein the immune distinct subject and/or subject with immunosenescence is a subject of 75 years of age or older.
22. The method of any of the preceding paragraphs, wherein the immune distinct subject and/or subject with immunosenescence is a subject who has or is determined to have a reduced TNF response to immune stimuli.
23. The method of any of the preceding paragraphs, wherein the immune distinct subject and/or subject with immunosenescence is a subject who has or is determined to have a reduced IL-12 response to immune stimuli.
24. The method of paragraph 23, wherein the immune stimuli is lipopolysaccharide (LPS).
25. The method of any one of paragraphs 1-15, wherein the immune distinct subject is an infant.
26. The method of paragraph 25, wherein the immune distinct subject and/or infant is 2 years of age or younger.
27. The method of paragraph 25, wherein the immune distinct subject and/or infant is 1 year of age or younger.
28. The method of paragraph 25, wherein the immune distinct subject and/or infant is 28 days of age or younger.
29. The method of paragraph 25, wherein the immune distinct subject and/or infant is born preterm.
30. The method of any of the preceding paragraphs, wherein the immune distinct subject is immunocompromised, has an HIV infection, has AIDS, has received a transplant, is undergoing immunosuppression, is immunosuppressed, has an infection, is diabetic, has an IgG subclass deficiency, has a substance abuse disorder, or is obese.
31. The method of any of the preceding paragraphs, wherein the subject is a subject in a high density living environment.
32. The method of paragraph 31, wherein the high density living environment is an assisted living facility; a nursing home, a dormitory, or a hospital.
33. The method of any of the preceding paragraphs, wherein the subject is a subject who is:
   a) at least 55 years of age; and
   b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
34. The method of any of the preceding paragraphs, wherein the subject is a subject who is:
   a) at least 55 years of age; and
   b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, and/or is obese.
35. The method of any of paragraphs 33-34, wherein the subject is at least 60 years of age or older.
36. The method of any of paragraphs 33-34, wherein the subject is at least 65 years of age or older.
37. The method of any of paragraphs 33-34, wherein the subject is at least 70 years of age or older.
38. The method of any of paragraphs 33-34, wherein the subject is at least 75 years of age or older.
39. The method of any of the preceding paragraphs, wherein the subject is administered a) a dose of the first cytokine mRNA construct and b) a dose of the first antigen mRNA construct and/or the antigen polypeptide, antigen molecule, or killed or attenuated pathogenic agent, wherein the dose of the first cytokine mRNA construct is no more than 20% of the dose of the first antigen mRNA construct and/or the antigen polypeptide, antigen molecule, or killed or attenuated pathogenic agent by weight.
40. The method of any of the preceding paragraphs, wherein the subject is administered a) a dose of the first cytokine mRNA construct and b) a dose of the first antigen mRNA construct and/or the antigen polypeptide, antigen molecule, or killed or attenuated pathogenic agent, wherein the dose of the first cytokine mRNA construct is no more than 10% of the dose of the first antigen mRNA construct and/or the antigen polypeptide, antigen molecule, or killed or attenuated pathogenic agent by weight.
41. The method of any of the preceding paragraphs, wherein the subject is administered a) a dose of the first cytokine mRNA construct and b) a dose of the first antigen mRNA construct and/or the antigen polypeptide, antigen molecule, or killed or attenuated pathogenic agent, wherein the dose of the first cytokine mRNA construct is from 0.5% to 20% of the dose of the first antigen mRNA construct and/or the antigen polypeptide, antigen molecule, or killed or attenuated pathogenic agent by weight.
42. The method of any of the preceding paragraphs, wherein the subject is administered a) a dose of the first cytokine mRNA construct and b) a dose of the first antigen mRNA construct and/or the antigen polypeptide, antigen molecule, or killed or attenuated pathogenic agent, wherein the dose of the first cytokine mRNA construct is from 1% to 10% of the dose of the first antigen mRNA construct and/or the antigen polypeptide, antigen molecule, or killed or attenuated pathogenic agent by weight.
43. The method of any of the preceding paragraphs, wherein the subject is administered a) a dose of the first cytokine mRNA construct and b) a dose of the first antigen mRNA construct, wherein the dose of the first cytokine mRNA construct is no more than 20% of the dose of the first antigen mRNA construct by weight.
44. The method of any of the preceding paragraphs, wherein the subject is administered a) a dose of the first cytokine mRNA construct and b) a dose of the first antigen mRNA construct, wherein the dose of the first cytokine mRNA construct is no more than 10% of the dose of the first antigen mRNA construct by weight.
45. The method of any of the preceding paragraphs, wherein the subject is administered a) a dose of the first cytokine mRNA construct and b) a dose of the first antigen mRNA construct, wherein the dose of the first cytokine mRNA construct is from 0.5% to 20% of the dose of the first antigen mRNA construct by weight.
46. The method of any of the preceding paragraphs, wherein the subject is administered a) a dose of the first cytokine mRNA construct and b) a dose of the first antigen mRNA construct, wherein the dose of the first cytokine mRNA construct is from 1% to 10% of the dose of the first antigen mRNA construct by weight.
47. The method of any of the preceding paragraphs, wherein the subject is a human subject and is administered a dose of the first cytokine mRNA construct of from 0.10 μg to 10 μg.
48. The method of any of the preceding paragraphs, wherein the subject is a human subject and is administered a dose of the first cytokine mRNA construct of from 0.15 μg to 6 μg.
49. The method of any of the preceding paragraphs, wherein the subject is a human subject and is administered a dose of the first cytokine mRNA construct of from 0.3 μg to 3 μg.
50. The method of any of the preceding paragraphs, wherein the composition comprises at least 5× less of the antigen than is required to induce an immune response in the absence of the first cytokine mRNA.
51. The method of any of the preceding paragraphs, wherein the composition comprises at least 10× less of the antigen than is required to induce an immune response in the absence of the first cytokine mRNA.
52. The method of any of the preceding paragraphs, wherein the composition comprises at least 20× less of the antigen than is required to induce an immune response in the absence of the first cytokine mRNA.
53. The method of any of the preceding paragraphs, wherein the composition comprises at least 50× less of the antigen than is required to induce an immune response in the absence of the first cytokine mRNA.
54. The method of any of the preceding paragraphs, wherein the composition comprises at least 100× less of the antigen than is required to induce an immune response in the absence of the first cytokine mRNA.
55. The method of any of the preceding paragraphs, wherein the method comprises administering each of the one or more compositions to the subject no more frequently than once per year.
56. The method of any of the preceding paragraphs, wherein the method comprises administering each of the one or more compositions to the subject no more frequently than once every 2 years.
57. The method of any of the preceding paragraphs, wherein the method comprises administering each of the one or more compositions to the subject no more frequently than once every 3 years.
58. The method of any of the preceding paragraphs, wherein the method comprises administering each of the one or more compositions to the subject no more frequently than once every 4 years.
59. The method of any of the preceding paragraphs, wherein the method comprises administering each of the one or more compositions to the subject no more frequently than once every 5 years.
60. The method of any of the preceding paragraphs, wherein the first antigen mRNA construct antigen is an antigen of a first infectious organism and the method comprises administering a composition comprising any antigen from the first infectious organism to the subject no more frequently than once per year.
61. The method of any of the preceding paragraphs, wherein the first antigen mRNA construct antigen is an antigen of a first infectious organism and the method comprises administering a composition comprising any antigen from the first infectious organism to the subject no more frequently than once every 2 years.
62. The method of any of the preceding paragraphs, wherein the first antigen mRNA construct antigen is an antigen of a first infectious organism and the method comprises administering a composition comprising any antigen from the first infectious organism to the subject no more frequently than once every 3 years.
63. The method of any of the preceding paragraphs, wherein the first antigen mRNA construct antigen is an antigen of a first infectious organism and the method comprises administering a composition comprising any antigen from the first infectious organism to the subject no more frequently than once every 4 years.
64. The method of any of the preceding paragraphs, wherein the first antigen mRNA construct antigen is an antigen of a first infectious organism and the method comprises administering a composition comprising any antigen from the first infectious organism to the subject no more frequently than once every 5 years.
65. The method of any of the preceding paragraphs, wherein the proinflammatory cytokine is selected from the group consisting of: IL-12; IL-2; IL-4; IL-5; IL-6; IL-8; IL-10; IL-13; IL-27; IL-1 beta; TGFbeta; IFNy; IFNa; IFNI3; TNFa; CCL2; CCL3; CCL4; CCL5; CCL8; CXCL12; GM-CSF; and a subunit, dimer, heterodimer, derivative, fragment, agonist or homologue thereof.
66. The method of any of the preceding paragraphs, wherein the proinflammatory cytokine is IL-12 or a subunit, dimer, heterodimer, derivative, fragment, agonist or homologue thereof.
67. The method of paragraph 55, wherein the first ORF comprises a sequence at least 90% identical to SEQ ID NO: 59.
68. The method of any of the preceding paragraphs, wherein the proinflammatory cytokine is IL-12 or a subunit, of human, and other mammalian homology.
69. The method of any of the preceding paragraphs, wherein the one or more compositions further comprise one or more further cytokine mRNA constructs, each comprising a further open reading frame (ORF), wherein each further ORF encodes a proinflammatory cytokine distinct from the proinflammatory cytokine encoded by the first ORF.
70. The method of paragraph 58, wherein the composition comprises 1-9 further cytokine mRNA constructs.
71. The method of any of the preceding paragraphs, wherein the first cytokine mRNA construct further comprises one or more further open reading frames (ORFs), wherein each further ORF encodes a proinflammatory cytokine distinct from the proinflammatory cytokine encoded by the first ORF.
72. The method of paragraph 60, wherein the first cytokine mRNA construct comprises 1-9 further ORFs encoding a proinflammatory cytokine distinct from the proinflammatory cytokine encoded by the first ORF.
73. The method of any of paragraphs 58-61, wherein the first ORF encodes IL-12 or a subunit, derivative, fragment, agonist or homologue thereof and the one or more further ORFs encode IL-2; IL-4; IL-5; IL-6; IL-8; IL-10; IL-13; IL-27; IL-1β; TGFβ; IFNγ; IFNα; IFNβ; TNFα; CCL2; CCL3; CCL4; CCL5; CCL8; CXCL12; GM-CSF; or a subunit, derivative, fragment, agonist or homologue thereof.
74. The method of any of the preceding paragraphs, wherein the composition further comprises one or more further antigen mRNA constructs, each comprising a further open reading frame (ORF), wherein each further ORF encodes an antigen distinct from the antigen encoded by the second ORF.
75. The method of paragraph 63, wherein the composition comprises 1-9 further antigen mRNA constructs.
76. The method of any of the preceding paragraphs, wherein the antigen mRNA construct further comprises one or more further open reading frames (ORFs), wherein each further ORF encodes an antigen distinct from the antigen encoded by the second ORF.
77. The method of paragraph 65, wherein the composition comprises 1-9 further ORFs encoding an antigen distinct from the antigen encoded by the second ORF.
78. The method of any one of paragraphs 63-66, wherein the composition comprises a plurality of ORFs encoding a plurality of antigens, and the plurality of antigens comprises multiple antigens from a first organism.
79. The method of any one of paragraphs 63-67, wherein the composition comprises a plurality of ORFs encoding a plurality of antigens, and the plurality of antigens comprises one or more antigens from a first organism and one or more antigens from one or more further organisms.
80. The method of paragraph 68, wherein the composition comprises a plurality of ORFs encoding a plurality of antigens, and the plurality of antigens comprises one or more antigens from a coronavirus and one or more antigens from an influenza virus.
81. The method of paragraph 68, wherein the composition comprises a plurality of ORFs encoding a plurality of antigens, and the plurality of antigens comprises one or more spike protein antigens from a coronavirus and one or more antigens from an influenza virus.
82. The method of any of the preceding paragraphs, wherein the antigen is an antigen of an infectious organism and whereby transmission of the infectious organism to or by the subject is reduced as compared to administration of a composition not comprising the cytokine mRNA construct.
83. The method of any of the preceding paragraphs, wherein the antigen is a pathogenic microbial protein or an epitope containing fragment thereof.
84. The method of paragraph 72, wherein the pathogenic microbial protein is selected from the group consisting of: a viral protein; a bacterial protein; a fungal protein; a parasite protein; and a prion.
85. The method of any of the preceding paragraphs, wherein the antigen comprises a viral protein or an epitope containing fragment thereof.
86. The method of any of paragraph 74, wherein the antigen comprises a coronavirus spike protein.
87. The method of any of paragraph 74, wherein the antigen comprises a coronavirus receptor binding domain (RBD) protein.
88. The method of any of paragraph 74, wherein the antigen comprises a variant coronavirus spike protein.
89. The method of any of paragraph 74, wherein the antigen comprises a variant coronavirus receptor binding domain protein.
90. The method of any one of paragraphs 75-78, wherein the coronavirus spike protein is a MERS-COV spike or RBD protein.
91. The method of any one of paragraphs 75-78, wherein the coronavirus spike protein is a SARS-COV-1 spike or RBD protein.
92. The method of any one of paragraphs 75-78, wherein the coronavirus spike protein is a SARS-COV-2 spike or RBD protein.
93. The method of paragraph 74, wherein the antigen comprises an influenza protein or a variant thereof, or an epitope containing fragment thereof.
94. The method of paragraph 82, wherein the influenza protein is selected from the group consisting of a hemagglutinin, a neuraminidase, a matrix-2 and/or a nucleoprotein.
95. The method of paragraph 82, wherein the influenza protein is selected from type A influenza, a type B influenza, or a subtype of type A influenza of H1, H2, H3, H4, H5, H6, H7, H8, H9, H10, H11, H12, H13, H14, H15 or H16.
96. The method of paragraph 74, wherein the antigen comprises a respiratory syncytial virus (RSV) protein, or a variant thereof, or an epitope containing fragment thereof.
97. The method of paragraph 85, wherein the protein of the respiratory syncytial virus is the F glycoprotein or the G glycoprotein.
98. The method of paragraph 74, wherein the antigen comprises a Human Immunodeficiency Virus (HIV) protein or an epitope containing fragment thereof.
99. The method of paragraph 87, wherein the HIV protein is the glycoprotein 120 neutralizing epitope or glycoprotein 145.
100. The method of paragraph 72, wherein the antigen comprises a protein from the Mycobacterium tuberculosis bacterium or an epitope containing fragment thereof.
101. The method of paragraph 89, wherein the protein from the Mycobacterium tuberculosis bacterium is selected from ESAT-6, Ag85B, TB10.4, Rv2626 and/or RpfD-B.
102. The method of any of the preceding paragraphs, wherein one or more of the first, second, or further ORFs is operatively linked to at least one untranslated region (UTR), wherein each UTR comprises at least a first organ protection sequence (OPS), wherein each OPS comprises at least two micro-RNA (miRNA) target sequences, and wherein each of the at least two miRNA target sequences are optimised to hybridise with a corresponding miRNA sequence.
103. The method of paragraph 91, wherein each ORF of the composition is operatively linked to a UTR comprising at least one OPS.
104. The method of any one of paragraphs 91-92, wherein each OPS of the composition independently comprises at least three, at least four, or at least five miRNA target sequences.
105. The method of any one of paragraphs 91-92, wherein each OPS of the composition independently comprises at least three miRNA target sequences which are all different from each other.
106. The method of any one of paragraphs 91-94, wherein the first and second ORFs are operatively linked to the same OPS.
107. The method of any one of paragraphs 91-94, wherein the first and second ORFs are operatively linked to different OPSs.
108. The method of any one of paragraphs 91-96, wherein the OPS linked to the first ORF and the OPS linked to the second ORF comprise the same miRNA target sequences.
109. The method of any one of paragraphs 91-96, wherein the OPS linked to the first ORF and the OPS linked to the second ORF each comprise at least one miRNA target sequence not comprised by the other OPS.
110. The method of any one of paragraphs 91-96, wherein the OPS linked to the first ORF and the OPS linked to the second ORF each comprise at least three miRNA target sequences not comprised by the other OPS.
111. The method of any one of paragraphs 91-99, wherein the OPS operatively linked to the second ORF comprises miRNA sequences selected to protect one or more organs or tissues selected from the group consisting of muscle, liver, brain, breast, endothelium, pancreas, colon, kidney, lungs, spleen and skin, heart, gastrointestinal organs, reproductive organs, and esophagus.
112. The method of any one of paragraphs 91-100, wherein the OPS operatively linked to the first ORF comprises miRNA sequences selected to protect one or more organs or tissues selected from the group consisting muscle, liver, brain, breast, endothelium, pancreas, colon, kidney, lungs, spleen and skin.
113. The method of any one of paragraphs 91-100, wherein the OPS operatively linked to the first ORF comprises miRNA sequences selected to protect one or more organs selected from the group consisting of muscle, liver, kidney, lungs, spleen, skin, heart, gastrointestinal organs, reproductive organs, and esophagus.
114. The method of any one of paragraphs 91-102, wherein one or more of the OPS independently comprises:
   a) at least two miRNA target sequences selected from one or more sequences that bind to: miRNA-122; miRNA-125; miRNA-199; miRNA-124a; miRNA-126; miRNA-98; Let7 miRNA family; miRNA-375; miRNA-141; miRNA-142; miRNA-148a/b; miRNA-143; miRNA-145; miRNA-194; miRNA-200c; miRNA-203a; miRNA-205; miRNA-1; miRNA-133a; miRNA-206; miRNA-34a; miRNA-192; miRNA-194; miRNA-204; miRNA-215; miRNA-30 family; miRNA-877; miRNA-4300; miRNA-4720; and/or miRNA-6761;
   b) sequences selected from one or more of SEQ ID NOs: 44-57;
   c) at least two miRNA target sequences selected from sequences capable of binding with miRNA-1, miRNA133a, miRNA206, miRNA-122, miRNA203a, miRNA205, miRNA200c, miRNA30a, and/or let7a/b;
   d) at least two miRNA target sequences selected from one or more sequences that bind to: miRNA-1, miRNA-122, miRNA-30a, miRNA-203a, let7b, miRNA-126, and/or miRNA-192;
   e) at least two miRNA target sequences selected from sequences capable of binding with miRNA-1, miRNA-122, miR-30a and/or miR-203a;
   f) miRNA target sequences capable of binding with miRNA-1, miRNA-122, miRNA-30a and miRNA-203a;
   g) miRNA target sequences capable of binding with let7b, miRNA-126, and miRNA-30a;
   h) miRNA target sequences capable of binding with miRNA-122, miRNA-192, and miRNA-30a; or
   i) miRNA target sequences capable of binding with miRNA-192, miRNA-30a, and miRNA-124, and two miRNA target sequences capable of binding with miRNA 122.
115. The method of any one of paragraphs 91-103, wherein the OPS operatively linked to the second ORF comprises miRNA target sequences capable of binding with miRNA-1, miRNA-122, miR-30a and/or miR-203a; and the OPS operatively linked to the first ORF comprises miRNA target sequences capable of binding with miRNA-122, miRNA-126, miRNA-192, and/or miRNA 30a.
116. The method of any of the preceding paragraphs, wherein the administration is intravenous, subcutaneous, intramuscular, intranasal, intra-arterial, or via inhalation.
117. The method of any of the preceding paragraphs, wherein the first, second, and/or further mRNA constructs are comprised within or adsorbed to an in vivo delivery composition.
118. The method of paragraph 106, wherein the delivery composition comprises delivery vectors selected from the group consisting of: a particle, such as a polymeric particle; a liposome; a lipidoid particle; and a viral vector.
119. The method of any of the preceding paragraphs, wherein the disease is caused by a coronavirus, an intracellular pathogen, a latent infection, an active infection, an influenza virus, respiratory syncytial virus (RSV), human immunodeficiency virus (HIV), varicella zoster virus (VZV), or Mycobacterium tuberculosis; and/or one or more of the antigens are a coronavirus, an intracellular pathogen, a latent infection, an active infection, an influenza virus, respiratory syncytial virus (RSV), human immunodeficiency virus (HIV), varicella zoster virus (VZV), plasmodium (Malaria), Streptococcus pneumoniae, Streptococcus pyogenes, Yersinia pestis, Haemophilus influenzae, Staphylococcus aureus, Pseudomonas aeruginosa, Bordetella pertussis, Ebola virus, Lassa virus, Middle East Respiratory Syndrome coronavirus, SARS-COV-1, SARS-COV-2, SARS-COV-2 variants of concerns, Marburg virus, Nipah virus, Rift Valley Fever virus, Chikungunya virus or Mycobacterium tuberculosis antigen.
120. The method of any of the preceding paragraphs, wherein the disease is caused by a coronavirus and/or one or more of the antigens are a coronavirus antigen.
121. The method of paragraph 109, wherein the coronavirus is MERS-COV virus.
122. The method of paragraph 109, wherein the coronavirus is SARS-COV-1 virus.
123. The method of paragraph 109, wherein the coronavirus is SARS-COV-2 virus.
124. A combination comprising:
   a) at least one dose of first cytokine mRNA construct comprising a first open reading frame (ORF), wherein the first ORF encodes a proinflammatory cytokine; and
   b) at least one dose of one or more of:
      i) a first antigen mRNA construct comprising a second open reading frame (ORF), wherein the second ORF encodes an antigen; and
      ii) an antigen polypeptide, antigen molecule, or killed or attenuated pathogenic agent;
wherein:
   each dose of the first cytokine mRNA construct is no more than 20% of each dose of the first antigen mRNA construct and/or the antigen polypeptide, antigen molecule, or killed or attenuated pathogenic agent by weight; and/or each dose of the first cytokine mRNA construct of from 0.10 μg to 10 μg.
125. The combination of paragraph 124, for use in inducing an immune response in an immune distinct subject.
126. The combination of paragraph 124, for use in treating or preventing a disease in an immune distinct subject.
127. The combination of paragraph 124, for use in immunizing an immune distinct subject.
128. The combination of paragraph 125, wherein the immune response comprises an increase in IL-12 in the subject.
129. The combination of paragraph 125, wherein the immune response comprises an increase in the active IL-12 heterodimer (referred to as ‘p70’) in the subject.
130. The combination of paragraph 125, wherein the immune response comprises an increase in Ig levels in the subject.
131. The combination of paragraph 130, wherein the Ig is IgG2, IgG3, or IgG2a.
132. The combination of paragraph 131, wherein the IgG2a is IgG2a that specifically binds the antigen.
133. The combination of paragraph 125, wherein the immune response comprises a CD4+ T cell response in the subject.
134. The combination of paragraph 125, wherein the immune response comprises a CD8+ T cell response in the subject.
135. The combination of paragraph 125, wherein the immune response comprises a NK cell response in the subject.
136. The combination of paragraph 125, wherein the immune response comprises a Th1 response in the subject.
137. The combination of paragraph 125, wherein the immune response stimulates the production of an interferon gamma (IFNγ) response from T cells in the subject.
138. The combination of paragraph 125, wherein the immune response initiates phagocytosis via the Fc region of each IgG subclass via improved affinity for phagocyte membrane Fc-gamma-receptors (FcγR)
139. The combination of paragraph 125, wherein the immune response comprises immunization of the subject against the antigen or an organism comprising the antigen.
140. The combination of any one of paragraphs 125-139, wherein the immune distinct subject is a subject with immunosenescence.
141. The combination of any one of paragraphs 125-140, wherein the immune distinct subject and/or subject with immunosenescence is a subject of 55 years of age or older.
142. The combination of any one of paragraphs 125-141, wherein the immune distinct subject and/or subject with immunosenescence is a subject of 60 years of age or older.
143. The combination of any one of paragraphs 125-142, wherein the immune distinct subject and/or subject with immunosenescence is a subject of 65 years of age or older.
144. The combination of any one of paragraphs 125-143, wherein the immune distinct subject and/or subject with immunosenescence is a subject of 70 years of age or older.
145. The combination of any one of paragraphs 125-144, wherein the immune distinct subject and/or subject with immunosenescence is a subject of 75 years of age or older.
146. The combination of any one of paragraphs 125-145, wherein the immune distinct subject and/or subject with immunosenescence is a subject who has or is determined to have a reduced TNF response to immune stimuli.
147. The combination of any one of paragraphs 125-146, wherein the immune distinct subject and/or subject with immunosenescence is a subject who has or is determined to have a reduced IL-12 response to immune stimuli.
148. The combination of paragraph 147, wherein the immune stimuli is lipopolysaccharide (LPS).
149. The combination of any one of paragraphs 125-139 or 146-148, wherein the immune distinct subject is an infant.
150. The combination of paragraph 149, wherein the immune distinct subject and/or infant is 2 years of age or younger.
151. The combination of paragraph 149, wherein the immune distinct subject and/or infant is 1 year of age or younger.
152. The combination of paragraph 149, wherein the immune distinct subject and/or infant is 28 days of age or younger.
153. The combination of paragraph 149, wherein the immune distinct subject and/or infant is born preterm.
154. The combination of any one of paragraphs 125-153, wherein the immune distinct subject is immunocompromised, has an HIV infection, has AIDS, has received a transplant, is undergoing immunosuppression, is immunosuppressed, has an infection, is diabetic, has an IgG subclass deficiency, has a substance abuse disorder, or is obese.
155. The combination of any one of paragraphs 125-153, wherein the subject is a subject in a high density living environment.
156. The combination of paragraph 155, wherein the high density living environment is an assisted living facility; a nursing home, a dormitory, or a hospital.
157. The combination of any one of paragraphs 125-156, wherein the subject is a subject who is:
   a) at least 55 years of age; and
   b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.
158. The combination of any one of paragraphs 125-156, wherein the subject is a subject who is:
   a) at least 55 years of age; and
   b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, and/or is obese.
159. The combination of any one of paragraphs 157-158, wherein the subject is at least 60 years of age or older.
160. The combination of any one of paragraphs 157-159, wherein the subject is at least 65 years of age or older.
161. The combination of any one of paragraphs 157-160, wherein the subject is at least 70 years of age or older.
162. The combination of any one of paragraphs 157-161, wherein the subject is at least 75 years of age or older.
163. The combination of any one of paragraphs 124-162, wherein each dose of the first cytokine mRNA construct is no more than 10% of each dose of the first antigen mRNA construct and/or the antigen polypeptide, antigen molecule, or killed or attenuated pathogenic agent by weight.
164. The combination of any one of paragraphs 124-163, wherein each dose of the first cytokine mRNA construct is from 0.5% to 20% of each dose of the first antigen mRNA construct and/or the antigen polypeptide, antigen molecule, or killed or attenuated pathogenic agent by weight.
165. The combination of any one of paragraphs 124-164, wherein each dose of the first cytokine mRNA construct is from 1% to 10% of each dose of the first antigen mRNA construct and/or the antigen polypeptide, antigen molecule, or killed or attenuated pathogenic agent by weight.
166. The combination of any one of paragraphs 124-165, wherein each dose of the first cytokine mRNA construct is no more than 20% of each dose of the first antigen mRNA construct by weight.
167. The combination of any one of paragraphs 124-166, wherein each dose of the first cytokine mRNA construct is no more than 10% of each dose of the first antigen mRNA construct by weight.
168. The combination of any one of paragraphs 124-167, wherein each dose of the first cytokine mRNA construct is from 0.5% to 20% of each dose of the first antigen mRNA construct by weight.
169. The combination of any one of paragraphs 124-168, wherein each dose of the first cytokine mRNA construct is from 1% to 10% of each dose of the first antigen mRNA construct by weight.
170. The combination of any one of paragraphs 124-169, wherein each dose of the first cytokine mRNA construct is from 0.10 μg to 10 μg.
171. The combination of any one of paragraphs 124-170, wherein each dose of the first cytokine mRNA construct is from 0.15 μg to 6 μg.
172. The combination of any one of paragraphs 124-171, wherein each dose of the first cytokine mRNA construct is from 0.3 μg to 3 μg.
173. The combination of any one of paragraphs 124-172, wherein the composition comprises at least 5× less of the antigen than is required to induce an immune response in the absence of the first cytokine mRNA.
174. The combination of any one of paragraphs 124-173, wherein the composition comprises at least 10× less of the antigen than is required to induce an immune response in the absence of the first cytokine mRNA.
175. The combination of any one of paragraphs 124-174, wherein the composition comprises at least 20× less of the antigen than is required to induce an immune response in the absence of the first cytokine mRNA.
176. The combination of any one of paragraphs 124-175, wherein the composition comprises at least 50× less of the antigen than is required to induce an immune response in the absence of the first cytokine mRNA.
177. The combination of any one of paragraphs 124-176, wherein the composition comprises at least 100× less of the antigen than is required to induce an immune response in the absence of the first cytokine mRNA.
178. The combination of any one of paragraphs 125-177, wherein the method comprises administering each of the one or more compositions to the subject no more frequently than once per year.
179. The combination of any one of paragraphs 125-177, wherein the method comprises administering each of the one or more compositions to the subject no more frequently than once every 2 years.
180. The combination of any one of paragraphs 125-177, wherein the method comprises administering each of the one or more compositions to the subject no more frequently than once every 3 years.
181. The combination of any one of paragraphs 125-177, wherein the method comprises administering each of the one or more compositions to the subject no more frequently than once every 4 years.
182. The combination of any one of paragraphs 125-177, wherein the method comprises administering each of the one or more compositions to the subject no more frequently than once every 5 years.
183. The combination of any one of paragraphs 125-177, wherein the first antigen mRNA construct antigen is an antigen of a first infectious organism and the method comprises administering a composition comprising any antigen from the first infectious organism to the subject no more frequently than once per year.
184. The combination of any one of paragraphs 125-177, wherein the first antigen mRNA construct antigen is an antigen of a first infectious organism and the method comprises administering a composition comprising any antigen from the first infectious organism to the subject no more frequently than once every 2 years.
185. The combination of any one of paragraphs 125-177, wherein the first antigen mRNA construct antigen is an antigen of a first infectious organism and the method comprises administering a composition comprising any antigen from the first infectious organism to the subject no more frequently than once every 3 years.
186. The combination of any one of paragraphs 125-177, wherein the first antigen mRNA construct antigen is an antigen of a first infectious organism and the method comprises administering a composition comprising any antigen from the first infectious organism to the subject no more frequently than once every 4 years.
187. The combination of any one of paragraphs 125-177, wherein the first antigen mRNA construct antigen is an antigen of a first infectious organism and the method comprises administering a composition comprising any antigen from the first infectious organism to the subject no more frequently than once every 5 years.
188. The combination of any one of paragraphs 124-187, wherein the proinflammatory cytokine is selected from the group consisting of: IL-12; IL-2; IL-4; IL-5; IL-6; IL-8; IL-10; IL-13; IL-27; IL-1 beta; TGFbeta; IFNy; IFNa; IFNI3; TNFa; CCL2; CCL3; CCL4; CCL5; CCL8; CXCL12; GM-CSF; and a subunit, dimer, heterodimer, derivative, fragment, agonist or homologue thereof.
189. The combination of any one of paragraphs 124-188, wherein the proinflammatory cytokine is IL-12 or a subunit, dimer, heterodimer, derivative, fragment, agonist or homologue thereof.
190. The combination of paragraph 124-189, wherein the first ORF comprises a sequence at least 90% identical to SEQ ID NO: 59.
191. The combination of any one of paragraphs 124-190, wherein the proinflammatory cytokine is IL-12 or a subunit, of human, and other mammalian homology.
192. The combination of any one of paragraphs 124-191, wherein the one or more compositions further comprise one or more further cytokine mRNA constructs, each comprising a further open reading frame (ORF), wherein each further ORF encodes a proinflammatory cytokine distinct from the proinflammatory cytokine encoded by the first ORF.
193. The combination of paragraph 192, wherein the composition comprises 1-9 further cytokine mRNA constructs.
194. The combination of any one of paragraphs 124-193, wherein the first cytokine mRNA construct further comprises one or more further open reading frames (ORFs), wherein each further ORF encodes a proinflammatory cytokine distinct from the proinflammatory cytokine encoded by the first ORF.
195. The combination of paragraph 194, wherein the first cytokine mRNA construct comprises 1-9 further ORFs encoding a proinflammatory cytokine distinct from the proinflammatory cytokine encoded by the first ORF.
196. The combination of any one of paragraphs 124-195, wherein the first ORF encodes IL-12 or a subunit, derivative, fragment, agonist or homologue thereof and the one or more further ORFs encode IL-2; IL-4; IL-5; IL-6; IL-8; IL-10; IL-13; IL-27; IL-1β; TGFβ; IFNγ; IFNα; IFNβ; TNFα; CCL2; CCL3; CCL4; CCL5; CCL8; CXCL12; GM-CSF; or a subunit, derivative, fragment, agonist or homologue thereof.
197. The combination of any one of paragraphs 124-196, wherein the composition further comprises one or more further antigen mRNA constructs, each comprising a further open reading frame (ORF), wherein each further ORF encodes an antigen distinct from the antigen encoded by the second ORF.
198. The combination paragraph 197, wherein the composition comprises 1-9 further antigen mRNA constructs.
199. The combination of any one of paragraphs 124-198, wherein the antigen mRNA construct further comprises one or more further open reading frames (ORFs), wherein each further ORF encodes an antigen distinct from the antigen encoded by the second ORF.
200. The combination of paragraph 199, wherein the composition comprises 1-9 further ORFs encoding an antigen distinct from the antigen encoded by the second ORF.
201. The combination of any one of paragraphs 124-200, wherein the composition comprises a plurality of ORFs encoding a plurality of antigens, and the plurality of antigens comprises multiple antigens from a first organism.
202. The combination of any one of paragraphs 124-201, wherein the composition comprises a plurality of ORFs encoding a plurality of antigens, and the plurality of antigens comprises one or more antigens from a first organism and one or more antigens from one or more further organisms.
203. The combination of paragraph 202, wherein the composition comprises a plurality of ORFs encoding a plurality of antigens, and the plurality of antigens comprises one or more antigens from a coronavirus and one or more antigens from an influenza virus.
204. The combination of paragraph 202, wherein the composition comprises a plurality of ORFs encoding a plurality of antigens, and the plurality of antigens comprises one or more spike protein antigens from a coronavirus and one or more antigens from an influenza virus.
205. The combination of any one of paragraphs 124-204, wherein the antigen is an antigen of an infectious organism and whereby transmission of the infectious organism to or by the subject is reduced as compared to administration of a composition not comprising the cytokine mRNA construct.
206. The combination of any one of paragraphs 124-205, wherein the antigen is a pathogenic microbial protein or an epitope containing fragment thereof.
207. The combination of paragraph 206, wherein the pathogenic microbial protein is selected from the group consisting of: a viral protein; a bacterial protein; a fungal protein; a parasite protein; and a prion.
208. The combination of any one of paragraphs 124-205, wherein the antigen comprises a viral protein or an epitope containing fragment thereof.
209. The combination of paragraph 208, wherein the antigen comprises a coronavirus spike protein.
210. The combination of paragraph 208, wherein the antigen comprises a coronavirus receptor binding domain (RBD) protein.
211. The combination of paragraph 208, wherein the antigen comprises a variant coronavirus spike protein.
212. The combination of paragraph 208, wherein the antigen comprises a variant coronavirus receptor binding domain protein.
213. The combination of paragraph 209, wherein the coronavirus spike protein is a MERS-COV spike or RBD protein.
214. The combination of paragraph 209, wherein the coronavirus spike protein is a SARS-COV-1 spike or RBD protein.
215. The combination of paragraph 209, wherein the coronavirus spike protein is a SARS-COV-2 spike or RBD protein.
216. The combination of paragraph 208, wherein the antigen comprises an influenza protein or a variant thereof, or an epitope containing fragment thereof.
217. The combination of paragraph 216, wherein the influenza protein is selected from the group consisting of a hemagglutinin, a neuraminidase, a matrix-2 and/or a nucleoprotein.
218. The combination of paragraph 216, wherein the influenza protein is selected from type A influenza, a type B influenza, or a subtype of type A influenza of H1, H2, H3, H4, H5, H6, H7, H8, H9, H10, H11, H12, H13, H14, H15 or H16.
219. The combination of paragraph 208, wherein the antigen comprises a respiratory syncytial virus (RSV) protein, or a variant thereof, or an epitope containing fragment thereof.
220. The combination of paragraph 219, wherein the protein of the respiratory syncytial virus is the F glycoprotein or the G glycoprotein.
221. The combination of paragraph 208, wherein the antigen comprises a Human Immunodeficiency Virus (HIV) protein or an epitope containing fragment thereof.
222. The combination of paragraph 221, wherein the HIV protein is the glycoprotein 120 neutralizing epitope or glycoprotein 145.
223. The combination of paragraph 206, wherein the antigen comprises a protein from the Mycobacterium tuberculosis bacterium or an epitope containing fragment thereof.
224. The combination of paragraph 223, wherein the protein from the Mycobacterium tuberculosis bacterium is selected from ESAT-6, Ag85B, TB10.4, Rv2626 and/or RpfD-B.
225. The combination of any one of paragraphs 124-224, wherein one or more of the first, second, or further ORFs is operatively linked to at least one untranslated region (UTR), wherein each UTR comprises at least a first organ protection sequence (OPS), wherein each OPS comprises at least two micro-RNA (miRNA) target sequences, and wherein each of the at least two miRNA target sequences are optimised to hybridise with a corresponding miRNA sequence.
226. The combination of paragraph 225, wherein each ORF of the composition is operatively linked to a UTR comprising at least one OPS.
227. The combination of any one of paragraphs 124-226, wherein each OPS of the composition independently comprises at least three, at least four, or at least five miRNA target sequences.
228. The combination of any one of paragraphs 124-226, wherein each OPS of the composition independently comprises at least three miRNA target sequences which are all different from each other.
229. The combination of any one of paragraphs 124-228, wherein the first and second ORFs are operatively linked to the same OPS.
230. The combination of any one of paragraphs 124-228, wherein the first and second ORFs are operatively linked to different OPSs.
231. The combination of any one of paragraphs 124-230, wherein the OPS linked to the first ORF and the OPS linked to the second ORF comprise the same miRNA target sequences.
232. The combination of any one of paragraphs 124-228 and 230, wherein the OPS linked to the first ORF and the OPS linked to the second ORF each comprise at least one miRNA target sequence not comprised by the other OPS.
233. The combination of any one of paragraphs 124-228, 230, and 232, wherein the OPS linked to the first ORF and the OPS linked to the second ORF each comprise at least three miRNA target sequences not comprised by the other OPS.
234. The combination of any one of paragraphs 124-233, wherein the OPS operatively linked to the second ORF comprises miRNA sequences selected to protect one or more organs or tissues selected from the group consisting of muscle, liver, brain, breast, endothelium, pancreas, colon, kidney, lungs, spleen and skin, heart, gastrointestinal organs, reproductive organs, and esophagus.
235. The combination of any one of paragraphs 124-233, wherein the OPS operatively linked to the first ORF comprises miRNA sequences selected to protect one or more organs or tissues selected from the group consisting muscle, liver, brain, breast, endothelium, pancreas, colon, kidney, lungs, spleen and skin.
236. The combination of any one of paragraphs 124-233, wherein the OPS operatively linked to the first ORF comprises miRNA sequences selected to protect one or more organs selected from the group consisting of muscle, liver, kidney, lungs, spleen, skin, heart, gastrointestinal organs, reproductive organs, and esophagus.
237. The combination of any one of paragraphs 124-236, wherein one or more of the OPS independently comprises:
   a) at least two miRNA target sequences selected from one or more sequences that bind to: miRNA-122; miRNA-125; miRNA-199; miRNA-124a; miRNA-126; miRNA-98; Let7 miRNA family; miRNA-375; miRNA-141; miRNA-142; miRNA-148a/b; miRNA-143; miRNA-145; miRNA-194; miRNA-200c; miRNA-203a; miRNA-205; miRNA-1; miRNA-133a; miRNA-206; miRNA-34a; miRNA-192; miRNA-194; miRNA-204; miRNA-215; miRNA-30 family; miRNA-877; miRNA-4300; miRNA-4720; and/or miRNA-6761;
   b) sequences selected from one or more of SEQ ID NOs: 44-57;
   c) at least two miRNA target sequences selected from sequences capable of binding with miRNA-1, miRNA133a, miRNA206, miRNA-122, miRNA203a, miRNA205, miRNA200c, miRNA30a, and/or let7a/b;
   d) at least two miRNA target sequences selected from one or more sequences that bind to: miRNA-1, miRNA-122, miRNA-30a, miRNA-203a, let7b, miRNA-126, and/or miRNA-192;
   e) at least two miRNA target sequences selected from sequences capable of binding with miRNA-1, miRNA-122, miR-30a and/or miR-203a;
   f) miRNA target sequences capable of binding with miRNA-1, miRNA-122, miRNA-30a and miRNA-203a;
   g) miRNA target sequences capable of binding with let7b, miRNA-126, and miRNA-30a;
   h) miRNA target sequences capable of binding with miRNA-122, miRNA-192, and miRNA-30a; or
   i) miRNA target sequences capable of binding with miRNA-192, miRNA-30a, and miRNA-124, and two miRNA target sequences capable of binding with miRNA 122.
238. The combination of any one of paragraphs 124-236, wherein the OPS operatively linked to the second ORF comprises miRNA target sequences capable of binding with miRNA-1, miRNA-122, miR-30a and/or miR-203a; and the OPS operatively linked to the first ORF comprises miRNA target sequences capable of binding with miRNA-122, miRNA-126, miRNA-192, and/or miRNA 30a.
239. The combination of any one of paragraphs 124-238, wherein the administration is intravenous, subcutaneous, intramuscular, intranasal, intra-arterial, or via inhalation.
240. The combination of any one of paragraphs 124-239, wherein the first, second, and/or further mRNA constructs are comprised within or adsorbed to an in vivo delivery composition.
241. The combination of paragraph 124-240, wherein the delivery composition comprises delivery vectors selected from the group consisting of: a particle, such as a polymeric particle; a liposome; a lipidoid particle; and a viral vector.
242. The combination of any one of paragraphs 124-241, wherein the disease is caused by a coronavirus, an intracellular pathogen, a latent infection, an active infection, an influenza virus, respiratory syncytial virus (RSV), human immunodeficiency virus (HIV), varicella zoster virus (VZV), or Mycobacterium tuberculosis; and/or one or more of the antigens are a coronavirus, an intracellular pathogen, a latent infection, an active infection, an influenza virus, respiratory syncytial virus (RSV), human immunodeficiency virus (HIV), varicella zoster virus (VZV), plasmodium (Malaria), Streptococcus pneumoniae, Streptococcus pyogenes, Yersinia pestis, Haemophilus influenzae, Staphylococcus aureus, Pseudomonas aeruginosa, Bordetella pertussis, Ebola virus, Lassa virus, Middle East Respiratory Syndrome coronavirus, SARS-COV-1, SARS-COV-2, SARS-COV-2 variants of concerns, Marburg virus, Nipah virus, Rift Valley Fever virus, Chikungunya virus or Mycobacterium tuberculosis antigen.
243. The combination of any one of paragraphs 124-233, wherein the disease is caused by a coronavirus and/or one or more of the antigens are a coronavirus antigen.
244. The combination of paragraph 243, wherein the coronavirus is MERS-COV virus.
245. The combination of paragraph 243, wherein the coronavirus is SARS-COV-1 virus.
246. The combination of paragraph 243, wherein the coronavirus is SARS-COV-2 virus.

The technology described herein is further illustrated by the following examples which in no way should be construed as being further limiting.

### EXAMPLES

### Example 1

It is demonstrated herein that an mRNA encoding IL-12 encapsulated in lipid nanoparticles (LNP) for concomitant delivery alongside antigen elicited amplified Th1-polarization observed in human peripheral blood ex vivo (e.g., interleuking-12 (IL-12), interferon gamma (IFNγ)), induced bioactive gene translation, and enhanced Th1-polarized IgG2a antibody responses to mRNA encoding SARS-COV-2 Spike protein, and increased CD4+ Th1-polarized spike-specific T cell and CD8+ spike-specific T cell activity in adult and elder mice in vivo at a non-inferior to superior level as compared to licensed formulations available (Pfizer/BioNTech's BNT162b2/Comirnaty).

Adjuvants can enhance, prolong, and modulate immune responses to vaccinal antigens to maximize protective immunity, provide dose sparing effects reducing amount of vaccine required through lower dose or fewer immunizations, and can permit more effective immunization of vulnerable populations. In this context, the application of a single chain interleukin 12 (IL-12) constructed of a heterodimer IL-12p35 and p40 encapsulated in ionizable lipid nanoparticles was explored to determine if lipid nanoparticle delivery of mRNA could amplify immunity, benefiting from the immunological importance of IL-12 and the benefits of mRNA gene delivery.

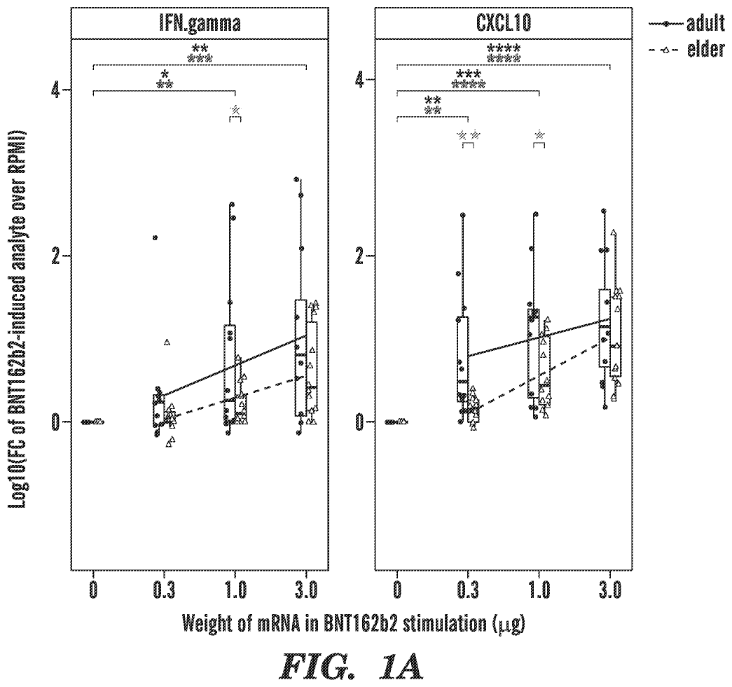

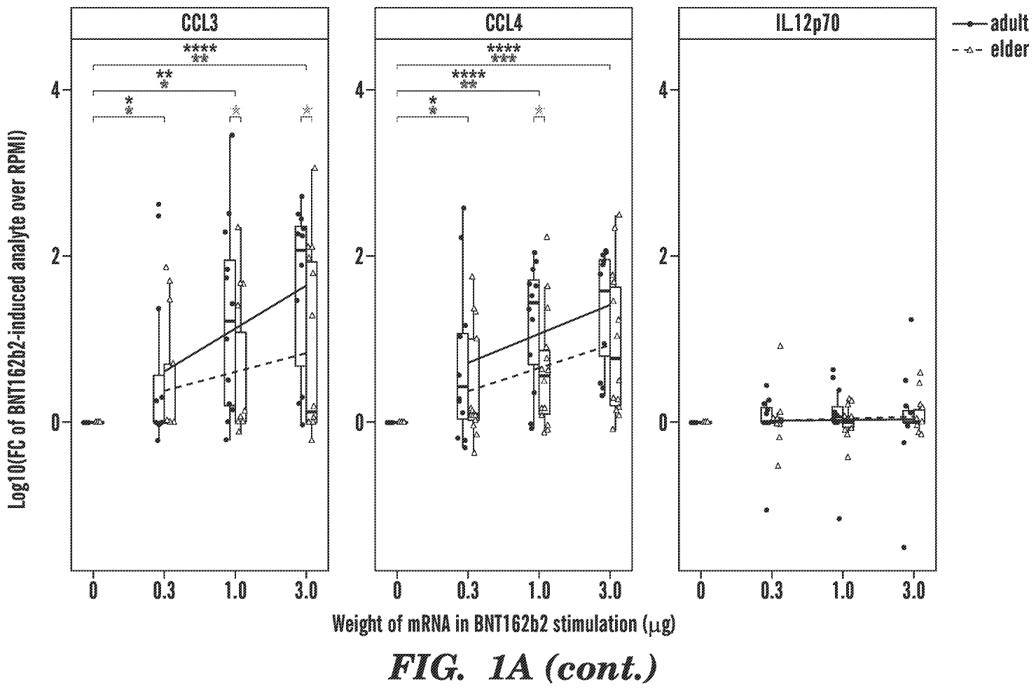

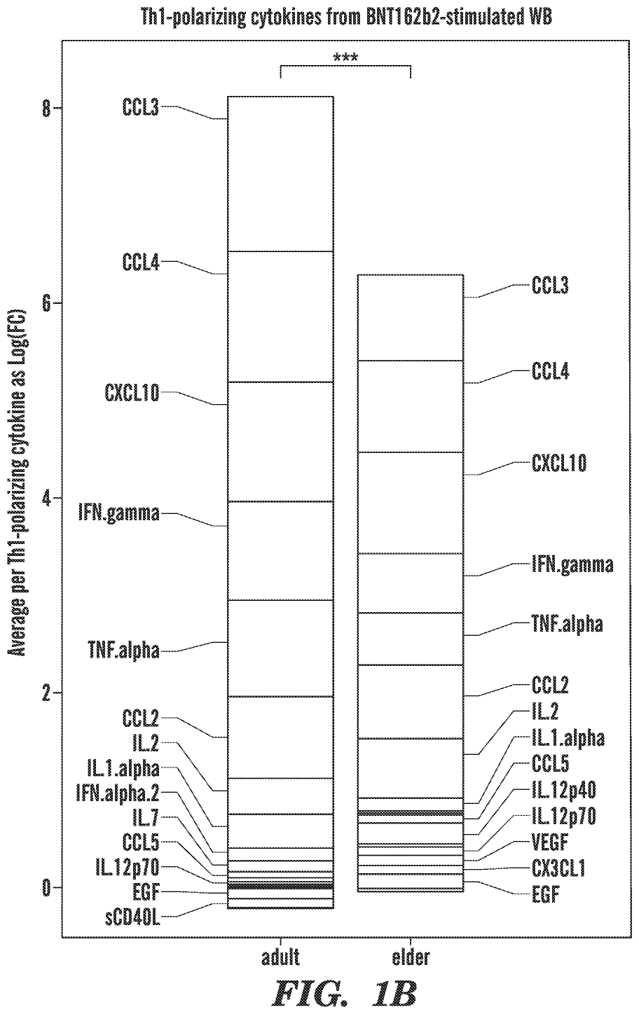

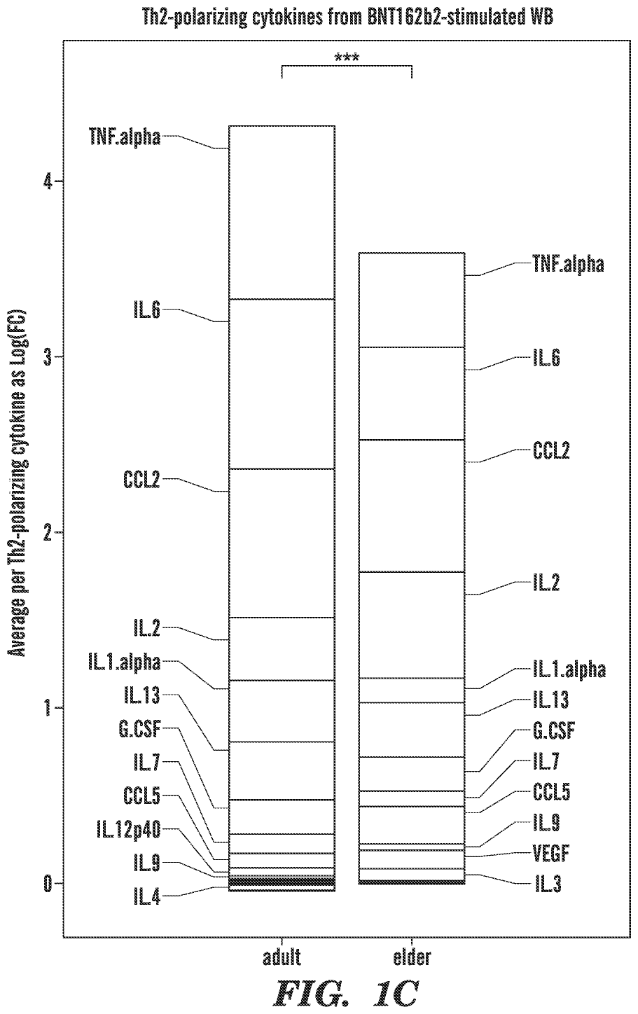

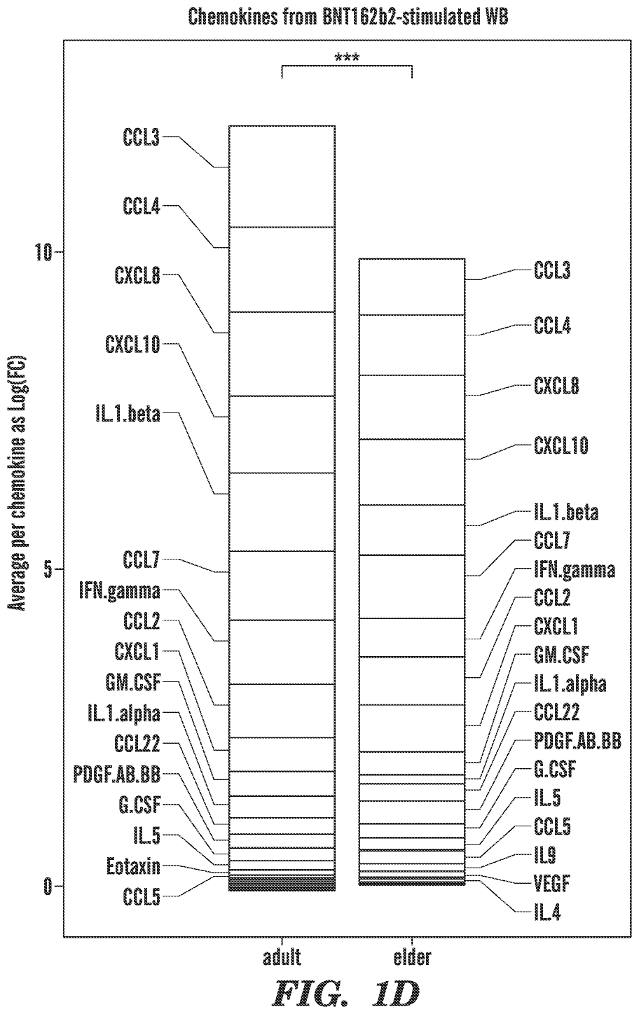

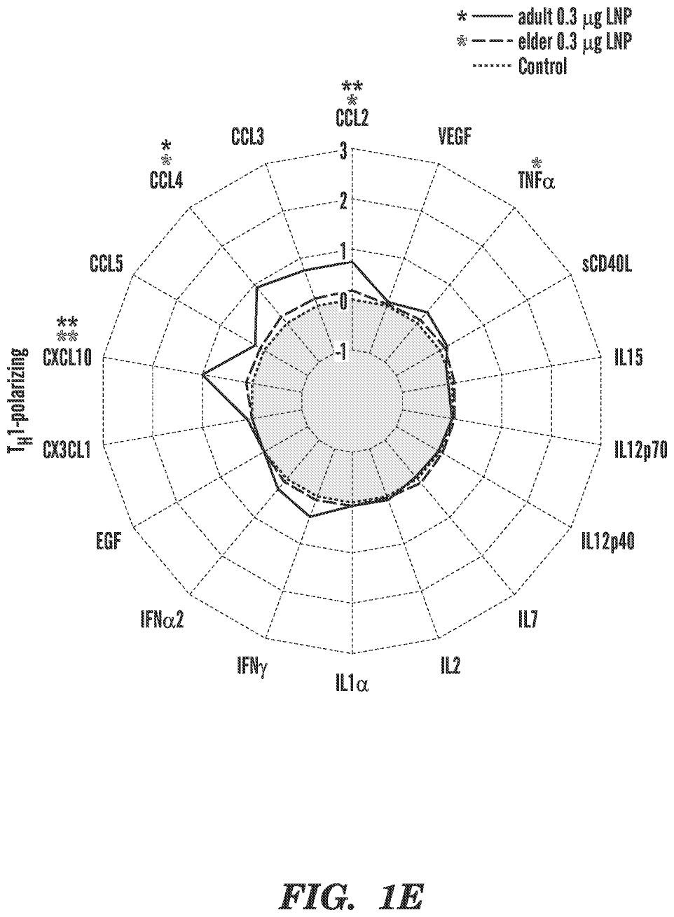

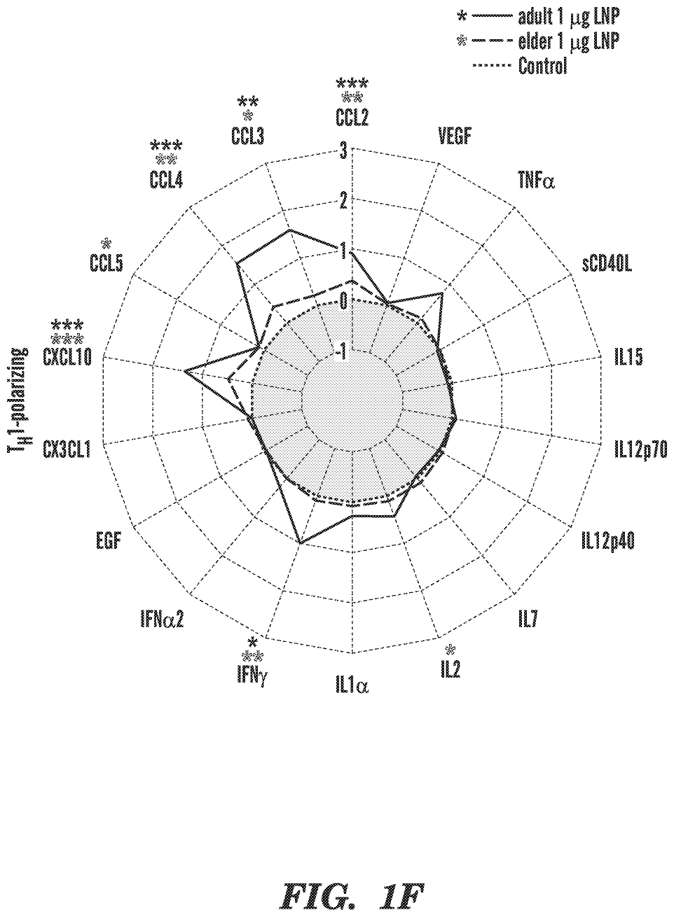

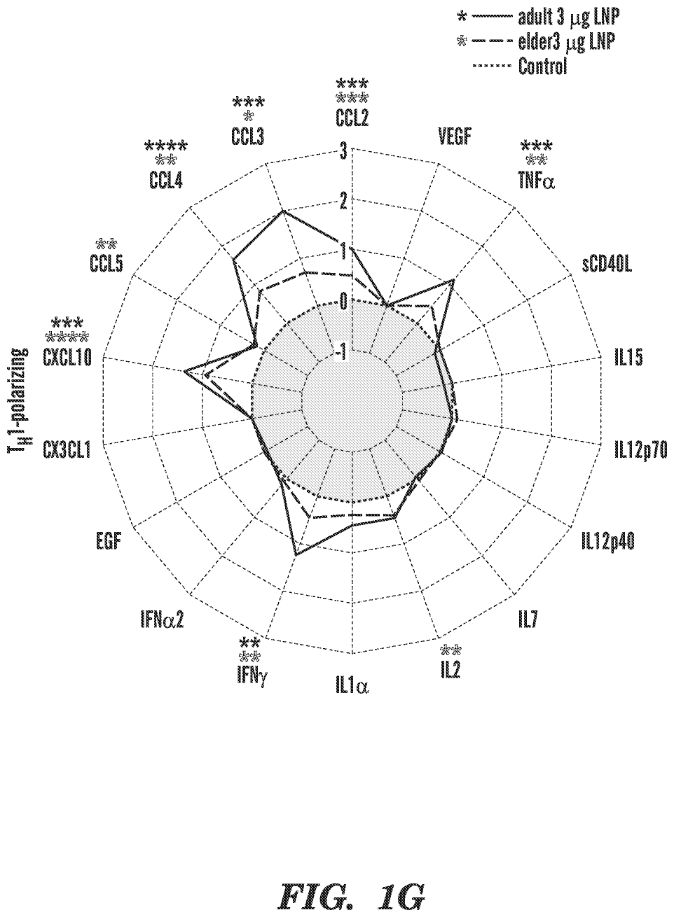

The ability to adjuvant is most impactful when utilizing or inducing cytokines and immunological pathways that are not already activated by the antigen delivery mechanism itself. Pfizer/BioNTech's BNT162b2 was assessed for the induction of 41 analytes in a human in vitro assay and found no induction of IL-12, while the same stimulation yielded significant induction of pro-inflammation and reactogenicity markers (e.g., IFNγ, CXCL10), indicating that a relevant dose was utilized (FIG. 1A).

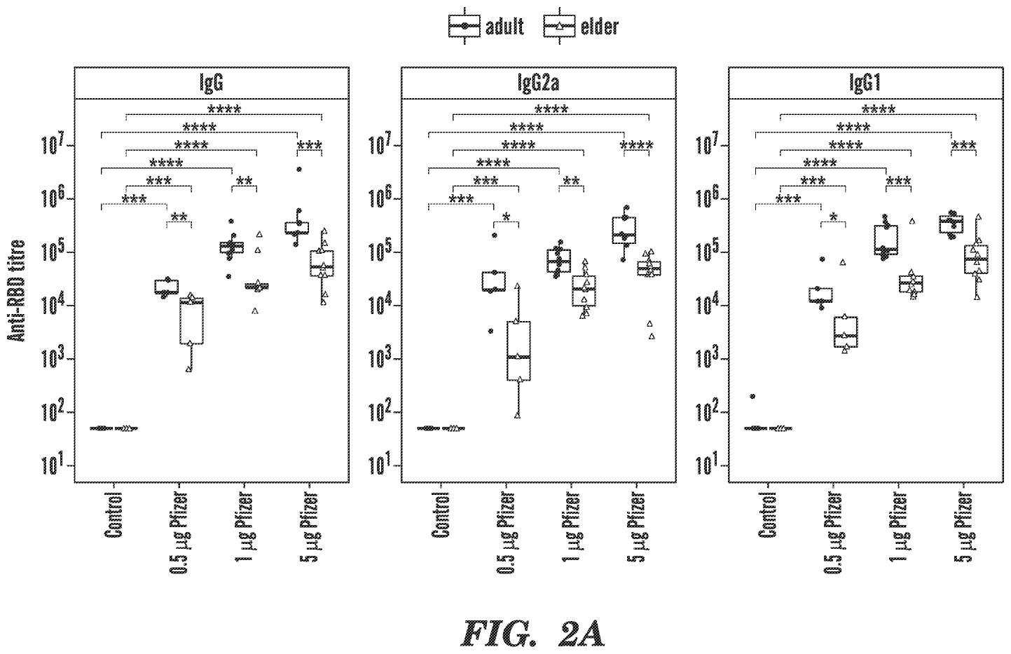

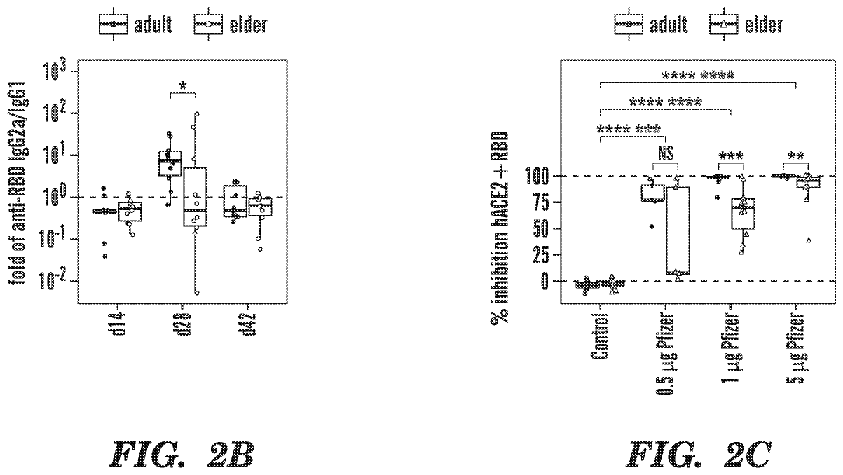

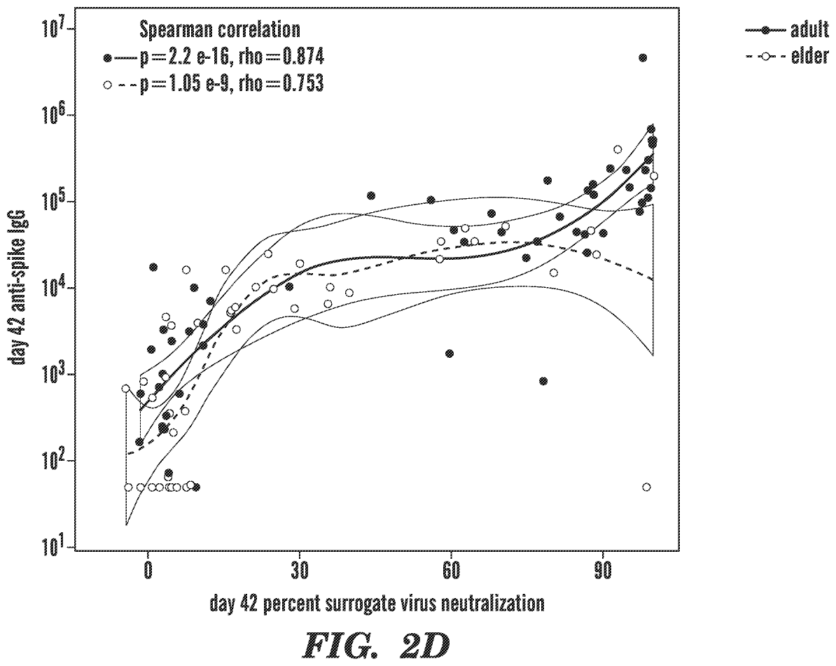

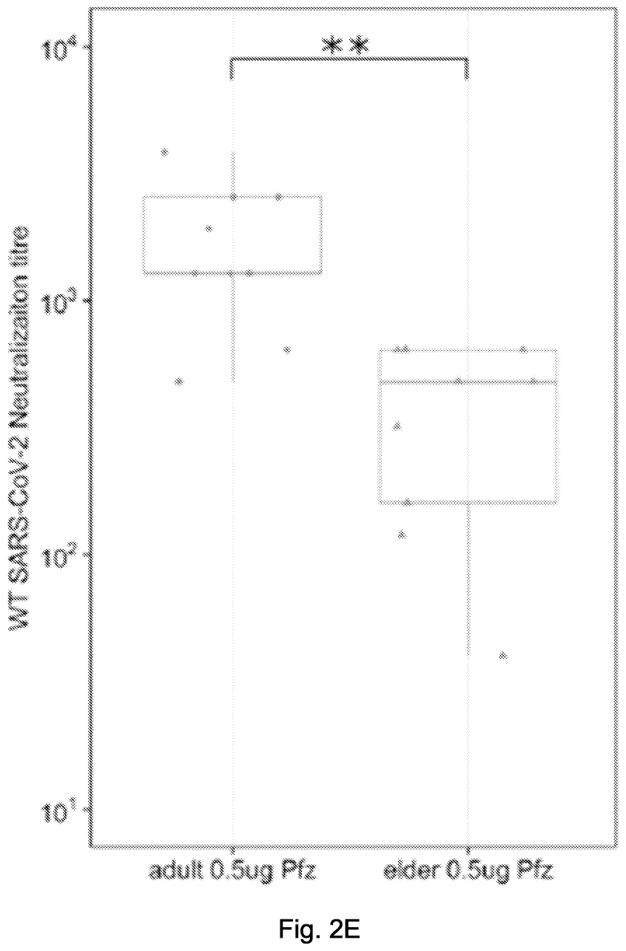

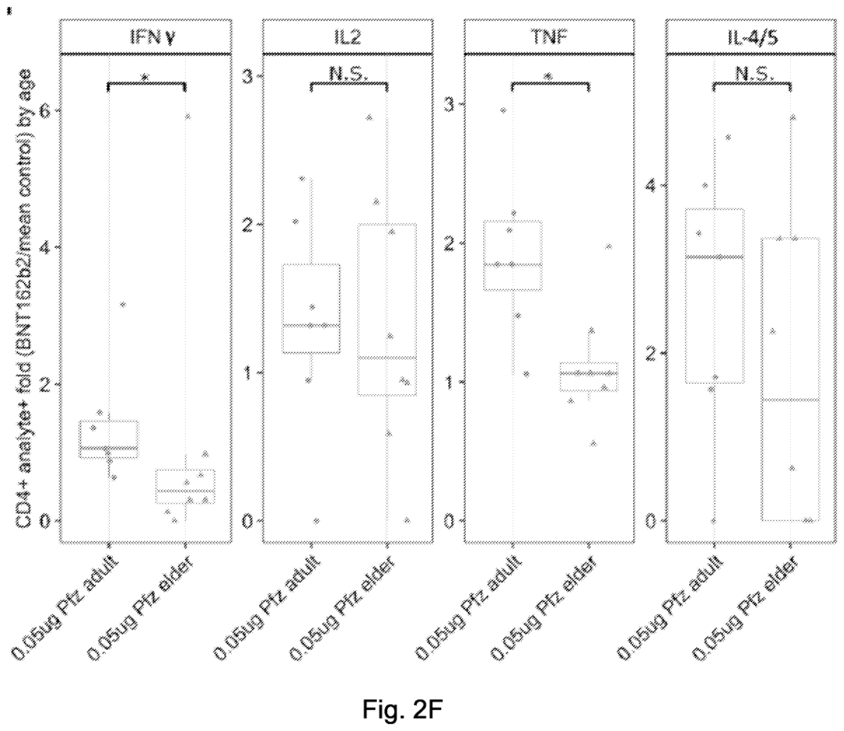

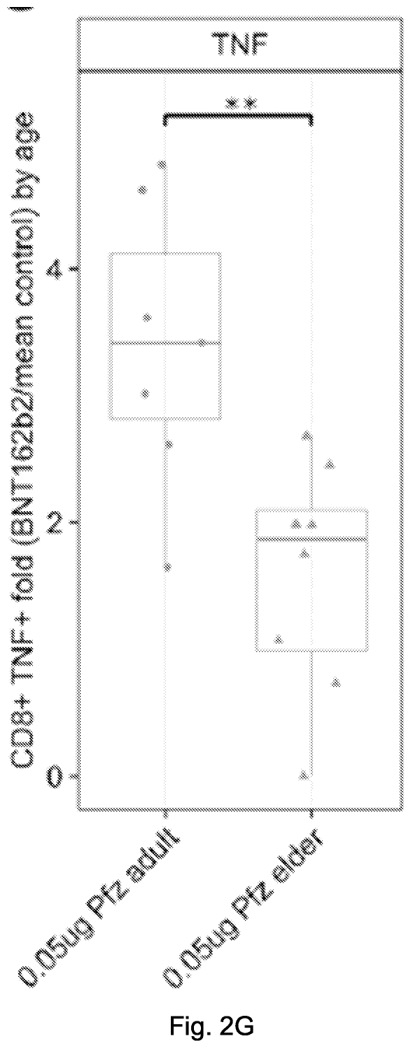

Immunosenescence in elders can result in dampened immune responses to vaccination and cause increased susceptibility to pathogens resulting in higher infection, morbidity, and mortality rates in elders. This can be driven by differential immune activation at exposure to the vaccine or infectious material, which can also be categorized into Th1- and Th2-polarizing activities and could be affected by the capacity to recruit immune populations to the site of vaccination through the release of chemokines. An effective Th1 response is required for sufficient cell-mediated immunity and generation of IgG2a antibodies to protect from infection, but impaired Th1 activation was observed in elder in vitro whole blood stimulation (FIGS. 1A-1B), establishing the need of an adjuvant for proper induction of human immunity to increase immunogenicity and immunity. Age-associated impairments of other functional roles (Th-2 polarization and chemotaxis) were also observed (FIG. 1C-1D). This immunosenescence was also observed in murine in vivo experiments where BALB/c mice were prime boost immunized with BNT162b2 (injections separated by 14 days), and day 42 post-prime anti-spike antibody levels were impaired in elders across multiple doses and antibody subtypes as compared to adult mice (FIG. 2A). The functional classes of the antibodies were Th-1 associated in adults and Th-2 associated in elders (FIG. 2B), with associated immunological impairment of lower capacity to neutralize the binding of the Receptor Binding Domain (RBD) of the spike antigen from binding human ACE-2 coated ELISA plates (FIG. 2C) providing further immunological evidence for the need to adjuvant elders. Elders and neonates follow similar immunological trajectories particularly with impaired immunological and efficacy responses to vaccination and will both benefit from IL-12 adjuvantation.

IL-12 adjuvantation can improve multiple immune functions, benefiting adults, neonates, and elders, including:
1) induction of proinflammatory cytokines (e.g., IFN-gamma (γ))
2) immunological guidance by inducing Th1 polarization
3) supplementing immunological responses not already active by current mRNA vaccine technologies
4) accelerated induction of immunity following primary immunization
5) adaptive immune cell differentiation (e.g., T cell)
6) amplification of cell cytotoxicity (e.g., increased natural killer and CD8 T cell frequency)
7) lead to chemotaxis of antigen presenting cells (e.g., Monocytes and DCs)
8) provide immunological memory support (e.g., CD4+ T cell priming of B cell antibody production)
9) increased T cell receptor breadth and avidity
10) overcoming age-specific elder and neonatal impaired IL-12 production
11) immune activation and recovery from disease (e.g., tuberculosis, E. cuniculi)
12) displayed efficacy in a plasmid delivery to augment immunogenicity to HIV's gag in a human vaccine study

The applicability of each functional role and benefit of IL-12 adjuvantation in the context of mRNA delivered antigen was confirmed. The delivery of IL-12 by mRNA and LNP encapsulation has not previously been observed and is a novel application of this technology. LNP have additive value compared to alternative recombinant protein or plasmid-based administration techniques. Delivery of mRNA for protein translation in the vaccinee's cells can result in natural protein folding, glycosylation, and function (compared to non-optimal folding or glycosylation in non-antigen presenting cells), reduced pyrogen contamination (e.g., reduced lipopolysaccharide content from the avoidance of bacterial expression), and targeted site-specific production of antigen and adjuvant (from LNP components, e.g., size, particle charge, PEGylation, cationic lipid used). LNP delivery can reduce systemic toxicity and therefore reducing subsequent vaccine reactogenicity permitting a greater dose of administration, potentially allowing for higher maximal immunity. Developing and evaluating adjuvants that amplify immunity in adults but can also protect neonates and elders have added value for the activation and elicitation of effective immune memory in otherwise immunologically impaired individuals but must be observed.

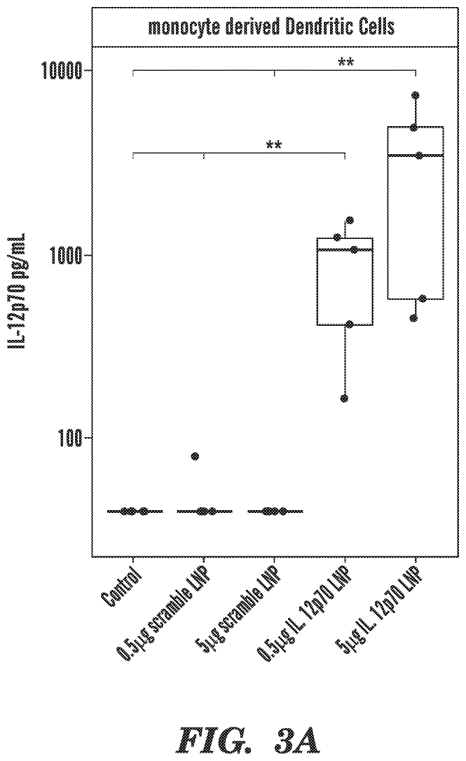

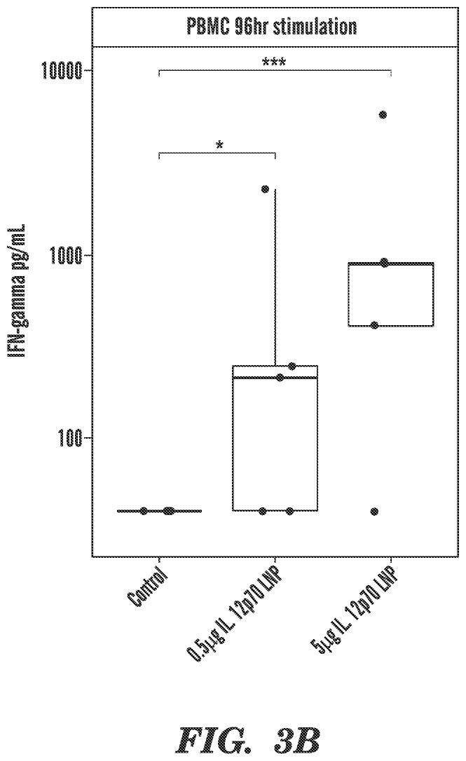

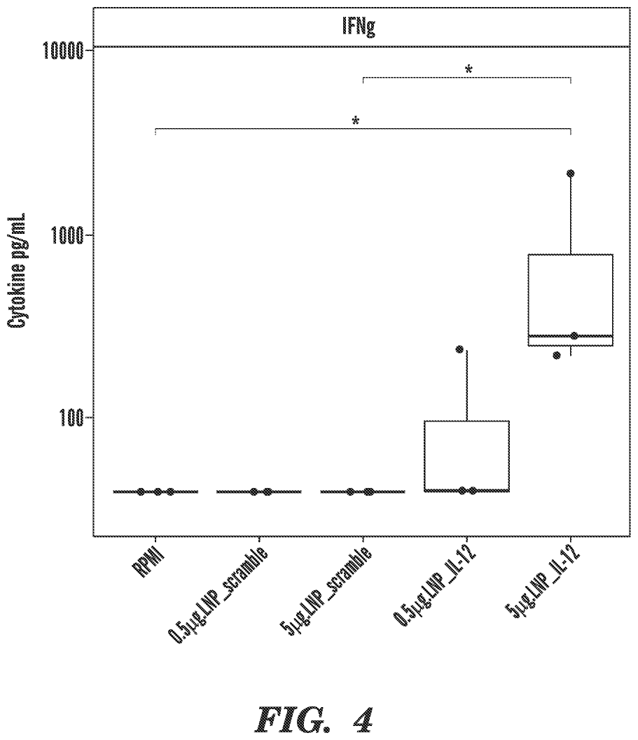

The effectiveness of an IL-12 adjuvant was tested using in vitro experiments in age-dependent immune modeling in adult and elder samples. Human evaluation of the delivery of IL12p70 was displayed in significant MoDC induction of IL-12p70 expression over control (RPMI and scramble mRNA) in all human adult samples (FIG. 3A). This indicated the ability to target antigen presenting cells to express the mRNA payload that would be required for effective immunization in humans, and that IL-12 production was not due to the material present in the LNP (e.g., mRNA induced TLR7/8 signaling, or LNP components like cholesterol induced immunogenicity). Evaluation of proper protein folding and glycosylation, or the bioactivity of the protein, was performed by measuring IL-12p70 induced IFNγ in a 96 hr PBMC stimulation (FIG. 3B). Significant IFNγ was observed indicating bioactive IL-12p70 expression in human samples. Gene expression in elders can be impaired due to many factors (e.g., decreased phagocytosis and decreased macropinocytosis required for gene transfection, etc.). Evaluation of CTx LNP expressing IL12p70 in elder samples was observed with PBMC stimulated over 96 hours. Stimulation of elder samples with 5 μg of IL-12 mRNA loaded in LNP significantly induced IFNγ over control (FIG. 4). In vitro evaluation can test applicability of the technology to humans, but induction of amplified adaptive immunity in vivo is an essential evaluation for adjuvanticity and required the adaptation of adult and elder murine models.

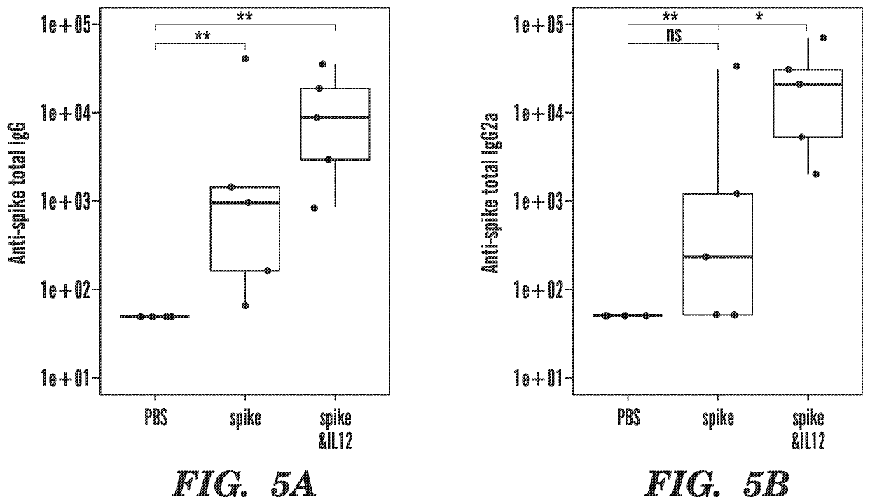

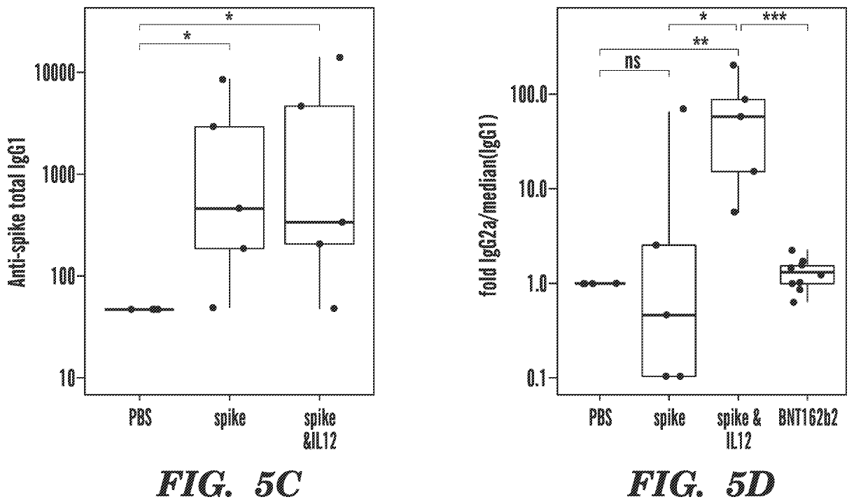

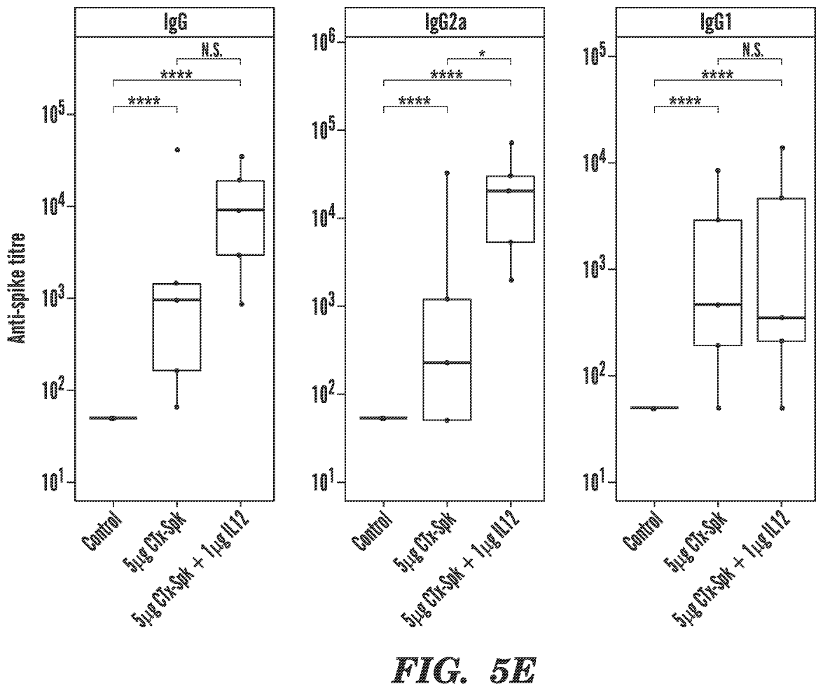

PVP immunization and humoral immunity models were applied to mRNA loaded LNP vaccination in mice, in vivo. Evaluation of induced immunogenicity, and IL-12 induced adjuvanticity of adult mice was evaluated. Intramuscular (IM)-immunized mice were given a booster immunization on day 14 post-prime, and serum was obtained on days 28 and 42 post-prime to evaluate spike protein specific total IgG, IgG2a (marker of Th-1 polarization), and IgG1 (marker of Th-2 polarization). By day 28 post-vaccination mice that received LNP with 5 μg mRNA encoding for SARS-COV-2 spike antigen (CTx-Spk) but without IL-12 adjuvantation, elicited significant induction of total IgG and IgG1, but not the Th-1 associated IgG2a over negative control (FIG. 5A-5C). Adjuvantation of this immune response with 1 μg mRNA encoding IL-12 was required for significant amplification of IgG2a immunogenicity over spike alone (3.7-fold greater induction). Fold induction of IgG2a over IgG1, a measure of Th1 and Th2 polarization, was observed and day 28 post-prime IL-12 adjuvanted anti-spike immunity was the only significant difference compared to PBS control (FIG. 5D), indicating successful Th1-guidance of the immune response. IL-12 adjuvantation resulted in a significant ˜100-fold induction over PBS, spike alone, or alternative delivery of the emergency use authorization BNT162b2 (Pfizer/BioNTech's SARS-COV-2 vaccine encoding spike), displaying the additive value of utilizing LNP delivered mRNA encoding IL-12 to adjuvant an immune response. This amplified immunity was observed 2 weeks later (FIG. 5E) on day 42 post-prime immunization and found to persist as 5 μg mRNA encoding spike+1 μg mRNA adjuvanted a 3.5-fold greater spike-specific IgG2a than non-adjuvanted spike mRNA alone.

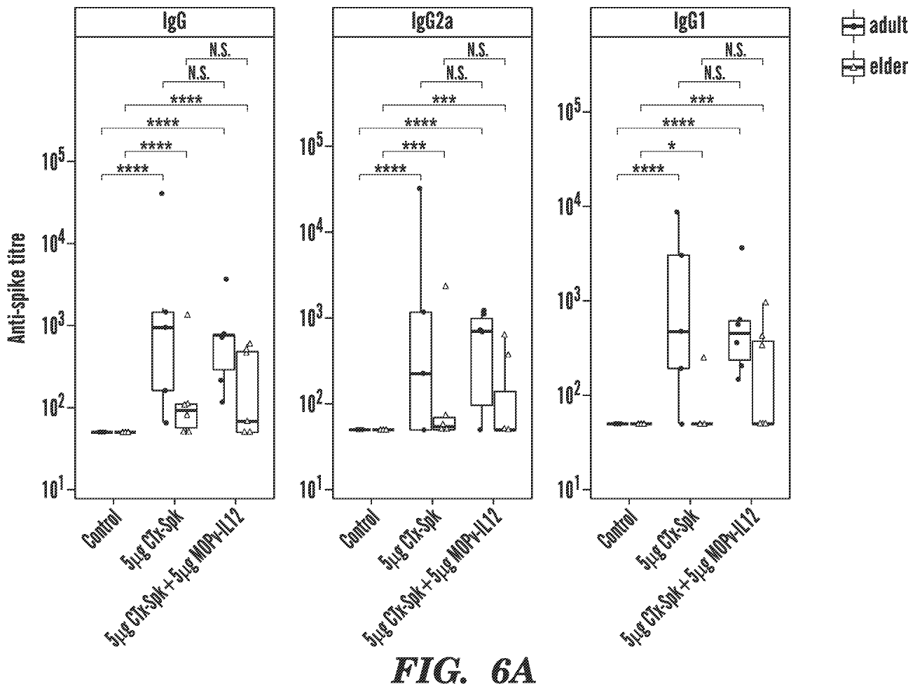

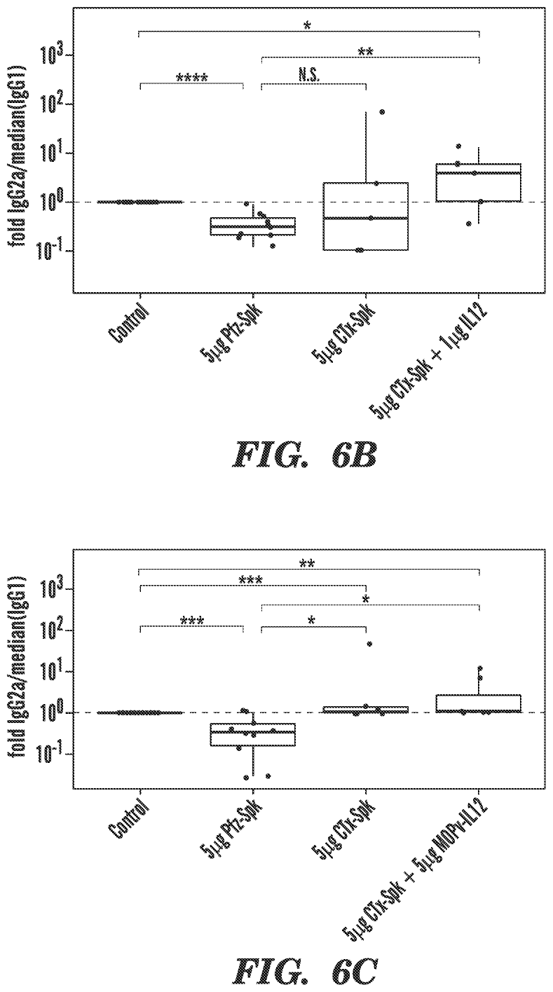

Adjuvantation of elder mice was then evaluated with 5 μg mRNA encoding spike (CTx-Spk)+5 μg mRNA encoding IL12 with 5 μg mRNA encoding spike+5 μg mRNA (MOPv-IL12). In both adult and elder mice, a significant induction of IgG, IgG2a, and IgG1 was induced with spike mRNA alone (FIG. 6A), and a non-significant trend towards adjuvanticity in adult IgG2a and in elder IgG, IgG2a, and IgG1 was observed with a shift towards greater antibody titres. Eliciting immunity in age-specific settings is important to display the ability to deliver antigen and gene targets in vivo. Obtaining the correct dose of mRNA encoding antigen or adjuvant is crucial, as other studies investigating alternative delivery systems for IL-12 adjuvantation observed a dose-dependent response where too low or too high of IL-12 can impair adjuvanticity. A displayed functional shift was observed in adult mice as immunization with either 5 μg mRNA encoding spike formulated by Pfizer or with the CTx-Spk formulation induced Th-2 polarization, but was rescued to Th-1 polarization with 1 μg IL-12 adjuvantation (FIG. 6B). Elder mice were similarly Th-2 polarized with 5 μg mRNA encoding spike formulated by Pfizer, but had a balanced immune response with 5 μg mRNA encoding spike formulation CTx-Spk, potentially due to a low limit of dilution limitation of non-adjuvanted titres obtained. Adjuvantation of elder responses with 5 μg MOPv-IL12 induced significantly greater Th-1 polarization than control or non-adjuvanted Pfizer-immunized mice, suggesting correct direction of, and biological effect on, the elder immune response. A goal of elder immunization is to successfully induce adult-like immunity.

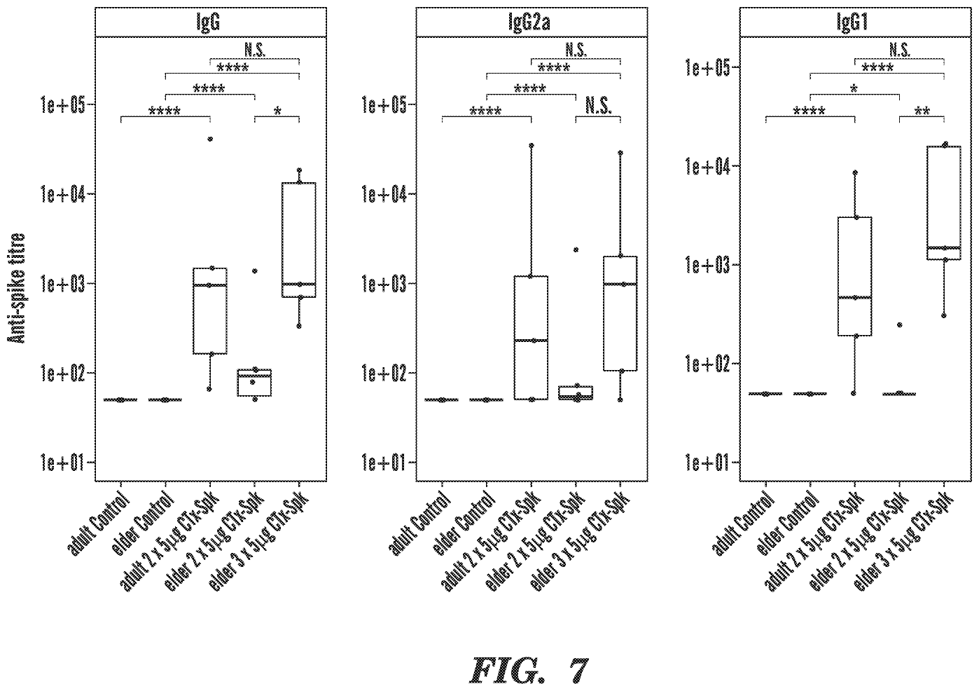

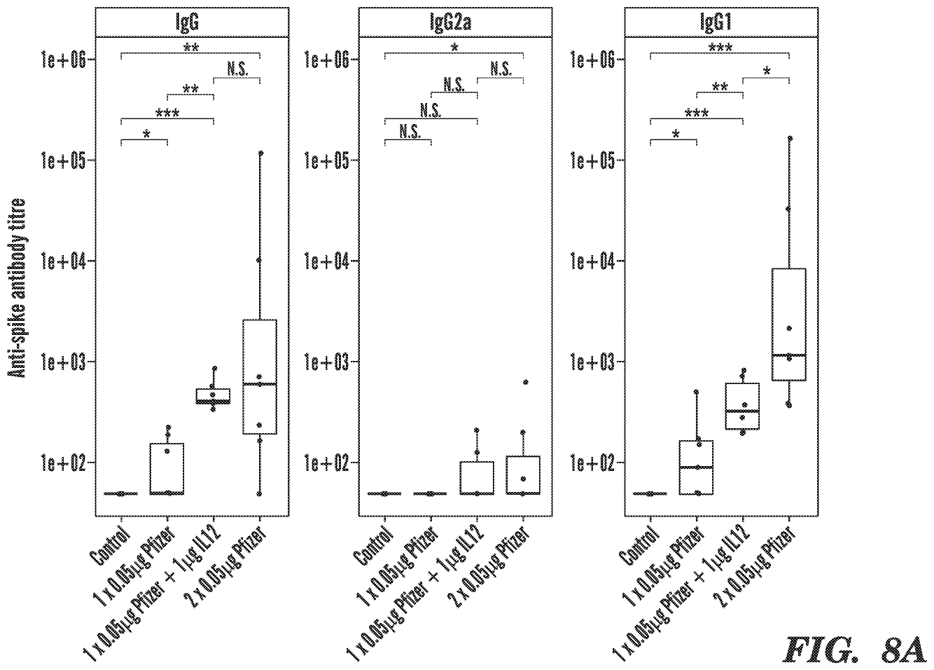

Efficacy of a third immunization was demonstrated in a prime-boost-boost model for elder mice where non-adjuvanted 5 μg mRNA encoding spike (CTx-Spk), elicited a significantly greater IgG and IgG1 than a 2 dose elder regimen, and non-inferior antibody IgG, IgG2a, and IgG1 titres compared to a 2-dose adult regimen (FIG. 7).

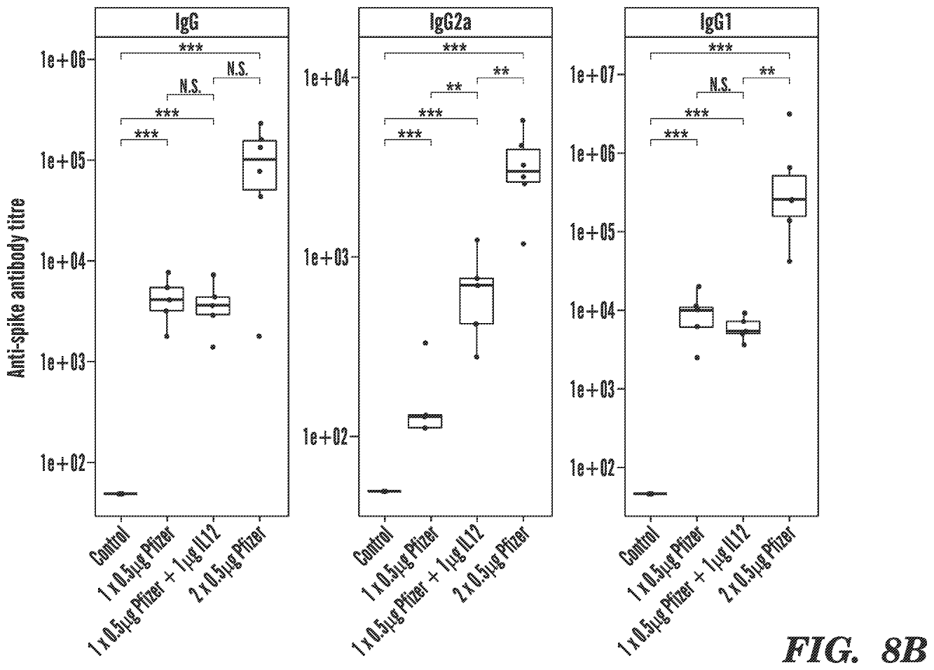

A goal of adjuvantation is to direct the immune response regardless of antigen source, and to immunize as few times as possible while eliciting significant immunity. mRNA encoding IL-12 (MOPv-IL12) was evaluated with a canonical mRNA vaccine, BNT162b2/Comirnaty (formulated by Pfizer/BioNTech), measuring the effect of adjuvantation on both singly immunized mice, and prime-boost immunized. Adult mice were administered either spike mRNA (Pfizer-formulated) alone, or admixed with IL12 mRNA (MOPv-IL12) in a single immunization and compared to 2 week separated prime-boost non-adjuvanted spike mRNA vaccinated mice to evaluate dose sparing effects. Serum antibody levels were observed on day 42 post-prime immunization. Adjuvantation of the single immunization with low-dose 0.05 μg of spike mRNA and 1 μg IL-12 mRNA displayed significant amplification of IgG, and IgG1 compared to non-adjuvant control (4.7- and 2.8-fold, respectively), and which elicited IgG titres that were non-inferior to a non-adjuvanted prime-boost immunized mouse (FIG. 8A). Adjuvantation of the single immunization of a 10× higher medium-dose 0.5 μg of spike mRNA with 1 μg IL-12 mRNA displayed significant amplification of IgG2a compared to non-adjuvant control (4.2-fold), with non-inferior IgG titres to a non-adjuvanted prime-boost immunized mouse (FIG. 8B). Each of the doses tested indicated repeatable ability to amplify immunity in single-immunized mice to levels comparable to a prime-boost immunization schedule, which would allow for fewer immunizations and subsequent dose sparing, faster immunity, and better vaccine uptake through increased vaccine schedule adherence.

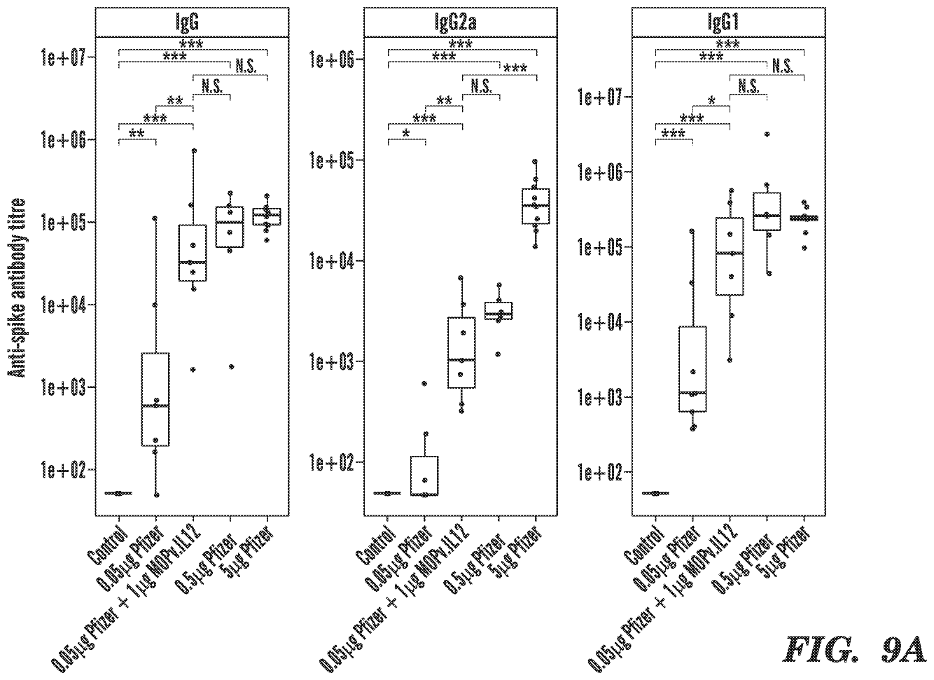

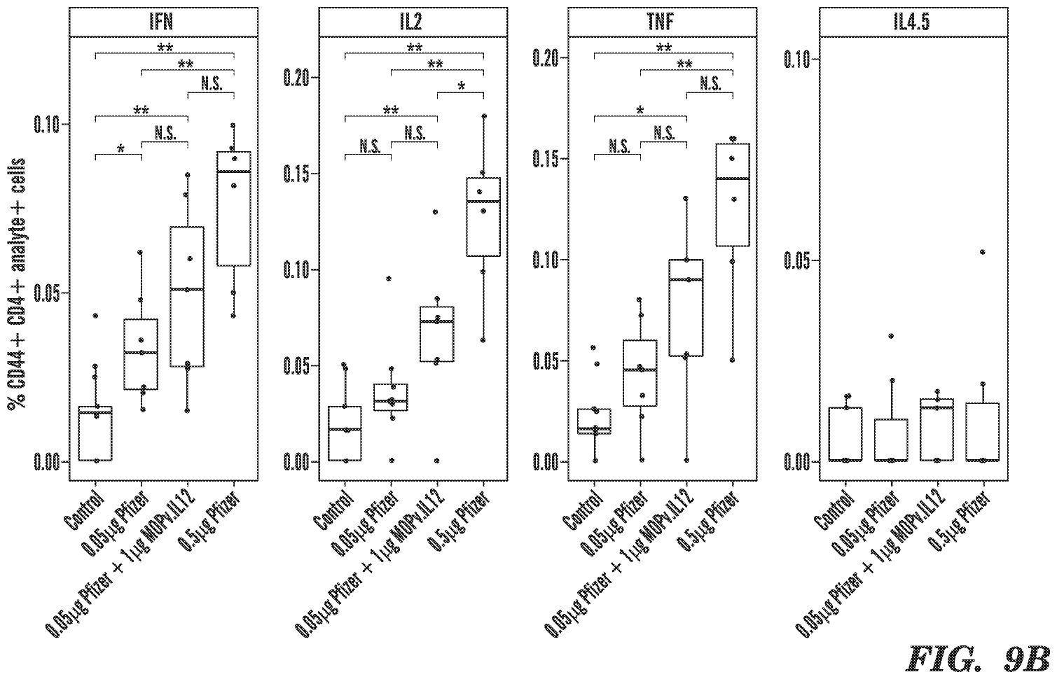

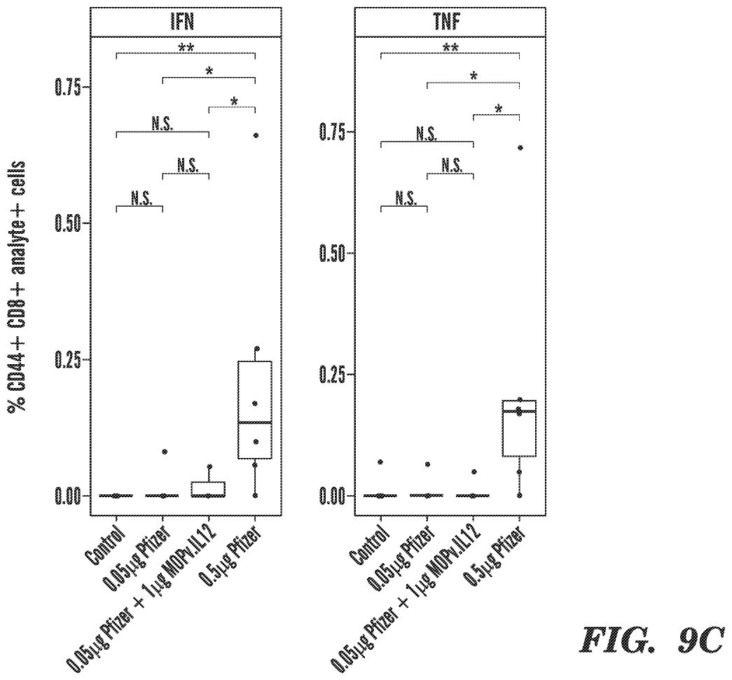

Another goal of adjuvantation is to amplify induced immunity of classical prime-boost immunizations to elicit greater anti-spike antibody titres and CD4/CD8 T cell responses which are immunological signatures believed to confer protection from SARS-COV-2. Mice were immunized following a 2-week separated prime-boost schedule, and serum anti-spike antibodies as well as flow cytometry quantification of CD4+ and CD8+ T cells by splenocyte restimulation with spike-specific peptides on day 42 post-prime immunization were measured. To evaluate the ability to dose spare the mRNA antigen a low-dose of mRNA encoding spike antigen was first tested by administering 0.05 μg mRNA encoding spike, formulated by Pfizer, ±1 μg MOPv-IL-12 mRNA (FIG. 9). Significantly greater IgG, IgG2a, and IgG1 responses were observed in IL-12 adjuvanted mice compared to non-adjuvanted spike-alone mice (8.2-, 13.4- and 6-fold, respectively, FIG. 9A). These responses were non-inferior to 10× and 100× greater antigen mRNA doses for IgG and IgG1, and non-inferior to a 10× greater antigen mRNA dose for IgG2a. Splenocyte restimulation had observed Th-1 polarization in both spike-alone and IL-12 adjuvanted spike groups with significant IFNγ cell positivity in both adjuvanted and non-adjuvanted groups compared to non-vaccinated controls, but significantly greater Th-1 responses were observed in IL-12 adjuvanted mice through induction of greater IL2+ and TNF+ cell positivity (FIG. 9B). CD4+ Th-2 polarization was not observed in either non-adjuvanted or adjuvanted mRNA conditions as IL4+ IL5+ cell positivity was unchanged (FIG. 9B), and CD8 responses were also unchanged (FIG. 8C).

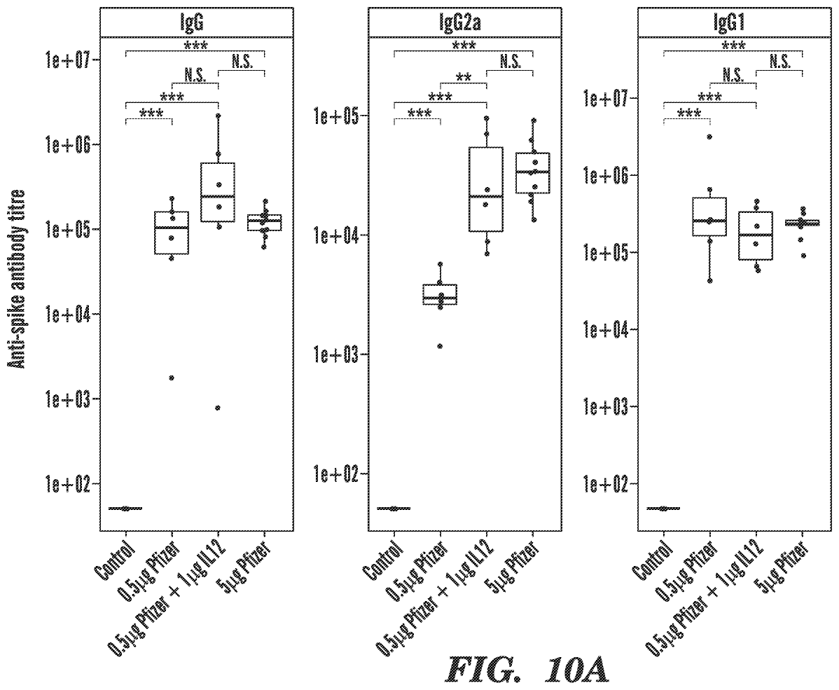

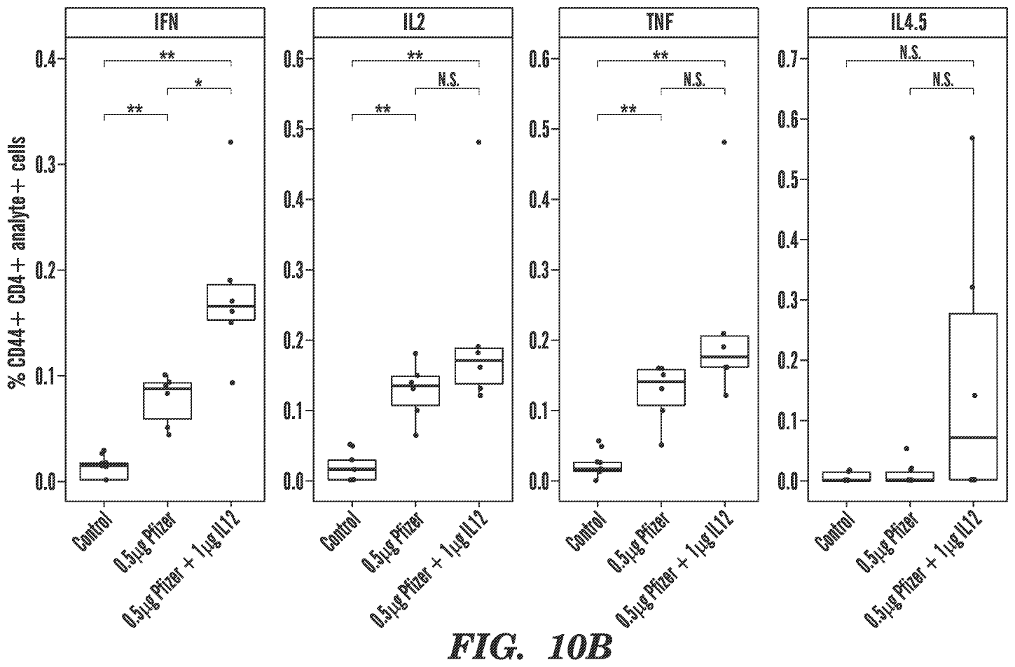

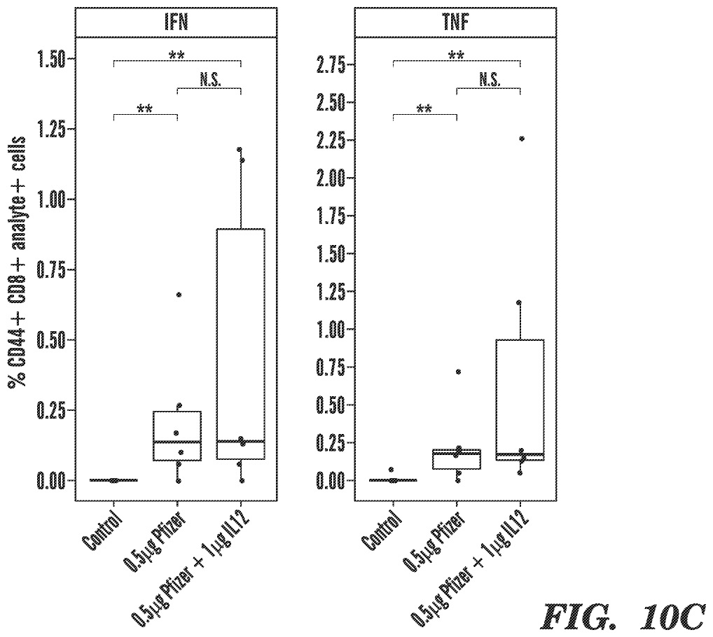

Adjuvantation of a 10× higher mRNA encoding spike (formulated by Pfizer) was evaluated with 1 μg mRNA encoding IL12 (MOPv-IL12) following the same schedule and readouts as in FIG. 9. IL-12 adjuvantation induced 11.8-fold significantly greater IgG2a than non-adjuvanted spike-alone control with non-inferior IgG, IgG2a, and IgG1 to a 10× higher dose of spike-alone vaccination (FIG. 10A). In splenocyte restimulation with spike-specific peptides cell positivity by flow cytometry indicated significantly greater IFN cell positivity in CD4+ cells of IL-12 adjuvanted mice compared to non-adjuvanted spike-alone immunized mice, generating a significant IL-12 adjuvant associated Th-1 polarization (FIG. 10B). A non-significant trend towards increased Th-2 polarization was also observed in CD4+ cells by some high responding IL4+ IL5+ CD4+ cell positivity which could indicate induction of a balanced immune response (FIG. 10B). Observance of CD8+ cytokine positivity post-splenocyte restimulation was observed in both non-adjuvanted spike-alone and IL-12-adjuvanted spike conditions with significant induction over non-vaccinated control, and with a non-significant trend towards greater IFN+ and TNF+ positivity in IL-12 adjuvanted compared to non-adjuvanted groups (FIG. 10C).

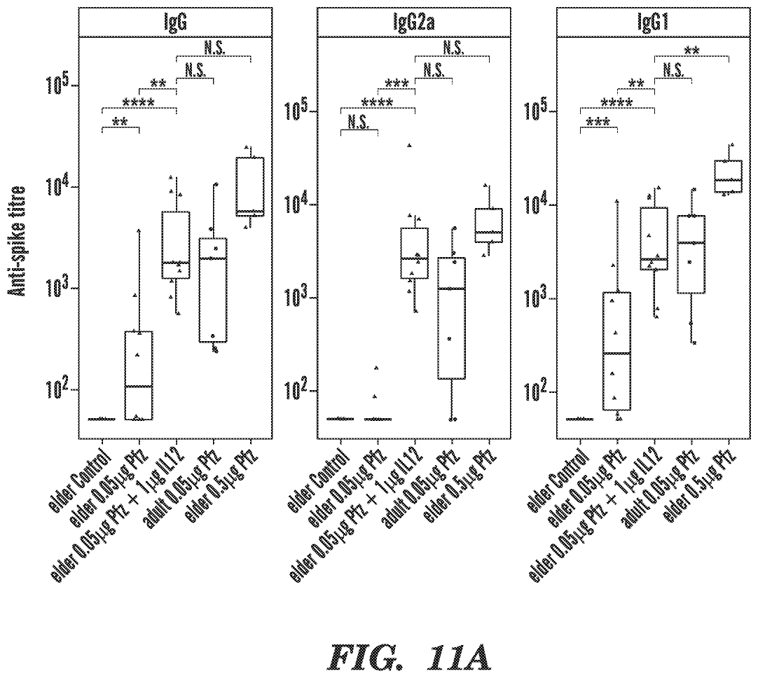

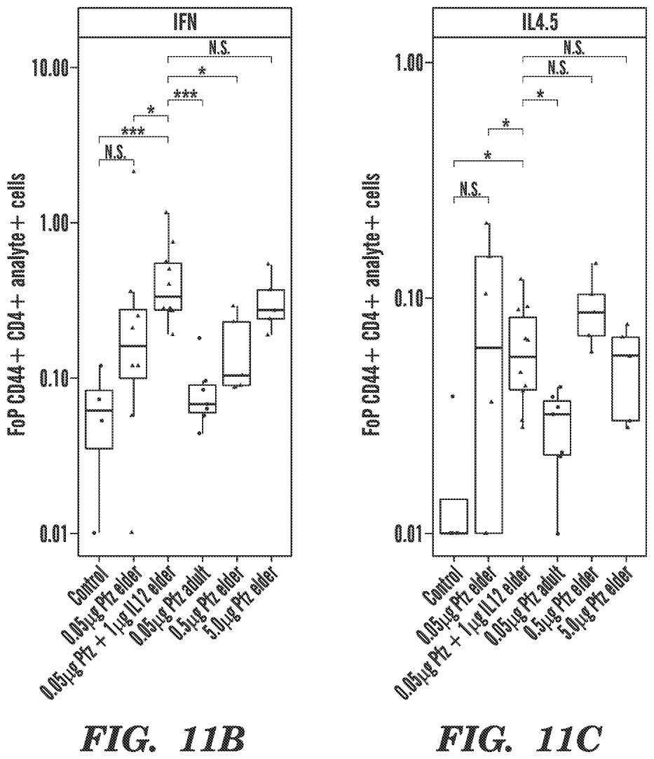

The effects of IL-12 adjuvantation in elder mice were assessed. Elder mice (>10 months old) were intramuscularly immunized following a prime-boost, 14 day separated schedule and compared to adult (˜6 week). Immunizations were with control dPBS, or 0.05 to 5.0 μg of encapsulated mRNA encoding spike protein (Pfizer's BNT162b2, ‘Pfz’) with or without 5 or 1 μg of encapsulated mRNA encoding a single-chain IL-12 heterodimer. On day 42 post-prime, 28 post-booster immunization, humoral immunity was evaluated in mouse sera for anti-spike antibody production, specifically total IgG (left), IgG2a (middle, Th1 marker), IgG1 (right, Th2 marker). Significant induction was observed over negative control, and adjuvantation over antigen-alone was observed in each antibody isotype (FIG. 11A). Antibody induction was non-inferior to adults administered the same antigen dose, supporting restored function, and were non-inferior to a 10× higher dose in elders for total IgG and IgG2a, supporting dose sparing. Splenocytes were collected on day 42 post-prime immunization, restimulated with spike-specific peptide, and a significant 2.1-fold greater frequency of parent (FoP) CD4⁺ cell IFN+ positivity was observed in adjuvanted versus non-adjuvanted groups (FIG. 11B). CD4+ T cell production of IL-4 and/or IL-5 (IL4.5) as markers of Th2 polarization indicated that IL-12 adjuvantation significantly induced more cell positivity than non-adjuvanted elder control and more than non-adjuvanted adult control (FIG. 11C). Adjuvant-amplified IL4.5 positivity was non-inferior to mice with 10× and 100× the antigen dose. These experiments demonstrate that IL-12 adjuvantation promotes robust immunity against SARS-COV-2 Spike in elder mice.

These experiments displayed an ability to adjuvant currently approved mRNA vaccine technology, utilizing novel delivery and formulation of a single-chain IL-12 adjuvant that can induce greater immunogenicity at two different doses of mRNA encoding spike, with displayed benefit to both humoral and cell-mediated responses that are believed to confer protection from SARS-COV-2.

The immunosenescence demonstrated herein establishes the need for adjuvantation in elders with:
1) Elder-impaired Th-1 polarized cytokine production in human whole blood ex vivo stimulation
2) Impaired elder mouse antibody titres compared to adult mice
3) Th-2 skewing of elder murine humoral immunity compared to the adult Th-1 polarization
4) Significant elder-impaired neutralization of receptor binding domain

Further described herein is the discovery that LNP delivery of mRNA encoding a single chain heterodimer IL-12p70 was:
1) capable of inducing Th1 polarization with cytokine production in human whole blood ex vivo
2) induced gene expression in antigen presenting cells (monocyte derived dendritic cells: MoDCs) in human, in vitro, in both adult and elder human participant samples
3) expressed bioactive IL-12 by inducing human IFNγ in vitro (96 hr PBMC stimulation)
4) demonstrated murine in vivo adjuvant activity by inducing 3.5-3.7-fold significantly greater antigen specific IgG2a on day 28 and day 42 post-prime immunization utilizing antigen and adjuvant mRNA encapsulated in LNP (MOPv-IL12)
5) induced immunity in elder mice, with a trend towards MOPv-IL-12 adjuvantation compared to adult control mice
6) significant Th-1 polarization in adult and elder mice with IL-12 adjuvantation
7) restored elder mouse immunogenicity to adult-like levels through a tertiary third non-adjuvanted vaccination in elder mice
8) amplified immunity in single-shot immunization of mice with an alternative antigen source, Pfizer/BioNTech's BNT162b2 compared to non-adjuvanted spike-alone mice, with increased IgG and IgG1 in IL-12 adjuvanted 0.05 μg BNT16262 mRNA and increased IgG2a in IL-12 adjuvanted 0.5 μg BNT162b2 mRNA
9) amplified adult immunity in prime-boost immunization of mice with a low dose 0.05 μg mRNA BNT162b2 adjuvanted with MOPv-IL12 inducing greater (6- to 13.4-fold) IgG, IgG2a, and IgG1, and amplified Th-1 polarized CD4 spike-specific T cells compared to non-vaccinated spike-alone
10) Amplified adult immunity in prime-boost immunization of mice with a medium dose 0.5 μg mRNA BNT162b2 adjuvanted with IL-12 inducing greater (5.4- to 11.8-fold) IgG2a, and IgG1, and amplified Th-1 polarized CD4 spike-specific T cells, and trend towards increased CD8 T cells compared to non-vaccinated spike-alone

It is contemplated herein that the methods and compositions described herein provide:
1) increased immunity (e.g., maximal immune response, quality of immune response)
2) increased Th1 immunity (e.g., IgG2a antibody production, CD4+ polarization)
3) decreased waning immunity
4) decreased number of doses required to induce immunity in elders
5) decreased dose required to elicit immunity (e.g., 1/10^{th} or 1/100^{th} the normal dose)
6) decreased reactogenicity (e.g., effective immunization with lower dose)
7) concomitant immune activation with multiple antigen sources (e.g., targeting multiple variants at once, or multiple pathogens at once)
8) effective elder immunization, as this adjuvant demonstrates robust activity in leukocytes from elderly individuals, developing vaccines tailored for this vulnerable population is crucial to ensure durable and robust protection (e.g., from viral respiratory diseases such as influenza and SARS-COV-2)
9) adjuvanticity against various antigenic targets (Spike as a model antigen of SARS-COV-2 that could be replaced with other pathogen's antigenic targets, e.g., SARS-COV-2 variants, tuberculosis, influenza, etc.)
10) increased antigen-specific affinity (e.g., T cell receptor, antibody affinity)
11) increased number of epitope targets following single or prime-boost immunization
12) increased antigen presenting cell mobilization to draining lymph nodes
13) increased antigen presentation capacity
14) increased germinal center functionality (e.g., formation and quality of presentation)

### Example 2

It is demonstrated herein that IL-12 biological adjuvantation leads to a more durable immune response. This technology can overcome one of the limitations of mRNA vaccination strategies, rapid waning immunity. The currently employed solution with non-adjuvanted mRNA vaccination is repeated immunization to maintain the level of immunogenicity required for protection, but this can lead to greater vulnerability during the period of waning immunity, and reduced vaccine uptake from people who do not get boosted, ultimately reducing vaccine conferred protection. Waning immunity can be modeled in murine models, which can also be employed to evaluate alternative methods to induce durable immunity. It is described herein that adjuvantation via co-delivery of cytokines/chemokines (e.g., single chain IL-12p70 heterodimer of IL-12p35+IL-12p40) with mRNA encoding the cytokine/chemokine encapsulated in lipid nanoparticles (LNP) to adjuvant mRNA-encoded antigen (e.g., SARS-COV-2 spike antigen, “Pfz”).

As described elsewhere herein, IL-12 led to a greater durability and greater induction of SARS-COV-2 spike-specific immunity at day 84 post-prime immunization. Herein, immunity in adult and elder mice is evaluated at 6 months, day 168, post-prime immunization to study long-term durability. The benefit of LNP delivered mRNA encoding IL-12p70 as an adjuvant was sustained, displaying significantly greater durable immunity, continued capacity to confer dose-sparing and amplified elder immunity, and a capacity to rescue non-responsive immune responses. Humoral immunity has been implicated as a correlate of protection, as a therapy with monoclonal antibody administration, and therefore these measures of amplified humoral immunity are expected to also result in greater protection.

The beneficial effects of cytokine adjuvantation include more durable immunity, inducing greater total IgG, IgG2a, and IgG1 in both adults and elders. Application of this experimental timeline to a human context is contemplated to confer immunity greater than the 6 months measured here, as mice develop and progress in an accelerated timeframe.

In summary, described herein is immunosenescence establishing the need for adjuvantation in both adult and elder mice, with:
A) Waning immunity in a young adult murine model across total and isotype-specific antibodies that are markers of T_{H}1 and T_{H}2 polarizing on day 168 post-prime immunization
B) Waning immunity in an aged/elder murine model across total and isotype-specific antibodies that are markers of T_{H}1 and T_{H}2 polarizing on day 168 post-prime immunization
C) Waning immunity resulting in the loss of detectability for a notable portion of adult and elder animals

The technology of cytokine encoding mRNA (e.g., IL-12p70) induced increase of immune durability confers advantages that could include:
1) Result in more sustained antigen-specific immunity in healthy young adult individuals 2) Prolong the period of protection available in humans extending beyond 3 months due to species differential kinetics
3) Guide immune responses with concomitant TH1 TH2 induction, or TH1 polarization
4) Permit dose sparing of antigen (protein or mRNA encoded) with a decreased dose required to elicit immunity (e.g., 1/10th or 1/100th the normal dose)
5) Activate immune responses that would otherwise be non-responsive
6) Permit vaccination programs targeted towards reducing reactogenicity and other negative side effects through dose sparing
7) Permit vaccination programs targeting other SARS-COV-2 variants of concern (e.g., Omicron) via dose-sparing enabling multiple antigen delivery
8) Permit vaccination programs targeting other SARS-COV-2 antigens of interest (e.g., Nucleocapsid) via dose-sparing enabling multiple antigen delivery
9) Permits vaccination programs targeting other pathogens that would not be amenable to the rapidly waning immunity observed with mRNA vaccination (e.g., Tuberculosis)
10) Permit vaccination programs targeting multiple pathogens at once, requiring dose sparing alongside durable immunity induction
11) A decreased number of injections required to induce long-lasting immunity
12) An increase in antigen-specific affinity (e.g., T cell receptor, antibody affinity)
13) An increased number of epitope targets following single or prime-boost immunization
14) Increased antigen presenting cell mobilization to draining lymph nodes
15) Increased antigen presentation capacity
16) Increased germinal center functionality (e.g., formation and quality of presentation)
17) Each of the above benefits are expected in immunocompromised individuals (e.g., neonatal, elderly, obese and diabetic, cancer patients, transplant recipients, individuals with immunodeficient conditions, etc.)

### Example 3

Described herein are multiple benefits from the use of a single chain mRNA encoding IL-12p70 to adjuvant antigen-specific responses and amplify vaccine induced immunity.

Demonstrated herein is immunosenescence which establishes the need for adjuvantation in elders with:
1) Elder-impaired TH1 polarized cytokine production in human whole blood ex vivo stimulation
2) Impaired elder mouse antibody titres compared to adult mice
3) TH2 skewing of elder murine humoral immunity compared to the adult TH1 polarization
4) Significant elder-impaired neutralization of receptor binding domain
5) Significant elder-impaired neutralization of RBD and SARS-COV-2

It is further described herein that LNP delivery of mRNA encoding a single chain heterodimer IL-12p70:
6) was capable of inducing TH1 polarization with cytokine production in human whole blood ex vivo
7) induced gene expression in antigen presenting cells (monocyte derived dendritic cells: MoDCs) in human, in vitro, in both adult and elder human participant samples
8) expressed bioactive IL-12 by inducing human IFN-gamma in vitro (96 hr PBMC stimulation)
9) demonstrated murine in vivo adjuvant activity by inducing 3.5-3.7-fold significantly greater antigen specific IgG2a on day 28 and day 42 post-prime immunization utilizing antigen and adjuvant mRNA encapsulated in LNP formulated by Combined Therapeutics
10) induced immunity in elder mice, with a trend towards MOPv-IL-12 adjuvantation compared to adult control mice
11) significant TH1 polarization in adult and elder mice with IL-12 adjuvantation
12) restored elder mouse immunogenicity to adult-like levels through a tertiary third non-adjuvanted vaccination in elder mice
13) amplified immunity in single-shot immunization of mice with an alternative antigen source, Pfizer/BioNTech's BNT162b2 compared to non-adjuvanted spike-alone mice, with increased IgG and IgG1 in IL-12 adjuvanted 0.05 μg BNT162b2 mRNA and increased IgG2a in IL-12 adjuvanted 0.5 μg BNT162b2 mRNA, and IL-12 adjuvanted 0.1 μg BNT162b2 responses by ELISPOT.
14) amplified adult immunity in prime-boost immunization of mice with a low dose 0.05 μg mRNA BNT162b2 adjuvanted with MOPv-IL12 inducing greater (6- to 13.4-fold) IgG, IgG2a, and IgG1, and amplified TH1 polarized CD4 spike-specific T cells compared to non-vaccinated spike-alone
15) Amplified adult immunity in prime-boost immunization of mice with a medium dose 0.5 μg mRNA BNT162b2 adjuvanted with IL-12 inducing greater (5.4- to 11.8-fold) IgG2a, and IgG1, and amplified TH1 polarized CD4+ spike-specific T cells, and trend towards increased CD8 T cells compared to non-vaccinated spike-alone

The data provided herein demonstrate increased durability of immunity combatting waning immunity; a mechanism of action for dose-sparing via increased DC maturation, DC chemotaxis, increased FDC area and density, increased BCZ area, and increased number of GC; and extended potency and human dose equivalency.

As described elsewhere herein, there is an opportunity to adjuvant mRNA vaccines, e.g., FIGS. 1-2. Adult and elder human participant samples had differential immune activation. Specifically, in vitro human whole blood stimulation with BNT16262 failed to induce IL-12p70, indicating that IL-12p70 adjuvantation could benefit both adult and elder individuals in mRNA formulations and immunizations. This opportunity to adjuvant identifies a lack of induction in both adult and elder participants when exposed to BNT162b2.

Furthermore, it was demonstrated there was less production of TH1 polarizing analytes in elder (>60 Y) participants compared to adults (18-50 Y). This was observed with direct impairments of IFNγ and CXCL10 production, and impairments in a broader analysis of observing total analyte induction of TH1 polarizing analytes. FIGS. 1A-1G demonstrates TH1 impairment via a Generalized Estimating Equations Generalized Linear Model (GEEGLM) analysis.

An in vivo murine model was employed and displayed age-associated impairments. There is a correlation of murine anti-spike IgG antibody with capacity to inhibit recombinant receptor binding domain (RBD) protein from binding to human angiotensin converting enzyme 2 (hACE2) observed similar significant correlations between age groups (FIG. 2D). This indicates that it is the degree of antibody induced, not quality, that controlled the capacity to neutralize a critical function of SARS-COV-2 infection. Elder mouse-impaired surrogate virus neutralization was further characterized in a true neutralization assay with elder mice displaying an impaired ability to prevent live Wuhan1 SARS-COV-2 from inducing cytopathic effects in an in vitro assay with Vero TMPRSS2 cells (FIG. 2E). Lastly, the humoral deficiencies in mice were paired with direct measurement of CD4+ T cell polarization with elder mouse CD4+ T cells inducing less IFNγ and TNF than adult mice (FIG. 2F), validating the observations in human in vitro assays, and in murine antibody isotope evaluation. Additionally, an impairment in murine CD8+ T cell TNF positivity was observed (FIG. 2G), another indication for the opportunity to adjuvant the elderly to restore adult-like function.

### Immune Durability:

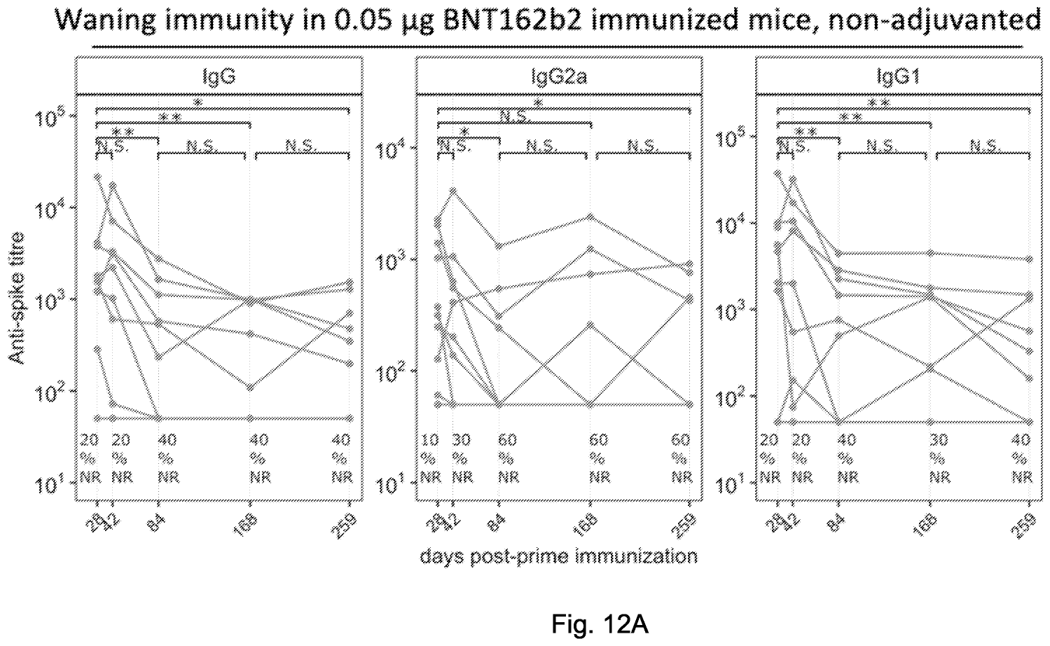

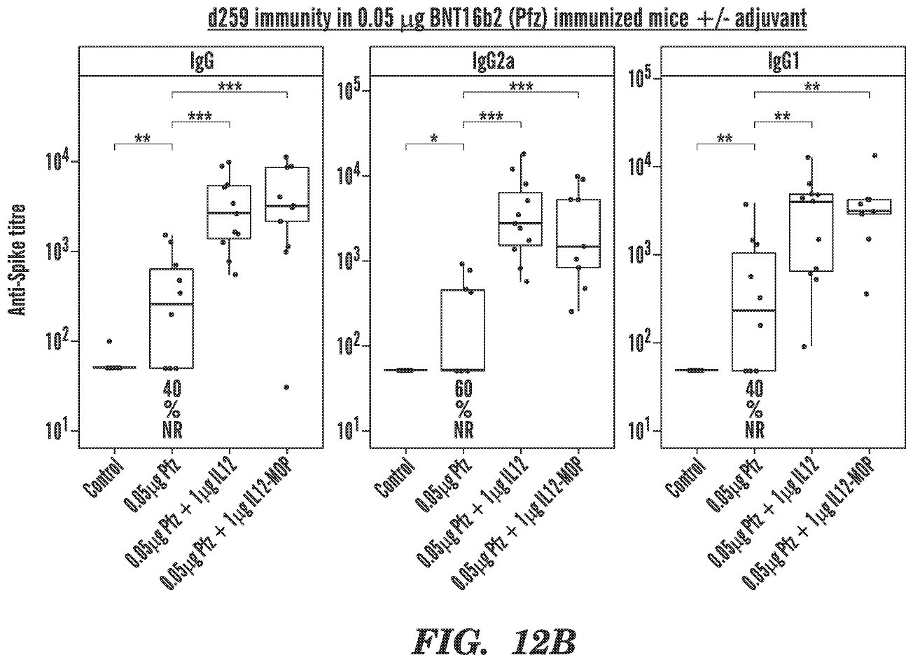

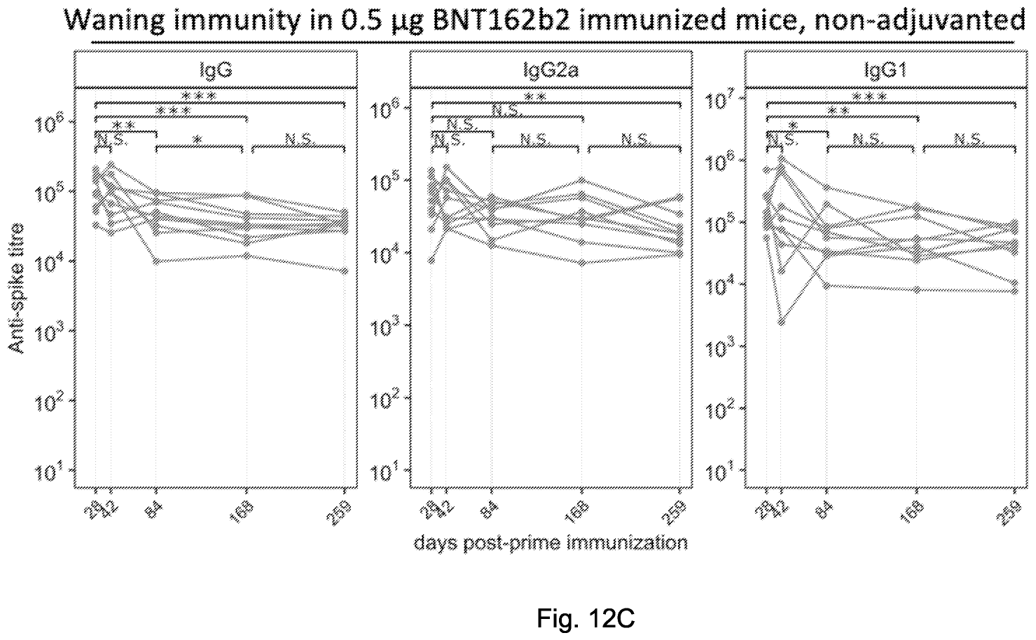

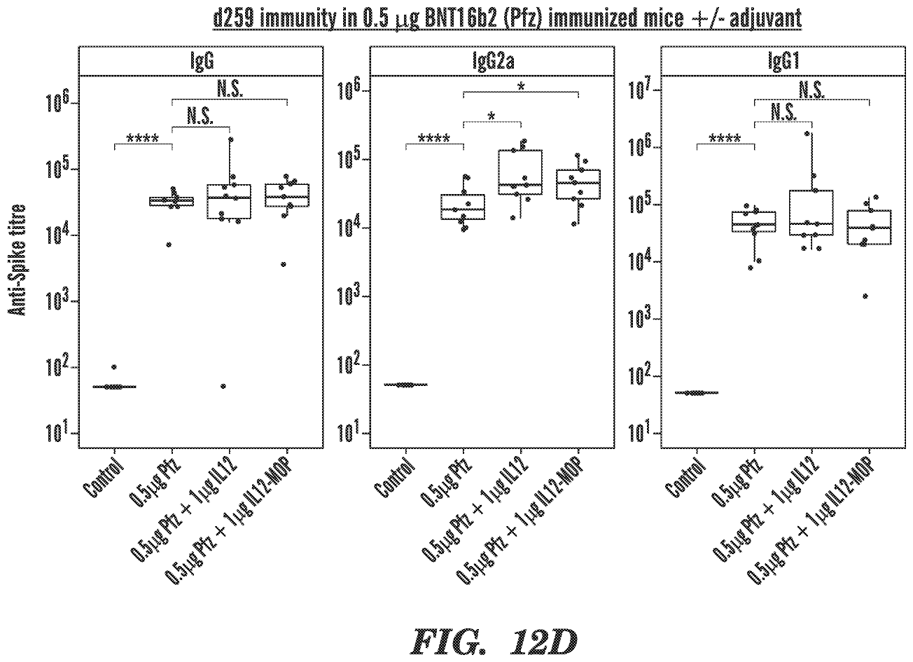

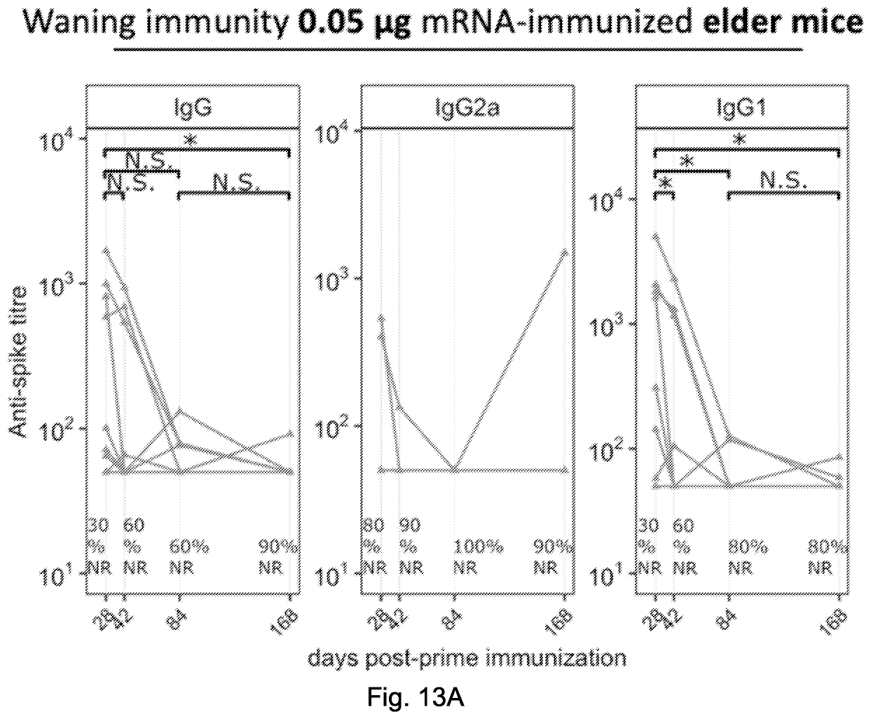

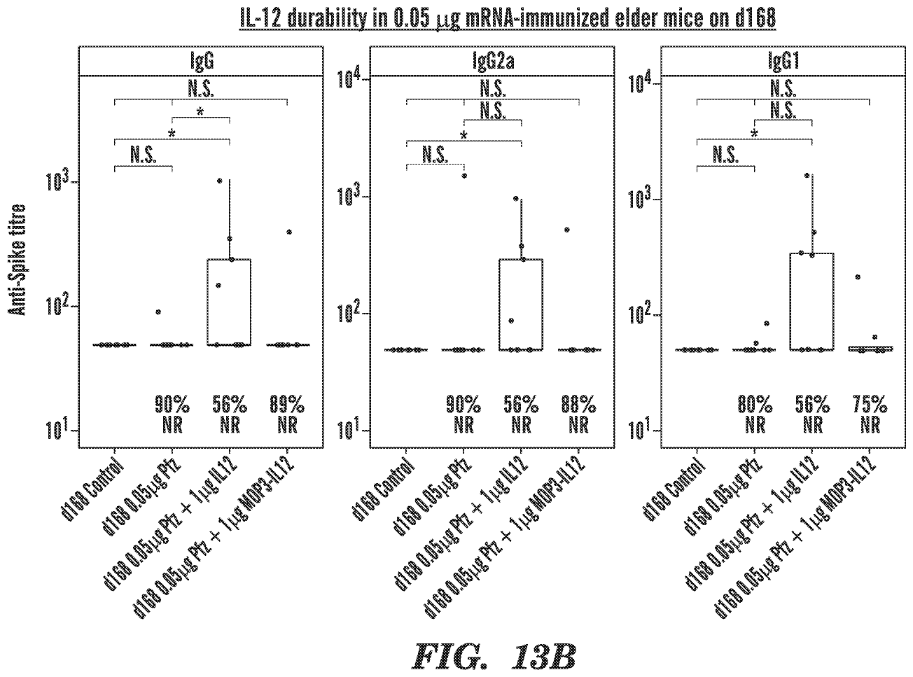

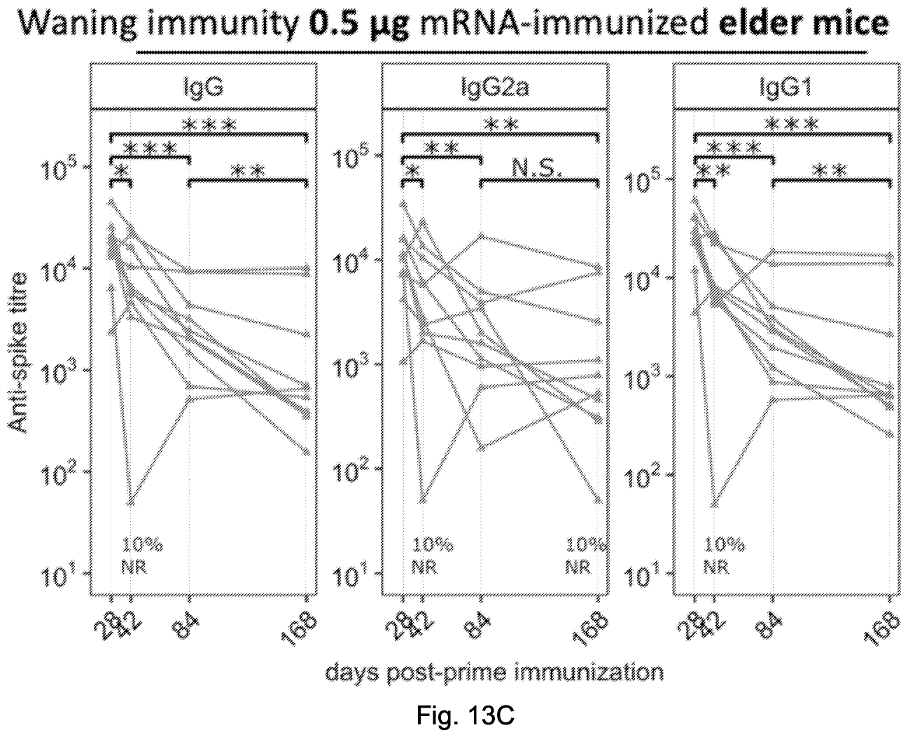

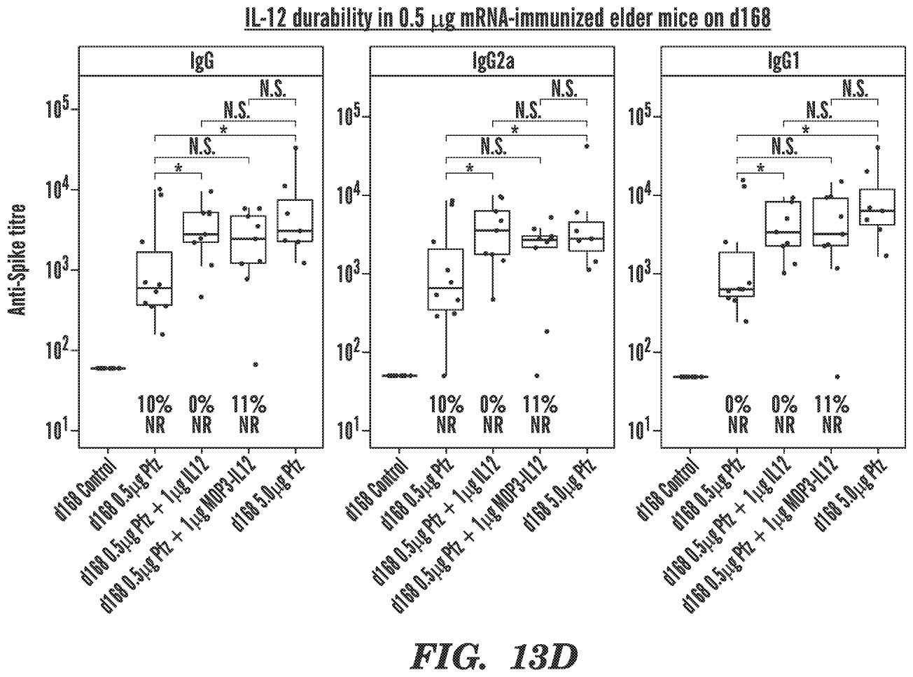

IL-12 adjuvantation was evaluated for amplification of immune durability (FIGS. 12-13). An admixture of BNT162b2 with mRNA encoding IL-12 (with or without Combined Therapeutic's Multi-Organ Protection, MOP, proprietary sequence) administered as a prime-boost immunization series separated by 14 days, led to amplified immunity that persisted through elderly/immune-distinct ages, e.g., at least 259 days (FIG. 12). Mice immunized with 0.05 μg mRNA in BNT162b2, without adjuvantation, had waning immunity (FIG. 12A). Adjuvantation with 1 μg of mRNA encoding IL-12, with or without MOP controlled expression, alongside an antigen dose of 0.05 μg of mRNA in BNT162b2-immunized adult mice resulted in sustained total IgG, IgG2a, and IgG1 through 259 days post-prime immunization (FIG. 12B). Importantly, IL-12 adjuvanted mice had 100% responsivity while non-adjuvanted were 40-60% non-responsive (NR), dependent on isotype. A 10× greater antigen dose, 0.5 μg mRNA in BNT162b2 similarly had waning immunity in total IgG, IgG2a, and IgG1 through 259 days post-immunization (FIG. 12C). IL-12 mRNA adjuvantation sustained greater IgG2a responses in mice immunized with 0.5 μg mRNA in BNT162b2 (FIG. 12D). Waning immunity is one of the current major concerns in mRNA vaccines, and adjuvantation with mRNA encoding IL-12 led to long term sustained immunity and sustained evidence of TH1 polarization where mRNA encoding antigen alone was unable to. This amplified immunogenicity will protect more from disease, based on associations of increased antibody responses correlating with increased inhibition of RBD binding human ACE2 (FIG. 2G). Furthermore, the IL-12 adjuvantation effect amplifying responses to BNT162b2 are expected to translate to other antigens, and increasing immune durability will be a critical checkpoint in enabling protective immune responses against continuing SARS-COV-2 variants, and to non-pandemic pathogens.

Age-dependent immune activation differences were observed post-vaccination. Triggering effective immunity in the elder age setting (>60 years in humans) is critical to confer protection, particularly because these age groups are often more susceptible to disease (e.g., age-related mortality in SARS-COV-2). To model ontogeny, elder mice were immunized (>10 months old) with 0.05 μg of non-adjuvanted mRNA (BNT162b2) and observed sera at day 28, 42, 84, and 168 post-prime immunization. Elder mice had significantly lower spike specific IgG on day 168 post-prime and lower IgG1 on days 84 and 168, compared to day 28 (FIG. 13A). IL-12 adjuvantation rescued elder immunogenicity, inducing a more durable immune response with significantly induced IgG, IgG2a, and IgG1 spike-specific humoral response compared to PBS-immunized and a trend to greater response than non-adjuvanted (FIG. 13C). Non-adjuvanted mice with waning immunity had 60-100% of mice be below the lower limit of detection, termed nonresponsive (NR), with 80-90% of non-adjuvanted mice with no detectable anti-spike IgG, IgG2a, or IgG1 antibodies on day 168 post-prime (FIG. 13A). IL-12 rescued this response, reducing the 0.05 μg immunized 80-90% NR elder mice to IL-12 adjuvanted 56% NR on day 168 post-prime immunization (FIG. 13B). The magnitude of IL-12 amplification of elder immunity was to a level that was non-inferior to adult mice immunized with the same antigen dose (without adjuvant) (FIG. 1B). As adult humans suffer less disease than elders (>60 years) it is anticipated that adult-like immunity would also confer greater protection. Other elder mice were administered a 10-fold higher antigen dose, 0.5 μg of mRNA encoding spike antigen without adjuvantation, and had significant waning immunity in total IgG, IgG2a, and IgG1 on days 42, 84, and 168 post-prime immunization (FIG. 13C). IL-12 adjuvantation of this 0.5 μg of mRNA encoding spike antigen conferred a more durable and greater IgG, IgG2a, and IgG1 production in elder mice (FIG. 13D). This amplified immune response conferred a dose-sparing effect, as IL-12 adjuvantation of 0.5 μg of mRNA was non-inferior to another 10-fold greater nonadjuvanted antigen dose of 5.0 μg of mRNA in elder mice. Dose sparing permits greater effective antigen doses in humans, circumventing maximum tolerized doses, and can permit multivalent immunizations.

### Mechanism of Action:

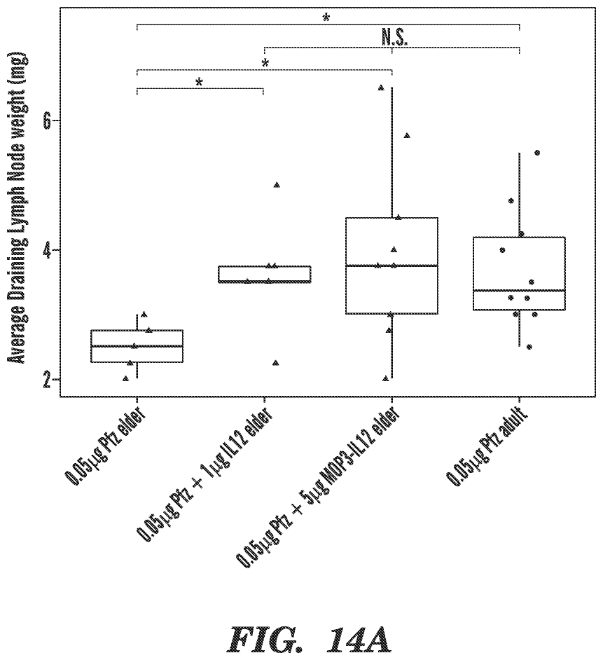

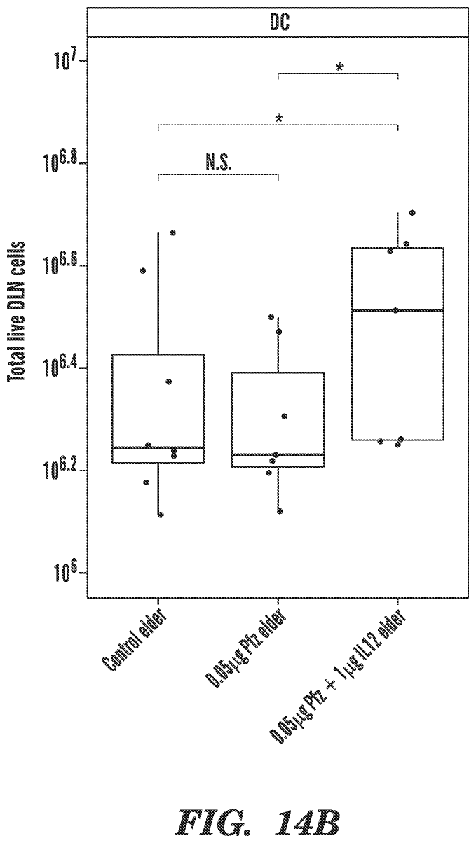

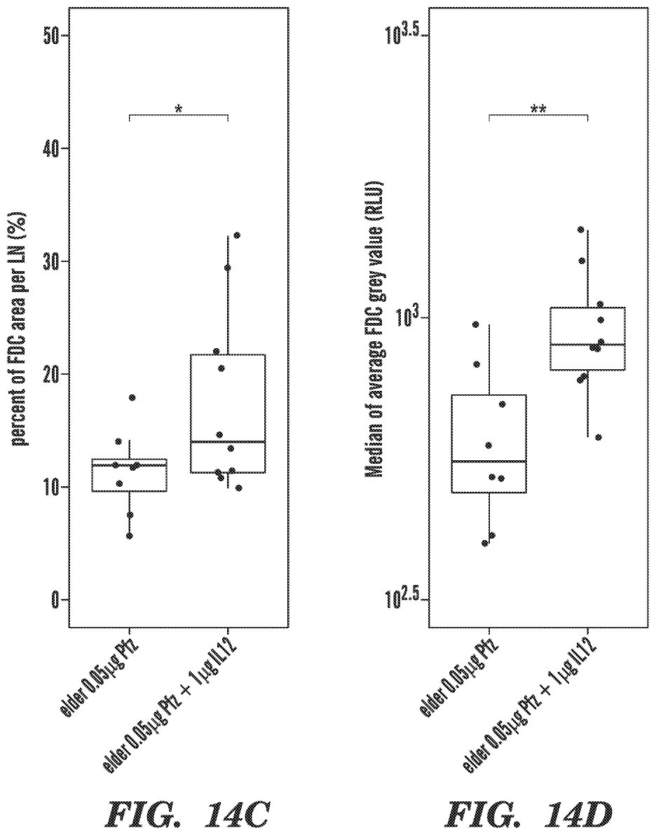

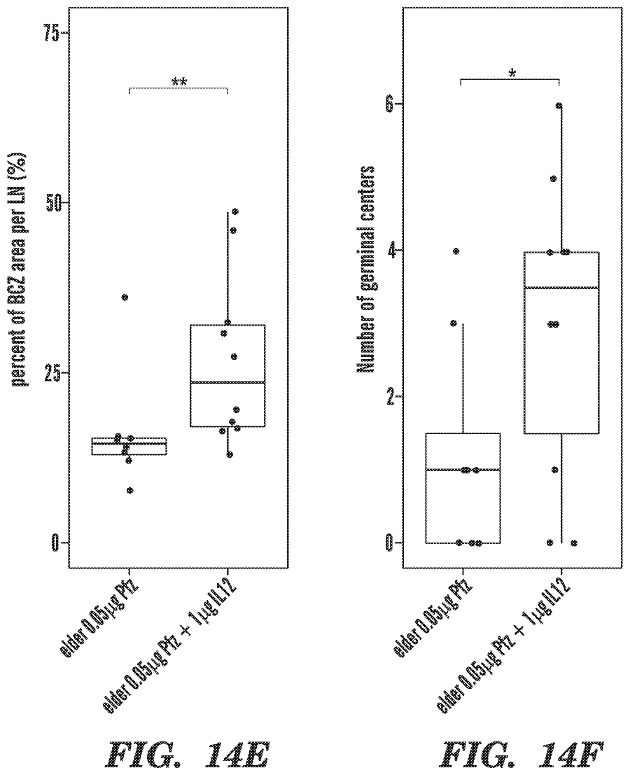

Mechanism of action is relevant to precision immunology. Murine lymph nodes were evaluated post-vaccination by histology and flow cytometry. As an indirect measure of immune activation, the average weight of a draining lymph node was significantly greater in IL-12 and IL-12-MOP adjuvanted mice compared to non-adjuvanted, 0.05 μg mRNA in BNT162b2 alone (FIG. 14A). Dendritic Cells (DC) are antigen presenting cells that prime T cells and induce subsequent germinal center reactions for B cell antigen screening, clonal expansion, and affinity maturation. These cells can also activate peripheral expansion of CD4+ and CD8+ T cells, and NK cells. IL-12 induced a significant lymph-node increase of DC (CD3-CD19-MHC.II+CD11c+CD14), quantified by flow cytometry on d9 post-booster immunization (FIG. 14B). Increased frequency indicates increased activation and chemotaxis of DC. B cell antigen specific selection relies on follicular dendritic (FDC) activity in the lymph node light zones. FDC regions were expanded, and density increased (median of average FDC gray value) in IL-12 adjuvanted compared to non-adjuvanted 0.05 μg mRNA in BNT162b2-alone groups (FIG. 14C-4D). Subsequently the B cell zone (BCZ) and number of germinal centers (GC) were also significantly increased in the IL-12 adjuvanted group (FIG. 14E-4F). Increases in BCZ, and secondary internal structures of number of GC are anticipated to permit dose-sparing and to increase clonal diversity via increased antigen-based B cell selection. In sum, investigation of draining lymph nodes indicated that IL-12 had amplified DC maturation and chemotaxis with increased lymph node frequency, increased FDC area and density, amplified BCZ area, and increased GC number. Each of these steps contribute to effective humoral immune responses.

### Extended Potency and Anticipated Human Equivalency:

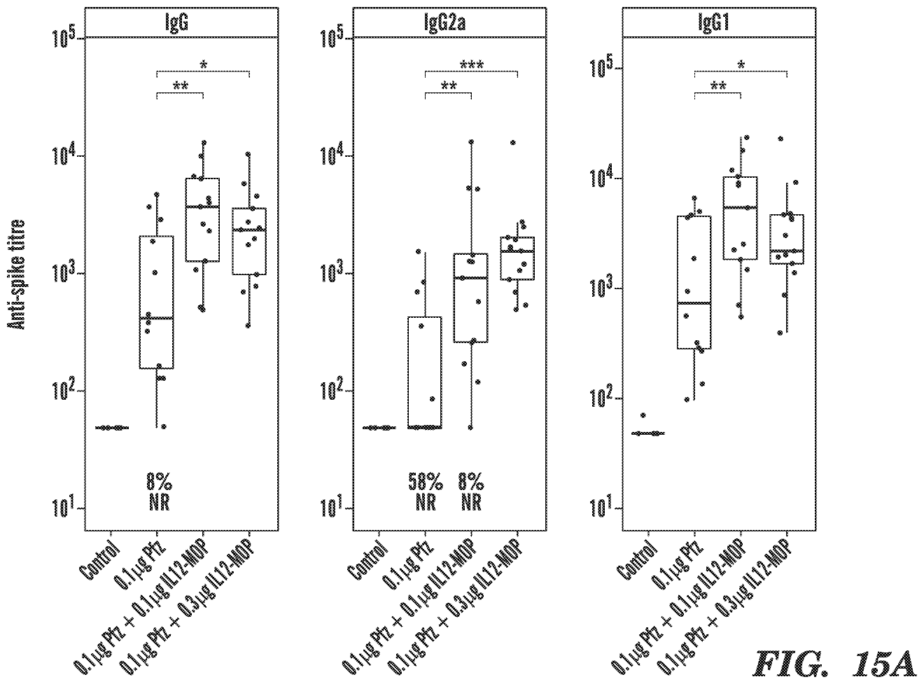

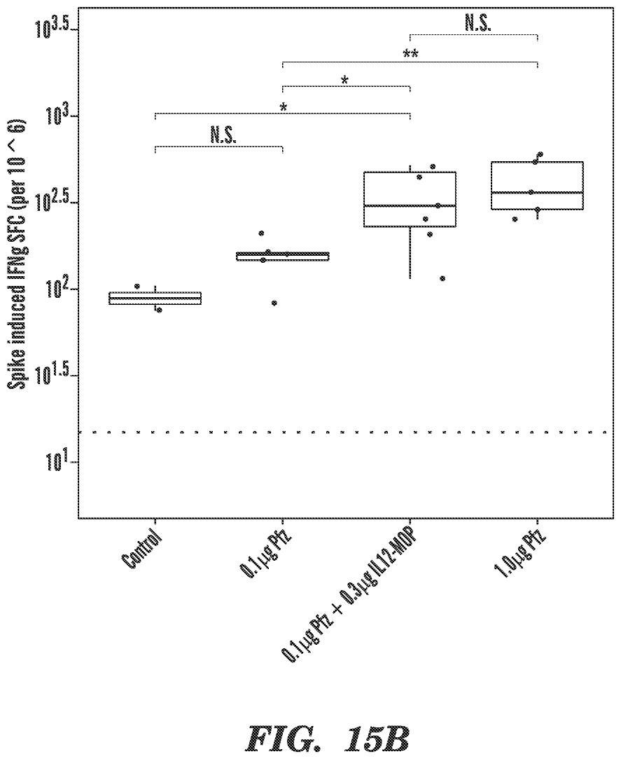

LNP delivery of mRNA encoding IL-12p70 can result in a positive feedback induction of IFNγ and lead to innate immune maturation, and adaptive immune support. The potency of this adjuvantation system was evaluated and it was found that a dose of 0.1 μg mRNA encoding IL-12 was still able to confer significant adjuvantation effects, with TH1-polarization characteristics, amplifying IgG and IgG2a over non-adjuvanted control (FIG. 15A). The lowest dose of IL-12 tested still conferred an adjuvantation effect, therefore even lower doses are expected to be an adjuvant. IL-12 impact on cell mediated immunity was evaluated by ELISPOT, with significantly greater IFNγ spot forming cell (SFC) formation with 1 μg IL-12-MOP adjuvantation of 0.1 μg of BNT162b2 (Pfz) compared to antigen alone (FIG. 15B). The level of induction brought the adjuvanted 0.1 μg antigen response to a non-inferior level compared to a 10× higher antigen dose, 1.0 μg mRNA in BNT162b2, conferring the capacity for dose sparing to induce greater cell mediated immunity in elder mice.

Throughout the experiments described herein doses of mRNA encoding IL-12 ranging from 0.1 to 5 μg of mRNA, with or without MOP, have displayed adjuvanticity for antigen doses ranging between 0.05 μg and 0.5 μg mRNA in BNT162b2. The formulations of the IL-12 adjuvant and BNT162b2 differ, specifically with different production companies (Combined Therapeutics, ‘CTx’, and Pfizer/BioNTech, ‘Pfz’, respectively), different components (e.g., cationic lipid), different mRNA encoding aspects (unmodified uridine in IL-12, and pseudouridine in BNT162b2), expression control (IL-12 with MOP, and no specific control in BNT162b2), and different mRNA encoded proteins (IL-12 vs spike). Without wishing to be bound by theory, these differences may impact the charged nature of LNP, the rate of phagocytosis and macropinocytosis, the recognition of LNP components, the protein translation rates, the longevity of mRNA translation, ultimately changing protein expression rates and duration. In separate experiments delivery of mRNA encoding spike protein was evaluated within the same LNP formulation as the IL-12 was delivered. It was found that a dose of 5 μg mRNA encoding spike formulated by CTx was roughly equivalent to 0.5 μg of mRNA in BNT162b2.

The mRNA encoding IL-12 adjuvant doses with greatest induction of immunity have been between 0.1 μg and 1 μg of mRNA, i.e., between 1/100th and 1/10th of the antigen dose. Lack of dose-dependent loss of adjuvantation means a wider adjuvantation range is observed, with 0.03 μg mRNA able to adjuvant, and a maximal 5 μg of mRNA having observed to adjuvant DC responses (a range of ˜ 1/200th to ⅕th the antigen dose). The human dose for BNT16262 is 30 μg, therefore a human IL-12 adjuvantation dose is within the range of 1/200th and ⅕th of this, corresponding to 0.15 μg and 6 μg of mRNA encoding IL-12, if similarly encapsulated and pseudouridine-protected.

SEQ ID NO: 200 IL-12α subunit isoform 1 amino acid sequence
1 mwppgsasqp ppspaaatgl hpaarpvslq crlsmcpars lllvatlvll dhlslarnlp
61 vatpdpgmfp clhhsqnllr avsnmlqkar qtlefypcts eeidheditk dktstveacl
121 pleltknesc Insretsfit ngsclasrkt sfmmalclss iyedlkmyqv efktmnakll
181 mdpkrqifld qnmlavidel mqalnfnset vpqkssleep dfyktkiklc illhafrira
241 vtidrvmsyl nas

SEQ ID NO: 201 IL-12α subunit isoform 1 mature peptide amino acid sequence
Rnlp vatpdpgmfp clhhsqnllr avsnmlqkar qtlefypcts eeidheditk dktstveacl
pleltknesc Insretsfit ngsclasrkt sfmmalclss iyedlkmyqv efktmnakll
mdpkrqifld qnmlavidel mqalnfnset vpqkssleep dfyktkiklc illhafrira
vtidrvmsyl nas

SEQ ID NO: 202 IL-12α subunit isoform 2 peptide amino acid sequence
1 mwppgsasqp ppspaaatgl hpaarpvslq crlsmcpars lllvatlvll dhlslarnlp
61 vatpdpgmfp clhhsqnllr avsnmlqkar qtlefypcts eeidheditk dktstveacl
121 pleltkngsc lasrktsfmm alclssiyed lkmyqvefkt mnakllmdpk rqifldqnml
181 avidelmqal nfnsetvpqk ssleepdfyk tkiklcillh afriravtid rvmsylnas

SEQ ID NO: 203 IL-12α subunit isoform 3 peptide amino acid sequence
1 mwppgsasqp ppspaaatgl hpaarpvslq crlsmcpars lllvatlvll dhlslarnlp
61 vatpdpgmfp clhhsqnllr avsnmlqkne sclnsretsf itngsclasr ktsfmmalcl
121 ssiyedlkmy qvefktmnak llmdpkrqif ldqnmlavid elmqalnfns etvpqkssle
181 epdfyktkik lcillhafri ravtidrvms ylnas

SEQ ID NO: 204 IL-12β subunit amino acid sequence
1 mchqqlvisw fslvflaspl vaiwelkkdv yvveldwypd apgemvvltc dtpeedgitw
61 tldqssevlg sgktltiqvk efgdagqytc hkggevlshs llllhkkedg iwstdilkdq
121 kepknktflr ceaknysgrf tcwwlttist dltfsvkssr gssdpqgvtc gaatlsaerv
181 rgdnkeyeys vecqedsacp aaeeslpiev mvdavhklky enytssffir diikpdppkn
241 lqlkplknsr qvevsweypd twstphsyfs ltfcvqvqgk skrekkdrvf tdktsatvic
301 rknasisvra qdryysssws ewasvpcs

SEQ ID NO: 205 IL-12β subunit mature peptide amino acid sequence
iwelkkdv yvveldwypd apgemvvltc dtpeedgitw
tldqssevlg sgktltiqvk efgdagqytc hkggevlshs llllhkkedg iwstdilkdq
kepknktflr ceaknysgrf tcwwlttist dltfsvkssr gssdpqgvtc gaatlsaerv
rgdnkeyeys vecqedsacp aaeeslpiev mvdavhklky enytssffir diikpdppkn
lqlkplknsr qvevsweypd twstphsyfs ltfcvqvqgk skrekkdrvf tdktsatvic
rknasisvra qdryysssws ewasvpcs

1. A method for inducing an immune response in an immune distinct subject, the method comprising administering to the subject one or more compositions comprising:
a) a first cytokine mRNA construct comprising a first open reading frame (ORF), wherein the first ORF encodes a proinflammatory cytokine; and
b) optionally, one or more of:
i) a first antigen mRNA construct comprising a second open reading frame (ORF), wherein the second ORF encodes an antigen; and
ii) an antigen polypeptide, antigen molecule, or killed or attenuated pathogenic agent.

2. (canceled)

3. (canceled)

4. The method of claim 1, wherein the immune response comprises at least one of:
an increase in IL-12 in the subject, an increase in Ig levels in the subject, a CD4+ T cell response in the subject, a CD8+ T cell response in the subject, a NK cell response in the subject, a Th1 response in the subject, phagocytosis via the Fc region of each IgG subclass via improved affinity for phagocyte membrane Fc-gamma-receptors (FcγR), or immunization of the subject against the antigen or an organism comprising the antigen.

5.-13. (canceled)

14. A method for treating or preventing a disease in an immune distinct subject, the method comprising administering to the subject one or more compositions comprising:
a) a first cytokine mRNA construct comprising a first open reading frame (ORF), wherein the first ORF encodes a proinflammatory cytokine; and
b) optionally, one or more of:
i) a first antigen mRNA construct comprising a second open reading frame (ORF), wherein the second ORF encodes an antigen; and
ii) an antigen polypeptide, antigen molecule, or killed or attenuated pathogenic agent.

15. A method for immunizing an immune distinct subject, the method comprising administering to the subject one or more compositions comprising:
a) a first cytokine mRNA construct comprising a first open reading frame (ORF), wherein the first ORF encodes a proinflammatory cytokine; and
b) optionally, one or more of:
i) a first antigen mRNA construct comprising a second open reading frame (ORF), wherein the second ORF encodes an antigen; and
ii) an antigen polypeptide, antigen molecule, or killed or attenuated pathogenic agent.

16. The method of claim 1,
wherein the immune distinct subject is a subject with immunosenescence.

17. The method of claim 1,
wherein the immune distinct subject and/or subject with immunosenescence is a subject of 55 years of age or older.

18.-21. (canceled)

22. The method of claim 1,
wherein the immune distinct subject is a subject who has or is determined to have a reduced TNF response to immune stimuli and/or is a subject who has or is determined to have a reduced IL-12 response to immune stimuli.

23.-25. (canceled)

26. The method of claim 1, wherein the immune distinct subject is 2 years of age or younger.

27.-29. (canceled)

30. The method of claim 1,
wherein the immune distinct subject is immunocompromised, has an HIV infection, has AIDS, has received a transplant, is undergoing immunosuppression, is immunosuppressed, has an infection, is diabetic, has an IgG subclass deficiency, has a substance abuse disorder, or is obese.

31.-32. (canceled)

33. The method of claim 1, wherein the subject is a subject who is:
a) at least 55 years of age; and
b) is at least one of the following: immunocompromised, infected with HIV, has AIDS, has received a transplant, is undergoing immunosuppression, is immunosuppressed, has an infection, is diabetic, has an IgG subclass deficiency, has a substance abuse disorder, is obese, and/or is living in a high density living environment.

34.-38. (canceled)

39. The method of claim 1, wherein the subject is administered a) a dose of the first cytokine mRNA construct and b) a dose of the first antigen mRNA construct and/or the antigen polypeptide, antigen molecule, or killed or attenuated pathogenic agent, wherein the dose of the first cytokine mRNA construct is no more than 20% of the dose of the first antigen mRNA construct and/or the antigen polypeptide, antigen molecule, or killed or attenuated pathogenic agent by weight; and/or
wherein the subject is a human subject and is administered a dose of the first cytokine mRNA construct of from 0.10 μg to 10 μg.

40.-64. (canceled)

65. The method of claim 1, wherein the proinflammatory cytokine is selected from the group consisting of: IL-12; IL-2; IL-4; IL-5; IL-6; IL-8; IL-10; IL-13; IL-27; IL-1 beta; TGFbeta; IFNy; IFNa; IFNI3; TNFa; CCL2; CCL3; CCL4; CCL5; CCL8; CXCL12; GM-CSF; and a subunit, dimer, heterodimer, derivative, fragment, agonist or homologue thereof.

66. The method of claim 1,
wherein the proinflammatory cytokine is IL-12 or a subunit, dimer, heterodimer, derivative, fragment, agonist or homologue thereof.

67. The method of claim 65, wherein the first ORF comprises a sequence at least 90% identical to SEQ ID NO: 59.

68. (canceled)

69. The method of claim 1,
wherein the one or more compositions further comprise one or more further cytokine mRNA constructs, each comprising a further open reading frame (ORF), wherein each further ORF encodes a proinflammatory cytokine distinct from the proinflammatory cytokine encoded by the first ORF; or
wherein the first cytokine mRNA construct further comprises one or more further open reading frames (ORFs), wherein each further ORF encodes a proinflammatory cytokine distinct from the proinflammatory cytokine encoded by the first ORF.

70. (canceled)

71. (canceled)

72. (canceled)

73. The method of claim 69, wherein the first ORF encodes IL-12 or a subunit, derivative, fragment, agonist or homologue thereof and the one or more further ORFs encode IL-2; IL-4; IL-5; IL-6; IL-8; IL-10; IL-13; IL-27; IL-1β; TGFβ; IFNγ; IFNα; IFNβ; TNFα; CCL2; CCL3; CCL4; CCL5; CCL8; CXCL12; GM-CSF; or a subunit, derivative, fragment, agonist or homologue thereof.

74. The method of claim 1,
wherein the composition further comprises one or more further antigen mRNA constructs, each comprising a further open reading frame (ORF), wherein each further ORF encodes an antigen distinct from the antigen encoded by the second ORF; and/or
wherein the antigen mRNA construct further comprises one or more further open reading frames (ORFs), wherein each further ORF encodes an antigen distinct from the antigen encoded by the second ORF.

75.-82. (canceled)

83. The method of claim 1,
wherein the antigen is a pathogenic microbial protein or an epitope containing fragment thereof.

84.-101. (canceled)

102. The method of claim 1,
wherein one or more of the first, second, or further ORFs is operatively linked to at least one untranslated region (UTR),
wherein each UTR comprises at least a first organ protection sequence (OPS), wherein each OPS comprises at least two micro-RNA (miRNA) target sequences, and wherein each of the at least two miRNA target sequences are optimised to hybridise with a corresponding miRNA sequence.

103.-118. (canceled)

119. The method of claim 14, wherein the disease is caused by a coronavirus, an intracellular pathogen, a latent infection, an active infection, an influenza virus, respiratory syncytial virus (RSV), human immunodeficiency virus (HIV), varicella zoster virus (VZV), or Mycobacterium tuberculosis; and/or one or more of the antigens are a coronavirus, an intracellular pathogen, a latent infection, an active infection, an influenza virus, respiratory syncytial virus (RSV), human immunodeficiency virus (HIV), varicella zoster virus (VZV), plasmodium (Malaria), Streptococcus pneumoniae, Streptococcus pyogenes, Yersinia pestis, Haemophilus influenzae, Staphylococcus aureus, Pseudomonas aeruginosa, Bordetella pertussis, Ebola virus, Lassa virus, Middle East Respiratory Syndrome coronavirus, SARS-COV-1, SARS-COV-2, SARS-COV-2 variants of concerns, Marburg virus, Nipah virus, Rift Valley Fever virus, Chikungunya virus or Mycobacterium tuberculosis antigen.

120.-123. (canceled)

124. A combination comprising:
a) at least one dose of first cytokine mRNA construct comprising a first open reading frame (ORF), wherein the first ORF encodes a proinflammatory cytokine; and
b) at least one dose of one or more of:
i) a first antigen mRNA construct comprising a second open reading frame (ORF), wherein the second ORF encodes an antigen; and
ii) an antigen polypeptide, antigen molecule, or killed or attenuated pathogenic agent;
wherein:
each dose of the first cytokine mRNA construct is no more than 20% of each dose of the first antigen mRNA construct and/or the antigen polypeptide, antigen molecule, or killed or attenuated pathogenic agent by weight; and/or
each dose of the first cytokine mRNA construct of from 0.10 μg to 10 μg.

125.-246. (canceled)
